(19) United States
(12) Patent Application Publication
(10) Pub. No.: US 2012/0040930 A1
(43) Pub. Date: Feb. 16, 2012

(54) TETRACYCLIC TERPENE SERIES COMPOUNDS, METHODS FOR PREPARING SAME, USES THEREOF AS MEDICINES AND PHARMACEUTICAL COMPOUNDS CONTAINING SAME

(76) Inventors: Catherine Guillou, Gif S/Yvette (FR); Jean-Yves Lallemand, Palaiseau (FR); Thibault Sauvaítre, Hecourt (FR); Jordi Molgo, Antony (FR); Denyse Herlem, Versailles (FR); Daniel Guenard, Montrouge (FR); Françoise Khuong-Huu, Paris (FR)

(21) Appl. No.: 11/815,248

(22) PCT Filed: Jan. 13, 2006
(86) PCT No.: PCT/EP2006/050200

§ 371 Date: Aug. 1, 2007

(30) Foreign Application Priority Data
Feb. 2, 2005 (FR) 05/01029

(51) Int. Cl. A61K 31/58; A61K 31/573; C07J 63/00; A61P 25/16; A61K 31/695; A61P 25/28; A61P 25/00; C07J 53/00; C07J 71/00

(52) U.S. Cl. 514/63; 552/510; 514/180; 544/245; 514/257; 564/217; 514/629; 564/222; 540/50; 514/176; 556/419; 544/89; 514/229.5; 540/55

(57) ABSTRACT

The invention concerns a triterpene alkaloid of general formula (I). The invention also concerns a method for making same and use thereof as medicine.

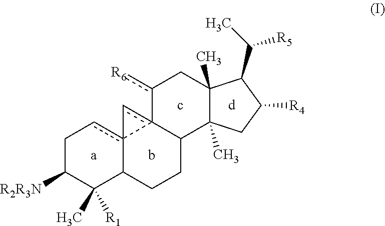

The present invention relates to a triterpenic alkaloid of following general formula I:

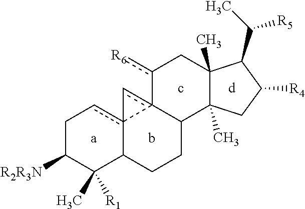

The present invention also relates to a method for manufacturing said triterpenic alkaloid and the use of same as a medicament.

In the past century, life expectancy has increased by 25 years. This aging of the population has been accompanied by an increase in age-related diseases, at the forefront of which is Alzheimer's disease. This disease affects 22 million people worldwide and represents more than 75% of dementia in the elderly. It affects 5% to 12% of those over 65 and 25% of those over 85. It is the fourth leading cause of death in the elderly. In France, 135,000 new cases arise each year and currently roughly 800,000 people are affected.

Alzheimer's disease is characterized by behavior and memory disorders. This disease, which primarily affects the elderly, is progressing rapidly in the industrialized world due to expanding life expectancies.

According to the cholinergic hypothesis, Alzheimer's-related memory disorders are associated with a loss of cholinergic functioning in the brain following a very large decrease in the neurotransmitter acetylcholine. Currently, symptomatic treatment of the disease consists of increasing the level of the neurotransmitter acetylcholine by inhibiting acetylcholinesterase (AChE), the enzyme responsible for acetylcholine degradation.

Among the four medicaments sold for the treatment of Alzheimer's disease, three are acetylcholinesterase inhibitors: Aricept® (donepezil, sold by Eisai), Exelon® (rivastigmine, sold by Novartis), Reminyl® (galanthamine, sold by Jansen). In 2004, Ebixa® (memantine), a gamma-aminobutyric acid (GABA) receptor antagonist that protects neurons against an elevated, prolonged calcium influx, was put on the market by Lundbeck.

Certain of the acetylcholinesterase inhibitors on the market are difficult to prepare and have side effects. In addition, no butyrylcholinesterase (BChE) inhibitor is currently on the market.

AChE, in addition to its catalytic functions, has unconventional properties.

Studies have shown that the acetylcholinesterase peripheral site is involved in the aggregation of β-amyloid protein, a protein that is highly neurotoxic in the case of Alzheimer's disease. Indeed, if molecules are able to interact with the AChE peripheral site, the aggregation of β-amyloid protein is significantly reduced. The synthesis of inhibitors that can interact simultaneously with active and peripheral AChE sites is thus of unquestionable interest.

Additionally, very recent data have shown that butyrylcholinesterase also plays an important role in the case of Alzheimer's disease by supporting β-amyloid protein aggregation. The use of BChE inhibitors may prevent this phenomenon.

Balearic box is a plant that is rich in steroidic alkaloids of tetracyclic triterpenic type. During a systematic screening of the combinatorial library at the Institut de Chimie des Substances Naturelles (ICSN, Gif sur Yvette, France), researchers identified a powerful acetylcholinesterase inhibitor (1). Compound 1 arises from the chemical conversion of an alkaloid, N-3-isobutyrylcycloxobuxidine-F (2), isolated from Balearic box (Buxus balearica Wild) according to a novel protocol (see examples 1 and 2).

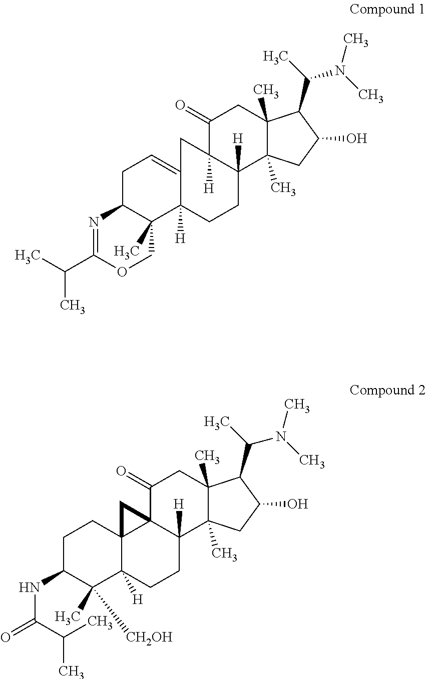

N-3-isobutyrylcycloxobuxidine-F (2) (D. Herlem-Gaulier and al, Bull. Soc. Chim. De France, 1966, 11, 3478-3486) was extracted from the leaves of Buxus balearica Wild during the 1960's by successive chromatographies on deactivated alumina with an elution gradient or by separation of the weak and strong bases of the crude alkaloids. In the latter case, tedious countercurrent separation was necessary to separate compound 2 from N-3-benzoylcycloxobuxidine-F (6b).

The general formula of N-3-isobutyrylcycloxobuxidine-F (2) as disclosed in the document by D. Herlem-Gaulier et al. (1966) contains an erroneous isomerism on carbon 4. Articles by J. Guilhem et al. (Tetrahedron Letters, 34, 2937-2938, 1975) and M. Sangare et al. (Tetrahedron Letters, 22 and 23, 1791-1794, 1975) describe the general formula of compound 2 in which the carbon 4 isomerism is corrected.

In addition, a ¹³C NMR study (125 MHz, CDCl₃; HMBC HMQC) of compound 2 made it possible to correct the chemical shift of carbons 1, 4, 5, 7, 17, 19 and 20 that were erroneous in the Sangare et al. publication.

Within the framework of the present invention, compound 2 has been isolated according to a new protocol that avoids the use of chromatographies (very labor-intensive with large quantities) as well as the countercurrent method mentioned above.

The method of the present invention makes it possible to envisage advantageous prospects for industrial scale-up.

In addition, synthesis of compound 1 has been described by pyrolysis of compound 2 in the presence of tetramethylammonium hydroxide at 210° C. under 0.01 mmHg (D. Herlem-Gaulier et al., Bull. Soc. Chimique de France, 1968, 2, 763-773; also see articles by J. Guilhem et al. (1975) and M. Sangare et al. (1975) for the correct carbon 4 isomerism).

Lastly, the inventors have discovered, in a surprising way, a new, simpler method for synthesizing compound 1 from compound 2 using a single step. Other compounds with AChE inhibiting activity have been prepared in a novel manner from alkaloid precursor 2, highly abundant in nature, isolated from Balearic box leaves.

Some of these compounds are already known but not for their therapeutic application.

Thus, the article by F. Khuong-Huu et al. (Bull. Soc. Chim. De France, 1, 258-262, 1969) described the compounds of the following formulas:

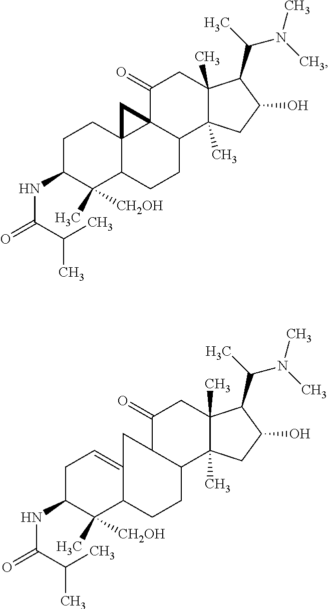

Similarly, the article by D. Herlem-Gaulier et al. (Bull. Soc. Chim. De France, 2, 763-773, 1968) described the compounds of the following formulas:

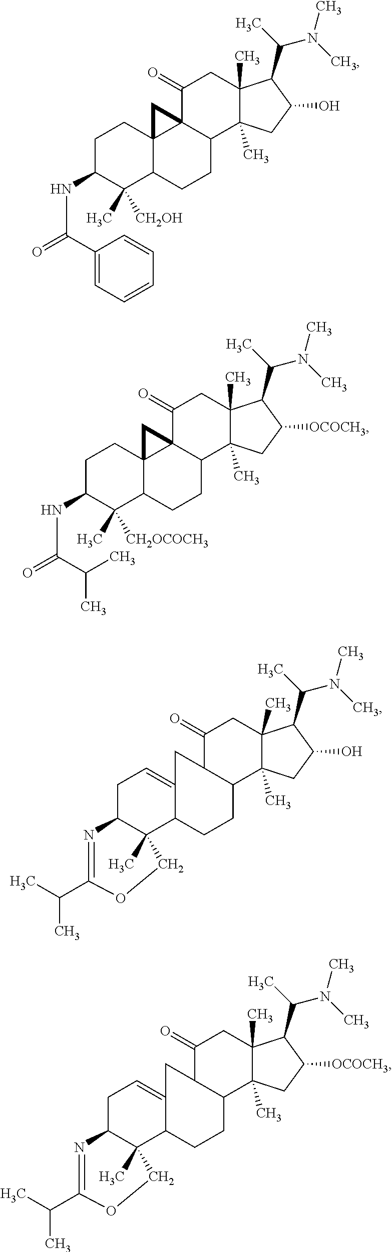

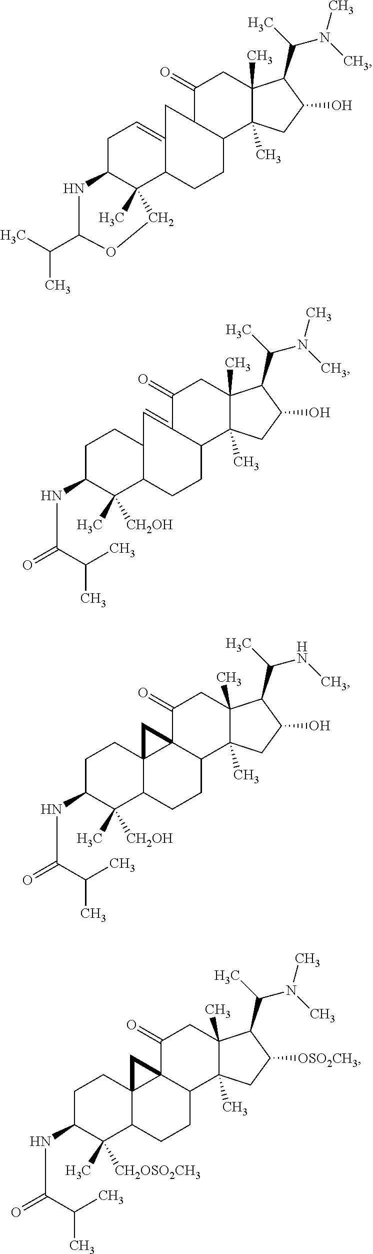

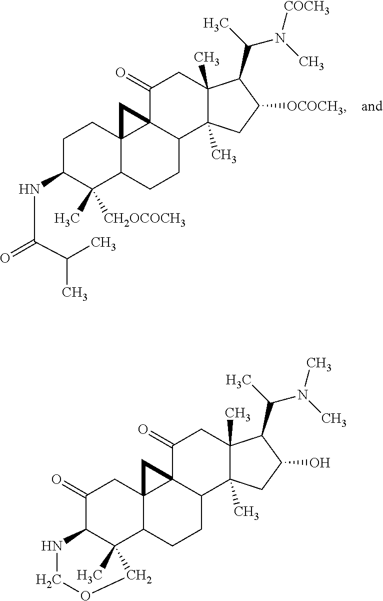

These compounds, as described in articles by F. Khuong-Huu et al. (1969) and D. Herlem-Gaulier et al. (1968), have an erroneous carbon 4 isomerism; articles by J. Guilhem et al. (1975) and M. Sangare et al. (1975) describe the correct isomerism.

The document by Benechie et al., Tetrahedron., 1976, 32:701-707 described in particular following compounds 4b and 5b:

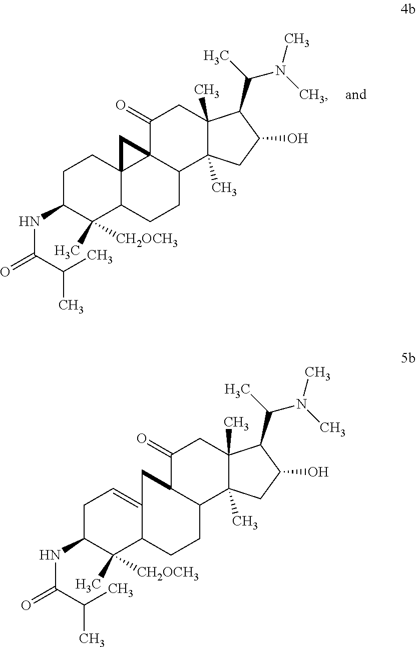

The inventors thus have discovered that the triterpenic alkaloids according to the present invention act as powerful inhibitors of acetylcholinesterase and butyrylcholinesterase and therefore can be used in the treatment of Alzheimer's disease and other diseases of the central nervous system as well as diseases of the peripheral nervous system. In addition, these alkaloids can be easily obtained on an industrial scale.

Thus, according to a first aspect, one object of the invention is a triterpenic alkaloid of following general formula I:

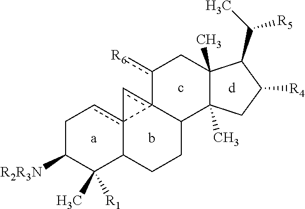

wherein
one and only one of the three dotted lines present in rings a and b represents a bond;
R₁ represents —CH₂X₁R₇, —CH═NR₈ or —CH═O wherein
X₁ represents O, NH or S;
R₇ represents a hydrogen atom, a C₁-C₁₀ alkyl, an alkyl (C₁-C₁₀) phenyl, an alkyl (C₁-C₁₀) carbonyl, an alkyl (C₁-C₁₀)-bis[trialkyl (C₁-C₁₀) silyl] or SO₂R₉, R₉ representing a C₁-C₁₀ alkyl or an alkyl (C₁-C₁₀) phenyl, provided that R₇ does not represent SO₂R₉ when X₁ represents S;
R₈ represents a hydrogen atom, a C₁-C₁₀ alkyl, an alkyl (C₁-C₁₀) phenyl, an alkyl (C₁-C₁₀) carbonyl, an alkyl (C₁-C₁₀)-bis[trialkyl (C₁-C₁₀) silyl] or SO₂R₉, R₉ representing a C₁-C₁₀ alkyl or an alkyl (C₁-C₁₀) phenyl;
   R₂ and R₃ represent independently of each other a hydrogen atom or

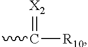

wherein X₂ represents O, NH or S and R₁₀ represents a hydrogen atom, a C₁-C₁₀ alkyl, a phenyl, a C₃-C₁₀ cycloalkyl, or an alkyl (C₁-C₁₀) phenyl, provided that at least one and only one of R₂ or R₃ represents a hydrogen atom,

or R₂ is absent, R₁ represents —CH₂X₁R₇, and —NR₃ and —X₁R₇ taken together represent

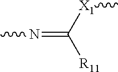

wherein R₁₁ represents a hydrogen atom, a C₁-C₁₀ alkyl, a phenyl, a C₃-C₁₀ cycloalkyl, an alkyl (C₁-C₁₀) phenyl,

or R₂ represents a hydrogen atom, R₁ represents —CH₂X₁R₇, and —NHR₃ and —X₁R₇ taken together represent

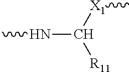

wherein R₁₁ represents a hydrogen atom, a C₁-C₁₀ alkyl, a phenyl, a C₃-C₁₀ cycloalkyl or an alkyl (C₁-C₁₀) phenyl,

or R₂ represents a hydrogen atom, R₁ represents —CH₂═NR₈, and —NHR₃ and ═NR₈ taken together represents

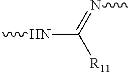

wherein R₁₁ represents a hydrogen atom, a C₁-C₁₀ alkyl, a phenyl, a C₃-C₁₀ cycloalkyl or an alkyl (C₁-C₁₀) phenyl;

R₄ represents a hydrogen atom, a C₁-C₁₀ alkyl or a —X₃R₁₂ radical wherein X₃ represents O, NH or S, advantageously O, and R₁₂ represents a hydrogen atom, an alkyl (C₁-C₁₀) carbonyl, an alkyl (C₁-C₁₀) phenyl, an alkyl (C₁-C₁₀) sulphonyl, an alkyl (C₁-C₁₀) phenyl sulphonyl or an alkyl (C₁-C₁₀)-dialkyl (C₁-C₁₀) silyl, advantageously a hydrogen atom;

R₅ represents the N-oxide group N⁺OCH₃R₁₃ or the group NCH₃R₁₃ wherein

R₁₃ represents a hydrogen atom, a C₃-C₁₀ cycloalkyl, a C₁-C₁₀ alkyl, a phenyl or a phthalimide, advantageously —CH3 and

[symbol] represents ═O or —OR₁₄ wherein R₁₄ represents a hydrogen atom, a C₁-C₁₀ alkyl, an alkyl (C₁-C₁₀) carbonyl, an alkyl (C₁-C₁₀) phenyl or an alkyl (C₁-C₁₀) sulphonyl;

or pharmaceutically acceptable addition salts, isomers, enantiomers or diastereoisomers thereof, as well as mixtures thereof,

except for compounds of the following formulas:

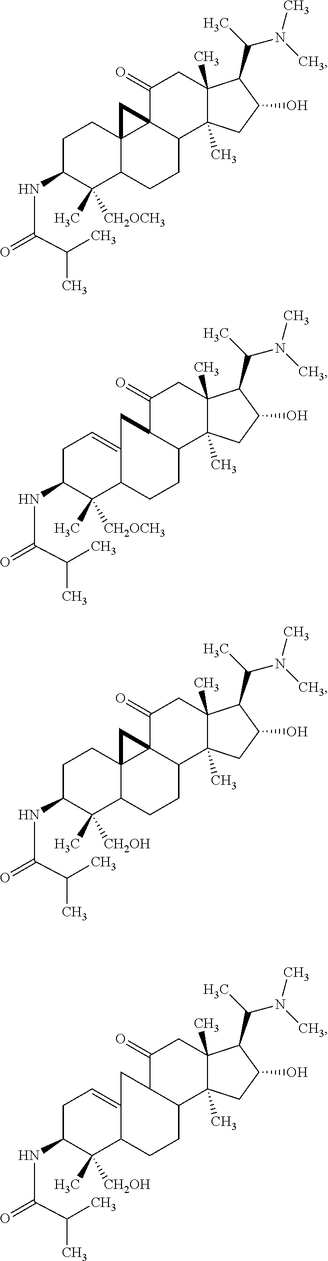

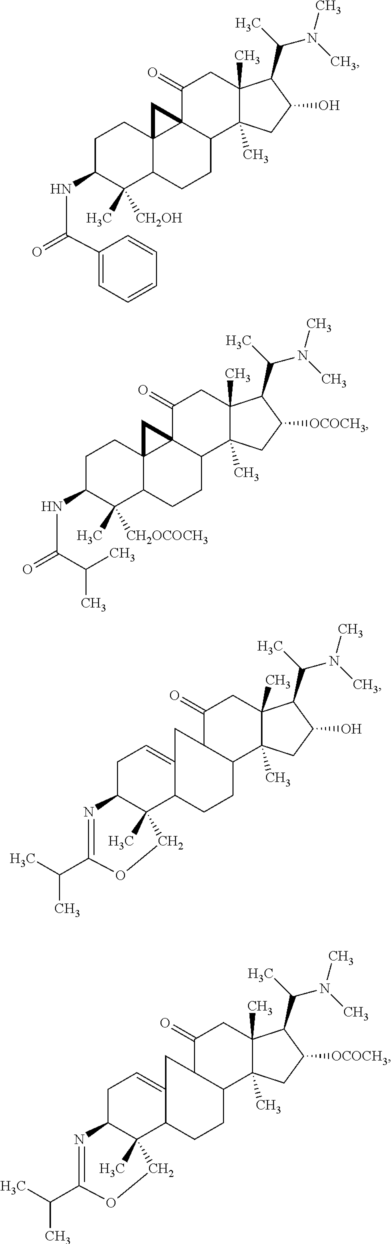

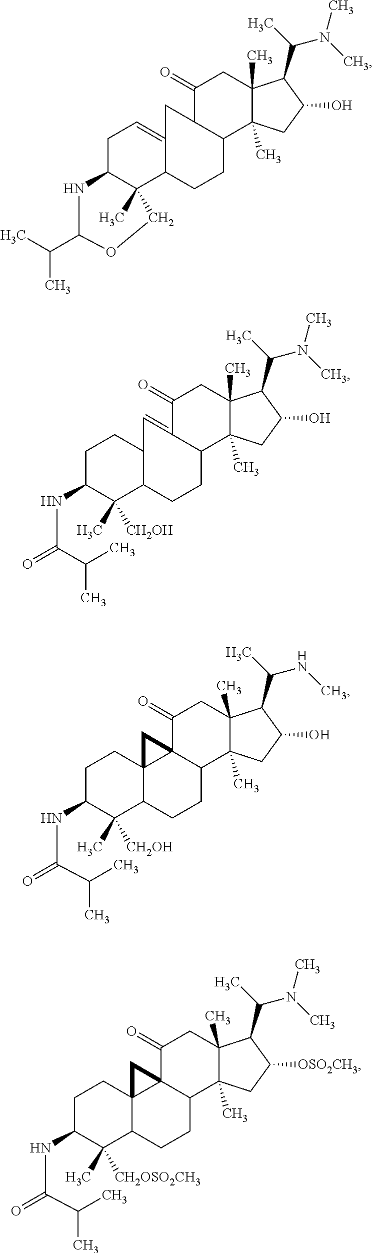

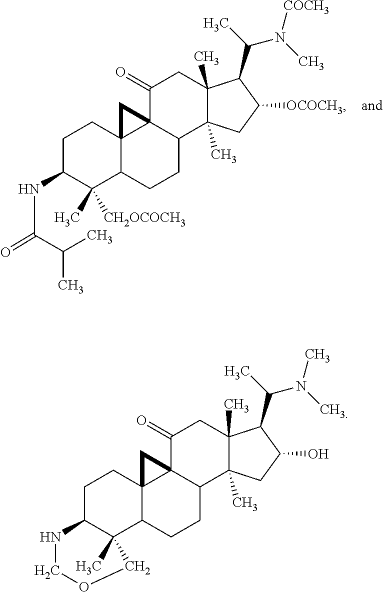

Within the framework of the present invention, “C₁-C₁₀ alkyl” means any linear or branched monovalent saturated hydrocarbon radical having from one to ten carbon atoms. Examples of alkyl radicals include, but are not limited to, methyl, ethyl, propyl, isopropyl, 1-ethylpropyl, sec-butyl, tert-butyl, N-butyl, n-pentyl, n-hexyl, etc.

“Alkyl (C₁-C₁₀) phenyl” means any R′R″ radical wherein R′ is a C₁-C₁₀ alkyl radical as defined herein, and R″ is a phenyl radical. Examples of alkyl (C₁-C₁₀) phenyl radicals include, but are not limited to, phenylethyl groups, 3-phenylpropyl groups, and the like.

“C₃-C₁₀ cycloalkyl” means any saturated monovalent carbocyclic radical consisting of one or more rings, preferably two rings, of three to 10 carbon atoms per ring. Examples of cycloalkyl radicals include, but are not limited to, cyclopropyl, cyclobutyl, 3-ethylcyclobutyl, cyclopentyl and cycloheptyl groups, and the like.

In the present invention, “alkyl (C₁-C₁₀) carbonyl” means any R—C(O)— radical wherein R is an alkyl radical as defined herein. Examples of alkyl (C₁-C₁₀) carbonyl radicals include, but are not limited to, acetyl, propionyl, n-butyryl, sec-butyryl, t-butyryl and iso-propionyl groups, and the like.

In the present invention, “alkyl (C₁-C₁₀) sulphonyl” means any —S(O)₂—R radical wherein R is an alkyl group as defined herein. Examples of alkylsulfonyls include, but are not limited to, methylsulfonyl groups, propylsulfonyl groups, and the like.

In the present invention, “isomers” means compounds that have identical molecular formulas but that differ by the arrangement of their atoms in space. Isomers that differ in the arrangement of their atoms in space are referred to as “stereoisomers.” Stereoisomers that are not mirror images of each other are referred to as “diastereoisomers,” and stereoisomers that are nonsuperimposable mirror images of each other are referred to as “enantiomers,” or sometimes “optical isomers.” A carbon atom bound to four nonidentical substituents is referred to as a “chiral center.”

A “chiral isomer” is a compound with a chiral center. It includes two enantiomer forms of opposite chirality and can exist as an individual enantiomer or as a mixture of enantiomers. A mixture containing equal quantities of individual enantiomer forms of opposite chirality is referred to as “racemic mixture.”

In the present invention, “pharmaceutically acceptable” means of use in the preparation of a pharmaceutical composition that is generally safe, nontoxic, not biologically or otherwise undesirable and that is acceptable for pharmaceutical use in animals and in humans.

“Pharmaceutically acceptable salts” of a compound means salts that are pharmaceutically acceptable, as defined herein, and that have the desired pharmacological activity of the parent compound. Such salts include:

(1) acid addition salts formed with inorganic acids such as hydrochloric acid, hydrobromic acid, sulfuric acid, nitric acid, phosphoric acid and the like; or formed with organic acids such as acetic acid, benzenesulfonic acid, benzoic acid, camphorsulfonic acid, citric acid, ethane-sulfonic acid, fumaric acid, glucoheptonic acid, gluconic acid, glutamic acid, glycolic acid, hydroxynaphthoic acid, 2-hydroxyethanesulfonic acid, lactic acid, maleic acid, malic acid, mandelic acid, methanesulfonic acid, muconic acid, 2-naphtalene-sulfonic acid, propionic acid, salicylic acid, succinic acid, dibenzoyl-L-tartaric acid, tartaric acid, p-toluenesulfonic acid, trimethylacetic acid, trifluoroacetic acid and the like; or

(2) salts formed when an acid proton present in the parent compound is either replaced by a metal ion, for example an alkaline metal ion, an alkaline-earth metal ion or an aluminum ion, or is coordinated with an organic or inorganic base. Acceptable organic bases include diethanolamine, ethanolamine, N-methylglucamine, triethanolamine, tromethamine and the like. Acceptable inorganic bases include aluminum hydroxide, calcium hydroxide, potassium hydroxide, sodium carbonate and sodium hydroxide.

Preferred pharmaceutically acceptable salts are salts formed from hydrochloric acid, trifluoroacetic acid, dibenzoyl-L-tartaric acid and phosphoric acid.

It should be understood that all references to pharmaceutically acceptable salts include solvent addition forms (solvates) or crystalline forms (polymorphs) as defined herein, of the same acid addition salt.

Advantageously, general formula I is such that the hydrogen atoms are arranged in the following manner:

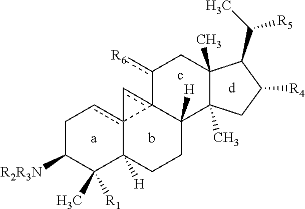

According to a specific embodiment, the triterpenic alkaloid according to the invention is such that R₁ represents —CH₂X₁R₇.

Preferably, the triterpenic alkaloid according to the invention is such that X₁ represents O.

According to a preferred embodiment, the triterpenic alkaloid according to the invention is represented by following general formula II:

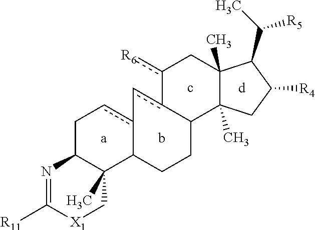

wherein one and only one of the two dotted lines present in rings a and b represents a bond;

R₄, R₅, R₁₁ and [symbol] are as defined above and X₁ is as defined above;

or the pharmaceutically acceptable addition salts, isomers, enantiomers or diastereoisomers thereof, as well as mixtures thereof.

Advantageously, general formula II is such that the hydrogen atoms are arranged in the following manner:

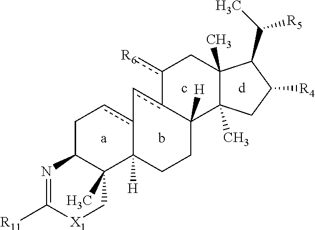

More preferably, the triterpenic alkaloid according to the invention is such that [symbol] represents ═O.

According to another preferred embodiment, the triterpenic alkaloid according to the invention is represented by following general formula III:

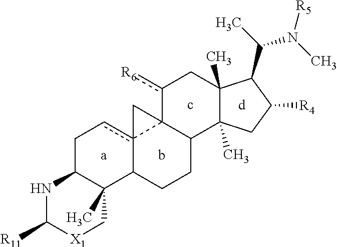

wherein one and only one of the two dotted lines present in rings a and b represents a bond; R₄, R₅ and R₁₁ are as defined above, X₁ is as defined above, and [symbol] is as defined above;

or pharmaceutically acceptable addition salts, isomers, enantiomers or diastereoisomers thereof, as well as mixtures thereof.

Advantageously, general formula III is such that the hydrogen atoms are arranged in the following manner:

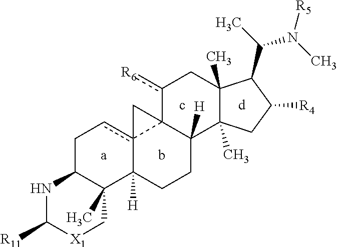

According to another embodiment, the triterpenic alkaloid is represented by following general formula VIII:

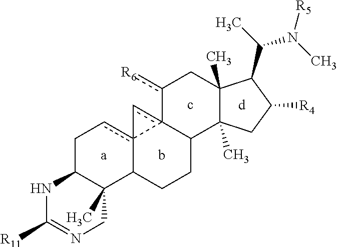

wherein one and only one of the three dotted lines present in rings a and b represents a bond; R₄, R₅ and R₁₁ are as defined previously, and [symbol] is as defined previously;

or the pharmaceutically acceptable addition salts, isomers, enantiomers and diastereoisomers thereof, as well as mixtures thereof.

According to another preferred embodiment, the triterpenic alkaloid according to the invention is represented by following general formula IV:

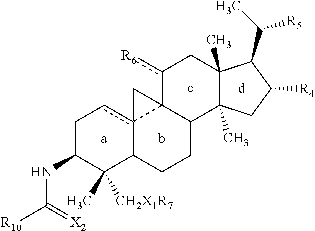

wherein one and only one of the two dotted lines present in rings a and b represents a bond, R₄, R₅, R₇, X₂ and R₁₀ are as defined above, X₁ is as defined above, and [symbol] is as defined above;

or the pharmaceutically acceptable addition salts, isomers, enantiomers and diastereoisomers thereof, as well as mixtures thereof.

Advantageously, general formula III is such that the hydrogen atoms are arranged in the following manner:

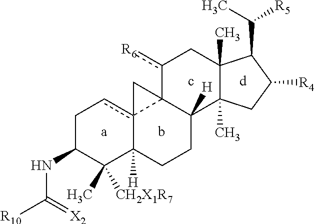

More preferably, the triterpenic alkaloid according to the invention is such that X₂ represents O.

Even more preferably, the triterpenic alkaloid according to the invention is such that R₁₀ or R₁₁ represents independently of the other a C₁-C₁₀ alkyl, advantageously branched.

Most preferably, the triterpenic alkaloid according to the invention is selected among one of the compounds of the following formulas:

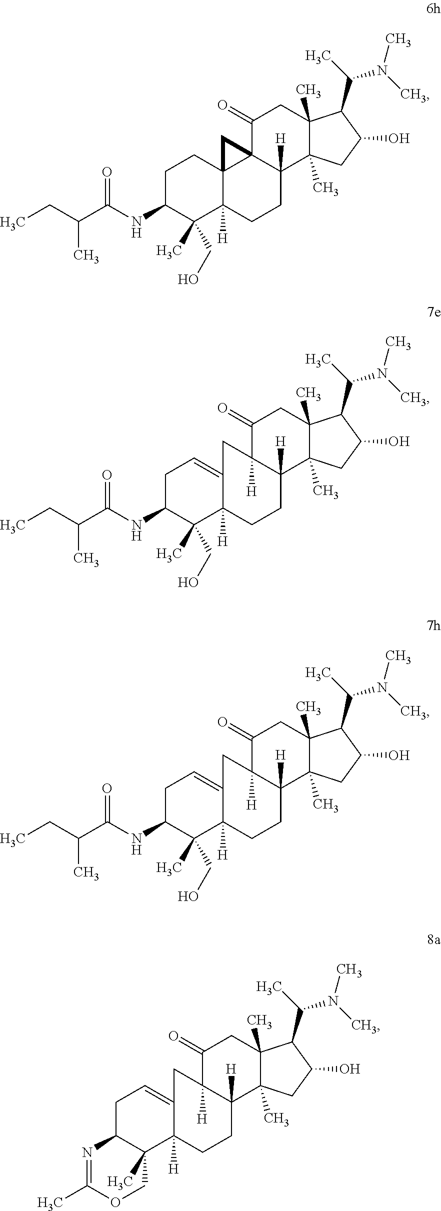

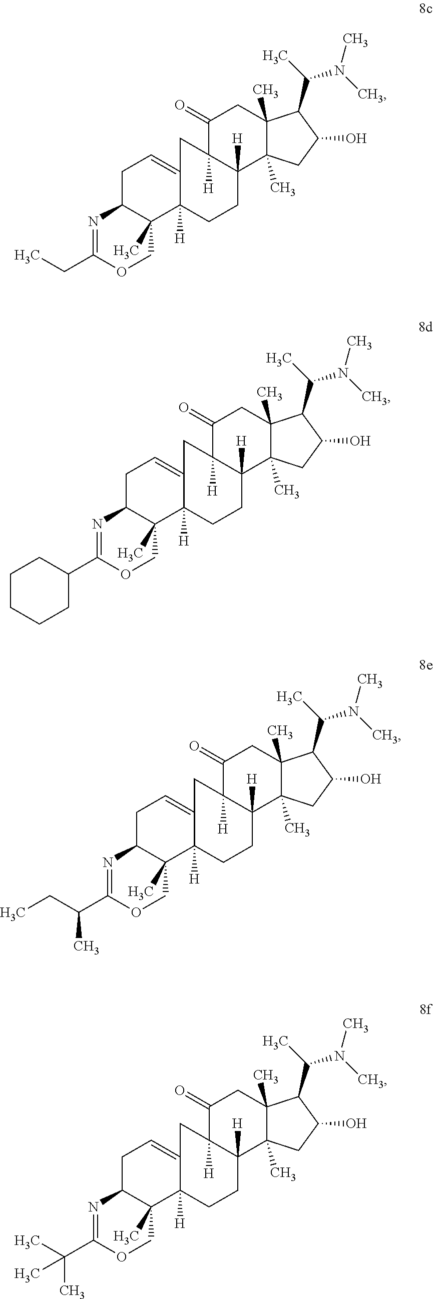

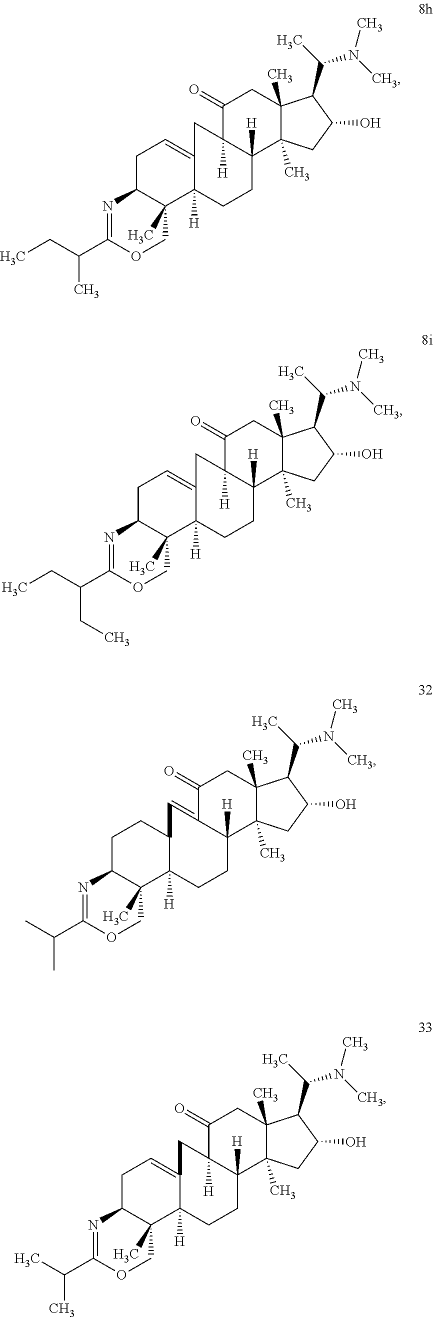

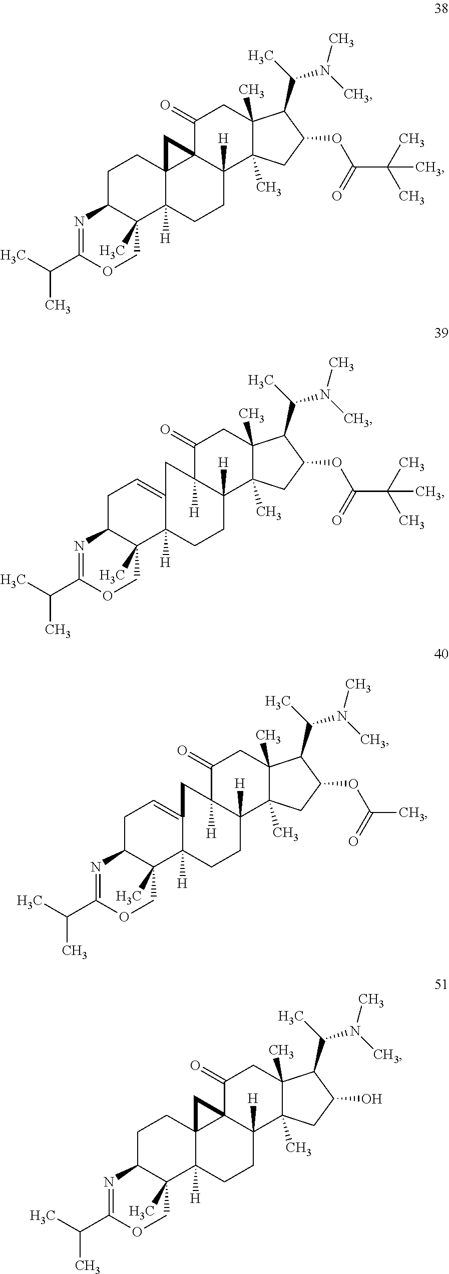

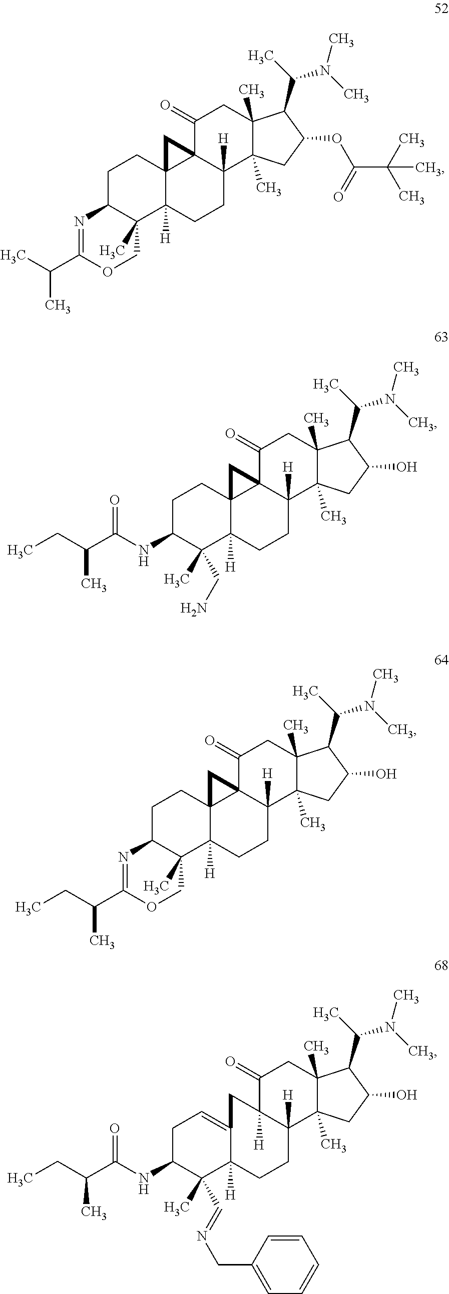

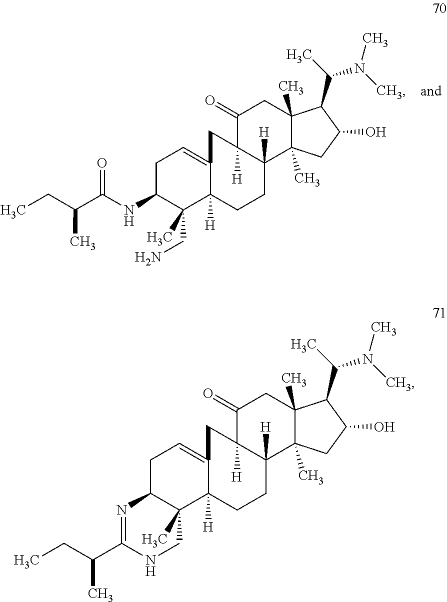

According to a second aspect, one object of the invention is a method for manufacturing the compound of following general formula IVb:

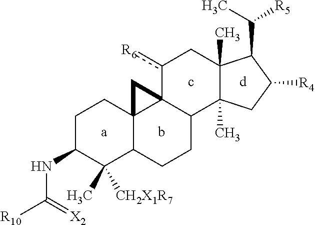

wherein R₄, R₅, R₇, R₁₀, X₁ and [symbol] are as defined above and X₂ represents O, by reaction of the compound of following formula Vb:

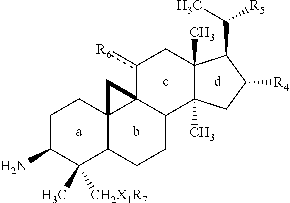

wherein R₄, R₅, R₇, X₁ and [symbol] are as defined above, with the anhydride of formula R₁₀(CO)₂O, wherein R₁₀ is as defined above, advantageously in methanol.

Advantageously, general formula IVb is such that the hydrogen atoms are arranged in the following manner:

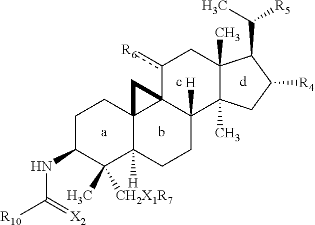

Advantageously, general formula Vb is such that the hydrogen atoms are arranged in the following manner:

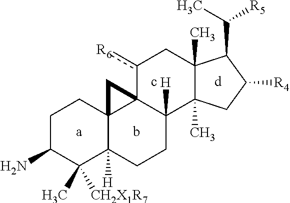

According to one specific embodiment, the compound of formula Vb is obtained by acid hydrolysis, advantageously in MeOH with H₂SO₄, of the compound of following formula VI:

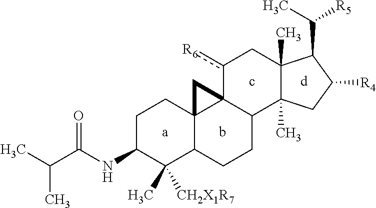

wherein R₄, R₅, R₇, X₁ and [symbol] are as defined above.

According to one specific embodiment, the method for manufacturing the compound of general formula IVb according to the invention is such that the compound of general formula VI has the following formula (2):

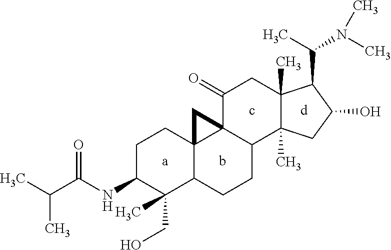

Advantageously, general formula VI is such that the hydrogen atoms are arranged in the following manner:

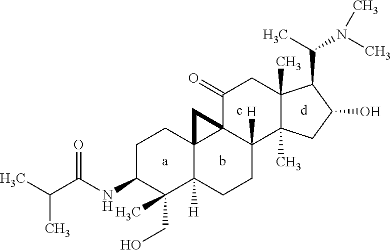

According to a third aspect, one object of the invention is a method for manufacturing the compound of formula (2) comprising the following successive steps:

a) alkalization of the ground, dried leaves of Buxus balearica Wild,

b) extraction and maceration in a CH₂Cl₂/EtOH mixture to obtain a solid extract,

c) extraction in dichloromethane with a pH between 5.8 and 6 to obtain a crude extract of the compound of formula (2),

d) purification of the compound of formula (2), advantageously by recrystallization in acetone.

Thus, according to the novel extraction protocol, the crude alkaloids are fractioned by extraction at different pH in buffered media. Compound 2 is separated from the other alkaloids by extraction at pH=5.8-6.0. Thus from 11 kg of ground, dried leaves of Buxus balearica Wild, 140 g of crude N-3-isobutyrylcycloxobuxidine-F (2) were recovered, and 100 g of pure compound 2 was isolated after recrystallization in acetone. The yield of the extraction was very good (0.9%).

According to a fourth aspect, one object of the invention is a method for manufacturing compounds of following general formula IIa:

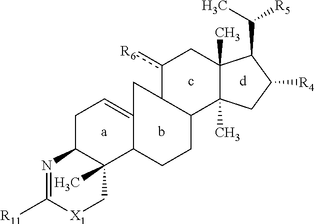

wherein R₄, R₅, R₁₁, X₁ and [symbol] are as defined above, comprising the following successive steps:

a) pyrolysis of the compound of formula IVb, wherein R₁₀=R₁₁ and R₄, R₅, R₁₁, X₁ and [symbol] are as defined above, and R₇ and X₂ are as defined above, at a pressure between 10⁻³ mmHg and 10⁻¹ mmHg, advantageously at a pressure of 0.03 mmHg, at a temperature between 230° C. and 350° C., advantageously between 235° C. and 270° C. for 3-24 hours, advantageously for 3 hours.

b) thermolysis of the mixture obtained in step a), at a pressure between 10⁻³ mmHg and 10⁻¹ mmHg, advantageously at a pressure of 0.03 mmHg, at a temperature between 230° C. and 350° C., advantageously between 235° C. and 270° C., and in the presence of tetraalkylammonium hydroxide (alkyl=methyl, ethyl, or butyl), advantageously tetraethylammonium hydroxide.

Advantageously, general formula IIa is such that the hydrogen atoms are arranged in the following manner:

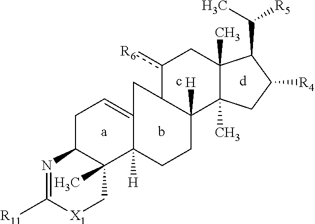

According to a fifth aspect, one object of the invention is a method for manufacturing the compound of general formula IIa, wherein R₅ represents —CH₃, R₄ represents —OH, [symbol] represents ═O, X₁ represents O, R₁₁ represents —CH(CH₃)₂, comprising the following successive steps:

a) pyrolysis of the compound of formula (2) at a pressure between 10⁻³ mmHg and 10⁻¹ mmHg, advantageously 0.03 mmHg, at a temperature between 200° C. and 350° C., advantageously 240° C.,

b) thermolysis of the mixture obtained in step a), at a pressure between 10⁻³ mmHg and 10⁻¹ mmHg, advantageously 0.05 mmHg, at a temperature between 200° C. and 350° C., advantageously 240° C., in the presence of tetraalkylammonium hydroxide (alkyl=methyl, ethyl, butyl), advantageously tetraethylammonium hydroxide.

Thus, N-3-isobutyrylcycloxobuxidine-F (1) was synthesized in two steps from N-3-isobutyrylcycloxobuxidine-F (2). Thermolysis at 240° C. under 0.05 mmHg of compound 2 yielded a mixture (82/18) of N-3-isobutyrylcycloxobuxidine-F (1) and compound 3 resulting from the opening of the cyclopropane. This mixture is then transformed into compound 1 with a yield of 74% by heating at 240° C. under 0.05 mmHg in the presence of five equivalents of tetraalkylammonium hydroxide (alkyl=methyl, ethyl, butyl), advantageously tetraethylammonium hydroxide.

Alumina chromatography of the mixture of 1 and 3 makes it possible to transform said mixture into 3 only with a yield of 91%.

According to a sixth aspect, one object of the invention is a method for manufacturing the compound of general formula IIa, wherein R₅ represents —CH₃, R₄ represents —OH, [symbol] represents ═O, X₁ represents O and R₁₁ represents —CH(CH₃)₂, by heating at a temperature between 200° C. and 350° C., advantageously 210° C., the compound of formula 2 in ethylene glycol in the presence of NaOH.

Thus, N-3-isobutyrylcycloxobuxidine-F (1) can be obtained in a single step with a yield of 83% by heating compound 2 in ethylene glycol at 210° C. in the presence of soda.

According to a seventh aspect, one object of the invention is a method for manufacturing compounds of following general formula IVa:

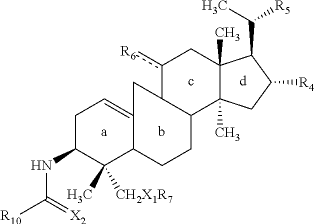

wherein R₄, R₅, R₇, R₁₀, X₁, X₂ and [symbol] are as defined above, comprising the following successive steps:

a) pyrolysis of the compound of formula IVb, wherein R₄, R₅, R₇, R₁₀, X₁, X₂ and [symbol] are as defined above, advantageously at a pressure between 10⁻³ mmHg and 10⁻¹ mmHg, advantageously 0.03 mmHg, at a temperature between 200° C. and 350° C., advantageously between 235° C. and 270° C. for 3-24 hours, advantageously for 3 hours.

b) alumina chromatography, or fractional crystallization, of the mixture obtained in step a).

Advantageously, general formula IVa is such that the hydrogen atoms are arranged in the following manner:

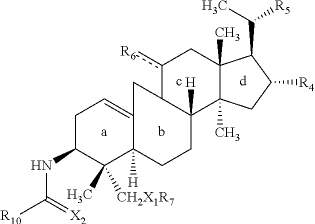

According to an eighth aspect, one object of the invention is a method for manufacturing compounds of following general formula IIIa:

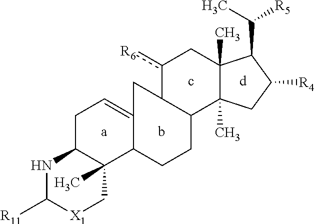

wherein R₄, R₅, R₁₁, X₁ and [symbol] are as defined above, comprising the following successive steps:

a) acid hydrolysis in methanol, advantageously with H₂SO₄ at 90° C. for 2 hours, then, after evaporation of the methanol at room temperature, at 80° C. for 1 hour, of the compound of general formula IVa, wherein R₄, R₅, X₁ and [symbol] are as defined above and R₇, R₁₀ and X₂ are as defined above, to obtain the compound of following formula Va:

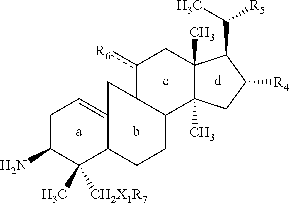

wherein R₄, R₅, X₁ and [symbol] are as defined above and R₇ is as defined above;

b) condensation, advantageously at 50° C., of the compound of formula Va with the compound of formula R₁₅CHO, wherein R₁₅CH represents R₁₁ and R₁₁ is as defined above.

Advantageously, general formula IIIa is such that the atoms of hydrogen and the R₁₁ radical are arranged in the following manner:

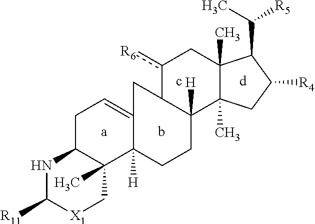

Advantageously, general formula Va is such that the hydrogen atoms are arranged in the following manner:

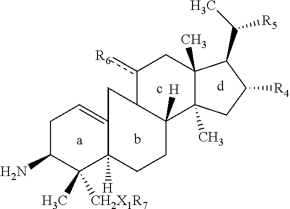

According to a ninth aspect, one object of the invention is a method for manufacturing the compound of following general formula IIIb:

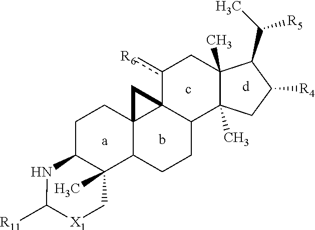

wherein R₄, R₅, R₁₁, X₁ and [symbol] are as defined above, by condensation, advantageously at 50° C., of the compound of formula Vb, wherein R₄, R₅, X₁ and [symbol] are as defined above, and R₇ is as defined above, with the compound of formula R₁₅CHO, wherein R₁₅CH represents R₁₁ and R₁₁ is as defined above.

Advantageously, general formula IIIb is such that the atoms of hydrogen and the R₁₁ radical are arranged in the following manner:

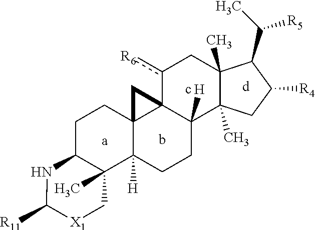

According to a tenth aspect, one object of the invention is a method for manufacturing the compound of following formula IIb:

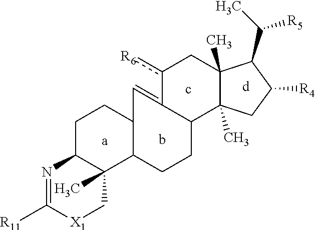

wherein R₄, R₅, R₁₁, X₁ and [symbol] are as defined above, by heating at a temperature between 200° C. and 350° C., advantageously at 300° C., in the presence of tetraethylammonium hydroxide, of the compound of following formula VII:

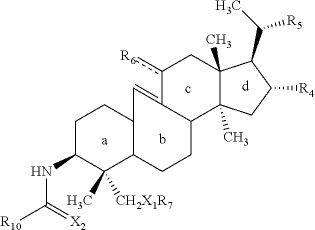

wherein R₁₀=R₁₁ and R₄, R₅, R₁₁, X₁ and [symbol] are as defined above and R₇ and X₂ are as defined above.

Advantageously, general formula IIb is such that the hydrogen atoms are arranged in the following manner:

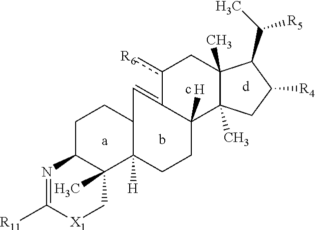

Advantageously, general formula VII is such that the hydrogen atoms are arranged in the following manner:

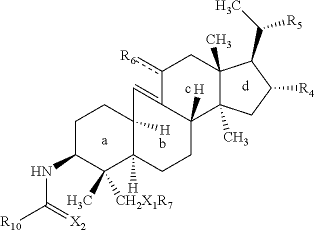

According to one specific embodiment, the compound of formula VII is obtained by reaction in the presence of a Lewis acid, advantageously TiCl₄ in dichloromethane, of the compound of formula IVb, wherein R₁₀=R₁₁, R₄, R₅, R₁₁, X₁ and [symbol] are as defined above and R₇ and X₂ are as defined above.

According to an eleventh aspect, one object of the invention is a method for manufacturing the compound of following general formula IVb:

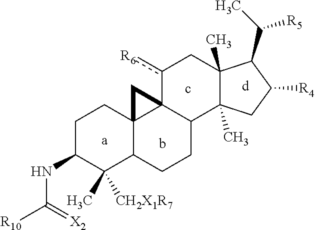

wherein R₅, R₁₀ and [symbol] are as defined above, R₄ represents —OR₁₂, wherein R₁₂ is as defined above, X₁ represents NH, R₇ represents H and X₂ represents O, comprising the following successive steps:
reaction of the compound of following general formula IX:

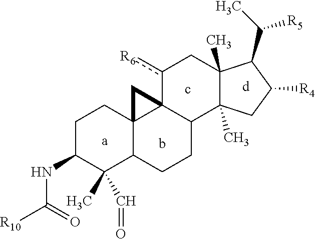

wherein R₅, R₁₀, R₁₂ and [symbol] are as defined in general formula IVb above, with benzylamine in the presence of anhydrous magnesium sulfate in dichloromethane to obtain the compound of following formula X:

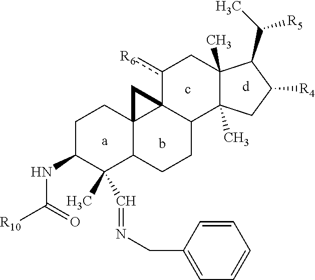

wherein R₅, R₁₀, R₁₂ and [symbol] are as defined in general formula IX above;
reaction of the compound of formula X with sodium cyanoborohydride and glacial acetic acid in methanol to obtain a compound of following formula XI:

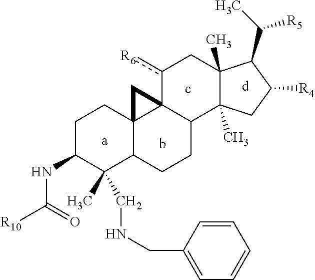

wherein R₅, R₁₀, R₁₂ and [symbol] are as defined in general formula X above;
reaction of the compound of formula XI, in the presence of 30% Pd/C catalyst in methanol, with ammonium formate or under hydrogen pressure with glacial acetic acid at pH 3 followed by alkalization with an ammonia solution to obtain the compound of formula IVb.

Preferably, the compound of formula IX, wherein R₁₂ does not represent a hydrogen atom, is obtained by the method comprising the following successive steps:
protection of the hydroxyl group of the compound of following general formula IVb:

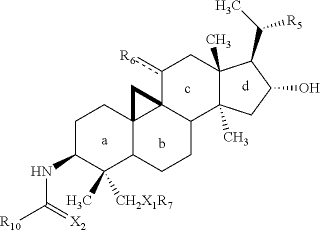

wherein R₅, R₁₀, and [symbol] are as defined above, R₇ represents H, and X₁ and X₂ represent O, by reaction with the compound of formula (R₁₂)₂O, wherein R₁₂ is as defined above but does not represent a hydrogen atom, in a pyridine/dichloromethane mixture to obtain the compound of following general formula IVb:

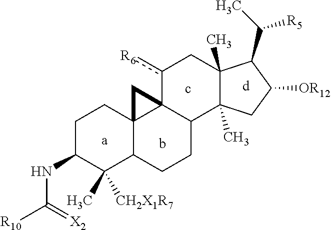

wherein R₅, R₁₀, and [symbol] are as defined above, R₇ represents H, R₁₂ is as defined above but does not represent a hydrogen atom, and X₁ and X₂ represent O;
reaction of the compound of formula IVb as defined above with the Dess-Martin periodinane in dichloromethane to obtain the compound of formula IX, wherein R₁₂ does not represent a hydrogen atom.

More preferably, the compound of formula IX, wherein R₁₂ represents a hydrogen atom, is obtained by the deprotection reaction of the compound of formula IX, wherein R₁₂ does not represent a hydrogen atom.

According to a twelfth aspect, one object of the invention is a method for manufacturing the compound of following formula IIc:

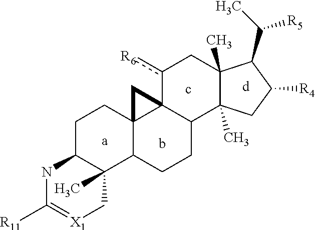

wherein R₅, R₁₁, and [symbol] are as defined above, X₁ represents NH, R₄ represents —OR₁₂ and R₁₂ is as defined above, by reaction of the compound of following general formula IVb:

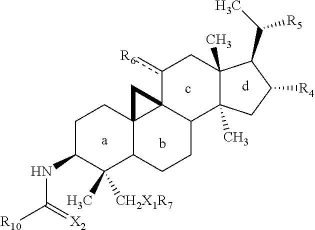

wherein R₁₀=R₁₁, R₅ and [symbol] are as defined above, R₄ represents —OR₁₂, wherein R₁₂ is as defined above, X₁ represents NH, R₇ represents H and X₂ represents O, with triethylamine in butanol.

According to a thirteenth aspect, one object of the invention is a method for manufacturing a compound of following formula VIII:

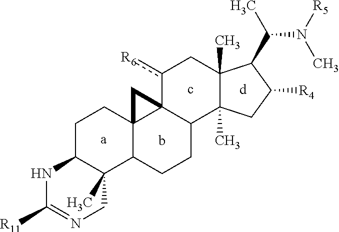

wherein R₅, R₁₁ and [symbol] are as defined above, R₄ represents —OR₁₂, and R₁₂ is as defined above, by reaction of the compound of following general formula IVb:

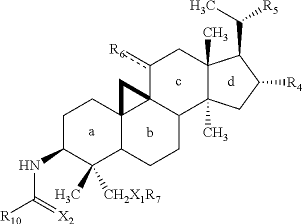

wherein R₁₀=R₁₁, R₅ and [symbol] are as defined above, R₄ represents —OR₁₂, wherein R₁₂ is as defined above, X₁ represents NH, R₇ represents H and X₂ represents O, with ammonium formate in the presence of 30% Pd/C catalyst in methanol.

According to a fourteenth aspect, one object of the invention is a triterpenic alkaloid of following general formula I:

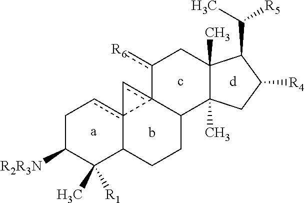

wherein
one and only one of the three dotted lines present in rings a and b represents a bond;
R₁ represents —CH₂X₁R₇, —CH═NR₈ or —CH═O wherein
X₁ represents O, NH or S;
R₇ represents a hydrogen atom, a C₁-C₁₀ alkyl, an alkyl (C₁-C₁₀) phenyl, an alkyl (C₁-C₁₀) carbonyl, an alkyl (C₁-C₁₀)-bis[trialkyl (C₁-C₁₀) silyl] or SO₂R₉, R₉ representing a C₁-C₁₀ alkyl or an alkyl (C₁-C₁₀) phenyl, provided that R₇ does not represent SO₂R₉ when X₁ represents S;
R₈ represents a hydrogen atom, a C₁-C₁₀ alkyl, an alkyl (C₁-C₁₀) phenyl, an alkyl (C₁-C₁₀) carbonyl, an alkyl (C₁-C₁₀)-bis[trialkyl (C₁-C₁₀) silyl] or SO₂R₉, R₉ representing a C₁-C₁₀ alkyl or an alkyl (C₁-C₁₀) phenyl;
   R₂ and R₃ represent independently of each other a hydrogen atom or

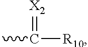

wherein X₂ represents O, NH or S and R₁₀ represents a hydrogen atom, a C₁-C₁₀ alkyl, a phenyl, a C₃-C₁₀ cycloalkyl, or an alkyl (C₁-C₁₀) phenyl, provided that at least one and only one of R₂ or R₃ represents a hydrogen atom,

or R₂ is absent, R₁ represents —CH₂X₁R₇, and —NR₃ and —X₁R₇ taken together represent

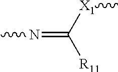

wherein R₁₁ represents a hydrogen atom, a C₁-C₁₀ alkyl, a phenyl, a C₃-C₁₀ cycloalkyl, an alkyl (C₁-C₁₀) phenyl,

or R₂ represents a hydrogen atom, R₁ represents —CH₂X₁R₇, and —NHR₃ and —X₁R₇ taken together represent

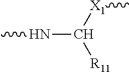

wherein R₁₁ represents a hydrogen atom, a C₁-C₁₀ alkyl, a phenyl, a C₃-C₁₀ cycloalkyl or an alkyl (C₁-C₁₀) phenyl,

or R₂ represents a hydrogen atom, R₁ represents —CH₂═NR₈, and —NHR₃ and ═NR₈ taken together represents

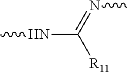

wherein R₁₁ represents a hydrogen atom, a C₁-C₁₀ alkyl, a phenyl, a C₃-C₁₀ cycloalkyl or an alkyl (C₁-C₁₀) phenyl;

R₄ represents a hydrogen atom, a C₁-C₁₀ alkyl or a —X₃R₁₂ radical wherein X₃ represents O, NH or S, advantageously O, and R₁₂ represents a hydrogen atom, an alkyl (C₁-C₁₀) carbonyl, an alkyl (C₁-C₁₀) phenyl, an alkyl (C₁-C₁₀) sulphonyl, an alkyl (C₁-C₁₀) phenyl sulphonyl or an alkyl (C₁-C₁₀)-dialkyl (C₁-C₁₀) silyl, advantageously a hydrogen atom;

R₅ represents the group N⁺OCH₃R₁₃ or the group NCH₃R₁₃ wherein

R₁₃ represents a hydrogen atom, a C₃-C₁₀ cycloalkyl, a C₁-C₁₀ alkyl, a phenyl or a phthalimide, advantageously —CH3 and

[symbol] represents ═O or —OR₁₄ wherein R₁₄ represents a hydrogen atom, a C₁-C₁₀ alkyl, an alkyl (C₁-C₁₀) carbonyl, an alkyl (C₁-C₁₀) phenyl or an alkyl (C₁-C₁₀) sulphonyl;

or pharmaceutically acceptable addition salts, isomers, enantiomers or diastereoisomers thereof, as well as mixtures thereof, for use as a medicament.

Advantageously, general formula I is such that the hydrogen atoms are arranged in the following manner:

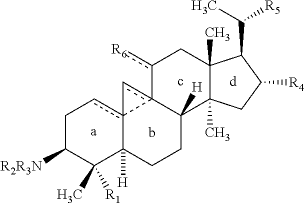

Advantageously, any triterpenic alkaloid according to the invention is useful as a medicament, including the compounds of general formulas II, III and IV.

The present invention comprises pharmaceutical compositions, or “medicaments,” comprising at least one compound of the present invention, or an individual isomer, a racemic or non-racemic mixture of isomers or a pharmaceutically acceptable salt, or a solvate thereof together with at least one pharmaceutically acceptable carrier, and optionally other therapeutic and/or prophylactic ingredients.

In general, the compounds of the present invention will be administered in a therapeutically effective quantity by any of the accepted modes of administration for agents which serve like uses. The suitable dosing range is typically from 1 mg to 500 mg per day, preferably from 1 mg to 100 mg per day, and more preferably from 1 mg to 30 mg per day, according to a number of factors such as the severity of the pathology to be treated, the age and relative health of the subject, the strength of the compound used, the route and form of administration, the indication at which administration is directed, and the preferences and experience of the prescribing physician. Those skilled in the treatment of such a pathology will be able, without over-burdensome experience and by relying on personal knowledge and the description of this application, to establish a therapeutically effective quantity of the compound of the present invention for a given pathology. In general, the compounds of the present invention will be administered as pharmaceutical formulations, including those suited for oral (including buccal and sublingual), rectal, nasal, topical, pulmonary, vaginal or parenteral (including intramuscular, intraarterial, intrathecal, subcutaneous and intravenous) administration or in a suitable form for administration by inhalation or insufflation. The preferred manner of administration is generally oral, using a daily dosing scheme that can be adjusted according to the degree of the condition.

A compound or compounds of the present invention, together with one or more conventional adjuvants, carriers or diluents, can be formed into pharmaceutical compositions and dosage units. Pharmaceutical compositions and galenical unit forms can be composed of conventional ingredients in conventional proportions, with or without other compounds or active ingredients, and the galenical unit forms can contain any appropriate effective quantity of the active product according to the daily dosing scheme used. Pharmaceutical compositions can be in the form of solids, such as tablets or filled capsules, semi-solids, powders, extended release formulations, or liquids such as solutions, suspensions, emulsions, elixirs or filled capsules for oral use; or in the form of suppositories for rectal or vaginal administration; or in the form of sterile injectable solutions for parenteral use. Consequently, formulations containing approximately one (1) milligram of active ingredient or, more broadly, approximately 0.01 to approximately one hundred (100) milligrams, by tablet, are suitable representative galenical unit forms.

Compounds of the present invention can be formulated in a wide variety of galenical forms for oral administration. Pharmaceutical compositions and galenical forms can include one or more compounds of the present invention, or pharmaceutically acceptable salts of same, as active components. Pharmaceutically acceptable carriers can be either solids or liquids. Preparations in solid form include powders, tablets, pills, capsules, suppositories and dispersible granules. A solid carrier can be one or more substances that can also act as diluents, flavoring agents, solubilizing agents, lubricating agents, suspension agents, binders, preservatives, tablet disintegration agents or encapsulating material. In powders, the carrier is generally a finely separated solid which is mixed with the finely separated active component. In tablets, the active component is generally mixed with a carrier having the necessary binding capacity in suitable proportions, and then compressed to the desired shape and size. Powders and tablets preferably contain approximately one (1) percent to approximately seventy (70) percent active compound. Suitable carriers include, but are not limited to, magnesium carbonate, magnesium stearate, talc, sugar, lactose, pectin, dextrin, starch, gelatin, gum tragacanth, methylcellulose, sodium carboxymethylcellulose, low melting-point wax, cocoa butter and the like. The term “preparation” means the formulation of the active compound with an encapsulating material, such as a carrier, forming a capsule wherein the active component, with or without a carrier, is surrounded by, and is in association with, a carrier. Similarly, tablets and lozenges are included. Tablets, powders, capsules, pills and lozenges can be in solid forms suitable for oral administration.

Other forms suitable for oral administration include preparations in liquid form including emulsions, syrups, elixirs, aqueous solutions and aqueous suspensions, or preparations in solid form intended to be converted just before use into preparations in liquid form. Emulsions may be prepared in solutions, for example, in aqueous solutions in propylene glycol, or may contain emulsifying agents such as, for example, lecithin, sorbitan monooleate or gum arabic. Aqueous solutions can be prepared by dissolving the active component in water and by adding suitable colorants, flavors, stabilizing agents and thickeners. Aqueous suspensions can be prepared by dispersing the finely separated active component in water with a viscous material, such as natural or synthetic gums, resins, methylcellulose, sodium carboxymethylcellulose, and other well-known suspension agents. Preparations in solid form include solutions, suspensions and emulsions, and can contain, in addition to the active component, colorants, flavors, stabilizers, buffers, artificial and natural sweeteners, dispersants, thickeners, solubilization agents and the like.

The compounds of the present invention can be formulated for parenteral administration (for example by injection, including bolus injection or continuous infusion) and can be provided in galenical unit form in vials, pre-filled syringes, small-volume infusions or multiple-dose containers with an added preservative. Compositions can take forms such as suspensions, solutions or emulsions in oil or aqueous vehicles, for example solutions in aqueous polyethylene glycol. Examples of carriers, diluents, solvents or oil or non-aqueous vehicles include propylene glycol, polyethylene glycol, vegetable oils (olive oil, for example), and injectable organic esters (ethyl oleate, for example), and can contain formulation agents such as preservatives, wetting agents, emulsifiers, suspension agents, stabilizers and/or dispersants. As a variation, the active ingredient can be in powder form, obtained by aseptic isolation of sterile solids or by lyophilization of a solution for constitution before use with a suitable vehicle, for example sterile nonpyrogenic water.

Compounds of the present invention can be formulated for topical administration on the skin in the form of pomades, creams or lotions, or as transdermal patches. Pomades and creams, for example, can be formulated with an aqueous or oil base with the addition of suitable thickening and/or jellifying agents. Lotions can be formulated with an aqueous or oil base and will also generally contain one or more emulsifiers, stabilizers, dispersants, suspension agents, thickeners or colorants. Formulations suitable for topical administration in the mouth include lozenges comprising the active agents in a flavored base, usually of sucrose and gum arabic or gum tragacanth; lozenges comprising the active ingredient in an inert base such as gelatin and glycerin or sucrose and gum arabic; and collutories comprising the active ingredient in a suitable liquid carrier.

Compounds of the present invention can be formulated for administration as suppositories. A low melting-point wax, such as a mixture of fatty acid glycerides or cocoa butter is first melted and the active component is dispersed homogeneously, for example by agitation. The molten homogeneous mixture is then poured into molds of convenient size and allowed to cool and solidify.

Compounds of the present invention can be formulated for nasal administration. Solutions or suspensions are applied directly in the nasal cavity by conventional means, for example with a dropper, a pipette or a sprayer. Formulations can be prepared in single-dose or multiple-dose forms. A dropper or pipette can be used by administering to the patient a predetermined suitable volume of the solution or suspension. A spray can be used, for example, by providing the patient with a measured-dose sprayer.

Compounds of the present invention can be formulated for administration by aerosol, particularly in the respiratory tract, including intranasal administration. In general, the compound will have a small particle size, for example roughly five (5) microns or less. Such a particle size can be obtained by means known to those skilled in the art, for example by micronization. The active ingredient is formed in a pressurized package with a suitable propellant, such as a chlorofluorocarbon (CFC), for example, dichlorodifluoromethane, trichlorofluoromethane or dichlorotetrafluoroethane, or carbon dioxide or another suitable gas. Conveniently, the aerosol can also contain a surfactant such as lecithin. The medicament dose can be controlled by a metering valve. As a variant, the active ingredients can be produced in dry powder form, for example a mixture of the powdered compound in a suitable powder base such as lactose, starch, starch derivatives such as hydroxypropylmethylcellulose, and polyvinylpyrrolidine (PVP). The powder carrier will form a gel in the nasal cavity. The powder composition can be provided in galenical unit form, for example in capsules or cartridges, of gelatin for example, or packets from which the powder can be administered by means of an inhaler.

Compounds of the present invention can be formulated in transdermal or subcutaneous medicament delivery devices. Such delivery systems are advantageous when extended release of the compound is necessary and when it is crucial that the patient follows the treatment plan. Compounds in transdermal delivery systems are frequently bound to a solid support that adheres to the skin. The compound of interest can also be combined with a substance to improve penetration, for example azone (1-dodecylazacyeloheptan-2-one). Extended release delivery systems are inserted subcutaneously in the subdermal tissue layer by surgery or injection. Subdermal implants encapsulate the compound in a soluble membrane in a liquid, such as silicone rubber, or a biodegradable polymer, such as lactic polyacid.

Pharmaceutical preparations are preferably in galenical unit form. In one such form, the preparation is subdivided into unit doses containing suitable quantities of the active component. The galenical unit form can be a packaged preparation, the package containing discrete quantities of the preparation, such as packaged tablets or capsules and powders in bottles or vials. Similarly, the galenical unit form itself can be a capsule, a tablet or a lozenge, or it can be a suitable number of any of the above in packaged form.

Other suitable pharmaceutical carriers and their formulations are described in Remington, The Science and Practice of Pharmacy 1995, edited by E. W. Martin, Mack Publishing Company, 19th edition, Easton, Pa.

According to a preferred embodiment, the triterpenic alkaloid for use as a medicament according to the invention is characterized wherein it is selected among one of the compounds of the following formulas:

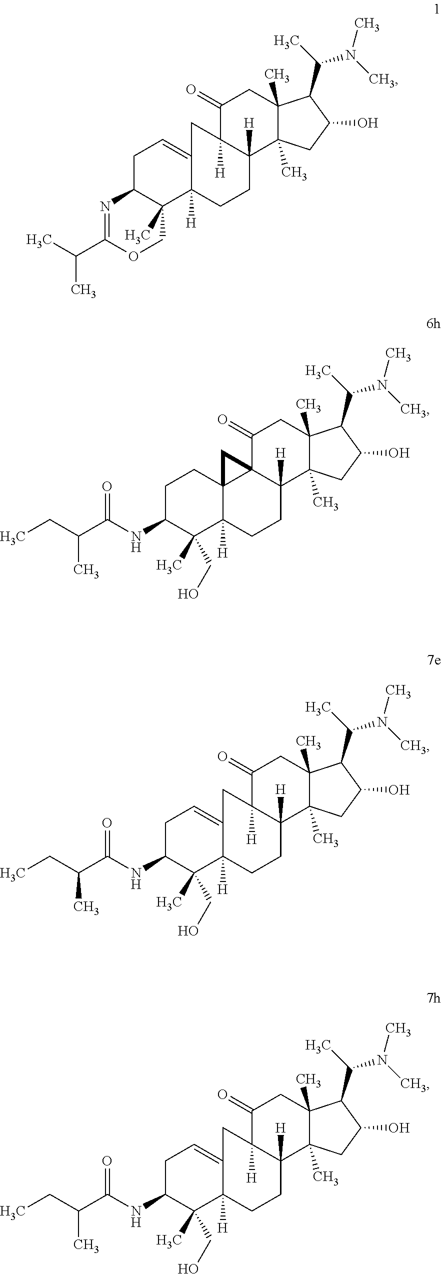

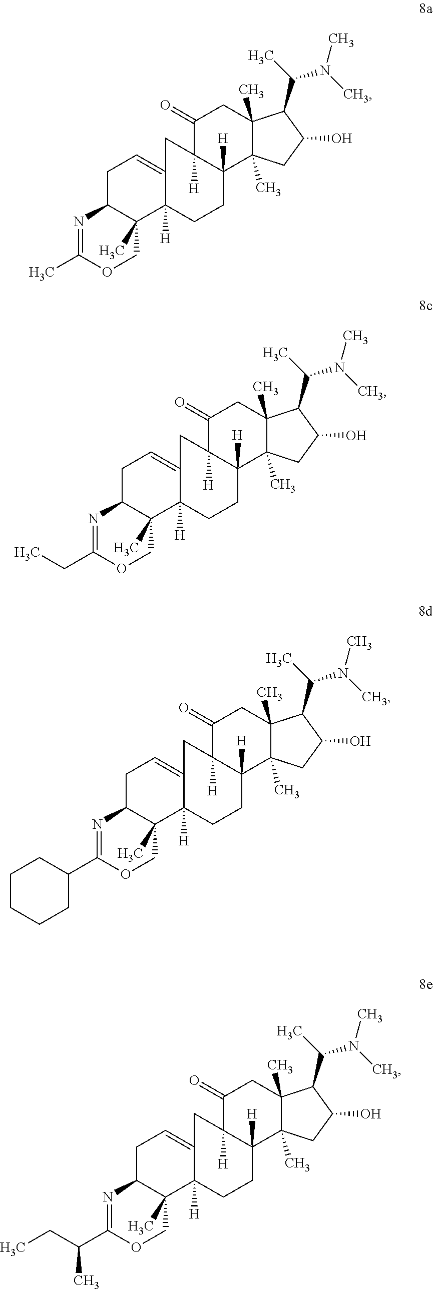

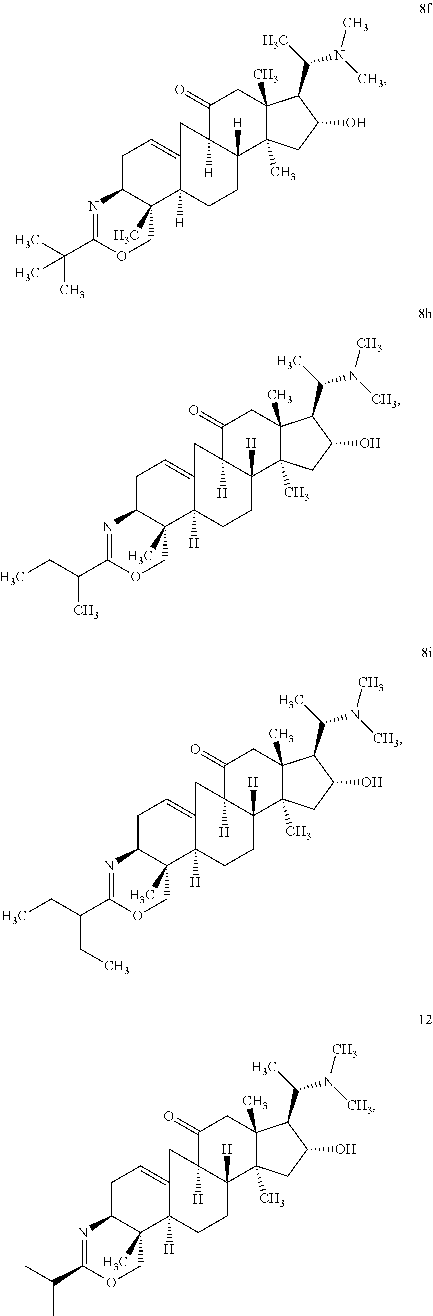

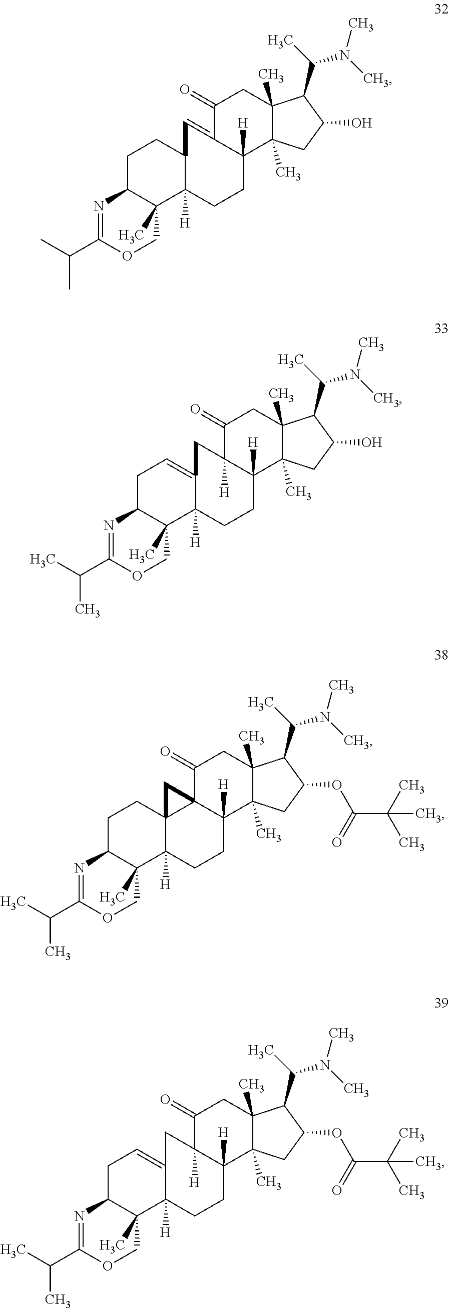

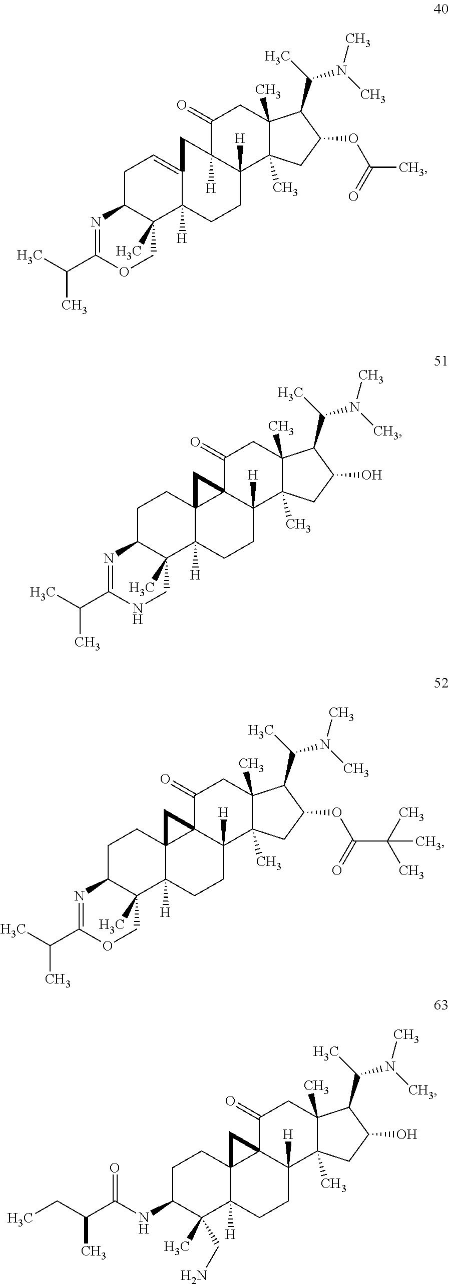

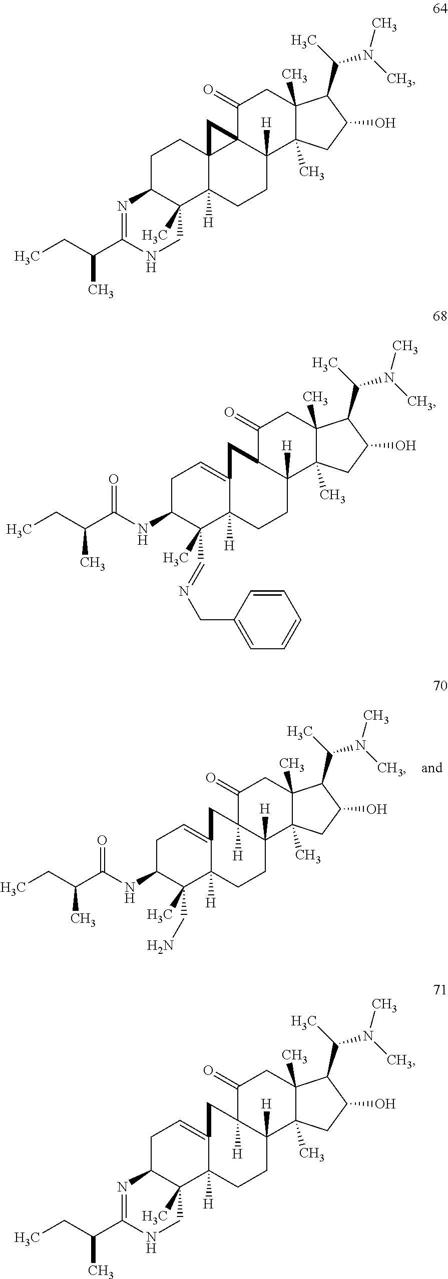

According to another preferred embodiment, the triterpenic alkaloid according to the invention is characterized wherein the medicament is intended to treat a subject suffering from a disease of the central or peripheral nervous system.

In the present invention, “subject” includes mammals and non-mammals. Mammals include any member of the mammalian class including, but not being limited to, humans, non-human primates such as chimpanzees and other monkeys and monkey species; farm animals such as cattle, horses, sheep, goats and pigs; domestic animals such as rabbits, dogs and cats; laboratory animals, including rodents such as rats, mice and guinea pigs; and the like. Examples of non-mammals include, but are not limited to, birds and the like.

In addition, the terms “to treat” or “treatment of” a pathological state as used in the present invention includes:

(1) to prevent the pathological state, namely to cause the clinical symptoms of the pathological state not to develop in a subject who may be exposed or predisposed to a pathological state, but who has not yet experienced or displayed the symptoms of the pathological state;

(2) to inhibit the pathological state, namely to stop the development of the pathological state or of its clinical symptoms; or

(3) to reduce the pathological state, namely to cause a temporary or permanent regression of the pathological state or its clinical symptoms.

In the present invention, “disease of the central or peripheral nervous system” means any deterioration of the health of the central or peripheral nervous system in a subject, attributed to internal or external causes, expressed by symptoms and signs, and appearing by disturbances of functioning or by lesions. Examples which can be cited, without however being limiting, include Alzheimer's disease, memory disorders associated with aging or with Alzheimer's disease, disorders associated with trisomy 21, Lewy body dementia, Parkinson's dementia, vascular dementia, delirium, traumatic brain injuries, myasthenia of congenital or autoimmune origin to release neuromuscular blocking agents acting on muscle post-synaptic receptors and any cholinergic syndrome wherein there is a reduction in the quantity of acetylcholinesterase released, epilepsy, brain tumor, neurodegenerative disease such as Tay-Sachs disease, Lesch-Nyhan syndrome, Huntington's disease, amyotrophic lateral sclerosis (Lou Gehrig's disease), Down syndrome, or peripheral neuropathies such as diabetic peripheral neuropathy.

According to a more preferred embodiment, the triterpenic alkaloid according to the invention is characterized wherein the disease of the central or peripheral nervous system is selected from the group comprised of Alzheimer's disease, memory disorders associated with aging or with Alzheimer's disease, disorders associated with trisomy 21, Lewy body dementia, Parkinson's dementia, vascular dementia, delirium, traumatic brain injuries, myasthenia of congenital or autoimmune origin to release neuromuscular blocking agents acting on muscle post-synaptic receptors and any cholinergic syndrome wherein there is a reduction in the quantity of acetylcholinesterase released.

Another object of the invention is the use of a triterpenic alkaloid according to the present invention to manufacture a medicament intended to treat a subject suffering from Alzheimer's disease, memory disorders associated with aging or with Alzheimer's disease, disorders associated with trisomy 21, Lewy body dementia, Parkinson's dementia, vascular dementia, delirium, traumatic brain injuries, myasthenia of congenital or autoimmune origin to release neuromuscular blocking agents acting on muscle post-synaptic receptors and any cholinergic syndrome wherein there is a reduction in the quantity of acetylcholinesterase released.

Lastly, one object of the invention is a method for treating a subject suffering from a disease of the central or peripheral nervous system which includes administration of a triterpenic alkaloid according to the present invention or a pharmaceutical composition comprising one such alkaloid.

The examples which follow are intended to illustrate the invention without limiting the scope of the invention in any way.

### EXAMPLES

### Example 1

### Isolation of N-3-isobutyrylcycloxobuxidine-F 2

### or

### (20S)-3-isobutyrylamino-9,19-cyclo-4α-hydroxymethyl-4,14α-dimethyl-20-dimethylamino-5α,9-pregnan-16α-ol-11-one 2

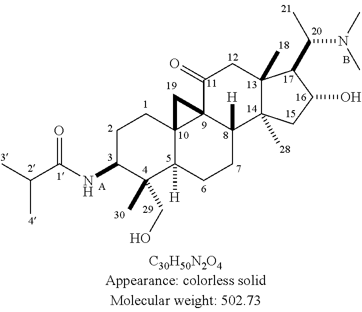

1.1. Total Alkaloids Extraction Protocol.

1.5 kg of ground, dried leaves of Buxus balearica Wild are alkalinized with 600 ml of a 30% ammonia solution. A first extraction is carried out, under agitation, with 10 l of a mixture of solvents CH₂Cl₂/EtOH (9/1) over night at room temperature. After filtration, five successive macerations are carried out with 10 l of a mixture of solvents CH₂Cl₂/EtOH (9/1), while agitating for three hours at room temperature between each filtration.

The recombined organic phases are evaporated under a vacuum to yield 240 g of a dark green extract. The extract is successively taken up with 7 l of dichloromethane and washed 10 times with 1 l of 2 N hydrochloric acid. The aqueous phases are alkalinized with a 30% ammonia solution, then extracted with 6×2 l of dichloromethane. The organic phases are evaporated under a vacuum to yield 98 g of a brown powder.

Using the substance remaining after six extractions/macerations, a soxhlet is prepared with dichloromethane for 20 hours until a negative Meyer test is obtained. Each extract is successively taken up with 7 l of dichloromethane and then washed 10 times with 1 l of 2 N hydrochloric acid. The aqueous phases are alkalinized with a 30% ammonia solution, then extracted with 6×2 l of dichloromethane. The organic phases are finally evaporated and yield 7 g of alkaloids. Thus, 105 g of total alkaloids were isolated with a yield of 7%.

This experiment was also carried out on an 11 kg batch of ground, dried leaves of Buxus balearica Wild at the I.C.S.N. extraction facility at Gif-sur-Yvette, France.

1.2. Isolation of N-3-isobutyrylcycloxobuxidine-F 2.

### Preparation of the Solutions:

4 liters of a 1.0 M AcOH/AcONa buffer solution, pH 5, are prepared as follows:

17.136 g (0.2348 mol) of glacial acetic acid and 99.296 g (0.7151 mol) of sodium acetate are dissolved in 1000 ml of permuted water.

Dissolution of Total Alkaloids in Four Aqueous Phases at pH=5.

A solution of 27.3 g of crude alkaloids in 500 ml of dichloromethane is washed in 4×1 l of buffer solution at pH=5. This organic phase is then successively washed with 1 liter of a 10% ammonia solution, 1 liter of a permuted water solution and 1 liter of a sodium chloride saturated aqueous solution, then dried on anhydrous magnesium sulfate and evaporated under reduced pressure. This yields a residue of 1.74 g.

### Progressive Neutralization Followed by Extractions: Alkaloid Fractionation.

Next, the pH of each aqueous phase containing the initial buffer solution, wherein the alkaloids are present, is increased by 1 pH unit by the addition of 10% ammonia. The aqueous phases are then successively extracted with 8×500 ml of dichloromethane until the alkaloids are exhausted from the aqueous phases. The 8 organic phases are then washed successively with 1 liter of a 10% ammonia solution, 1 liter of a permuted water solution and 1 liter of a sodium chloride saturated aqueous solution, then dried on anhydrous magnesium sulfate and evaporated under reduced pressure. Thus, 3.19 g of N-3-isobutyrylcycloxobuxidine-F 2 are extracted at pH=5.8 with a yield of 0.8%.

The pH is measured using a calibrated pH-meter.

Upon completion, the following mass analysis is obtained:

| | Mass (g) |
|---|---|
| Residue | 1.74 |
| pH | |
| 5 | 4.71 |
| 5.8 | 3.19 |
| 6.5 | 1.80 |
| 7.3 | 4.06 |
| 8.2 | 6.70 |
| 9.1 | 3.25 |
| | Total: 25.4, loss of 1.9 g |

This experiment was also carried out on an 11 kg batch of ground, dried leaves of Buxus balearica Wild at the I.C.S.N. extraction facility at Gif-sur-Yvette, France, which made it possible to isolate, after trituration of the extract at pH=6 in acetone, 100 g of N-3-isobutyrylcycloxobuxidine-F 2 with a yield of 0.9% whose spectroscopic characteristics are identical to those described by Herlem-Gaulier et al. (1968).

MP (° C.): 257

[α]_{D}=+69° (c=1.01; 25° C., CHCl₃)

Elemental analysis: calculated for C₃₀H₅₀N₂O₄%: C, 71.67; H, 10.02; N, 5.57; O, 12.73. measured: C, 71.51; H, 10.14; N, 5.34; O, 12.87.

IR (CHCl₃) υ(cm⁻¹): 3434 (OH, NH); 1690 (C═O); 1654 and 1513 (CONH); 1096 (C—OH).

Mass (IE): m/z: 502.1 (M⁺.); 72.0 (Me-CH═N⁺(CH₃)₂, base peak). (ESI): m/z: 503.4 ([M+H], 100); 504.4 (10).

HRMS (ESI) calculated for C₃₀H₅₁N₂O₄ m/z=503.3849; measured: 503.3852.

Note Concerning the Chemical Shift of Methyls Attached to the Nitrogen N_{B}:

At 243° K, the N_{B}(CH₃)₂ groups have as chemical shifts of 2.07 ppm and 2.30 ppm in the form of two singlets.

At 300° K, inversion of the nitrogen doublet is slow due to an intramolecular hydrogen bond between the nitrogen N_{B} and the proton of the alcohol attached at C₁₆. The N_{B}(CH₃)₂ groups then have as chemical shifts 2.20 ppm in the form of a broad singlet. Using ¹³C NMR, no peak is observed at this temperature.

### Example 2

### Isolation of N-3-isobutyrylcycloxobuxidine-F 1

### or

### (20S)-10(9->1)abeo-4,14α-dimethyl-20-dimethylamino-(2′-isopropyl-4′,4′-dihydro[3′,1′]oxazine)-5α,9-pregn-1(10)-en-16α-ol-11-one 1

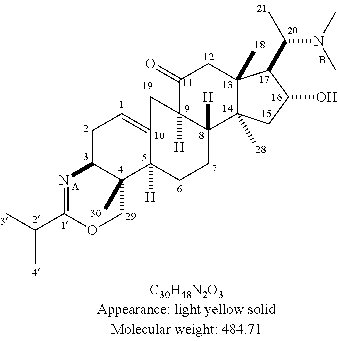

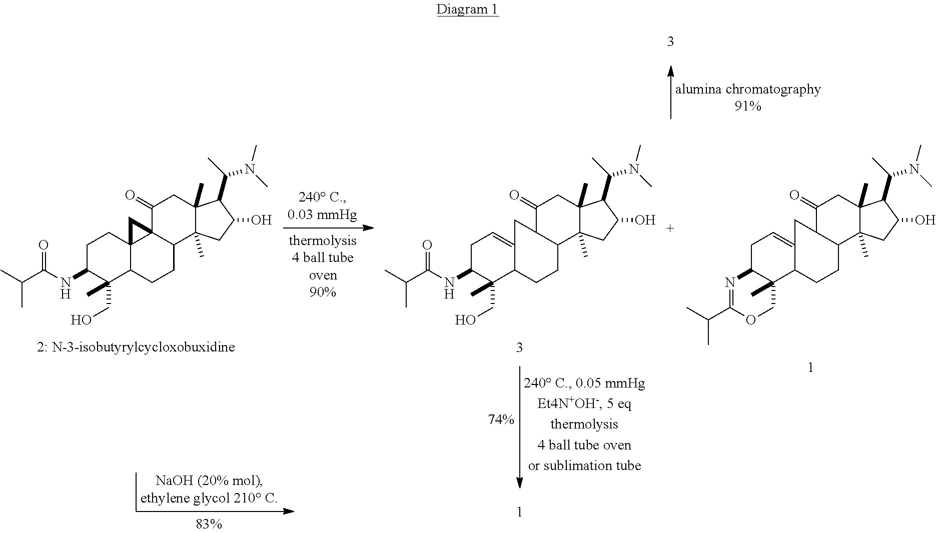

First Method: Ball Tube Oven or Sublimation Tube

100 mg (0.20 mmol, 1.0 eq) of N-3-isobutyrylcycloxobuxidine-F 2 are heated at 240° C. under 0.05 mmHg in a ball tube oven or a sublimation tube. A solid sublimate is obtained in 3 hours. The sublimate is a mixture of the opening product of cyclopropane 3 (18%) and N-3-isobutyrylcycloxobuxidine-F 1 (82%). To a solution of the sublimate in 2 ml of dichloromethane are added 515 mg (0.87 mmol, 5.0 eq) of a 25% tetraethylammonium hydroxide solution in methanol. After evaporation under a vacuum, the red residue heated at 240° C. under 0.05 mmHg for 3 hours yields 71.6 mg of 1 in the form of a light yellow crystalline sublimate with a yield of 74%. It is also possible to carry out this synthesis with 1.0 M tetrabutylammonium hydroxide in methanol.

Second Method: in Solution in Glycol

To a suspension of 50 mg (0.10 mmol, 1.0 eq) of N-3-isobutyrylcycloxobuxidine-F 2 in 5 ml of freshly distilled ethylene glycol is added 0.8 mg of soda (0.02 mmol, 0.2 eq). After 6 hours of heating at 215° C. under argon, the mixture is alkalinized with 60 ml of a 10% ammonia solution (pH=10) and extracted with 3×40 ml of dichloromethane. The recombined organic phases are successively dried on anhydrous sodium sulfate and evaporated under a vacuum to provide 40 mg of 1 in the form of a light yellow solid with a yield of 83%.

MP (° C.): 281.5

[α]_{D}=+71° (c=0.8; 25° C., CHCl₃)

Elemental analysis: calculated for C₃₀H₄₈N₂O₃: C, 74.34; H, 9.98; N, 5.78; O, 9.90. measured C, 74.13; H, 9.91; N, 5.67; O, 9.97.

IR (CHCl₃) υ(cm⁻¹): 3433 (OH.); 1693 (C═O); 1513 (C═H); 1043 (C—OH).

Mass (ESI): m/z: 485.3 ([M+H], 95); 486.3 (100); 487.4 (50); 503.4 (2).

HRMS (ESI) calculated for C₃₀H₄₉N₂O₃ m/z=485.3743; measured: 485.3721.

### Example 3

### 10(9->1)abeo-N-3-isobutyrylcycloxobuxi-1(10)-enedine-F 3

### or

### (20S)-10(9->1)abeo-4α-hydroxymethyl-3-isobutyrylamino-4,14α-dimethyl-20-dimethylamino-5α,9-pregn-1(10)-en-16α-ol-11-one 3

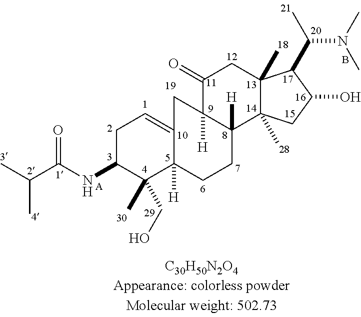

First Method: in Solution

A suspension of 250 mg (0.49 mmol, 1.0 eq) of N-3-isobutyrylcycloxobuxidine-F 2 in 20 ml of freshly distilled ethylene glycol on soda (concentration: 12 g/l) is heated at 160° C. for 6 hours under argon. The mixture is then alkalinized with 50 ml of a 10% ammonia solution (pH=10) and extracted with 3×40 ml of dichloromethane. The recombined organic phases are successively washed with 50 ml of a sodium chloride saturated aqueous solution, dried on anhydrous sodium sulfate and evaporated under a vacuum. The residue is chromatographed on a Merck activity II-III alumina column (eluent: 98/2 dichloromethane/methanol) to yield 105 mg of 3 in the form of a colorless powder after trituration in diethyl ether with a yield of 42% whose spectroscopic characteristics are identical to those described by Herlem-Gaulier, D. et al. (1968).

Second Method: in the Oven

100 mg (0.20 mmol, 1.0 eq) of N-3-isobutyrylcycloxobuxidine-F 2 are heated at 240° C. under 0.05 mmHg in a ball tube oven. A sublimate is obtained in 3 hours. The solid sublimate is a mixture of desired product 3 (18%) and N-3-isobutyrylcycloxobuxidine-F 1 (82%). The recombined residues (pellet and sublimate) are chromatographed on a Merck activity II-III alumina column (eluent: 98/2 dichloromethane/methanol) to yield 91 mg of 3 in the form of a colorless powder after trituration in acetone with a yield of 91% whose spectroscopic characteristics are identical to those described by Herlem-Gaulier, D. et al. (1968).

MP (° C.): 242

[α]_{D}=−46° (c=1.0; 25° C., CHCl₃)

Elemental analysis: calculated for C₃₀H₅₀N₂O₄.0.5H₂O: C, 70.45; H, 9.98; N, 5.47. measured: C, 70.84; H, 9.71; N, 5.21.

IR (CHCl₃) υ(cm⁻¹): 3433 (OH, NH); 1693 (C═O); 1652 and 1513 (CONH); 1043 (C—OH).

Mass (ESI): m/z: 503.3 ([H+H], 100); 504.3 (90).

HRMS (ESI) calculated for C₃₀H₅₁N₂O₄ m/z=503.3849; measured: 503.3852.

### Example 4

### Syntheses of N-3-isobutyrylcycloxobuxidine-F 1 analogs

In order to supplement the pharmacological studies, analogs of 1 were synthesized and their antiacetylcholinesterase and antibutyrylcholinesterase activities measured.

4.1. Synthesis of Compounds 5, 6, 7 and 8

The common precursor of these analogs is cycloxobuxidine 5, which is obtained by acid hydrolysis of the amide function of N-3-isobutyrylcycloxobuxidine-F 2 with a yield of 94% (diagram 2).

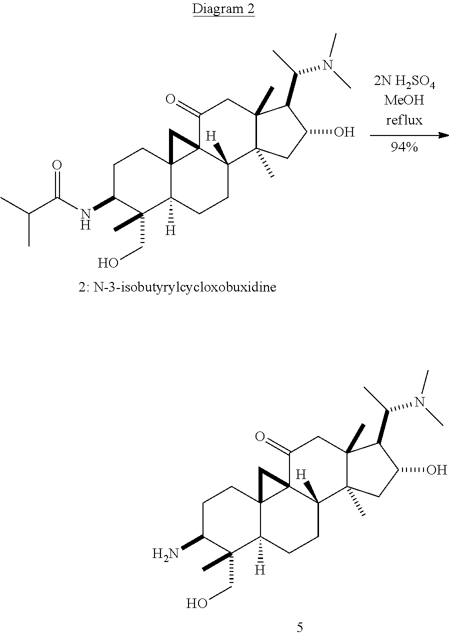

Amides 6 are obtained by reaction of amine 5 with various anhydrides. Pyrolysis of compounds 6 under 0.03 mmHg at high temperature (235° C. to 270° C.) quantitatively yields mixtures of compounds 7 and 8 in variable proportions. Chromatography of these mixtures (7 and 8) on basic alumina makes it possible to transform them into corresponding compound 7 by hydrolysis of the dihydrooxazine function present on compounds 8. Finally, compounds 8 were obtained by thermolysis of the mixture of corresponding compounds 7 and 8 under 0.03 mmHg at high temperature (235° C. to 270° C.) in the presence of tetraethylammonium hydroxyl with good yields (30%-94%; diagram 3 below):

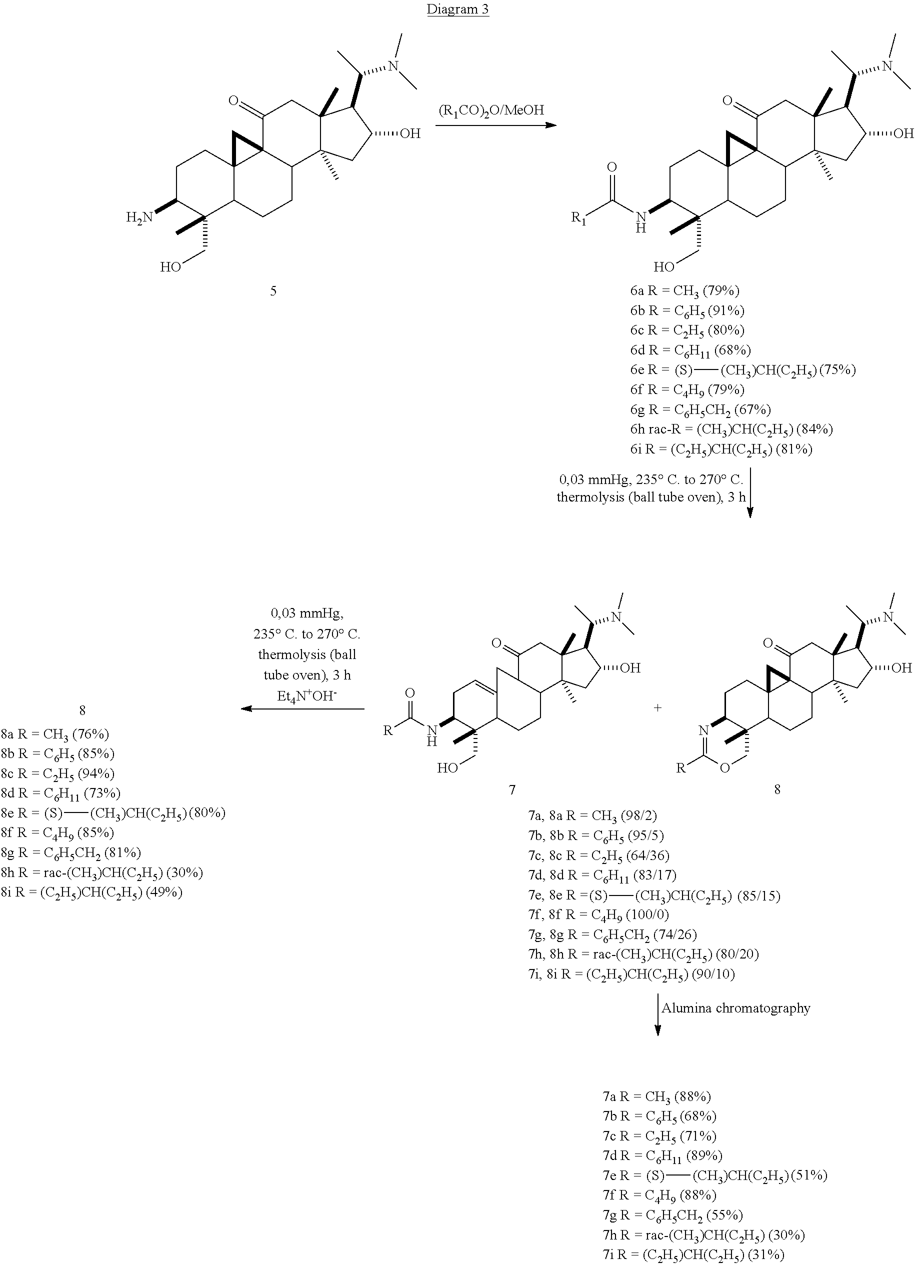

### Compound 5: cycloxobuxidine-F 5

### or

### (20S)-3-amino-9,19-cyclo-4α-hydroxymethyl-4,14α-dimethyl-20-dimethylamino-5α,9-pregnan-16α-ol-11-one 5

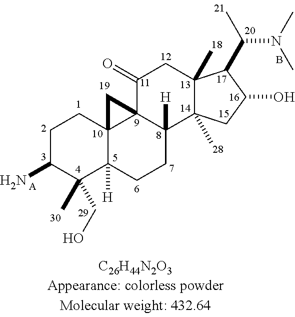

To a solution of 821 mg (1.63 mmol, 1.0 eq) of N-3-isobutyrylcyclobuxidine-F 2 in 30 ml of methanol are slowly added 30 ml (30.00 mmol, 18.4 eq) of a 2 N sulfuric acid aqueous solution at room temperature. After 3 hours of agitation at 90° C., the reaction mixture is cooled to room temperature and is evaporated under reduced pressure at greater than 25° C. The liquor obtained is heated for 1.5 hours at 70° C. It is yellow in color. The reaction mixture is alkalinized by 50 ml of a 10% ammonia solution and extracted with 3×40 ml of dichloromethane. The recombined organic phases are successively washed with 40 ml of a sodium chloride saturated aqueous solution, dried on anhydrous sodium sulfate and evaporated under a vacuum. The crude product is recrystallized in ethyl acetate. After filtration, washing and drying under reduced pressure, 657 mg of 5 in the form of a colorless solid are obtained with a yield of 94% whose spectroscopic characteristics are identical to those described by Herlem-Gaulier, D. et al. (1968).

MP (° C.): 242

[α]_{D}=+127° (c=0.6; 25° C., CHCl₃)

Elemental analysis: calculated for C₂₆H₄₃N₂O₄: C, 72.18; H, 10.25; N, 6.48; O, 11.09.

Measured: C, 71.99; H, 10.22; N, 6.31; O, 11.28.

IR (CHCl₃) υ(cm⁻¹): 3018 (OH, NH); 1680 (C═O); 1615 (C—NH); 1095 (C—OH).

Mass (ESI): m/z: 433.3 ([M+H], 100); 416.3 (10).

HRMS (ESI) calculated for C₂₆H₄₅N₂O₃ m/z=433.3430; measured: 433.3425.

### Compound 6a: N-3-acetylcycloxobuxidine-F 6a

### or

### (20S)-3-acetylamino-9,19-cyclo-4α-hydroxymethyl-4,14α-dimethyl-20-dimethylamino-5α,9-pregnan-16α-ol-11-one 6a

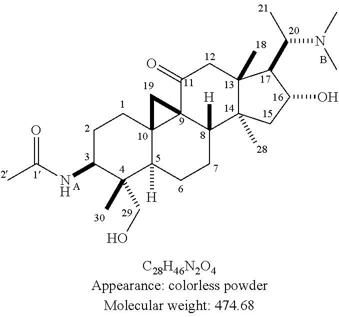

To a solution of 240 mg (0.55 mmol, 1.0 eq) of cycloxobuxidine-F 5 in 10 ml of methanol are added 63 ml (0.66 mmol, 1.2 eq) of acetic anhydride. After 5 hours of agitation at room temperature, the mixture is neutralized with several drops of a 10% sodium bicarbonate solution (pH=8), then alkalinized with 50 ml of a 10% ammonia solution (pH=10) and extracted with 3×40 ml of dichloromethane. The recombined organic phases are successively washed with 30 ml of a sodium chloride saturated aqueous solution, dried on anhydrous sodium sulfate and evaporated under a vacuum. The residue is chromatographed on a Merck activity II-III alumina column (eluent: 99.7/0.3 dichloromethane/methanol then 98/2 dichloromethane/methanol) to yield 210 mg of 6a in the form of a colorless powder after trituration in acetone with a yield of 79%.

MP (° C.): 255

[α]_{D}=+73° (c=0.98; 25° C., CHCl₃)

Elemental analysis: calculated for C₂₈H₄₆N₂O₄.0.5H₂O: C, 70.85; H, 9.77; N, 5.90. measured: C, 69.51; H, 9.61; N, 5.39.

IR (CHCl₃) υ(cm⁻¹): 3436 (OH, NH); 1656 (C═O); 1656 and 1513 (CONH); 1049 (C—OH).

Mass (ESI): m/z: 475.3 ([M+H], 100); 476.4 (15).

HRMS (ESI) calculated for C₂₈H₄₇N₂O₄ m/z=475.3536; measured: 475.3511.

### Compound 6b: N-3-benzoylcycloxobuxidine-F 6b

### or

### (20S)-3-benzoylamino-9,19-cyclo-4α-hydroxymethyl-4,14α-dimethyl-20-dimethylamino-5α,9-pregnan-16α-ol-11-one 6b

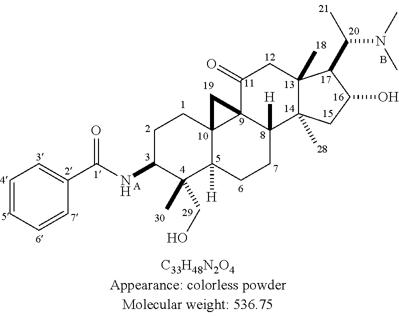

To a solution of 166 mg (0.38 mmol, 1.0 eq) of cycloxobuxidine-F 5 in 10 ml of methanol are added 95 mg (0.46 mmol, 1.1 eq) of benzoic acid anhydride. After 4 hours of agitation at room temperature, the mixture is neutralized with several drops of a 10% sodium bicarbonate solution (pH=8), then alkalinized with 50 ml of a 10% ammonia solution (pH=10) and extracted with 3×40 ml of dichloromethane. The recombined organic phases are washed with 30 ml of a sodium chloride saturated aqueous solution, dried on anhydrous sodium sulfate and evaporated under a vacuum. The residue is chromatographed on a Merck activity II-III alumina column (eluent: 99.8/0.2 dichloromethane/methanol then 98/2 dichloromethane/methanol) to yield 156 mg of 6b in the form of a colorless powder after trituration in acetone with a yield of 76% whose spectroscopic characteristics are identical to those described by Herlem-Gaulier, D. et al. (1968).

MP (° C.): 277

[α]_{D}=+52° (c=1.00; 25° C., CHCl₃)

Elemental analysis: calculated for C₃₃H₄₈N₂O₄.0.5H₂O: C, 72.64; H, 9.04; N, 13.12; O, 5.13. measured: C, 72.87; H, 9.02; N, 12.97; O, 5.14.

IR (CHCl₃) υ(cm⁻¹): 3438 (OH, NH); 1645 (C═O); 1628 and 1518 (CONH); 1040 (C—OH).

Mass (ESI): m/z: 537.4 ([M+H], 100); 538.4 (15).

HRMS (ESI) calculated for C₃₃H₄₉N₂O₄ m/z=537.3692; measured: 537.3679.

### Compound 6c: N-3-propionylcycloxobuxidine-F 6c

### or

### (20S)-9,19-cyclo-4α-hydroxymethyl-4,14-dimethyl-20-dimethylamino-3-propionylamino-5α,9-pregnan-16α-ol-11-one 6c

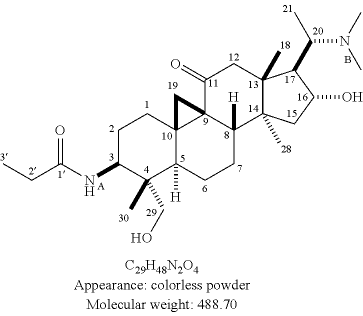

To a solution of 300 mg (0.69 mmol, 1.0 eq) of cycloxobuxidine-F 5 in 10 ml of methanol are added 98 ml (0.76 mmol, 1.1 eq) of propionic acid anhydride. After 4 hours of agitation at room temperature, the mixture is neutralized with several drops of a 10% sodium bicarbonate solution (pH=8), then alkalinized with 50 ml of a 10% ammonia solution (pH=10) and extracted with 3×40 ml of dichloromethane. The recombined organic phases are successively washed with 30 ml of a sodium chloride saturated aqueous solution, dried on anhydrous sodium sulfate and evaporated under a vacuum. The residue is chromatographed on a Merck activity II-III alumina column (eluent: 99.7/0.3/0.05 dichloromethane/methanol/ammonia then 98/2 dichloromethane/methanol) to yield 269 mg of 6c in the form of a colorless powder after trituration in acetone with a yield of 80%.

MP (° C.): 221

Elemental analysis: calculated for C₂₉H₄₈N₂O₄.0.5H₂O: C, 70.02; H, 9.85; N, 5.63. measured C, 70.63; H, 9.76; N, 5.21.

IR (CHCl₃) υ(cm⁻¹): 3434 (OH, NH); 1653 (C═O); 1653 and 1514 (CONH); 1049 (C—OH).

Mass (ESI): m/z: 489.3 ([M+H], 100); 295.2 (15); 490.3 (5).

HRMS (ESI) calculated for C₂₉H₄₉N₂O₄ m/z=489.3692; measured: 489.3655.

### Compound 6d: N-3-cyclohexanecarboxylcycloxobuxidine-F 6d

### or

### (20S)-3-(cyclohexanecarboxylamino)-9,19-cyclo-4α-hydroxymethyl-4,14α-dimethyl-20-dimethylamino-5α,9-pregnan-16α-ol-11-one 6d

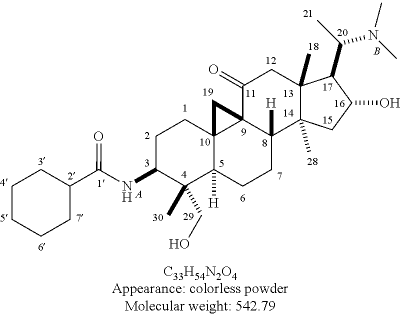

To a solution of 300 mg (0.69 mmol, 1.0 eq) of cycloxobuxidine-F 5 in 10 ml of methanol are added 182 mg (0.76 mmol, 1.1 eq) of cyclohexanecarboxylic acid anhydride. After 4 hours of agitation at room temperature, the mixture is neutralized with several drops of a 10% sodium bicarbonate solution (pH=8), then alkalinized with 50 ml of a 10% ammonia solution (pH=10) and extracted with 3×40 ml of dichloromethane. The recombined organic phases are successively washed with 30 ml of a sodium chloride saturated aqueous solution, dried on anhydrous sodium sulfate and evaporated under a vacuum. The residue is chromatographed on a Merck activity II-III alumina column (eluent: 99.7/0.3 dichloromethane/methanol then 98/2 dichloromethane/methanol) to yield 225 mg of 6d in the form of a colorless powder after trituration in acetone with a yield of 68%.

MP (° C.): 293

[α]_{D}=+61.9° (c=1.09; 25° C., CHCl₃)

Elemental analysis: calculated for C₃₃H₅₄N₂O₄: C, 73.02; H, 10.03; N, 5.16; O, 11.79. measured: C, 72.76; H, 9.94; N, 5.07; O, 11.68.

IR (CHCl₃) υ(cm⁻¹): 3434 (OH, NH); 1650 (C═O); 1650 and 1512 (CONH); 1050 (C—OH).

Mass (ESI): m/z: 543.4 ([M+H], 100); 544.5 (85).

HRMS (ESI) calculated for C₃₃H₅₅N₂O₄ m/z=543.4162; measured: 543.4128.

### Compound 6e: (S)—N-3-secbutyrylcycloxobuxidine-F 6e

### or

### (20S)-9,19-cyclo-4α-hydroxymethyl-4,14α-dimethyl-20-dimethylamino-3 (2S-methylbutyrylamino)-5α,9-pregnan-16α-ol-11-one 6e

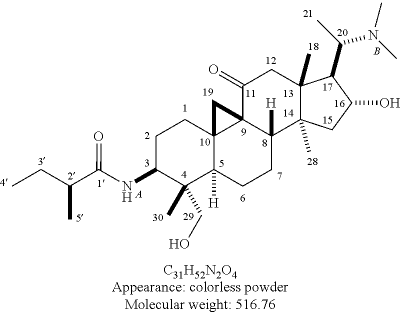

To a solution of 300 mg (0.69 mmol, 1.0 eq) of cycloxobuxidine-F 5 in 10 ml of methanol are added 151 ml (0.76 mmol, 1.1 eq) of (S)-2-methylbutyric acid anhydride. After 3 hours of agitation at room temperature, the mixture is neutralized with several drops of a 10% sodium bicarbonate solution (pH=8), then alkalinized with 50 ml of a 10% ammonia solution (pH=10) and extracted with 3×40 ml of dichloromethane. The recombined organic phases are successively washed with 30 ml of a sodium chloride saturated aqueous solution, dried on anhydrous sodium sulfate and evaporated under a vacuum. The residue is chromatographed on a Merck activity II-III alumina column (eluent: 99.5/0.5 dichloromethane/methanol then 98/2 dichloromethane/methanol) to yield 266 mg of 6e in the form of a colorless powder after trituration in acetone with a yield of 75%.

MP (° C.): 262

[α]_{D}=+ 59.0° (c=0.44; 21° C., CHCl₃)

Elemental analysis: calculated for C₃₁H₅₂N₂O₄.0.5H₂O: C, 70.85; H, 10.09; N, 5.33. measured C, 71.45; H, 9.88; N, 5.23.

IR (CHCl₃) υ(cm⁻¹): 3434 (OH, NH); 1651 (C═O); 1651 and 1511 (CONH); 1042 (C—OH).

Mass (ESI): m/z: 517.4 ([M+H], 100); 518.5 (75).

HRMS (ESI) calculated for C₃₁H₅₃N₂O₄ m/z=517.4005; measured: 517.4024.

### Compound 6f: N-3-pivalylcycloxobuxidine-F 6f

### or

### (20S)-9,19-cyclo-4a-hydroxymethyl-4,14α-dimethyl-20-dimethylamino-3 (2,2-dimethylpropionylamino)-5α,9-pregnan-16α-ol-11-one 6f

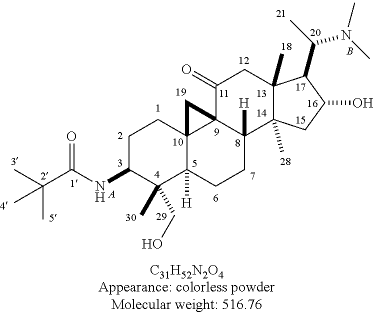

To a solution of 300 mg (0.69 mmol, 1.0 eq) of cycloxobuxidine-F 5 in 10 ml of methanol are added 156 ml (0.76 mmol, 1.1 eq) of pivalic acid anhydride. After 4 hours of agitation at room temperature, the mixture is neutralized with several drops of a 10% sodium bicarbonate solution (pH=8), then alkalinized with 50 ml of a 10% ammonia solution (pH=10) and extracted with 3×40 ml of dichloromethane. The recombined organic phases are successively washed with 40 ml of a sodium chloride saturated aqueous solution, dried on anhydrous sodium sulfate and evaporated under a vacuum. The residue is chromatographed on a Merck activity II-III alumina column (eluent: 99.5/0.5 dichloromethane/methanol then 98/2 dichloromethane/methanol) to yield 282 mg of 6f in the form of a colorless powder after trituration in acetone with a yield of 79%.

MP (° C.): 267

[α]_{D}=+69.9° (c=0.92; 25° C., CHCl₃)

Elemental analysis: calculated for C₃₁H₅₂N₂O₄.0.5H₂O: C, 70.85; H, 10.09; N, 5.33. measured: C, 71.29; H, 9.93; N, 5.04.

IR (CHCl₃) υ(cm⁻¹): 3454 (OH, NH); 1637 (C═O); 1637 and 1514 (CONH); 1046 (C—OH).

Mass (ESI): m/z: 517.4 ([M+H], 100); 518.5 (75).

HRMS (ESI) calculated for C₃₁H₅₃N₂O₄ m/z=517.4005; measured: 517.3986.

### Compound 6g: N-3-phenylacetylcycloxobuxidine-F 6g

### or

### (20S)-9,19-cyclo-4α-hydroxymethyl-4,14α-dimethyl-20-dimethylamino-3 (phenylacetylamino)-5α,9-pregnan-16α-ol-11-one 6g

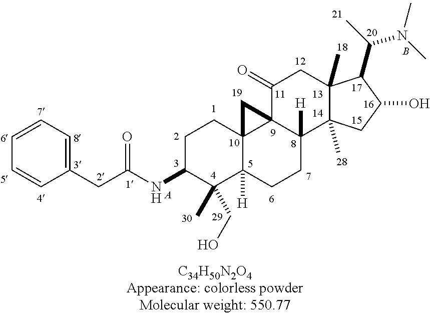

To a solution of 300 mg (0.69 mmol, 1.0 eq) of cycloxobuxidine-F 5 in 10 ml of methanol are added a suspension of 194 mg (0.76 mmol, 1.1 eq) of phenylacetic acid anhydride in 5 ml of methanol. After 4 hours of agitation at room temperature, the mixture is neutralized with several drops of a 10% sodium bicarbonate solution (pH=8), then alkalinized with 50 ml of a 10% ammonia solution (pH=10) and extracted with 3×30 ml of dichloromethane. The recombined organic phases are successively washed by 30 ml of a sodium chloride saturated aqueous solution, dried on anhydrous sodium sulfate and evaporated under a vacuum. The residue is chromatographed on a Merck activity II-III alumina column (eluent: 99.7/0.3 dichloromethane/methanol then 98/2 dichloromethane/methanol) to yield 255 mg of 6g in the form of a colorless powder after trituration in acetone with a yield of 67%.

MP (° C.): 265.5

[α]_{D}=+62.1° (c=0.84; 25° C., CHCl₃)

Elemental analysis: calculated for C₃₄H₅₀N₂O₄: C, 74.14; H, 9.15; N, 5.09; O, 11.62. measured: C, 73.98; H, 9.12; N, 4.97; O, 11.61.

IR (CHCl₃) υ(cm⁻¹): 3413 (OH, NH); 1650 (C═O); 1650 and 1516 (CONH); 1047 (C—OH).

Mass (ESI): m/z: 551.4 ([M+H], 100); 552.5 (90); 553.5 (10).

HRMS (ESI) calculated for C₃₄H₅₁N₂O₄ m/z=551.3849; measured: 551.3816.

### Compound 6h: N-3-secbutyrylcycloxobuxidine-F 6h

### or

### (20S)-9,19-cyclo-4α-hydroxymethyl-4,14α-dimethyl-20-dimethylamino-3 (2-methylbutyrylamino)-5α,9-pregnan-16α-ol-11-one 6h

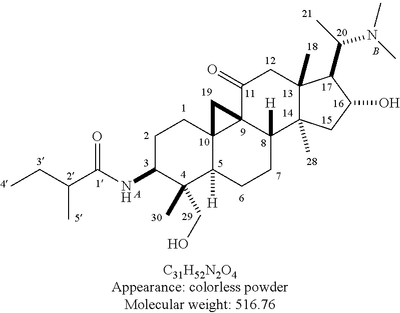

To a solution of 238 mg (0.55 mmol, 1.0 eq) of cycloxobuxidine-F 5 in 10 ml of methanol are added 113 mg (0.60 mmol, 1.1 eq) of 2-methylbutyric acid anhydride. After 5 hours of agitation at room temperature, the mixture is neutralized with several drops of a 10% sodium bicarbonate solution (pH=8), then alkalinized with 50 ml of a 10% ammonia solution (pH=10) and extracted with 3×40 ml of dichloromethane. The recombined organic phases are successively dried on anhydrous sodium sulfate and evaporated under a vacuum. The residue is chromatographed on a Merck activity II-III alumina column (eluent: 99.5/0.5 dichloromethane/methanol then 99/1 dichloromethane/methanol) to yield 238 mg of a mixture of two diastereoisomers (50/50) 6h in the form of a colorless powder after trituration in acetone with a yield of 84%.

MP (° C.): 255

Elemental analysis: calculated for C₃₁H₅₂N₂O₄: C, 72.05; H, 10.14; N, 5.42; O, 12.38. measured: C, 71.88; H, 10.34; N, 5.31; O, 11.87.

IR (CHCl₃) υ(cm⁻¹): 3436 (OH, NH); 1699 (C═O); 1661 and 1508 (CONH); 1082 (C—OH).

Mass (ESI): m/z: 517.4 ([M+H], 100); 518.4 (70).

HRMS (ESI) calculated for C₃₁H₅₃N₂O₄ m/z=517.4005; measured: 517.4001.

This is a racemic mixture of two diastereoisomers.

### Compound 6i: N-3-secpentyrylcycloxobuxidine-F 6i

### or

### (20S)-9,19-cyclo-4α-hydroxymethyl-4,14α-dimethyl-20-dimethylamino-3 (2-ethylbutyrylamino)-5α,9-pregnan-16α-ol-11-one 6i

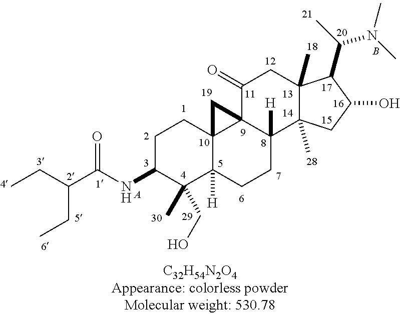

To a solution of 207 mg (0.48 mmol, 1.0 eq) of cycloxobuxidine-F 5 in 10 ml of methanol are added 113 mg (0.53 mmol, 1.1 eq) of 2-ethylbutyric acid anhydride. After 5 hours of agitation at room temperature, the mixture is neutralized with several drops of a 10% sodium bicarbonate solution (pH=8), then alkalinized with 50 ml of a 10% ammonia solution (pH=10) and extracted with 3×40 ml of dichloromethane. The recombined organic phases are successively dried on anhydrous sodium sulfate and evaporated under a vacuum. The residue is chromatographed on a Merck activity II-III alumina column (eluent: 99.5/0.5 dichloromethane/methanol then 99/1 dichloromethane/methanol) to yield 206 mg of 6i in the form of a colorless powder after trituration in acetone with a yield of 81%.

MP (° C.): 251

Elemental analysis: calculated for C₃₂H₅₄N₂O₄: C, 72.41; H, 10.25; N, 5.28; O, 12.06.

Measured: C, 72.14; H, 10.18; N, 5.23; O, 11.99.

IR (CHCl₃) υ(cm⁻¹): 3433 (OH, NH); 1690 (C═O); 1650 and 1511 (CONH); 1048 (C—OH).

Mass (ESI): m/z: 531.3 ([M+H], 100); 532.3 (10).

HRMS (ESI) calculated for C₃₂H₅₅N₂O₄ m/z=531.4162; measured: 531.4163.

### Compound 7a: 10(9->1)abeo-N-3-acetylcycloxobuxi-1(10)-enedine-F 7a

### or

### (20S)-10(9->1)abeo-3-acetylamino-4α-hydroxymethyl-4,14α-dimethyl-20-dimethylamino-5α,9-pregn-1(10)-en-16α-ol-11-one 7a

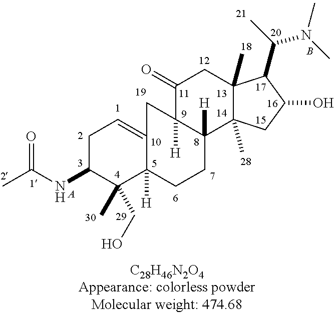

221 mg (0.46 mmol, 1.0 eq) of derivative N-3-acetylcycloxobuxidine-F 6a is heated at 240° C. under 0.03 mmHg in a ball tube oven. A product sublimates in hours. The solid sublimate is a mixture of the desired product 7a (98%) and dihydrooxazine 8a (2%). The sublimate is chromatographed on a Merck activity II-III alumina column (eluent: 99.7/0.3 dichloromethane/methanol) to yield 79.4 mg of 7a in the form of a colorless powder after trituration in acetone with a yield of 88%.

MP (° C.): 253

IR (CHCl₃) υ(cm⁻¹): 3435 (OH, NH); 1694 (C═O); 1654 and 1516 (CONH); 1043 (C—OH).

Mass (ESI): m/z: 475.3 ([M+H], 100); 476.3 (60).

HRMS (ESI) calculated for C₂₈H₄₇N₂O₄ m/z=475.3536; measured: 475.3548.

### Compound 7b: 10(9->1)abeo-N-3-benzoylcycloxobuxi-1(10)-enedine-F 7b

### or

### (20S)-10(9->1)abeo-3-benzoylamino-4α-hydroxymethyl-4,14α-dimethyl-20-dimethylamino-5α,9-pregn-1(10)-en-16α-ol-11-one 7b

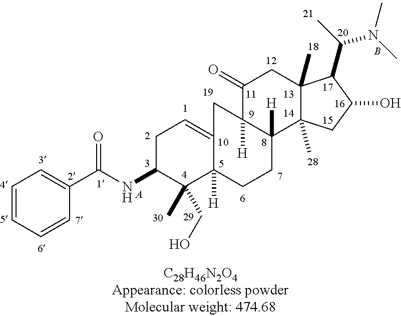

180 mg (0.33 mmol, 1.0 eq) of derivative N-3-benzoylcycloxobuxidine-F 6b are heated for 3 hours at 260° C. under 0.03 mmHg in a ball tube oven. No sublimate is obtained. The solid pellet is a mixture of the desired product 7b (95%) and dihydrooxazine 8b (5%). The pellet is chromatographed on a Merck activity II-III alumina column (eluent: 99.7/0.3 dichloromethane/methanol) to yield 126 mg of 7b in the form of a colorless powder after trituration in acetone with a yield of 70%.

MP (° C.): 289

IR (CHCl₃) υ(cm⁻¹): 3435 (OH, NH); 1693 (C═O); 1644 and 1519 (CONH); 1042 (C—OH).

Mass (ESI): m/z: 537.3 ([M+H], 100); 538.4 (80).

HRMS (ESI) calculated for C₃₃H₄₉N₂O₄ m/z=537.3692; measured: 537.3708.

### Compound 7c: 10(9->1)abeo-N-3-propionylcycloxobuxi-1(10)-enedine-F 7c

### or

### (20S)-10(9->1)abeo-4α-hydroxymethyl-4,14α-dimethyl-20-dimethylamino-3-propionylamino-5α,9-pregn-1(10)-en-16α-ol-11-one 7c

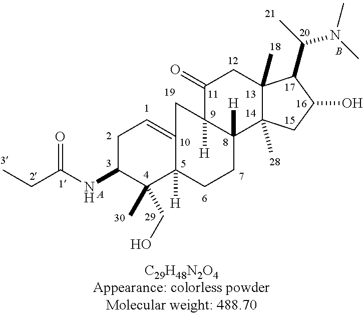

73.0 mg (0.15 mmol, 1.0 eq) of derivative N-3-propionylcycloxobuxidine-F 6c are heated at 240° C. under 0.03 mmHg in a ball tube oven. A sublimate is obtained in 3 hours. The solid sublimate is a mixture of the desired product 7c (64%) and dihydrooxazine 8c (36%). The sublimate is chromatographed on a Merck activity II-III alumina column (eluent: 99.7/0.3 dichloromethane/methanol) to yield 51.6 mg of 7c in the form of a colorless powder after trituration in acetone with a yield of 71%.

MP (° C.): 229.5

IR (CHCl₃) υ(cm⁻¹): 3433 (OH, NH); 1693 (C═O); 1653 and 1514 (CONH); 1044 (C—OH).

Mass (ESI): m/z: 489.2 ([M+H], 100); 490.3 (80).

HRMS (ESI) calculated for C₂₉H₄₉N₂O₄ m/z=489.3692; measured: 489.3687.

### Compound 7d: 10(9->1)abeo-N-3-cyclohexanecarboxylcycloxobuxi-1(10)-enedine-F 7d

### or

### (20S)-10(9->1)abeo-3-(cyclohexanecarboxyamino)-4α-hydroxymethyl-4,14α-dimethyl-20-dimethylamino-5α,9-pregn-1(10)-en-16α-ol-11-one 7d

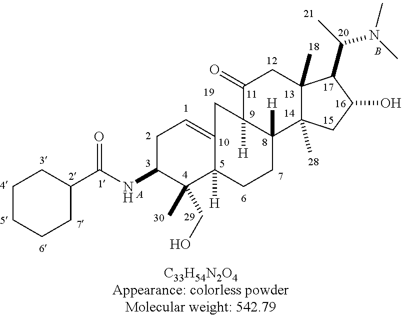

85.0 mg (0.15 mmol, 1.0 eq) of derivative N-3-cyclohexanecarboxylcycloxobuxidine-F 6d are heated at 260° C. under 0.03 mmHg in a ball tube oven. No sublimate is obtained after 3 hours. The pellet is a mixture of the desired product 7d (83%) and dihydrooxazine 8d (17%). The pellet is chromatographed on a Merck activity II-III alumina column (eluent: 99.7/0.3 dichloromethane/methanol) to yield 80.0 mg of 7d in the form of a colorless powder after trituration in acetone with a yield of 89%.

MP (° C.): 285

Elemental analysis: calculated for C₃₃H₅₄N₂O₄.0.5H₂O: C, 71.86; H, 9.98; N, 5.08. measured: C, 72.13; H, 9.91; N, 4.92.

IR (CHCl₃) υ(cm⁻¹): 3433 (OH, NH); 1693 (C═O); 1647 and 1512 (CONH); 1043 (C—OH).

Mass (ESI): m/z: 543.3 ([M+H], 100); 544.4 (70).

HRMS (ESI) calculated for C₃₃H₅₅N₂O₄ m/z=543.4162; measured: 543.4158.

### Compound 7e: 10(9->1)abeo-N-3-(S)-secbutyrylcycloxobuxi-1(10)-enedine-F 7e

### or

### (20S)-10(9->1)abeo-4α-hydroxymethyl-4,14α-dimethyl-20-dimethylamino-3 (S-2-methylbutyrylamino)-5α,9-pregn-1(10)-en-16α-ol-11-one 7e

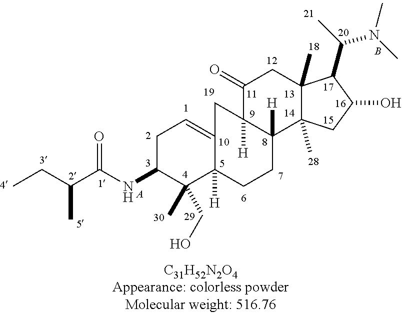

84.2 mg (0.16 mmol, 1.0 eq) of derivative N-3-(S)-secbutyrylcycloxobuxidine-F 6e is heated at 240° C. under 0.03 mmHg in a ball tube oven. A sublimate is obtained in 3 hours. The sublimate is a mixture of the desired product 7e (85%) and dihydrooxazine 8e (15%). The sublimate is chromatographed on a Merck activity II-III alumina column (eluent: 99.7/0.3 dichloromethane/methanol) to yield 80.0 mg of 7e in the form of a colorless powder after trituration in acetone with a yield of 95%.

MP (° C.): 219

Elemental analysis: calculated for C₃₁H₅₂N₂O₄: C, 72.05; H, 10.14; N, 5.42; O, 12.38. measured: C, 72.09; H, 10.06; N, 5.35; O, 12.32.

IR (CHCl₃) υ(cm⁻¹): 3433 (OH, NH); 1693 (C═O); 1649 and 1511 (CONH); 1043 (C—OH).

Mass (ESI): m/z: 517.3 ([M+H], 100); 518.4 (90).

HRMS (ESI) calculated for C₃₁H₅₃N₂O₄ m/z=517.4005; measured: 517.3980.

### Compound 7f: 10(9->1)abeo-N-3-pivalylcycloxobuxi-1(10)-enedine-F 7f

### or

### (20S)-10(9->1)abeo-4α-hydroxymethyl-4,14α-dimethyl-20-dimethylamino-3 (2,2-dimethylpropionylamino)-5α,9-pregn-1(10)-en-16α-ol-11-one 7f

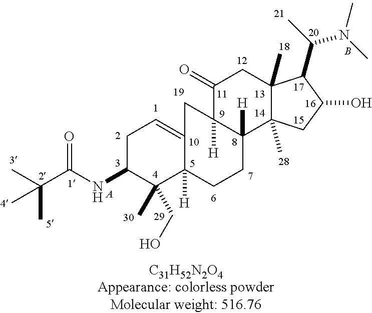

72.2 mg (0.14 mmol, 1.0 eq) of derivative N-3-pivalylcycloxobuxidine-F 6f are heated at 250° C. under 0.03 mmHg in a ball tube oven. A sublimate is obtained in 3 hours. The sublimate, containing 100% of the desired product 7f, is then chromatographed on a Merck activity II-III alumina column (eluent: 99.7/0.3 dichloromethane/methanol) to yield 63.4 mg of 7f in the form of a colorless powder after trituration in acetone with a yield of 88%.

MP (° C.): 270

Elemental analysis: calculated for C₃₁H₅₂N₂O₄: C, 72.05; H, 10.14; N, 5.42; O, 12.38. measured C, 71.91; H, 10.16; N, 5.37; O, 12.11.

IR (CHCl₃) υ(cm⁻¹): 3454 (OH, NH); 1693 (C═O); 1639 and 1515 (CONH); 1044 (C—OH).

Mass (ESI): m/z: 517.3 ([M+H], 100); 518.4 (60).

HRMS (ESI) calculated for C₃₁H₅₃N₂O₄ m/z=517.4005; measured: 517.3990.

### Compound 7g: 10(9->1)abeo-N-3-phenylacetylcycloxobuxi-1(10)-enedine-F 7g

### or

### (20S)-10(9->1)abeo-4α-hydroxymethyl-4,14α-dimethyl-20-dimethylamino-3 (phenylacetylamino)-5α,9-pregn-1(10)-en-16α-ol-11-one 7g

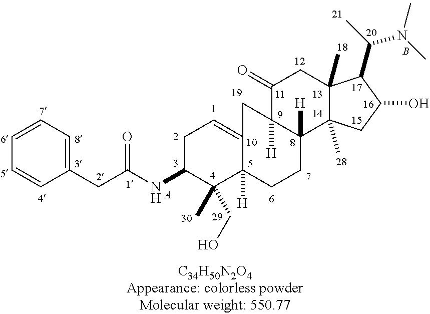

101.8 mg (0.18 mmol, 1.0 eq) of derivative N-3-phenylacetylcycloxobuxidine-F 6g are heated at 270° C. under 0.03 mmHg in a ball tube oven. No sublimate is obtained after three hours. The pellet is a mixture of the desired product 7g (74%) and dihydrooxazine 8g (26%). The pellet is chromatographed on a Merck activity II-III alumina column (eluent: 99.7/0.3 dichloromethane/methanol) to yield 40.6 mg of 7g in the form of a colorless powder after trituration in acetone with a yield of 55%.

MP (° C.): 261

IR (CHCl₃) υ(cm⁻¹): 3411 (OH, NH); 1693 (C═O); 1648 and 1519 (CONH); 1044 (C—OH).

Mass (ESI): m/z: 551.3 ([M+H], 100); 552.3 (70).

HRMS (ESI) calculated for C₃₄H₅₁N₂O₄ m/z=551.3817; measured: 551.3849.

### Compound 7h: 10(9->1)abeo-N-3-secbutyrylcycloxobuxi-1(10)-enedine-F 7h

### or

### (20S)-10(9->1)abeo-4α-hydroxymethyl-4,14α-dimethyl-20-dimethylamino-3 (2-methylbutyrylamino)-5α,9-pregn-1(10)-en-16α-ol-11-one 7h

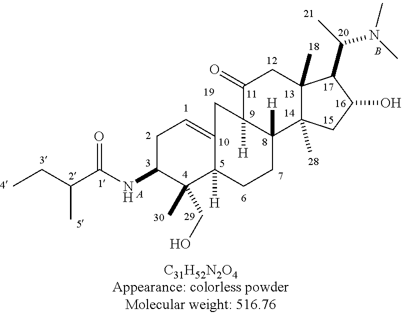

40.0 mg (0.07 mmol, 1.0 eq) of derivative N-3-secbutyrylcycloxobuxidine-F 7h are heated at 250° C. under 0.08 mmHg in a ball tube oven. A sublimate is obtained in 3 hours. The sublimate is a mixture of the desired product 7h (80%) and dihydrooxazine 8h (20%). The sublimate is chromatographed on a Merck activity II-III alumina column (eluent: 99/1 dichloromethane/methanol) to yield 12.0 mg of 7h in the form of a colorless powder after trituration in acetone with a yield of 30%.

IR (CHCl₃) υ(cm⁻¹): 3433 (OH, NH); 1693 (C═O); 1650 and 1511 (CONH); 1012 (C—OH).

Mass (ESI): m/z: 517.3 ([M+H], 100); 518.4 (40); 499.4 (5).

HRMS (ESI) calculated for C₃₁H₅₃N₂O₄ m/z=517.4005; measured: 517.4011.

This is a racemic mixture of two diastereoisomers.

### Compound 7i: 10(9->1)abeo-N-3-secpentyrylcycloxobuxi-1(10)-enedine-F 7i

### or

### (20S)-10(9->1)abeo-4α-hydroxymethyl-4,14α-dimethyl-20-dimethylamino-3 (2-ethylbutyrylamino)-5α,9-pregn-1(10)-en-16α-ol-11-one 7i

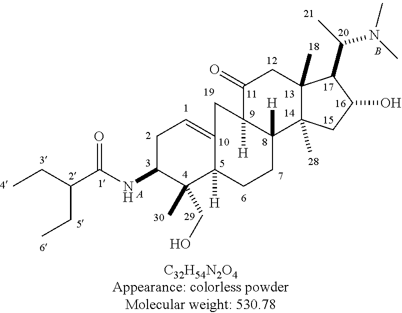

40.0 mg (0.08 mmol, 1.0 eq) of derivative N-3-secpentyrylcycloxobuxidine-F 7i are heated at 250° C. under 0.08 mmHg in a ball tube oven. A sublimate is obtained in 3 hours. The sublimate is a mixture of the desired product 7i (90%) and dihydrooxazine 8i (10%). The sublimate is chromatographed on a Merck activity II-III alumina column (eluent: 99/1 dichloromethane/methanol) to yield 12.0 mg of 7i in the form of a colorless powder after trituration in acetone with a yield of 31%.

IR (CHCl₃) υ(cm⁻¹): 3433 (OH, NH); 1693 (C═O); 1649 and 1510 (CONH); 1042 (C—OH).

Mass (ESI): m/z: 531.3 ([M+H], 100); 532.4 (50).

HRMS (ESI) calculated for C₃₂H₅₅N₂O₄ m/z=531.4162; measured: 531.4173.

### Compound 8a: (20S)-10(9->1)abeo-4,14α-dimethyl-20-dimethylamino(2′-methyl-4′,4′-dihydro[3′,1′]oxazin)-5α,9-pregn-1(10)-en-16α-ol-11-one 8a

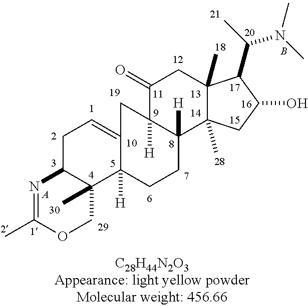

171 mg (0.36 mmol, 1.0 eq) of derivative N-3-acetylcycloxobuxidine-F 6a are dissolved in a minimum of dichloromethane (2 ml) and then 1059 mg (1.80 mmol, 5.0 eq) of a 25% tetraethylammonium hydroxide solution in methanol are added. The solution is yellow in color. The solvents are evaporated under reduced pressure. The dark red solid is dried then heated at 240° C. under 0.03 mmHg in a ball tube oven. A sublimate is obtained in 3 hours. The pellet melts and yellows. The sublimate is recrystallized in acetone and 125 mg of 8a in the form of a light yellow powder are isolated with a yield of 76%.

MP (° C.): 284

Elemental analysis: calculated for C₂₈H₄₄N₂O₃: C, 73.64; H, 9.71; N, 6.13. measured C, 73.25; H, 9.56; N, 5.98.

IR (CHCl₃) υ(cm⁻¹): 3331 (OH, NH); 1694 (C═O); 1672 (C═N); 1039 (C—OH).

Mass (ESI): m/z: 457.4 ([M+H], 100); 458.4 (5).

HRMS (ESI) calculated for C₂₈H₄₅N₂O₃ m/z=457.3430; measured: 457.3470.

### Compound 8b: (20S)-10(9->1)abeo-4,14α-dimethyl-20-dimethylamino(2′-phenyl-4′,4′-dihydro[3′,1′]oxazin)-5α,9-pregn-1(10)-en-16α-ol-11-one 8b

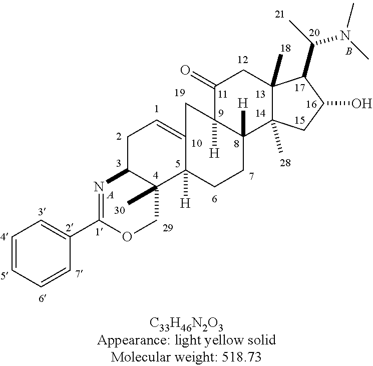

118 mg (0.22 mmol, 1.0 eq) of derivative N-3-benzoylcycloxobuxidine-F 6b are dissolved in a minimum of dichloromethane (2 ml) and then 648 mg (1.10 mmol, 5.0 eq) of a 25% tetraethylammonium hydroxide solution in methanol are added. The solution is colorless. The solvents are evaporated under reduced pressure. The dark red solid is dried then heated at 275° C. under 0.03 mmHg in a ball tube oven. No product sublimates in hours. The pellet melts and yellows. The product contained in the pellet is recrystallized in acetone and 97 mg of 8b in the form of a light yellow powder are isolated with a yield of 85%.

Elemental analysis: calculated for C₃₃H₄₆N₂O₃: C, 73.64; H, 9.71; N, 6.13. measured: C, 73.25; H, 9.56; N, 5.98.

IR (CHCl₃) υ(cm⁻¹): 3376 (OH, NH); 1694 (C═O); 1650 (C═N); 1027 (C—OH).

Mass (ESI): m/z: 519.4 ([M+H], 100); 520.4 (10).

HRMS (ESI) calculated for C₃₃H₄₇N₂O₃ m/z=519.3587; measured: 519.3610.

### Compound 8c: (20S)-10(9->1)abeo-4,14α-dimethyl-20-dimethylamino(2′-ethyl-4′,4′-dihydro[3′,1′]oxazin)-5α,9-pregn-1(10)-en-16α-ol-11-one 8c

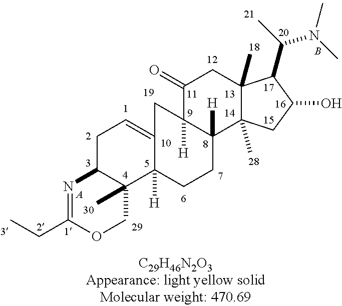

83 mg (0.17 mmol, 1.0 eq) of N-3-propionylcycloxobuxidine-F 6c are heated at 240° C. under 0.03 mmHg in a ball tube oven. A sublimate is obtained in 3 hours. The sublimate consists of a mixture of the opening product of cyclopropane 7c (64%) and dihydrooxazine 8c (36%). To a solution of the sublimate in 2 ml of dichloromethane are added 500 mg (0.85 mmol, 5.0 eq) of a 25% tetraethylammonium hydroxide solution in methanol. After evaporation under a vacuum, the red residue heated at 240° C. under 0.03 mmHg for 3 hours yields 75 mg of 8c in the form of a light yellow crystallized sublimate with a yield of 94%.

MP (° C.): 282

Elemental analysis: calculated for C₂₉H₄₆N₂O₃.0.25H₂O: C, 73.34; H, 9.79; N, 5.90. measured: C, 73.08; H, 9.74; N, 5.91.

IR (CHCl₃) υ(cm⁻¹): 3363 (OH, NH); 1694 (C═O); 1667 (C═N); 1040 (C—OH).

Mass (ESI): m/z: 471.3 ([M+H], 100); 472.4 (70).

HRMS (ESI) calculated for C₂₉H₄₇N₂O₃ m/z=471.3587; measured: 471.3577.

### Compound 8d: (20S)-10(9->1)abeo-4,14α-dimethyl-20-dimethylamino(2′-cyclohexyl-4′,4′-dihydro[3′,1′]oxazin)-5α,9-pregn-1(10)-en-16α-ol-11-one 8d

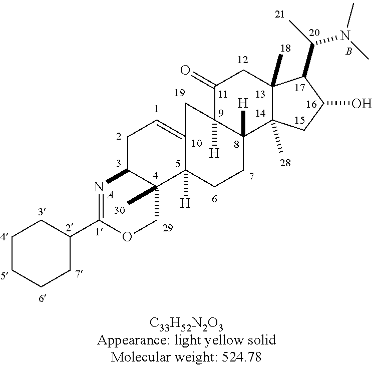

225 mg (0.41 mmol, 1.0 eq) of N-3-cyclohexanecarboxylcycloxobuxidine-F 6d are heated at 260° C. under 0.03 mmHg in a ball tube oven. No sublimate is obtained in 3 hours. The product contained in the pellet consists of a mixture of the opening product of cyclopropane 7d (83%) and dihydrooxazine 8d (17). To a solution of the residue comprised of the mixture previously obtained in 2 ml of dichloromethane are added 1085 mg (1.84 mmol, 5.0 eq) of a 25% tetraethylammonium hydroxide solution in methanol. After evaporation under a vacuum, the red residue heated at 260° C. under 0.03 mmHg for 3 hours yields 150 mg of 8d in the form of a light yellow solid in the pellet with a yield of 69%.

Elemental analysis: calculated for C₃₃H₅₂N₂O₃: C, 75.53; H, 9.91; N, 5.14. measured: C, 75.74; H, 9.91; N, 5.14.

IR (CHCl₃) υ(cm⁻¹): 3433 (OH, NH); 1693 (C═O); 1659 (C═N); 1040 (C—OH).

Mass (ESI): m/z: 525.3 ([M+H], 100); 526.4 (40); 543.4 (50).

HRMS (ESI) calculated for C₃₃H₅₃N₂O₃ m/z=525.4056; measured: 525.4041.

### Compound 8e: (20S)-10(9->1)abeo-4,14α-dimethyl-20-dimethylamino(2′-(S-1-methylpropyl)-4′,4′-dihydro[3′,1′]oxazin)-5α,9-pregn-1(10)-en-16α-ol-11-one 8e

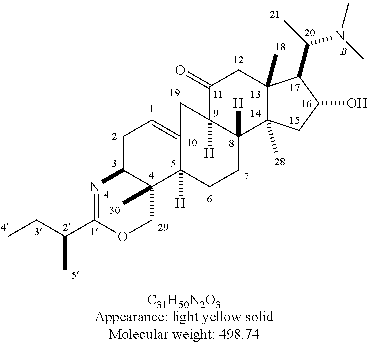

63 mg (0.12 mmol, 1.0 eq) of N-3-(S)-secbutyrylcycloxobuxidine-F 6e is heated at 253° C. under 0.04 mmHg in a ball tube oven. A sublimate is obtained in 3 hours. The pellet consists of the opening product of cyclopropane 7e while the sublimate is a mixture of product 7e (85%) and dihydrooxazine 8e (15%). To a solution comprised of the pellet and sublimate previously obtained in 2 ml of dichloromethane are added 364 mg (0.60 mmol, 5.0 eq) of a 25% tetraethylammonium hydroxide solution in methanol. After evaporation under a vacuum, the red residue heated at 253° C. under 0.04 mmHg for 3 hours yields 49 mg of 8e in the form of a sublimated light yellow solid with a yield of 80%.

IR (CHCl₃) υ(cm⁻¹): 3433 (OH, NH); 1694 (C═O); 1659 (C═N); 1040 (C—OH).

Mass (ESI): m/z: 499.4 ([M+H], 100); 500.4 (50).

HRMS (ESI) calculated for C₃₁H₅₁N₂O₃ m/z=499.3900; measured: 499.3917.

### Compound 8f: (20S)-10(9->1)abeo-4,14α-dimethyl-20-dimethylamino(2′-(2,2-dimethylethyl)-4′,4′-dihydro[3′,1′]oxazin)-5α,9-pregn-1(10)-en-16α-ol-11-one 8f

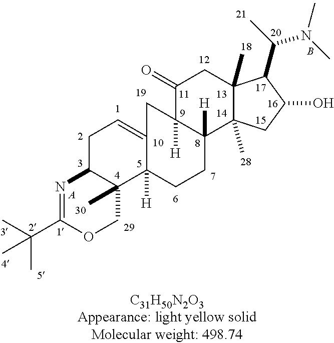

73 mg (0.14 mmol, 1.0 eq) of N-3-pivalylcycloxobuxidine-F 6f are heated at 250° C. under 0.03 mmHg in a ball tube oven. No sublimate is obtained. The pellet is 100% comprised of the opening product of cyclopropane 7f. To a solution of the sublimate in 2 ml of dichloromethane are added 416 mg (0.70 mmol, 5.0 eq) of a 25% tetraethylammonium hydroxide solution in methanol. After evaporation under a vacuum, the red residue heated at 250° C. under 0.03 mmHg for 3 hours yields 60.4 mg of 8f in the form of a sublimated light yellow solid with a yield of 85%.

IR (CHCl₃) υ(cm⁻¹): 3373 (OH, NH); 1693 (C═O); 1655 (C═N); 1015 (C—OH).

Mass (ESI): m/z: 499.4 ([M+H], 100); 500.4 (40).

HRMS (ESI) calculated for C₃₁H₅₁N₂O₃ m/z=499.3900; measured: 499.3868.

### Compound 8g: (20S)-10(9->1)abeo-4,14α-dimethyl-20-dimethylamino-(2′-(1-benzyl)-4′,4′-dihydro[3′,1′]oxazine)-5α,9-pregn-1(10)-en-16α-ol-11-one 8g

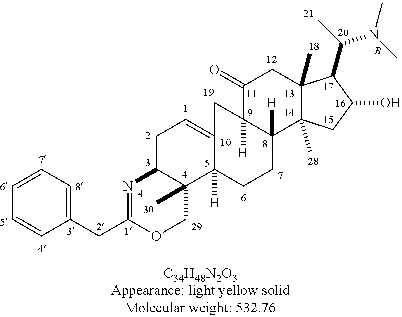

240 mg (0.43 mmol, 1.0 eq) of N-3-phenylacetylcycloxobuxidine-F 6g are heated at 260° C. under 0.03 mmHg in a ball tube oven. No sublimate is obtained in 3 hours. The product contained in the pellet consists of a mixture of the opening product of cyclopropane 7g (74%) and dihydrooxazine 8g (26%). To a solution comprised of the mixture previously obtained in 2 ml of dichloromethane are added 926 mg (1.57 mmol, 5.0 eq) of a 25% tetraethylammonium hydroxide solution in methanol. After evaporation under a vacuum, the red residue heated at 260° C. under 0.03 mmHg for 3 hours yields 125 mg of 8g in the form of a light yellow solid in the pellet with a yield of 55%.

Elemental analysis: calculated for C₃₄H₄₈N₂O₃ 1.5H₂O: C, 72.98; H, 9.12; N, 5.00. measured C, 73.11; H, 8.98; N, 4.74.

IR (CHCl₃) υ(cm⁻¹): 3411 (OH, NH); 1694 (C═O); 1665 (C═N); 1039 (C—OH).

Mass (ESI): m/z: 533.3 ([M+H], 100); 534.4 (50).

HRMS (ESI) calculated for C₃₄H₄₉N₂O₃ m/z=533.3743; measured: 533.3747.

### Compound 8h: (20S)-10(9->1)abeo-4,14α-dimethyl-20-dimethylamino(2′-(1-methylpropyl)-4′,4′ dihydro[3′,1′]oxazin)-5α,9-pregn-1(10)-en-16α-ol-11-one 8h

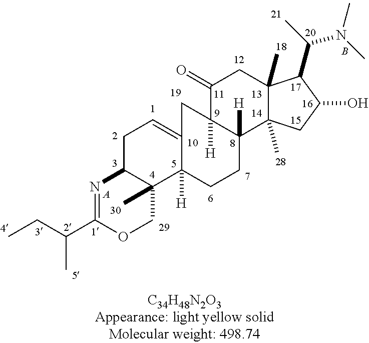

105 mg (0.20 mmol, 1.0 eq) of N-3-secbutyrylcycloxobuxidine-F 6f are heated at 257° C. under 0.10 mmHg in a ball tube oven. A sublimate is obtained in 3 hours. The sublimate consists of a mixture of the desired product 7f (85%) and dihydrooxazine 8f (15%). To a solution of the residue (pellet and sublimate) in 2 ml of dichloromethane are added 597 mg (1.01 mmol, 5.0 eq) of a 25% tetraethylammonium hydroxide solution in methanol. After evaporation under a vacuum, the red residue heated at 260° C. under 0.1 mmHg for 5 hours yields 30.0 mg of a mixture of two diastereoisomers (50/50) 8f in the form of a sublimated light yellow solid with a yield of 30%.

IR (CHCl₃) υ(cm⁻¹): 3361 (OH); 1695 (C═O); 1651 (C═N); 1097 (C—OH).

Mass (ESI): m/z: 499.3 ([M+H], 100); 500.4 (20); 559.4 (40); 250.2 (20).

HRMS (ESI) calculated for C₃₁H₅₁N₂O₃ m/z=499.3900; measured: 499.3900.

This is a racemic mixture of two diastereoisomers.

### Compound 8i: (20S)-10(9->1)abeo-4,14α-dimethyl-20-dimethylamino(2′-(1-ethylpropyl)-4′,4′-dihydro[3′,1′]oxazin)-5α,9-pregn-1(10)-en-16α-ol-11-one 8i

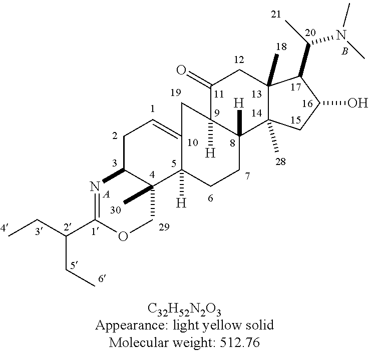

85 mg (0.14 mmol, 1.0 eq) of N-3-secpentyrylcycloxobuxidine-F 6i are heated at 250° C. under 0.08 mmHg in a ball tube oven. The starting product partially sublimates in 3 hours. The sublimate consists of a mixture of the opening product of cyclopropane 7i (90%) and dihydrooxazine 8i (10%). To a solution of the residue (pellet and sublimate) in 2 ml of dichloromethane are added 472 mg (0.80 mmol, 5.0 eq) of a 25% tetraethylammonium hydroxide solution in methanol. After evaporation under a vacuum, the red residue heated at 260° C. under 0.1 mmHg for 3 hours yields 40.0 mg of 8i in the form of a sublimated light yellow solid with a yield of 49%.

IR (CHCl₃) υ(cm⁻¹): 3287 (OH); 1694 (C═O); 1658 (C═N); 1167 (C—OH).

Mass (ESI): m/z: 513.3 ([M+H], 100); 514.3 (80); 531.4 (10); 257.2 (20); 257.7 (10).

HRMS (ESI) calculated for C₃₂H₅₃N₂O₃ m/z=513.4056; measured: 513.4045.

4.2. Syntheses of Compounds 9, 11 and 12 (Tetrahydrooxazines)

1′,2′,4′,4′-tetrahydrooxazines 9 and 10 were prepared in a novel fashion. The condensation of isobutyraldehyde with cycloxobuxidine-F 5 in 1,4-dioxane at 50° C. leads to the formation of 9 with a yield of 30% (diagram 4).

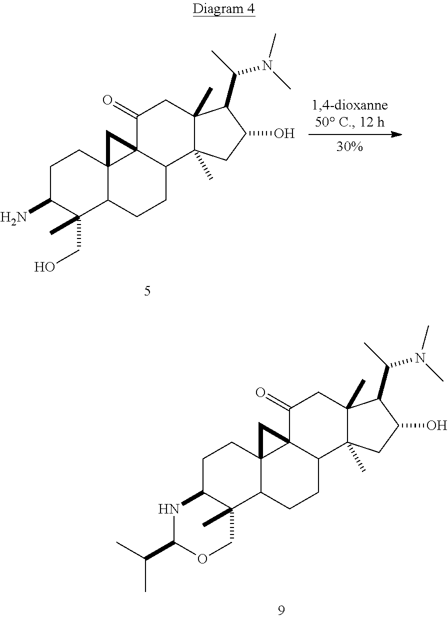

Compound 11 is obtained in two steps from amide 3. In acid medium, in the presence of 2 N sulfuric acid and methanol, the isobutyraldehyde group migrates on the primary alcohol to yield the corresponding ester 10 which is then hydrolyzed (diagram 5).

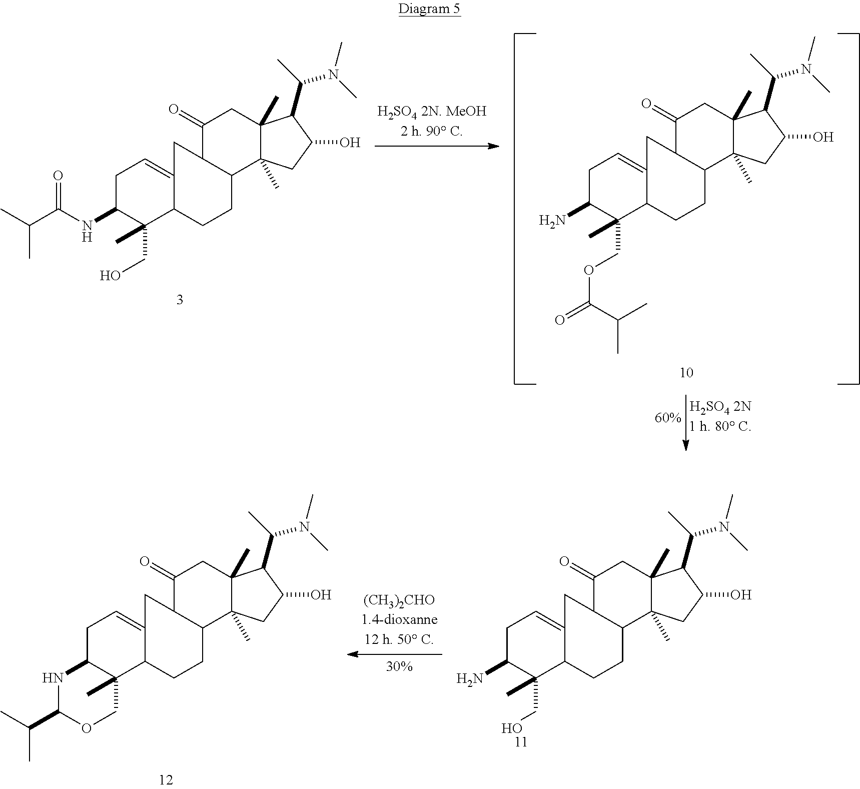

### Compound 9: (20S)-9,19-cyclo-4,14α-dimethyl-20-dimethylamino(2′β-isopropyl-1′,2′,4′,4′-tetrahydro[3′,1′]oxazin)-5α,9-pregnan-16α-ol-11-one 9

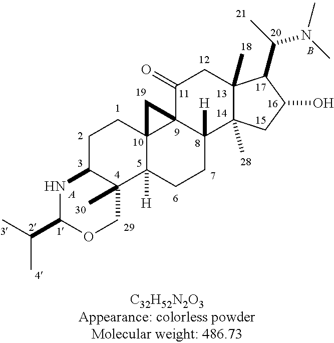

To a suspension of 87 mg (0.20 mmol, 1.0 eq) of derivative cycloxobuxidine-F 5 in 15 ml of 1,4-dioxane are added 37 ml (0.40 mmol, 2.0 eq) of isobutyraldehyde. After 12 hours of agitation at 50° C., the reaction mixture is cooled to room temperature and evaporated under reduced pressure. The liquor obtained is alkalinized with 30 ml of a 10% sodium bicarbonate solution and extracted with 3×40 ml of dichloromethane. The recombined organic phases are successively dried on anhydrous sodium sulfate and evaporated under a vacuum. The crude product (105 mg) is triturated in diethyl ether. After filtration, washing and drying under reduced pressure, 28 mg of 9 in the form of a colorless powder are obtained with a yield of 30%.

IR (CHCl₃) υ(cm⁻¹): 3319 (OH, NH); 1663 (C═O); 1096 (C—OH).

Mass (ESI): m/z: 487.3 ([M+H], 100); 488.4 (30).

HRMS (ESI) calculated for C₃₀H₅₁N₂O₃ m/z=487.3900; measured: 487.3897.

### Compound 11: 10(9->1)abeocycloxobuxi-1(10)-enedine-F 11

### or

### (20S)-10(9->1)abeo-3-amino-4α-hydroxymethyl-4,14α-dimethyl-20-dimethylamino-5α,9-pregn-1(10)-en-16α-ol-11-one 11

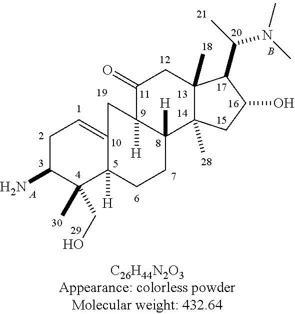

250 mg (0.50 mmol, 1.0 eq) of N-3-isobutyrylcycloxobuxidine-F 2 are heated at 245° C. under 0.1 mmHg in a ball tube oven. A sublimate is obtained in 3 hours. The sublimate is a mixture of the opening product of cyclopropane 3 (18%) and dihydrooxazine 1 (82%). The remaining pellet consists of the opening product of cyclopropane 3. To a solution of 250 mg of the residue comprised of the pellet and sublimate previously obtained in 15 ml of methanol is added dropwise 36 ml of a 2 N sulfuric acid aqueous solution at room temperature. After 2 hours of agitation at 90° C., the reaction mixture is cooled to room temperature and evaporated under reduced pressure at greater than 25° C. The liquor obtained is heated for 1 hour at 80° C. It is yellow in color. The reaction mixture is alkalinized with 40 ml of a 10% ammonia solution and extracted with 3×40 ml of dichloromethane. The recombined organic phases are successively dried on anhydrous sodium sulfate and evaporated under a vacuum. The crude product is triturated in ethyl acetate. After filtration, washing and drying under reduced pressure, 128 mg of 11 in the form of a colorless solid are obtained with a yield of 60% whose spectroscopic characteristics are identical to those described by Herlem-Gaulier, D. et al. (1968).

MP (° C.): 266

Elemental analysis: calculated for C₂₆H₄₄N₂O₃.0.5H₂O: C, 70.71; H, 10.27; N, 6.34; O, 12.68. measured: C, 70.67; H, 10.31; N, 6.19; O, 12.08.

IR (CHCl₃) υ(cm⁻¹): 3383 (OH, NH); 1693 (C═O); 1602 (C—NH); 1038 (C—OH).

Mass (ESI): m/z: 433.3 ([M+H], 100).

HRMS (ESI) calculated for C₂₆H₄₅N₂O₃ m/z=433.3430; measured: 433.3415.

### Compound 12: (20S)-10(9->1)abeo-4,14α-dimethyl-20-dimethylamino(2′β-isopropyl-1′,2′,4′,4′-tetrahydro[3′,1′]oxazin)-5α,9-pregn-1(10)-en-16α-ol-11-one 12

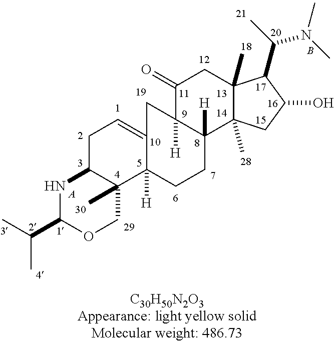

To a suspension of 85 mg (0.20 mmol, 1.0 eq) of derivative 10(9->1)abeocycloxobuxi-1(10)-enedine-F 11 in 10 ml of 1,4-dioxane is added 37 ml (0.40 mmol, 2.0 eq) of isobutyraldehyde. After 12 hours of agitation at 50° C., the reaction mixture is cooled to room temperature and evaporated under reduced pressure. The liquor obtained is alkalinized with 40 ml of a 10% sodium bicarbonate solution and extracted with 3×40 ml of dichloromethane. The recombined organic phases are successively dried on anhydrous sodium sulfate and evaporated under a vacuum. The crude product (94 mg) is triturated in diethyl ether. After filtration, washing and drying under reduced pressure, 40 mg of 12 in the form of a colorless powder are obtained with a yield of 42% whose spectroscopic characteristics are identical to those described by Herlem-Gaulier, D. et al. (1968).

MP (° C.): 253

Elemental analysis: calculated for C₃₀H₅₀N₂O₃: C, 74.03; H, 10.35; N, 5.76; O, 9.86. measured: C, 73.38; H, 10.54; N, 5.63; O, 9.93.

IR (CHCl₃) υ(cm⁻¹): 3442 (OH, NH); 1693 (C═O); 1044 (C—OH).

Mass (ESI): m/z: 487.4 ([M+H], 100).

HRMS (ESI) calculated for C₃₀H₅₁N₂O₃ m/z=487.3900; measured: 487.3909.

### Example 5

### Synthesis of Compounds 13 to 32

Synthesis of Compounds Having a Modified Function at Position 29:

Compounds having an imine or ester function at position 29 were prepared from alcohol 2.

Compound 2 in the presence of benzyl bromide yielded monobenzyl compound 27 at position 29. Monoesterified compounds 13 and 17 on the alcohol function at position 16 were obtained by the action of acetic or pivalic anhydride on compound 2. The primary alcohol function of compounds 13 and 17 is oxidized into corresponding aldehyde 14 and 18. These aldehydes yield imines 16 and 19 by reaction with benzylamine.

It should be noted that the two primary and secondary alcohol functions of compound 2 are esterified by the action of pivalyl chloride to form compound 20 (diagram 6).

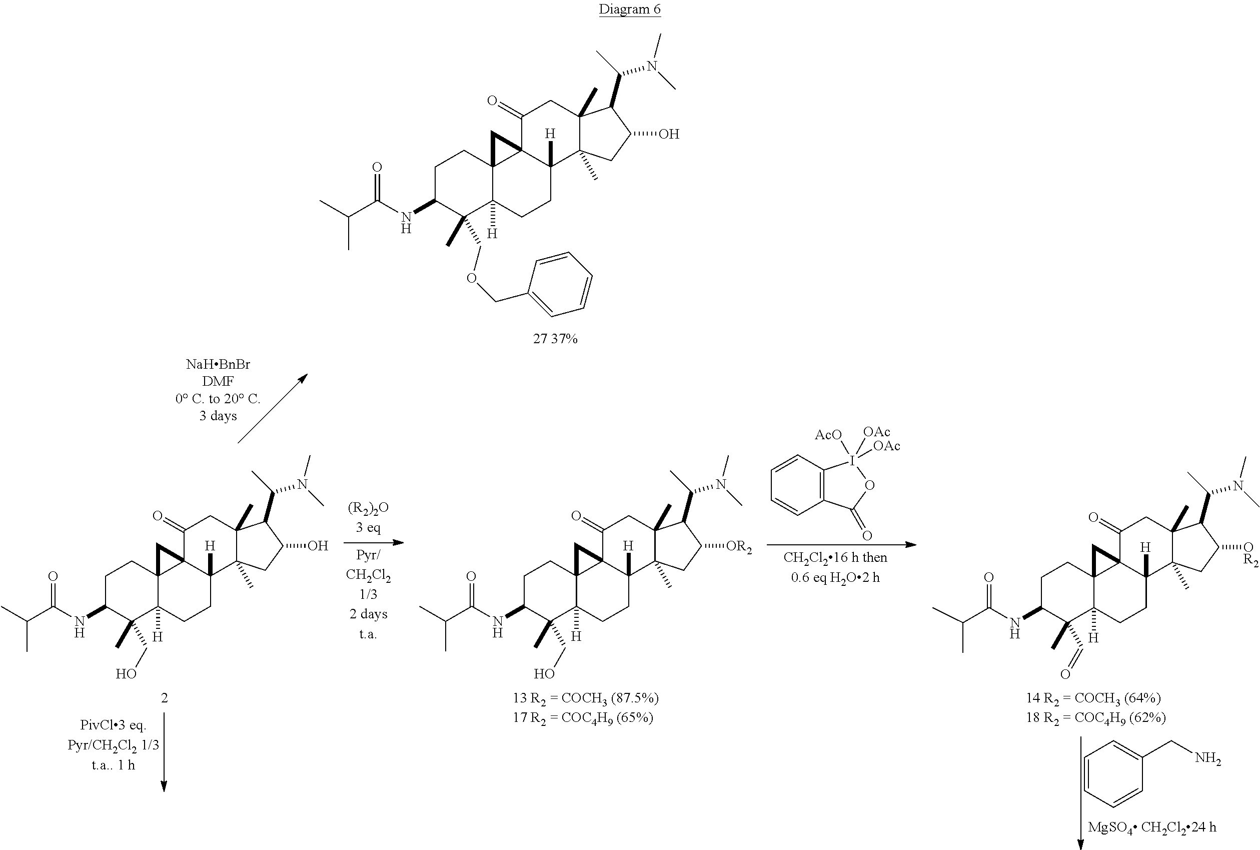

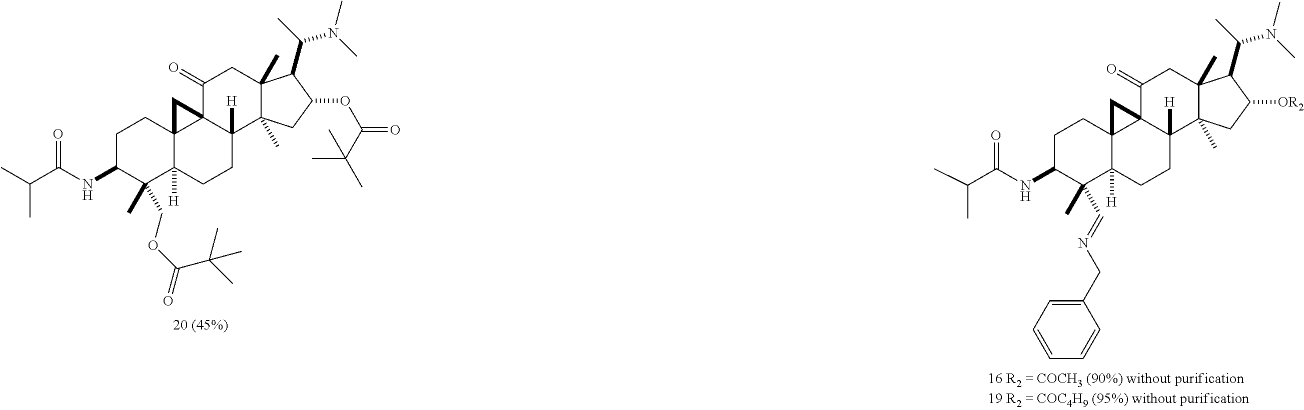

The primary alcohol function of esters 13 and 17 was transformed into sulfonate by the action of tosyl chloride or methanesulfonyl chloride to form products 22 to 25 after prior protection of the tertiary amine function at position 20 in the form of salt by reaction with p-toluenesulfonic acid (diagram 7).

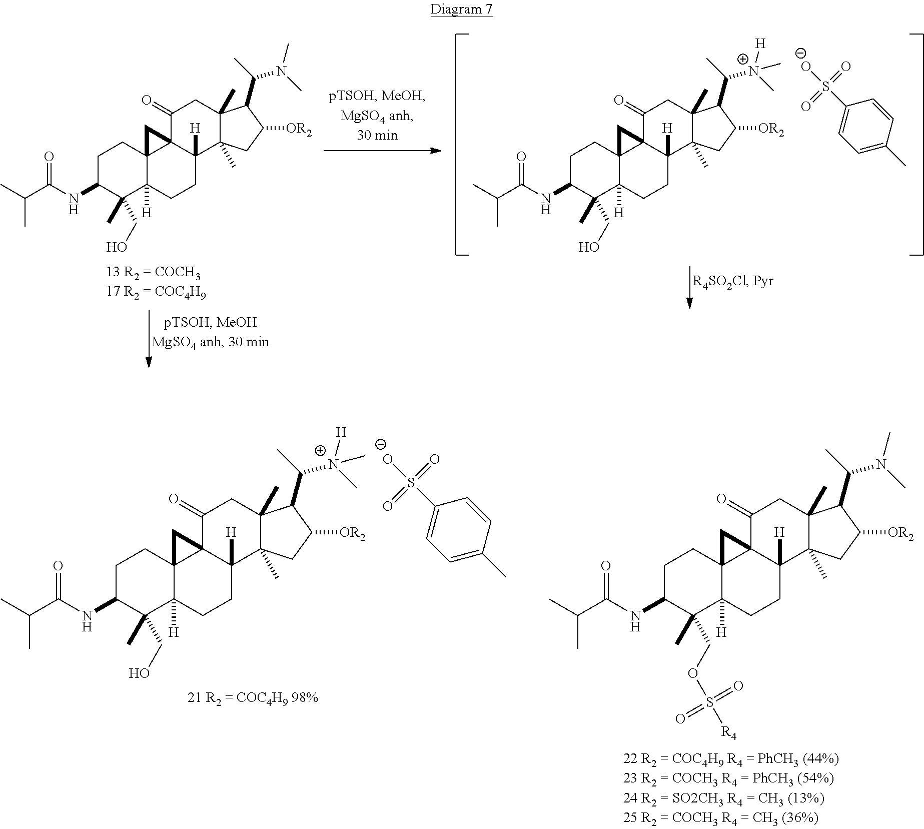

Reduction of the Ketone into 11.

The ketone function of compound 5 was reduced diastereoselectively by the action of LiAlH₄ to form alcohol 28 (diagram 8).

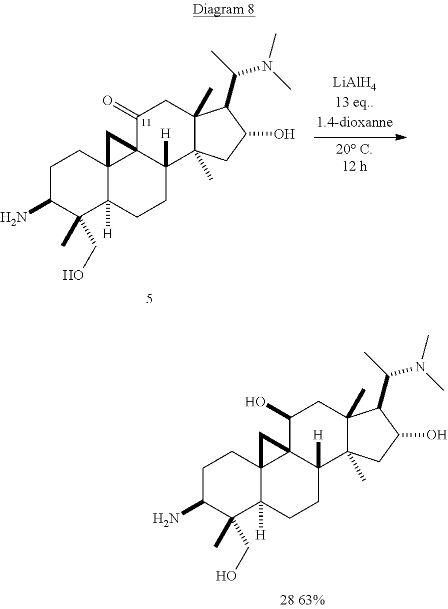

### Synthesis of Nor-N-isobutyrylcyclobuxidine-F 2 30

Compound 30 was prepared in two steps from N-isobutyrylcycloxobuxidine-F 2. The nitrogen atom of 2 is oxidized into N-oxide by the action of p-chloroperbenzoic acid to lead to compound 29 with a yield of 79%. This compound then undergoes non-classical demethylation in the presence of iron salts to yield demethylated compound 30 with a yield of 76% (diagram 9).

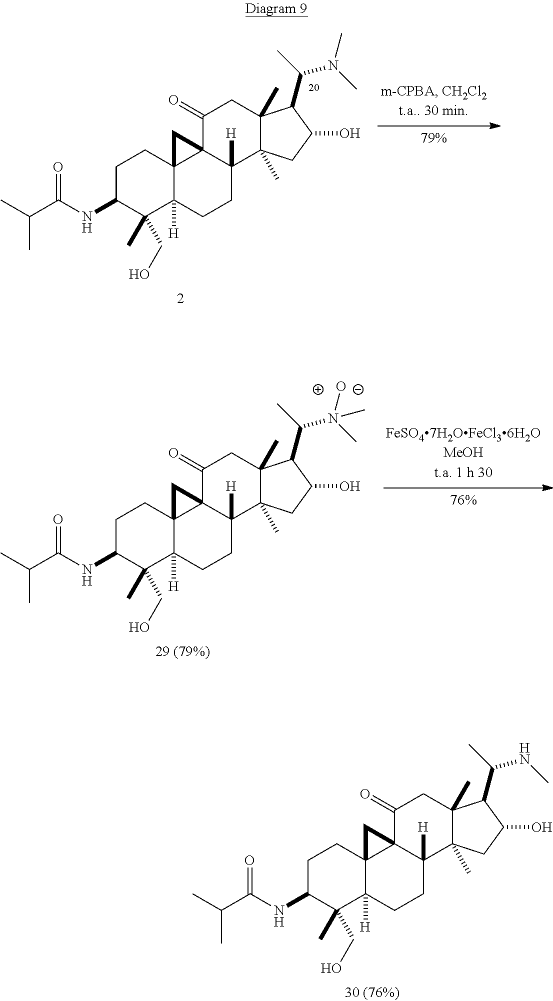

Synthesis of an Isomer of 1

The synthesis of an isomer compound of 1 wherein an α-ketone, β-ethylene function is present was carried out in two steps. The first step is a cyclopropane opening reaction in the presence of a Lewis acid. The second step consists of the formation of the dihydrooxazine ring. The enone 31 thus obtained is then heated at high temperature in the presence of triethylammonium hydroxide to yield pentacyclic compound 32.

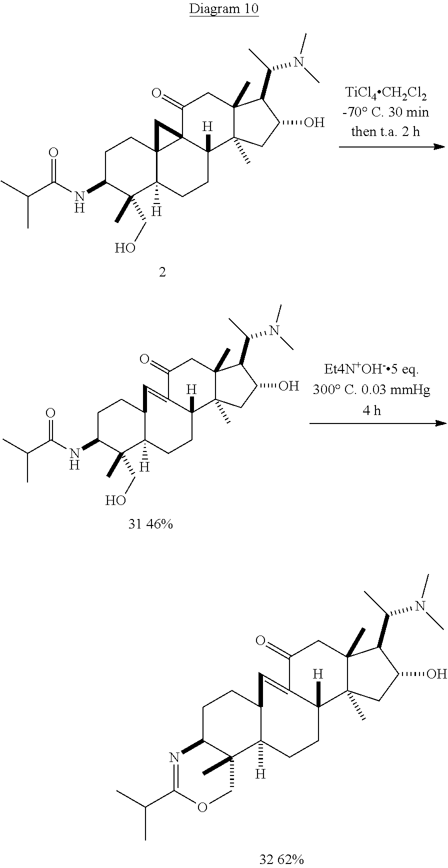

### Compound 13: (20S)-16α-acetyl-3-isobutyrylamino-9,19-cyclo-4α-hydroxymethyl-4,14α-dimethyl-20-dimethylamino-5α,9-pregnan-11-one 13

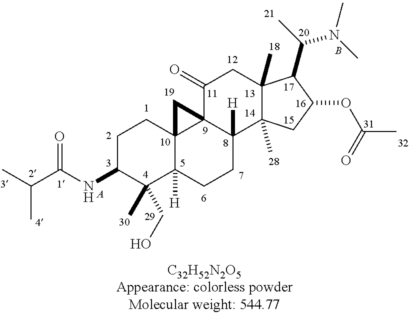

To a solution of 1007 mg (2.0 mmol, 1.0 eq) of N-3-isobutyrylcycloxobuxidine-F 2 in 100 ml of dichloromethane and 30 ml of pyridine, 568 μl (6.0 mmol, 3.0 eq) of acetic anhydride are added. The solution is light yellow. After 2 days of agitation at room temperature, the mixture is co-evaporated in the presence of 1,2-dichloroethane.

The residue thus obtained is purified by silica gel chromatography (eluent: 9/1 dichloromethane/methanol) to yield 820 mg of 13 in the form of a colorless powder after recrystallization in acetone with a yield of 75%.

MP (° C.): 312

Elemental analysis: calculated for C₃₂H₅₂N₂O₅%: C, 70.55; H, 9.62; N, 5.14; O, 14.68. actual %: C, 70.64; H, 9.84; N, 4.89; O, 14.48.

IR (diamond) υ(cm⁻¹): 3276 (OH, NH); 1729 (C═O ester) and 1667 (C═O); 1632 and 1555 (CONH); 1044 (C—OH).

Mass (ESI): m/z: 545.4 ([M+H], 100); 546.4 (5); 503.4 (60).

HRMS (ESI) calculated for C₃₂H₅₃N₂O₅ m/z=545.3954; actual: 545.3960.

### Compound 14: (20S)-16α-acetyl-3-isobutyrylamino-9,19-cyclo-4α-formyl-4,14α-dimethyl-20-dimethylamino-5α,9-pregnan-11-one 14

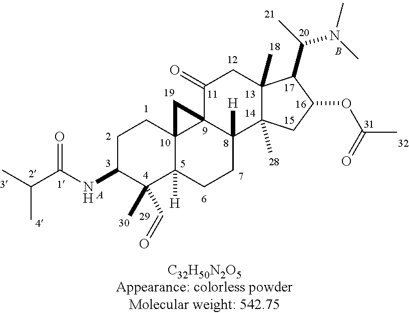

To a solution of 200 mg (0.36 mmol, 1.0 eq) of 16-O-acetyl-N-3-isobutyrylcycloxobuxidine-F 13 in 20 ml of dichloromethane are added 209 mg (0.47 mmol, 1.3 eq) of the Dess-Martin periodinane. The solution clouds and becomes pink. After 16 hours of agitation at room temperature, the mixture is alkalinized with 40 ml of a 10% ammonia solution (pH=10) and extracted with 3×40 ml of dichloromethane. The recombined organic phases are successively dried on anhydrous sodium sulfate and evaporated under a vacuum.

The residue thus obtained is purified by silica gel flash chromatography (eluent: 95/5 dichloromethane/methanol) to yield 127 mg of 14 in the form of a colorless powder with a yield of 64%.

IR (CHCl₃)

υ(cm⁻¹): 3497 (OH, NH); 2773 (CHO); 1728 (C═O); 1656 and 1517 (CONH); 1096 (C—OH).

Mass

(ESI): m/z: 543.4 ([M+H], 100); 501.4 (5).

HRMS (ESI) calculated for C₃₂H₅₁N₂O₅ m/z=543.3798; actual: 543.3784.

### Compound 16: (20S)-16α-acetyl-3-isobutyrylamino-9,19-cyclo-4α-benzyliminomethyl-4,14α-dimethyl-20-dimethylamino-5α,9-pregnan-11-one 16

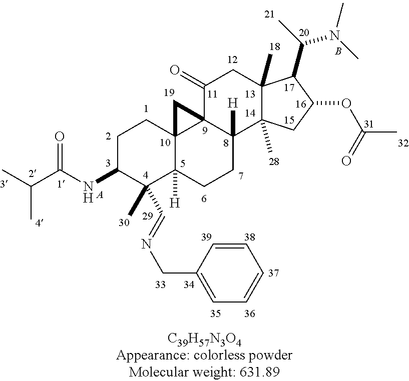

Under argon, to a suspension of 60 mg (0.11 mmol, 1.0 eq) of aldehyde 14 in 2 ml of dichloromethane are added 50 mg of anhydrous magnesium sulfate and 12 μl (0.11 mmol, 1.0 eq) of benzylamine. After 16 hours of agitation at room temperature, the mixture is filtered and then evaporated under a vacuum to yield 65 mg of 16 in the form of a colorless powder with a yield of 92%.

IR (diamond)

υ(cm⁻¹): 2939 (NH, C═N); 1730 (C═O ester); 1659 (C═O, CONH, C═C and C═N); 1532 (CONH); 1245 (C═C).

Mass

(ESI): m/z: 632.4 ([M+H], 50); 633.5 (5); 590.5 (10); 545.3 (100); 546.4 (50); 503.3 (80); 504.4 (10).

HRMS (ESI) calculated for C₃₉H₅₈N₃O₄ m/z=632.4427; actual: 632.4443.

### Compound 17: (20S)-16α-pivalyl-3-acetylamino-9,19-cyclo-4α-hydroxymethyl-4,14α-dimethyl-20-dimethylamino-5α,9-pregnan-11-one 17

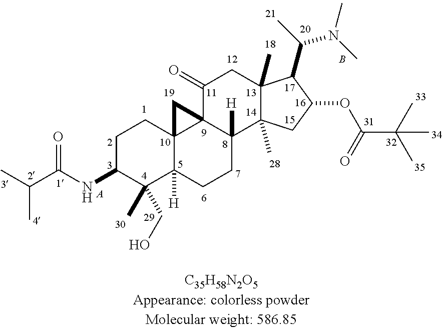

To a solution of 400 mg (0.80 mmol, 1.0 eq) of N-3-isobutyrylcycloxobuxidine-F 2 in 2.0 ml of pyridine are added 0.48 ml (2.40 mmol, 3.0 eq) of pivalic anhydride. The solution is light yellow. After 5 hours of agitation at 90° C., the mixture is co-evaporated in the presence of 1,2-dichloroethane.

The residue thus obtained is purified by silica gel flash chromatography (eluent: 9/1 dichloromethane/methanol) to yield 400 mg of 17 in the form of a colorless powder with a yield of 85%.

IR (diamond)

υ(cm⁻¹): 3434, 2972 (OH, NH); 1711 (C═O ester); 1655 and 1514 (C═O and CONH); 1175 (C—OH).

Mass

(ESI): m/z: 587.5 ([M+H], 100); 588.4 (5).

HRMS (ESI) calculated for C₃₅H₅₉N₂O₅ m/z=587.4424; actual: 587.4417.

### Compound 18: (20S)-16α-pivalyl-3-isobutyrylamino-9,19-cyclo-4α-formyl-4,14α-dimethyl-20-dimethylamino-5α,9-pregnan-11-one 18

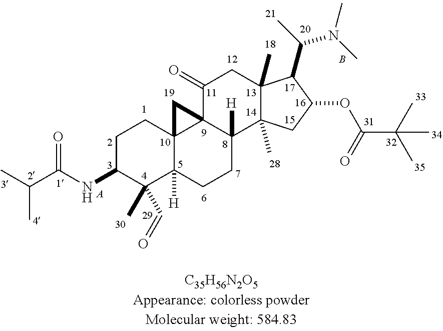

To a solution of 74 mg (0.12 mmol, 1.0 eq) of 16-O-pivalyl-N-3-isobutyrylcycloxobuxidine-F 17 in 5 ml of dichloromethane are added 72 mg (0.16 mmol, 1.3 eq) of the Dess-Martin periodinane. The solution clouds and becomes pink. After 16 hours of agitation at room temperature, the mixture is alkalinized with 40 ml of a 10% ammonia solution (pH=10) and extracted with 3×30 ml of dichloromethane. The recombined organic phases are successively dried on anhydrous sodium sulfate and evaporated under a vacuum.

The residue thus obtained is purified by silica gel flash chromatography (eluent: 95/5 dichloromethane/methanol) to yield 44 mg of 18 in the form of a colorless powder with a yield of 62%.

IR (diamond)

υ(cm⁻¹): 2966 (NH, CHO); 1720 (C═O ester); 1665 and 1528 (C═O and CONH).

Mass

(ESI): m/z: 585.4 ([M+H], 100); 586.4 (45).

HRMS (ESI) calculated for C₃₅H₅₇N₂O₅ m/z=585.4267; actual: 585.4281.

### Compound 19: (20S)-16α-pivalyl-3-isobutyrylamino-9,19-cyclo-4α-benzyliminomethyl-4,14α-dimethyl-20-dimethylamino-5α,9-pregnan-11-one 19

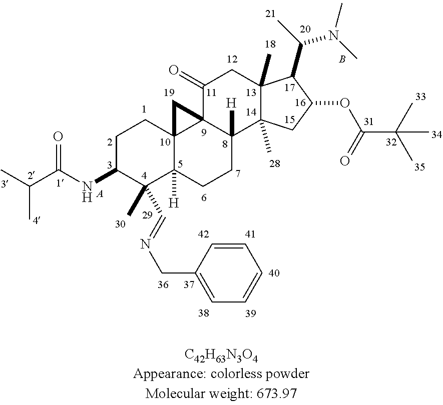

Under argon, to a suspension of 113 mg (0.19 mmol, 1.0 eq) of aldehyde 18 in 3 ml of dichloromethane are added 94 mg of anhydrous magnesium sulfate and 21 μl (0.19 mmol, 1.0 eq) of benzylamine. After 24 hours of agitation at room temperature, the mixture is filtered and then evaporated under a vacuum to yield 119 mg of 19 in the form of a colorless powder with a yield of 92%.

IR (diamond)

υ(cm⁻¹): 2966 (NH); 1720 (C═O ester); 1662 and 1532 (C═O and CONH); 1156 (C═C).

Mass

(ESI): m/z: 674.4 ([M+H], 100); 675.5 (10).

HRMS (ESI) calculated for C₄₂H₆₄N₃O₄ m/z=674.4897; actual: 674.4885.

### Compound 20: (20S)-16α-pivalyl-3-isobutyrylamino-9,19-cyclo-4α-pivalylmethyl-4,14α-dimethyl-20-dimethylamino-5α,9-pregnan-11-one 20

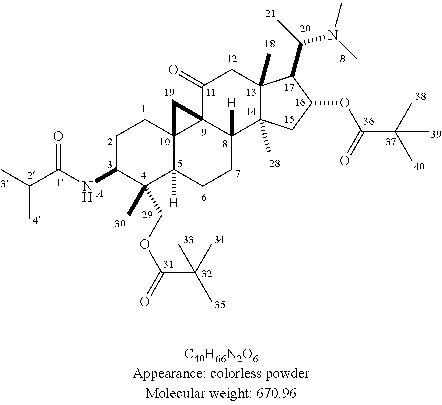

To a solution of 400 mg (0.80 mmol, 1.0 eq) of N-3-isobutyrylcycloxobuxidine-F 2 in 2.0 ml of pyridine are added dropwise 0.3 ml (2.40 mmol, 3.0 eq) of pivalyl chloride at room temperature. The reaction is exothermic. The solution clouds and then becomes light yellow. The precipitate formed redissolves in 30 minutes. After 1 hour of agitation at room temperature, the mixture is co-evaporated in the presence of 1,2-dichloroethane.

The residue thus obtained is purified by silica gel flash chromatography (eluent: 9/1 dichloromethane/methanol) to yield 240.5 mg of 20 in the form of a colorless powder with a yield of 45%.

IR (diamond)

υ(cm⁻¹): 3440, 2974 (OH, NH); 1715 (C═O ester); 1668 and 1507 (C═O and CONH); 1171 (C—OH).

Mass

(ESI): m/z: 671.5 ([M+H], 100); 672.5 (5).

HRMS (ESI) calculated for C₄₀H₆₇N₂O₆ m/z=671.4999; actual: 671.4985.

### Compound 21: (20S)-16α-acetyl-3-isobutyrylamino-9,19-cyclo-4α-hydroxymethyl-4,14α-dimethyl-20-dimethylhydrogenoamino-5α,9-pregnan-11-one tosylate 21

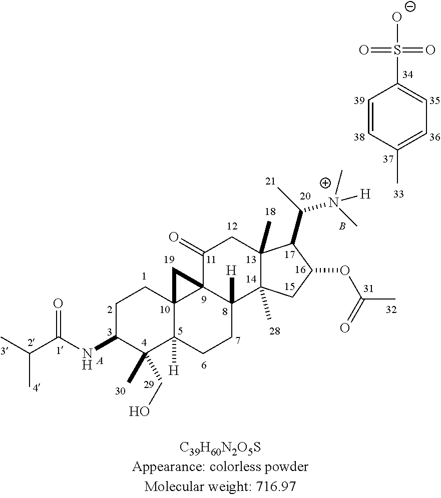

To a solution of 20 mg (0.04 mmol, 1.0 eq) of 16-O-acetyl-N-3-isobutyrylcycloxobuxidine-F 2 in 2.0 ml of methanol are added 7 mg (0.04 mmol, 1.0 eq) of p-toluenesulfonic acid, in the presence of a spatula tip of anhydrous sodium sulfate, at room temperature. After 30 minutes of agitation at room temperature, the suspension is filtered and evaporated under reduced pressure to yield 26 mg of 21 in the form of a colorless powder with a yield of 99%.

IR (diamond)

υ(cm⁻¹): 3492, 2931 (OH, NH); 1732 (C═O); 1642 and 1546 (CONH); 1120 (C—OH); 1381 and 1170 (S═O); 1032 (C—O); 816 (S—O).

Mass

(ESI): m/z: 545.4 ([M+H], 100); 546.4 (5); 503.4 (50); 413.3 (10).

HRMS (ESI) calculated for C₃₂H₅₃N₂O₅+m/z=545.3954; actual: 545.3939.

### Compound 22: (20S)-16α-pivalyl-3-isobutyrylamino-9,19-cyclo-4α-tosylmethyl-4,14α-dimethyl-20-dimethylamino-5α,9-pregnan-11-one 22

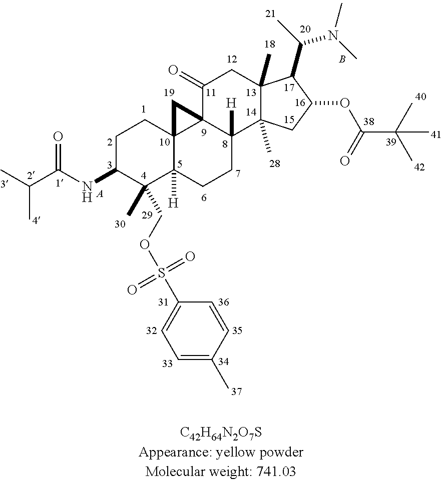

To a solution of 100 mg (0.17 mmol, 1.0 eq) of 16-O-pivalyl-N-3-isobutyrylcycloxobuxidine-F 17 and 32 mg (0.17 mmol, 1.0 eq) of p-toluenesulfonic acid in 3 ml of methanol is added a spatula of anhydrous sodium sulfate. The suspension is agitated for 30 minutes and then filtered and evaporated under a vacuum.

To a solution of the salt previously obtained in 3 ml of pyridine are added 99 mg (0.52 mmol, 3.0 eq) of tosyl chloride. The solution is lemon-yellow in color. After 5 hours of agitation at 90° C., the solution is evaporated under a vacuum in the presence of 1,2-dichloroethane. The residue is taken up with 30 ml of dichloromethane, washed with 30 ml of a 10% sodium hydrogen carbonate solution (pH=8) and extracted with 3×30 ml of dichloromethane. The recombined organic phases are dried on anhydrous sodium sulfate and evaporated under a vacuum.

The residue is chromatographed on a preparative silica plate (eluent: 9/1 dichloromethane/methanol) to yield 55 mg of 22 in the form of a yellow powder with a yield of 44%.

### Compound 23: (20S)-16α-acetyl-3-isobutyrylamino-9,19-cyclo-4α-hydroxytosylmethyl-4,14α-dimethyl-20-dimethylamino-5α,9-pregnan-11-one 23

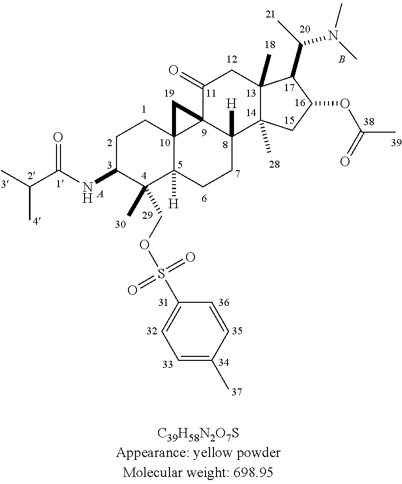

To a solution of 100 mg (0.18 mmol, 1.0 eq) of 16-O-acetyl-N-3-isobutyrylcycloxobuxidine-F 13 and 35 mg (0.18 mmol, 1.0 eq) of p-toluenesulfonic acid in 1.5 ml of methanol is added a spatula of anhydrous sodium sulfate. The suspension is agitated for 30 minutes and then filtered and evaporated under a vacuum.

To a solution of the salt previously obtained in 2.5 ml of pyridine is added 91 mg (0.48 mmol, 2.6 eq) of tosyl chloride. The solution is lemon-yellow in color. After 5 hours of agitation at 100° C., the solution is evaporated under a vacuum in the presence of 1,2-dichloroethane. The residue is taken up with 20 ml of dichloromethane, washed with 30 ml of a 10% sodium hydrogen carbonate solution (pH=8) and extracted with 3×20 ml of dichloromethane. The recombined organic phases are dried on anhydrous sodium sulfate and evaporated under a vacuum.

The residue is purified on 0.75 g of cellulose acetate and then 0.75 g of florysil (magnesium trisilicate).

Lastly, the residue is purified by silica gel flash chromatography (eluent: 9/1 dichloromethane/methanol) to yield 64 mg of 23 in the form of a yellow powder with a yield of 50%.

IR (diamond)

υ(cm⁻¹): 2932 (NH); 1729 (C═O ester); 1667 (CO, CONH and C═C); 1526 (CONH), 1244, (C═C); 1357 and 1174 (S═O); 1095 (C—O); 835 (S—O).

Mass

(ESI): m/z: 699.2 ([M+H], 100); 700.3 (30); 657.3 (45); 587.3 (30).

HRMS (ESI) calculated for C₃₉H₅₉N₂O₇S m/z=699.4043; actual: 639.4039.

### Compound 27: (20S)-3-isobutyrylamino-9,19-cyclo-4α-benzylhydroxymethyl-4,14α-dimethyl-20-dimethylamino-29-tosyl-5α,9-pregnan-16α-ol-11-one 27

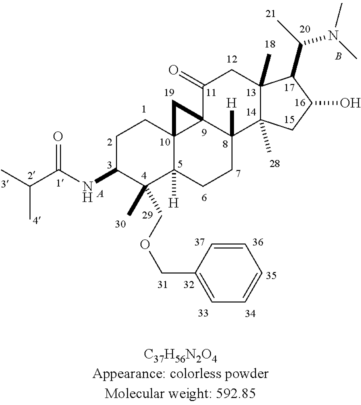

Under argon, to a 0° C. solution of 300 mg (0.59 mmol, 1.0 eq) of N-3-isobutyrylcycloxobuxidine-F in 2 ml of dimethylformamide is added 0.22 ml (1.80 mmol, 3.0 eq) of benzyl bromide. The solution is agitated for 10 minutes at 0° C. Next, 60 mg of sodium hydride (1.5 mmol, 2.5 eq) are added little by little. After 24 hours of agitation at room temperature, the sodium hydride excess is eliminated with methanol and the solution is concentrated in the presence of toluene.

The residue is taken up with 20 ml of dichloromethane, washed with 30 ml of a 10% sodium hydrogen carbonate solution (pH=8) and extracted with 3×20 ml of dichloromethane. The recombined organic phases are dried on anhydrous sodium sulfate and evaporated under a vacuum.

The residue is purified by silica gel flash chromatography (eluent: 9/1 dichloromethane/methanol) to yield 131 mg of 27 in the form of a colorless powder with a yield of 37%.

IR (diamond)

υ(cm⁻¹): 3321, 2932 (OH, NH); 1649 (C═O and C═C); 1649 and 1531 (CONH); 1225 (C═C); 1092 (C—OH).

Mass

(ESI): m/z: 593.4 ([M+H], 100); 594.5 (10).

HRMS (ESI) calculated for C₃₇H₅₇N₂O₄ m/z=593.4318; actual: 593.4311.

### Compound 28: (20S)-3-amino-9,19-cyclo-4α-hydroxymethyl-4,14α-dimethyl-20-dimethylamino-5α,9-pregnan-16α,11-diol-11-one 28

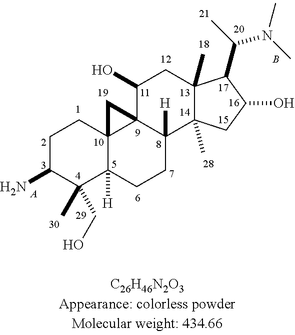

Under argon, to a solution of 300 mg (0.69 mmol, 1.0 eq) of amine 5 in 25 ml of 1,4-dioxane are added 353 mg (9.01 mmol, 13.0 eq) of aluminum and lithium hydride. After 12 hours of agitation at room temperature, the mixture is hydrolyzed with 0.35 ml of water, then 0.35 ml of 6 N soda and 1.05 ml of water. The gray suspension lightens and is filtered and then evaporated under a vacuum to yield 190 mg of 28 in the form of a colorless powder after trituration in diethyl ether with a yield of 63%.

IR (CHCl₃)

υ(cm⁻¹): 3382 (OH, NH); 1037 (C—OH).

Mass

(ESI): m/z: 435.4 ([M+H], 100); 436.4 (5); 418.4 (10).

HRMS (ESI) calculated for C₂₆H₄₇N₂O₃ m/z=435.3587; actual: 435.3581.

### Compound 29: (20S)-3-isobutyrylamino-9,19-cyclo-4α-hydroxymethyl-4,14α-dimethyl-20-dimethylazinoyl-5α,9-pregnan-16α,11-ol-11-one 29

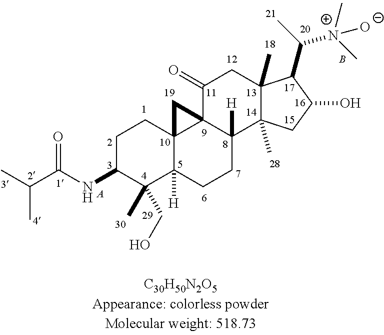

To a solution of 202 mg (0.40 mmol, 1.0 eq) of N-3-isobutyrylcycloxobuxidine-F 2 in 6 ml of dichloromethane are added 95 mg (0.55 mmol, 1.4 eq) of m-chloroperbenzoic acid. After 30 minutes of agitation at room temperature, the mixture is alkalinized with 40 ml of a 10% ammonia solution (pH=10) and extracted with 3×40 ml of dichloromethane. The recombined organic phases are successively dried on anhydrous sodium sulfate and evaporated under a vacuum.

The residue thus obtained is triturated in diethyl ether to yield 165 mg of 29 in the form of a colorless powder with a yield of 79%.

IR (CHCl₃)

υ(cm⁻¹): 3434 (OH, NH); 1655 (C═O); 1655 and 1513 (CONH); 1049 (C—OH).

Mass

(ESI): m/z: 519.3 ([M+H], 100); 520.3 (10); 480.3 (20); 440.3 (30); 370.3 (20); 353.2 (10); 341.2 (25); 323.2 (25).

HRMS (ESI) calculated for C₃₀H₅₁N₂O₅ m/z=519.3798; actual: 519.3770.

### Compound 30: (20S)-3-isobutyrylamino-9,19-cyclo-4α-hydroxymethyl-4,14α-dimethyl-20-methylamino-5α,9-pregnan-16α-ol-11-one 30

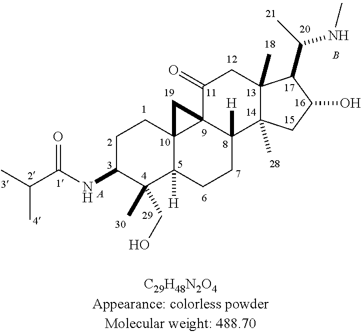

To a solution of 29.5 mg (0.06 mmol, 1.0 eq) of N-oxide 29 in 3 ml of methanol are added 32 mg (0.11 mmol, 2.0 eq) of iron II sulfate heptahydrate and 15 mg (0.06 mmol, 1.0 eq) of iron III chloride hexahydrate. The solution is orange. After 1.5 hours of agitation at room temperature the mixture is evaporated under a vacuum. The residue is alkalinized with 10 ml of a 10% sodium carbonate solution. A greenish precipitate forms which is filtered on cellulose acetate. The filtrate is rinsed with water and then with dichloromethane. The filtered solution is acidified by 30 ml of a 1.0 M acetic acid/sodium acetate buffer solution (pH=4.8). After 15 minutes of agitation the solution is alkalinized with 30 ml of a 10% ammonia solution (pH=10) and extracted with 5×40 ml of dichloromethane. The recombined organic phases are successively washed with 20 ml of a sodium chloride saturated aqueous solution, dried on anhydrous sodium sulfate and evaporated under a vacuum.

The residue is triturated in diethyl ether to yield 21 mg of 30 in the form of a colorless powder with a yield of 76%.

IR (CHCl₃)

υ(cm⁻¹): 3434 (OH, NH); 1655 (C═O); 1655 and 1514 (CONH); 1093 (C—OH).

Mass

(ESI): m/z: 489.3 ([M+H], 100); 503.4 (40).

HRMS (ESI) calculated for C₂₉H₄₉N₂O₄ m/z=489.3692; actual: 489.3688.

### Compound 31: (20S)-9(10->19)abeo-4α-hydroxymethyl-3-isobutyrylamino-4,14α-dimethyl-20-dimethylamino-5α,9-pregn-1(10)-en-16α-ol-11-one 31

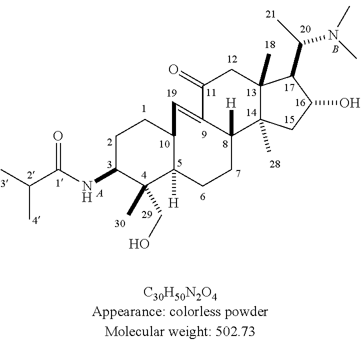

Under argon, to a solution of 400 mg (0.80 mmol, 1.0 eq) of N-3-isobutyrylcycloxobuxidine-F 2 in 30 ml of dichloromethane are added dropwise 2.6 ml (24.0 mmol, 30.0 eq) of titanium chloride at −78° C. The solution becomes yellow. After 30 minutes of agitation at −78° C., the solution is agitated at room temperature. The mixture is dark green in color.

After 2 hours of agitation at room temperature, the mixture is alkalinized with 100 ml of a 10% ammonia solution (pH=10) and extracted with 3×75 ml of dichloromethane. The recombined organic phases are successively washed with a sodium chloride saturated solution, dried on anhydrous sodium sulfate and evaporated under a vacuum.

The residue thus obtained is purified by alumina chromatography (eluent: 99.7/0.3 dichloromethane/methanol) to yield 185 mg of 31 in the form of a colorless powder with a yield of 46%.

MP (° C.): 312

Elemental analysis: for C₃₀H₅₀N₂O₄

Calculated for C₃₀H₅₀N₂O₄.0.5H₂O %: C, 70.41; H, 10.05; N, 5.47

Actual %: C, 70.84; H, 9.71; N, 5.21

IR (CHCl₃)

υ(cm⁻¹): 3434 (OH, NH); 1675 (C═O); 1651 (C═C and CONH); 1514 (CONH); 1040 (C—OH).

Mass

(ESI): m/z: 503.3 ([M+H], 100); 504.4 (90).

HRMS (ESI) calculated for C₃₀H₅₁N₂O₄ m/z=503.3849; actual: 503.3843.

### Compound 32: (20S)-9(10->19)abeo-4,14α-dimethyl-20-dimethylamino(2′-isopropyl-4′,4′-dihydro[3′,1′]oxazin)-5α,9-pregn-1(10)-en-16α-ol-11-one 32

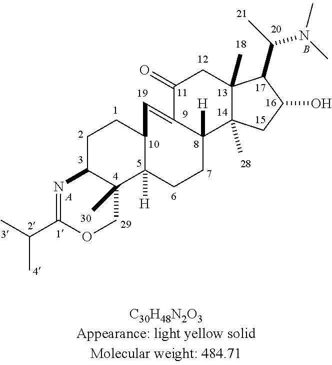

To a solution of 100 mg (0.20 mmol, 1.0 eq) of amide 31 in 2 ml of dichloromethane are added 586 mg (1.00 mmol, 5.0 eq) of a 25% tetraethylammonium hydroxide solution in methanol. After evaporation under a vacuum, the red residue heated at 300° C. under 0.03 mmHg for 4 hours yields 60 mg of 32 in the form of a non-sublimated light yellow solid with a yield of 62%.

Elemental analysis: for C₃₀H₄₈N₂O₃

Calculated for C₃₀H₄₈N₂O₃.0.5H₂O %: C, 72.98; H, 10.00; N, 5.67

Actual %: C, 72.61; H, 9.65; N, 5.64

Mass

(ESI): m/z: 485.4 ([M+H], 100); 486.4 (10); 503.4 (20).

HRMS (ESI) calculated for C₃₀H₄₂N₂O₃ m/z=485.3743; actual: 485.3702.

### Example 6

### Biological Results

Antiacetylcholinesterase (AChE isolated from Electrophorus electricus) and antibutyrylcholinesterase (BChE isolated from human serum) activities were determined by using the spectroscopic method of Ellman (Ellman, G. L. et al.; Biochem. Pharmacol. 1961, 7, 88-95). The results are summarized in table 1.

The values given for galanthamine, tacrine and eserine act as references (360 and 74 for anti-AChE activity, and 18,600, 58 and 800 for anti-BchE activity).

TABLE 1
In vitro inhibition of acetylcholinesterase
(AChE) isolated from Electrophorus electricus
and of human serum butyrylcholinesterase (BChE).

| | AChE | | | AChE | |
|---|---|---|---|---|---|
| | IC₅₀ | | | IC₅₀ | |
| Compound | (nM) | BChE | Compound | (nM) | BChE |
| 1 | 31 | >1000 | 8e | 13 | >1000 |
| 2 | >10000 | >10000 | 8f | 225 | >10000 |
| 3 | 9380 | 7407 | 8g | 4205 | >10000 |
| 5 | >5000 | >30000 | 8h | 27 | 1535 |
| 6b | >10000 | 2215 | 8i | 102 | 380 |
| 6d | >10000 | >5000 | 9 | 2377 | >10000 |
| 6g | >5000 | 30000 | 11 | 1498 | >10000 |
| 6h | >100000 | 121 | 12 | 823 | >10000 |
| 7b | >10000 | 1878 | 13 | >10000 | >1000 |
| 7d | >1000 | 4022 | 14 | >1000 | >1000 |
| 7c | >1000 | >10000 | 19 | >1000 | 1277 |
| 7e | 105 | >10000 | 23 | 4368 | >100000 |
| 7f | 4832 | >1000 | 27 | 16768 | 5197 |
| 7g | >1000 | >10000 | 28 | >1000 | 2446 |
| 7h | 120 | 1926 | 29 | 1418 | >1000 |
| 7i | 3270 | >10000 | 32 | 112 | 1444 |
| 8a | 676 | >10000 | | | |
| 8b | 7648 | >10000 | | | |
| 8c | 607 | >10000 | | | |
| 8d | 403 | 375 | | | |
| galanthamine | 360 | 18600 | | | |
| tacrine | 74 | 58 | | | |
| eserine | | 800 | | | |

### Example 7

### Study of Compound 8e at the Vertebrate Skeletal Neuromuscular Junction

Compound 8e has proved to possess a powerful in vitro acetylcholinesterase inhibiting effect, as demonstrated above (table 1). To verify the effectiveness and specificity of compound 8e on a cholinergic synapse model the inventors tested these effects on isolated living vertebrate (frog and mouse) neuromuscular preparations, using conventional cellular electrophysiology techniques.

Compound 8e in amounts between 1-25 μM produces an extension of synaptic response half-times (motor end-plate potentials caused by nerve stimulation and spontaneous motor end-plate miniature potentials). This extension of synaptic potentials is observed when the mouse neuromuscular preparations are placed in a normal survival medium (Krebs-Ringer) containing (in mM) NaCl, 151; KCl, 5; MgCl₂, 1; CaCl₂, 2; glucose, 11; and HEPES-NaOH, 5 (pH 7.4); and when the survival medium contains low concentrations of CaCl₂ (0.4-1 mM) and high concentrations of MgCl₂ (4-8 mM) to reduce the release of acetylcholine. The extension of synaptic potentials at the neuromuscular junction reflects the mean lifespan of synaptic channels opened by acetylcholine during their interactions with skeletal muscle nicotinic receptors. Inhibition of acetylcholinesterase increases acetylcholine concentration at the synaptic cleft, which allows acetylcholine to bind repeatedly with these receptors, thus extending the duration of synaptic responses. The electrophysiological results obtained confirm that compound 8e has a clear anticholinesterase effect on isolated mouse neuromuscular preparations. Similar effects were observed on frog neuromuscular junctions.

At concentrations that extend the duration of synaptic potentials, and even at higher concentrations (160-200 μM), compound 8e does not have a significant effect on muscle fiber resting potentials. Thus, the inhibition of acetylcholinesterase by this compound is not accompanied by depolarization of the muscle membrane.

From a pharmacological point of view, the inventors first tested the ability of compound 8e to modify nerve axon conduction. The results obtained indicate that this molecule, in amounts between 1 μM and 300 μM, does not apparently modify nerve conduction in mouse neuromuscular preparations.

Secondly, the inventors determined, using electrophysiology, the ability of compound 8e to interact with muscle nicotinic acetylcholine receptors. The results obtained show that compound 8e, at a concentration of 80 μM, causes a decrease in amplitude and blocking of motor end-plate miniature potentials (which reflect the release of an acetylcholine quantum). Moreover, synaptic responses (motor end-plate potentials), recorded in response to stimulation of the motor nerve, and composed of approximately 40 acetylcholine quanta, are also 80% blocked by 160 μM of compound 8e, on both frog and mouse neuromuscular junctions. The totality of these results demonstrates that compound 8e blocks the interaction of acetylcholine with its muscle receptor sites. These results are not surprising, because most anticholinesterase agents also block muscle nicotinic acetylcholine receptors. However, it should be noted that the muscle nicotinic acetylcholine receptor blocking effect occurs at significantly higher concentrations than those required to inhibit skeletal neuromuscular junction acetylcholinesterase, as was observed for other acetylcholinesterase inhibitors, such as galanthamine and tacrine.

Yet compound 8e is active as an AChE inhibitor at concentrations greater than 13 nM. It can thus be used in an amount that makes it possible to inhibit AChE without blocking muscle nicotinic AChE receptors. Thus, the triterpenic alkaloids according to the invention do not exhibit side effects when used in an amount that inhibits AChE.

### Example 8

### Synthesis of Compounds 33 to 76

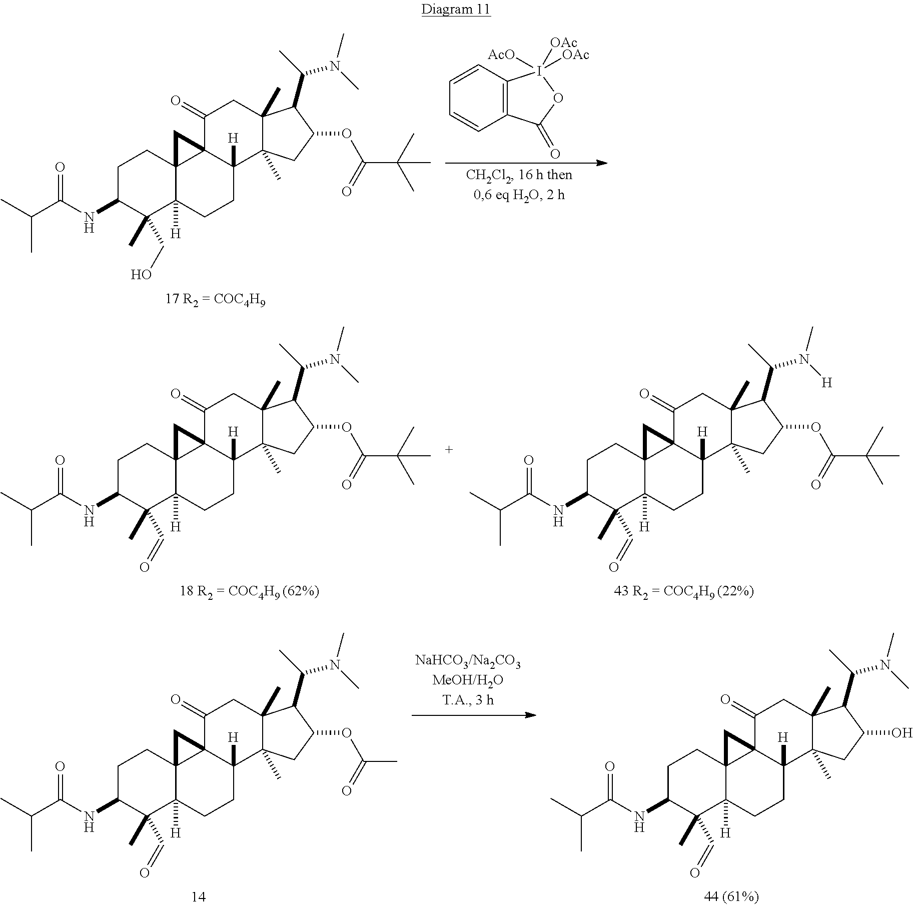

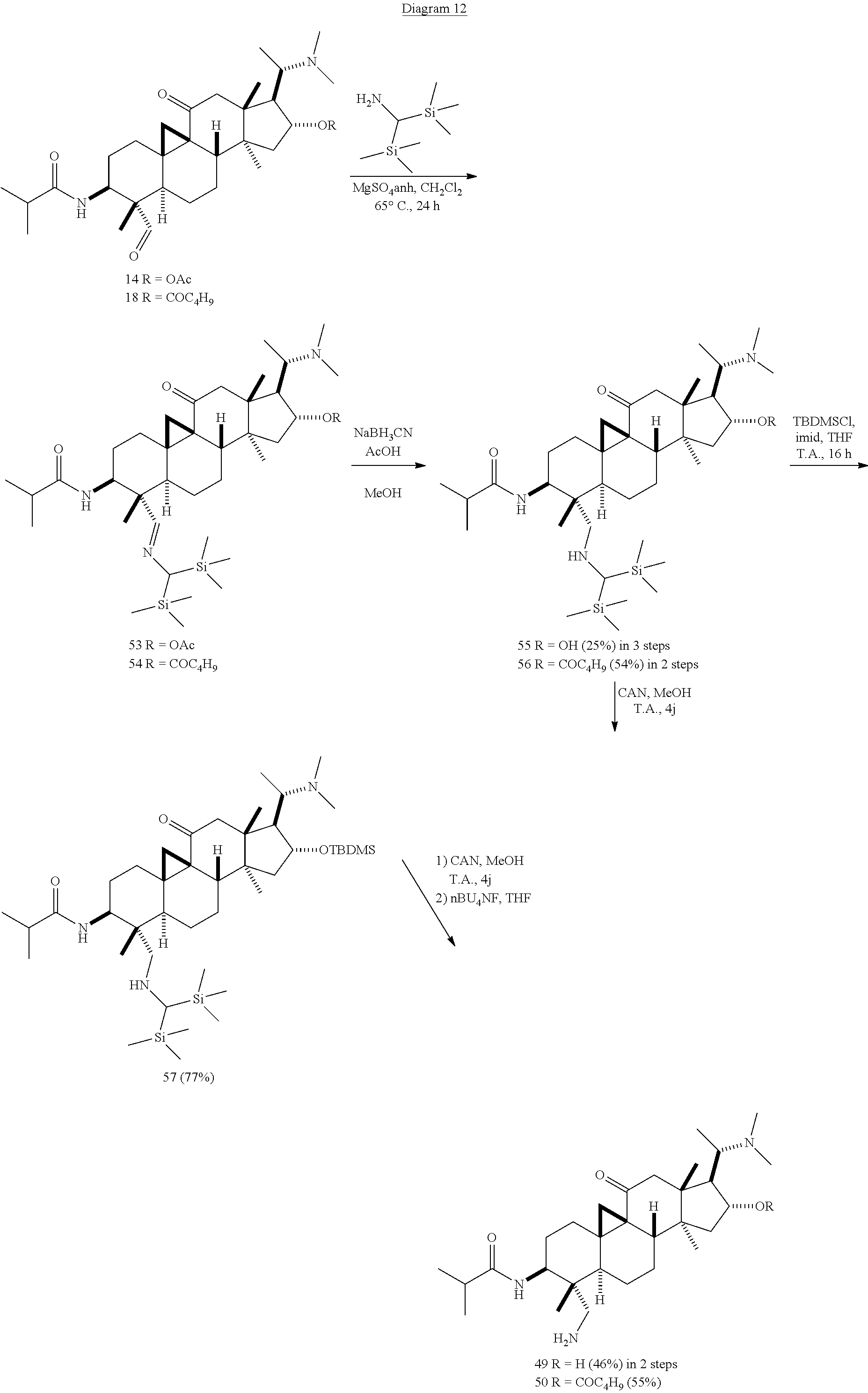

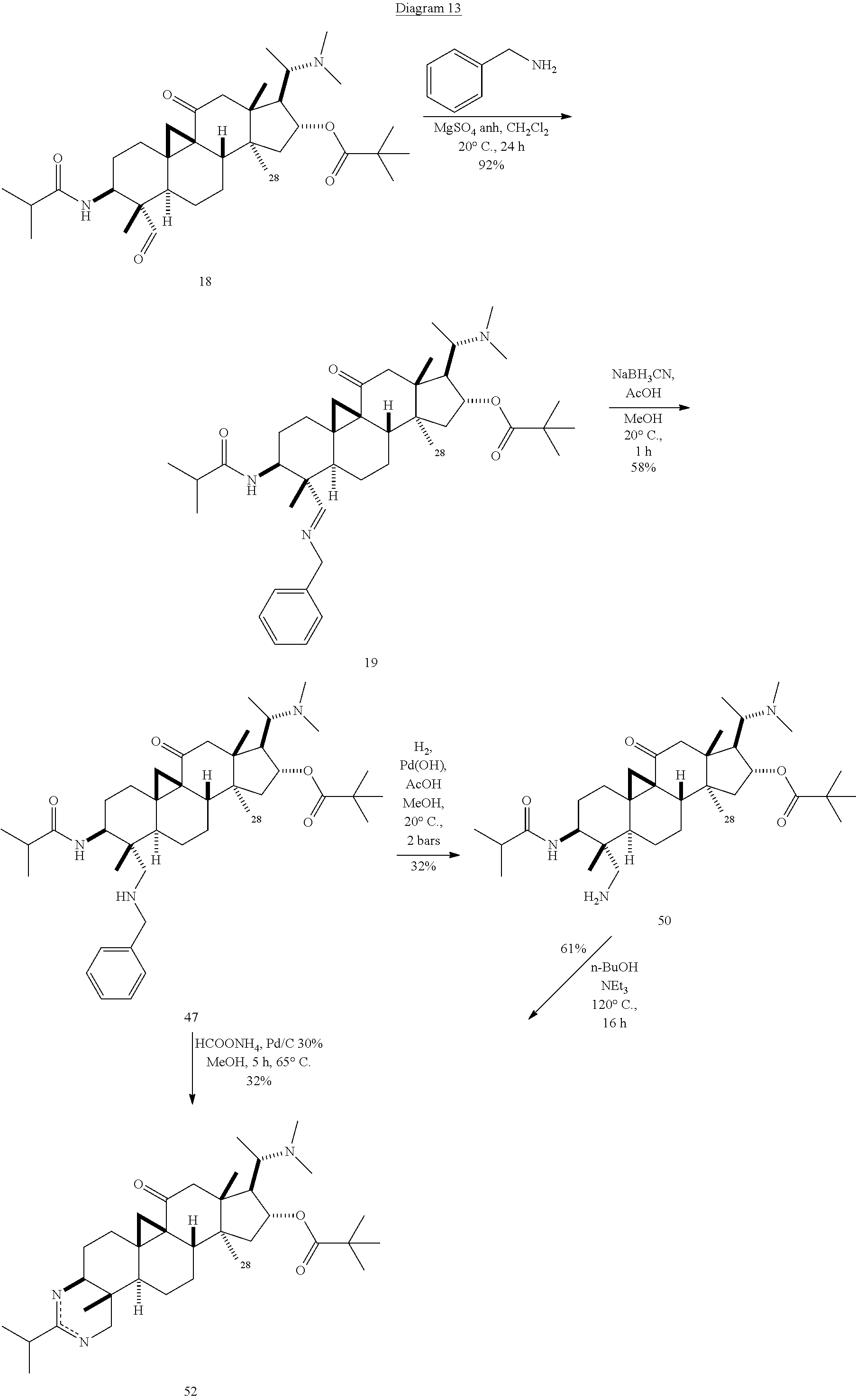

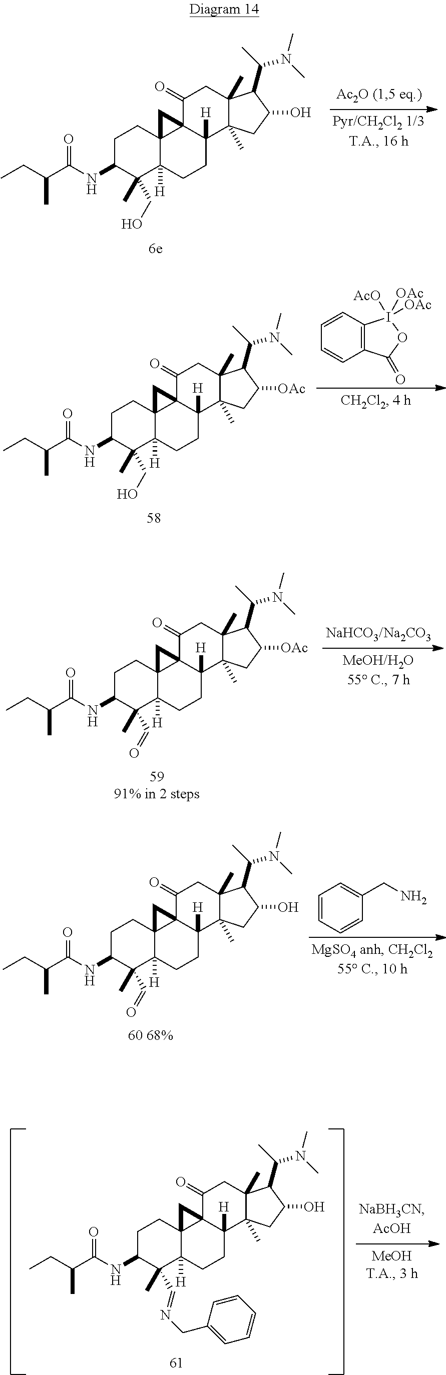

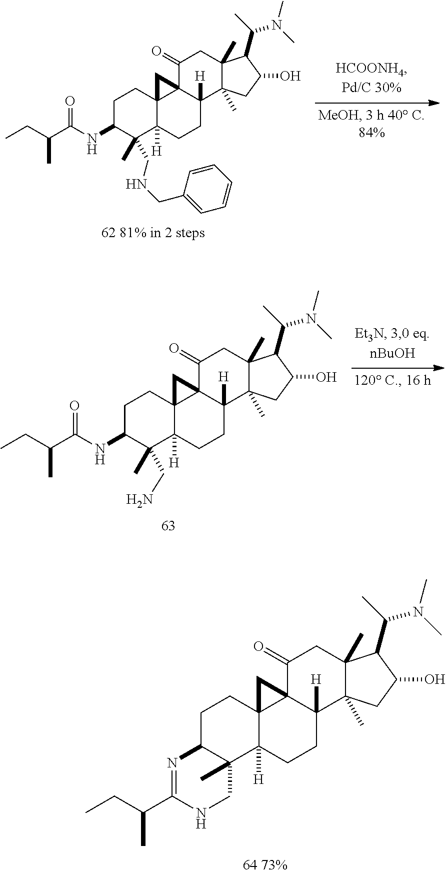

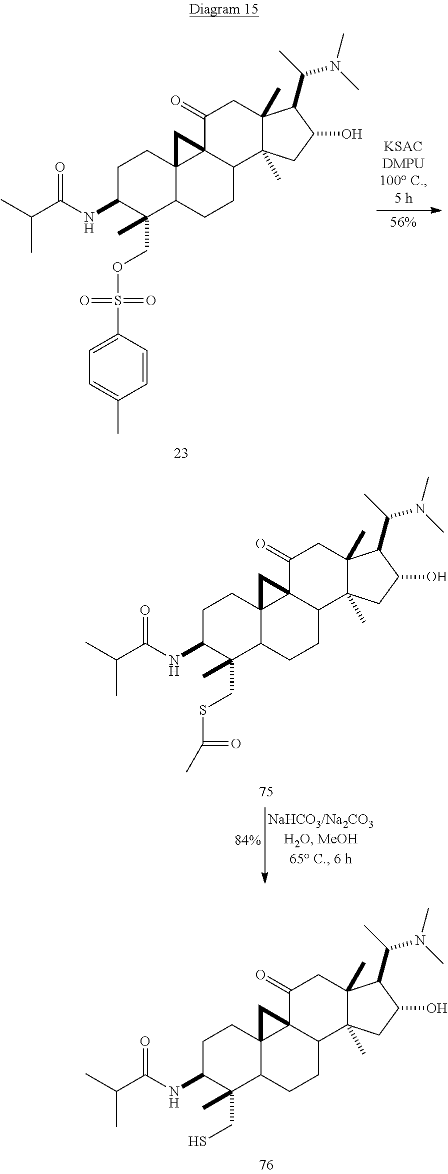

### Compound 33: (20S)-10(9->1)abeo-20-methylamino-4β,14α-dimethyl-(2′-isopropyl-3,29-dihydro-4H-[1,3]-oxazine)-5α,9β-pregn-1(10)-en-16α-ol-11-one 33

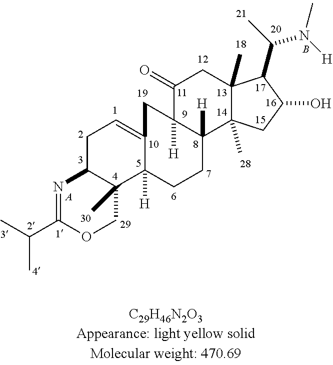

100 mg (0.20 mmol, 1.0 eq) of Nor-N-3-isobutyrylcycloxobuxidine-F 30 are heated at 273° C. under 0.09 mmHg in a ball tube oven or a sublimation tube. A solid sublimate is obtained in 3 hours. To a solution of the sublimate in 2 ml of dichloromethane are added 600 mg (1.02 mmol, 5.0 eq) of a 25% tetraethylammonium hydroxide solution in methanol. After evaporation under a vacuum, the red residue heated at 273° C. under 0.09 mmHg for 3 hours yields 56 mg of 33 in the form of a light yellow amorphous sublimate with a yield of 60%.

IR (CHCl₃) υ(cm⁻¹): 3367 (OH, NH); 2964 (C—H); 1697 (C═O); 1667 (C═N and C═C); 1074 (C—O).

### Compound 34: (20S)-20-dimethylamino-3β-(isobutyrylamino)-9,19-cyclo-4α-hydroxymethyl-4β,14α-dimethyl-5α,9β-pregnan-11β,16α-diol-11-one 34

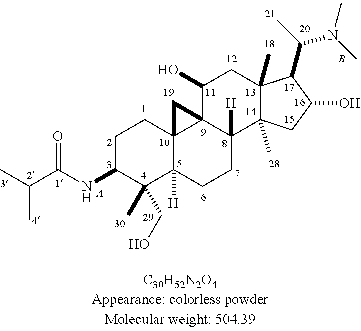

To a solution of 127 mg (0.29 mmol, 1.0 eq) of amine 28 in 0.7 ml of dichloromethane and 0.3 ml of methanol are added 51 μl (0.30 mmol, 1.05 eq) of isobutyric anhydride. The solution is light yellow. After 6 hours of agitation at room temperature, the mixture is alkalinized with 30 ml of a 10% ammonia solution (pH=10) and extracted with 3×30 ml of dichloromethane. The recombined organic phases are successively dried on anhydrous sodium sulfate and evaporated under a vacuum.

The residue thus obtained is precipitated in diethyl ether to yield 110 mg of 34 in the form of a colorless powder with a yield of 75%.

Elemental analysis: for C₃₀H₅₂N₂O₄.0.5H₂O

Calculated for C₃₀H₅₂N₂O₄.0.5H₂O %: C, 70.14; H, 10.40; N, 5.45

Measured %: C, 74.21; H, 10.19; N, 5.14

IR (diamond) υ(cm⁻¹): 3275 (OH, NH); 2932 (CH, CH₂); 1633 and 1551 (CONH); 1039 (C—O).

MS (ESI): m/z: 505.4 ([M+H], 100); 506.4 (30).

HRMS (ESI) calculated for C₃₀H₅₃N₂O₄ m/z=505.4005; measured: 505.3992.

### Compound 35: (20S)-20-dimethylamino-3β-((S)-2-methylbutyrylamino)-9,19-cyclo-4α-hydroxymethyl-4β,14α-dimethyl-5α,9β-pregnan-11β,16α-diol-11-one 35

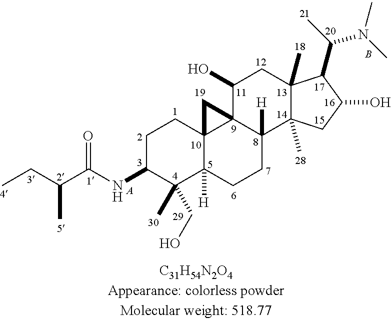

To a solution of 131 mg (0.30 mmol, 1.0 eq) of amine 28 in 0.7 ml of dichloromethane and 0.3 ml of methanol are added 67 μl (0.31 mmol, 1.05 eq) of (S)-2-methylbutyric anhydride. The solution is light yellow. After 6 hours of agitation at room temperature, the mixture is alkalinized with 30 ml of a 10% ammonia solution (pH=10) and extracted with 3×30 ml of dichloromethane. The recombined organic phases are successively dried on anhydrous sodium sulfate and evaporated under a vacuum.

The residue thus obtained is precipitated in diethyl ether to yield 124 mg of 35 in the form of a colorless powder with a yield of 80%.

Elemental analysis: for C₃₁H₅₄N₂O₄.0.5H₂O

Calculated for C₃₁H₅₄N₂O₄.0.5H₂O %: C, 70.55; H, 10.50; N, 5.31

Measured %: C, 74.57; H, 10.52; N, 4.77

IR (diamond) υ(cm⁻¹): 3305 (OH, NH); 2932 (CH, CH₂); 1641 and 1537 (CONH); 1038 (C—O).

MS (ESI): m/z: 519.4 ([14+H], 100); 520.4 (10); 603.5 (10).

HRMS (ESI) calculated for C₃₁H₅₅N₂O₄ m/z=519.4162; measured: 519.4140.

### Compound 36: (20S)-3β-isobutyrylamino-20-dimethylamino-9,19-cyclo-4β,14α-dimethyl-4α-hydroxymethyl-16α-tosyl-5α,9β-pregnan-11-one 36

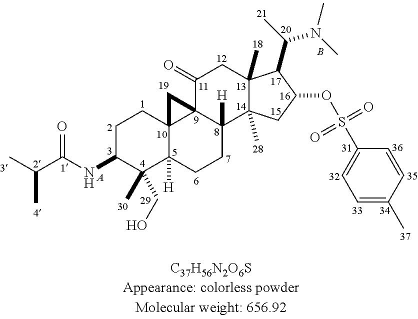

Under argon, to a solution of 52 mg (0.10 mmol, 1.0 eq) of N-3-isobutyrylcycloxobuxidine-F 2 and 7 mg (0.07 mmol, 0.7 eq) of dimethylaminopyridine in 3 ml of pyridine are added 60 mg (0.30 mmol, 3.0 eq) of tosyl chloride. After 12 hours of agitation at room temperature, the solution is co-evaporated in the presence of 1,2-dichloroethane. The residue is taken up with 30 ml of dichloromethane, washed with 30 ml of a 10% sodium hydrogen carbonate solution (pH=8) and extracted with 3×30 ml of dichloromethane. The recombined organic phases are dried on anhydrous sodium sulfate and evaporated under a vacuum.

The residue is chromatographed on a preparative silica plate (eluent: 9/1 dichloromethane/methanol) to yield 54 mg of 36 in the form of a yellow powder with a yield of 80%.

IR (CHCl₃) υ(cm⁻¹): 3434 (OH, NH); 1662 (C═O); 1662 and 1511 (CONH); 1096 (C—OH); 1354 and 1173 (C-OTs).

MS (ESI): m/z: 503.4 ([M+H], 100); 504.4 (10).

HRMS (ESI) calculated for C₃₇H₅₇N₂O₆S m/z=657.3937; measured: 657.3954.

¹H NMR (300 MHz, CDCl₃), δppm:

0.53 (3H, s, H₃₀); 0.72 (3H, d, J=6.0 Hz, H₂₁); 0.76 (3H, s, H₁₈); 0.84 (1H, m, H₆β); 1.04 (1H, d, J=4.0 Hz, H₁₉α); 1.12 and 1.14 (6H, 2d, J=6.6 Hz, H_{3′} and H_{4′}); 1.16 (3H, s, H₂₈); 1.24-1.67 (5H, m, H₁α, H₂β, H₇α, H₇β and H₂β); 1.52 (1H, d, J=4.0 Hz, H₁₉); 1.78 (6H, s, N_{B}—CH₃); 1.89-2.03 (5H, m, H₁₅α, H₂α, H₆α, H₈β, and H₅α); 2.14-2.19 (2H, m, H₁₅β, and H₁₇α); 2.27-2.50 (3H, m, H_{2′}, H₂₀, H₁β); 2.41 (3H, s, H₃₇); 2.35 and 2.43 (4H, 2d, J=17.3 Hz, H₁₂α and H₁₂β); 2.94 (1H, dd, J=12.6 Hz and 13.4 Hz, H₂₉ₐ); 3.30 (1H, d, J=12.6 Hz, H_{29b}); 3.94 (1H, m, H₃α); 4.76 (1H, m, OH); 4.90 (1H, m, H₁₆β); 5.59 (1H, d, J=8.9 Hz, N_{A}H); 7.28 and 7.73 (4H, system AB, H_{Ar}=32, 33, 35 and 36).

¹³C NMR (75.5 MHz, CDCl₃), δppm:

9.9 (C₂₁); 11.1 (C₃₀); 17.7 (C₁₈); 18.3 (C₆); 19.4 and 19.5 (C_{3′} and C_{4′}); 20.1 (C₂₈); 21.6 (C₃₇); 24.3 (C₇); 27.4 and 27.6 (C₂ and C₁); 30.8 (C₁₉); 33.8 (C₉); 35.6 (C_{2′}); 38.0 (C₁₀); 39.8 (N_{B}—CH₃); 41.1 and 41.4 (C₅ and C₈); 42.6 (C₁₅); 44.1 (C₄); 44.5 (C₁₃); 47.4 (C₁₄); 50.6 (C₃); 51.6 (C₁₂); 55.6 (C₁₇); 59.2 (C₂₀); 63.9 (C₂₉); 88.1 (C₁₆); 127.9 and 129.2 (C_{Ar}=32, 33, 35 and 36); 135.0 and 143.9 (C_{Ar}=31 and 34); 178.6 (C_{1′}); 210.8 (C₁₁).

### Compound 37: (20S)-3β-isobutyrylamino-20-dimethylamino-9,19-cyclo-4β,14α-dimethyl-4α-hydroxymethyl-16α-mesyl-5α,9β-pregnan-11-one 37

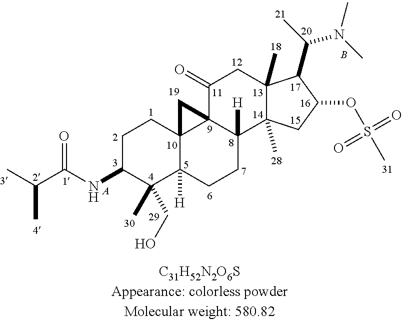

To a solution of 52 mg (0.10 mmol, 1.0 eq) of N-3-isobutyrylcycloxobuxidine-F 2 and 9 mg (0.07 mmol, 0.7 eq) of 4-dimethylaminopyridine in 3.0 ml of pyridine are added 24 μl (0.31 mmol, 3.0 eq) of mesyl chloride. The solution is yellow in color. After 16 hours of agitation at room temperature, the solution is co-evaporated with 1,2-dichloroethane. The residue is taken up with 20 ml of dichloromethane, washed with 25 ml of permuted water and extracted with 3×30 ml of dichloromethane. The recombined organic phases are dried on anhydrous sodium sulfate and evaporated under a vacuum.

The residue is chromatographed on a preparative silica plate (eluent: 9/1 dichloromethane/methanol) to yield 50 mg of 37 in the form of a colorless powder with a yield of 83%.

IR (diamond) υ(cm⁻¹): 2963 (CH, CH₂); 1654 (C═O); 1654 and 1512 (CONH); 1261 (C-OMs); 1351, 1169 (OMs); 1097 (C—O).

MS (ESI): m/z: 581.3 ([M+H], 100); 582.3 (60); 583.4 (10).

HRMS (ESI) calculated for C₃₁H₅₃N₂O₆S m/z=581.3624; measured: 581.3607.

¹H NMR (300 MHz, CDCl₃/D₂O), δppm: 0.57 (3H, s, H₃₀); 0.85 (3H, s, H₁₈); 0.88 (3H, d, J=6.9 Hz, H₂₁); 0.95 (1H, m, H₆β); 1.06 (1H, d, J=3.0 Hz, H₁₉); 1.17 and 1.19 (6H, 2d, J=6.4 Hz, H_{3′} and H_{4′}); 1.21 (3H, s, H₂₈); 1.26 (1H, m, H₁α); 1.30-1.53 (3H, m, H₇α, H₁₅α and H₇β); 1.58 (1H, d, J=3.0 Hz, H₁₉α); 1.54-1.75 (3H, m, H₂β, H₂α and H₆α); 1.95-2.23 (3H, m, H₈β, H₁₅β and H₅α); 2.17 (6H, bs, N_{B}—CH₃); 2.29 (1H, dd, J=5.5 Hz and 11.3 Hz, H₁₇α); 2.40 (1H, dq, J=6.4 Hz, H_{2′}); 2.39 (1H, d, J=17.1 Hz, H₁₂β); 2.46 (1H, m, H₁β); 2.51 (1H, m, H₂₀); 2.56 (1H, d, J=17.1 Hz, H₁₂α); 2.96 (1H, d, 12.8 Hz, H₂₉ₐ); 3.02 (3H, s, H₃₁); 3.34 (1H, d, J=12.8 Hz, H_{29b}); 4.00 (1H, ddd, J=4.5 Hz, 8.8 Hz and 10.6 Hz, H₃α); 4.83 (1H, m, H₁₆β); 5.35 (1H, d, J=9.0 Hz, N_{A}H).

¹³C NMR (75.5 MHz, CDCl₃/D₂O), δppm: 10.0 (C₂₁); 11.2 (C₃₀); 17.6 (C₁₈); 18.4 (C₆); 19.5 and 19.6 (C_{3′} and C_{4′}); 20.2 (C₂₈); 24.5 (C₇); 27.6 (C₂); 27.7 (C₁); 31.1 (C₁₉); 33.9 (C₉); 35.8 (C_{2′}); 37.3 (C₃₁); 38.2 (C₁₀); 41.2 (C₅); 41.7 (C₈); 44.1 (C₁₅); 44.5 (C₁₃); 44.6 (C₄); 47.4 (C₁₄); 50.7 (C₃); 51.6 (C₁₂); 55.1 (C₁₇); 59.9 (C₂₀); 64.0 (C₂₉); 88.5 (C₁₆); 178.5 (C_{1′}); 210.8 (C₁₁).

### Compound 38: (20S)-20-dimethylamino-9,19-cyclo-4β,14α-dimethyl-16α-pivalyl-(2′-isopropyl-3,29-dihydro-4H-[1,3]-oxazine)-5α,9β-pregn-11-one 38

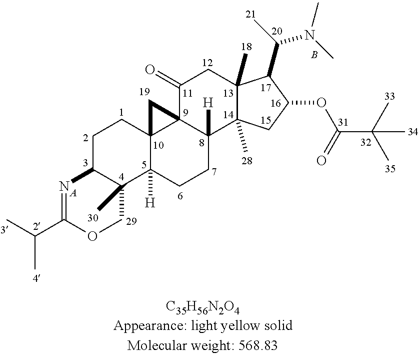

Under argon, to a solution of 412 mg (0.55 mmol, 1.0 eq) of tosylate 22 in 36 ml of DMF, are added 65 mg (0.66 mmol, 1.2 eq) of sodium diformylamidure. After 5 hours of agitation at 130° C., the mixture is alkalinized with 30 ml of a 10% sodium bicarbonate solution (pH=8) and extracted with 3×20 ml of diethyl ether. The recombined organic phases are successively washed with 2×20 ml of a 10% sodium bicarbonate solution, dried on anhydrous sodium sulfate and evaporated under a vacuum to yield 300 mg of 38 in the form of a light yellow solid with a yield of 94%.

IR (CHCl₃) υ(cm⁻¹): 2965 (C—H); 1730 (C═O ester); 1667 (C═N and C═C); 1157 (C—O).

MS (ESI): m/z: 569.4 ([M+H], 100); 693.5 (25); 763.5 (5).

HRMS (ESI) calculated for C₃₅H₅₇N₂O₄ m/z=569.4318; measured: 569.4330.

¹H NMR (300 MHz, CDCl₃), δppm: 0.83 (3H, s, H₁₈); 0.87 (3H, d, J=6.4 Hz, H₂₁); 0.92 (3H, s, H₃₀); 1.06 and 1.07 (6H, 2d, J=7.0 Hz, H_{3′} and H_{4′}); 1.15 (1H, m, H₆β); 1.21 (1H, d, J=4.0 Hz, H₁₉); 1.23 (3H, s, H₂₈); 1.35 (1H, m, H₁α); 1.37-1.53 (3H, m, H₇α, H₁₅α and H₇β); 1.56 (1H, d, J=4.0 Hz, H₁₉α); 1.53-1.58 (3H, m, H₂β); 1.95-2.19 (3H, m, H₆α, H₈β, H₁₅β, H₁₇α and H₅α); 2.24 (6H, bs, N_{B}—CH₃); 2.26 (1H, dq, J=7.0 Hz, H_{2′}); 2.31 (1H, d, J=17.1 Hz, H₁₂β); 2.42 (1H, m, H₁β); 2.52 (1H, d, J=17.3 Hz, H₁₂α); 2.62 (1H, dq, J=6.4 Hz and 10.9 Hz, H₂₀); 4.09 (1H, ddd, J=2.1 Hz, 7.3 Hz and 9.7 Hz, H₁₆β); 4.31 (1H, ddd, J=4.2 Hz, 8.9 Hz and 12.4 Hz, H₃α); 5.29 (1H, d, J=9.1 Hz, N_{A}H); 9.40 (1H, s, H₂₉).

¹³C NMR (75.5 MHz, CDCl₃), δppm: 7.8 (C₃₀); 10.1 (C₂₁); 18.0 (C₁₈); 19.4 and 19.9 (C_{3′} and C_{4′}); 20.8 (C₂₈); 21.2 (C₆); 24.1 (C₇); 27.5 (C₂); 27.6 (C₁); 29.6 (C₁₉); 34.1 (C₉); 35.7 (C_{2′}); 35.9 (C₁₀); 41.5 (C₂₀); 41.9 (C₅); 42.8 (C₁₅); 44.6 (C₁₃); 47.2 (C₁₄); 49.7 (C₃); 51.5 (C₁₂); 56.0 (C₁₇); 57.0 (C₄); 62.2 (C₂₀); 78.3 (C₁₆); 176.4 (C_{1′}); 203.4 (C₂₉); 210.9 (C₁₁).

### Compound 39: (20S)-10(9->1)abeo-20-dimethylamino-4β,14α-dimethyl-16α-pivalyl-(2′-isopropyl-3,29-dihydro-4H-[1,3]-oxazine)-5α,9β-pregn-1(10)-en-11-one 39

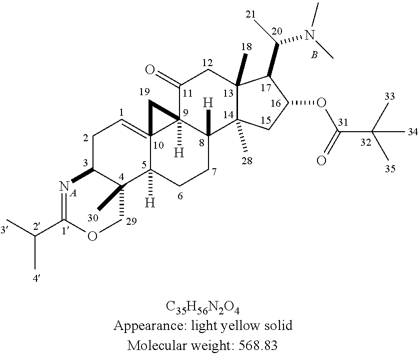

Under argon, to a solution of 50 mg (0.10 mmol, 1.0 eq) of dihydro-4H-(1,3)-oxazin 1 in 1 ml of pyridine, are added 13 μl (0.10 mmol, 1.0 eq) of pivalyle chloride. After 2.5 hours of agitation at room temperature, the mixture is alkalinized with 30 ml of a 10% ammonia solution (pH=10) and extracted with 3×30 ml of dichloromethane. The recombined organic phases are successively dried on anhydrous sodium sulfate and evaporated under a vacuum to provide 45 mg of 39 in the form of a light yellow solid with a yield of 77%.

IR (CHCl₃) υ(cm⁻¹): 1720 (C═O ester); 1698 (C═O); 1671 (C═N and C═C); 1158 (C—O).

MS (ESI): m/z: 569.4 ([14+H], 100); 570.4 (30); 485.4 (5); 285.2 (10).

HRMS (ESI) calculated for C₃₅H₅₇N₂O₄ m/z=569.4318; measured: 569.4301.

¹H NMR (500 MHz, CDCl₃), δppm: 0.65 (3H, s, H₁₈); 0.68 (3H, s, H₃₀); 0.78 (3H, d, J=6.9 Hz, H₂₁); 1.04 and 1.06 (6H, 2d, J=6.9 Hz, H_{3′} and H_{4′}); 1.19 (3H, s, H₂₈); 1.23-1.29 (2H, m, H₆β and H₇α); 1.26 (1H, dd, J=5.7 Hz and 14.7 Hz, H₁₅α); 1.53 (1H, dd, J=6.9 Hz and 12.0 Hz, H₆α); 1.62 (1H, ddd, J=3.8 Hz, 10.4 Hz and 10.7 Hz, H₈β); 1.72 (1H, m, H₂β); 1.79-1.83 (3H, m, H₅α, H₇β and H₉α); 1.98 (1, m, H₁₅β); 2.02 (1H, m, H₁₉); 2.06 (6H, s, N_{B}—CH₃); 2.20 (1H, dd, J=5.6 Hz and 11.4 Hz, H₁₇α); 2.28 (1H, m, H₂α); 2.34 (1H, dq, J=6.9 Hz, H_{2′}); 2.36 (1H, d, J=15.8 Hz, H₁₂β); 2.38 (1H, m, H₂₀); 2.52 (1H, bd, J=15.8 Hz, H₁₂α); 3.16 (1H, dd, J=6.3 Hz and 11.7 Hz, H₃α); 3.29 (1H, d, J=15.8 Hz, H₁₉α); 3.67 (1H, d, J=10.1 Hz, H₂₉α); 3.99 (1H, d, J=10.1 Hz, H₂₉); 4.98 (1H, dd, J=5.7 Hz and 13.2 Hz, H₁₆β); 5.53 (1H, m, H₁).

¹³C NMR (125 MHz, CDCl₃), δppm: 9.62 (C₃₀); 10.03 (C₂₁); 17.84 (C₁₈); 19.77 (C_{3′} and C_{4′}); 19.84 (C₂₈); 24.61 (C₆); 27.17 (C₃₃, C₃₄ and C₃₅); 30.73 (C₂); 33.20 (C₄); 33.52 (C₇); 34.31 (C_{2′}); 37.11 (C₁₉); 38.54 (C₃₂); 40.64 (N_{B}—CH₃); 43.26 (C₁₅); 46.41 (C₁₄); 47.11 (C₁₃); 49.56 (C₅); 49.82 (C₈); 50.31 (C₁₂); 50.32 (C₉); 54.07 (C₁₇); 55.65 (C₃); 59.61 (C₂₀); 74.74 (C₂₉); 78.94 (C₁₆); 121.87 (C₁); 137.08 (C₁₀); 162.70 (C_{1′}); 177.63 (C₃₁); 211.23 (C₁₁).

### Compound 40: (20S)-10(9->1)abeo-16α-acetyl-20-dimethylamino-4β,14α-dimethyl-(2′-isopropyl-3,29-dihydro-4H-[1,3]-oxazine)-5α,9β-pregn-1(10)-en-11-one 40

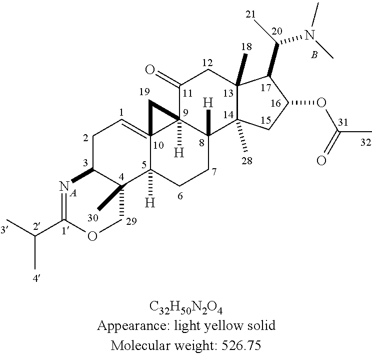

Under argon, to a solution of 50 mg (0.10 mmol, 1.0 eq) of dihydro-4H-(1,3)-oxazin 1 in 1 ml of pyridine, are added 8 μl (0.11 mmol, 1.0 eq) of acetyl chloride. After 2.5 hours of agitation at room temperature, the mixture is alkalinized with 30 ml of a 10% ammonia solution (pH=10) and extracted with 3×30 ml of dichloromethane. The recombined organic phases are successively dried on anhydrous sodium sulfate and evaporated under a vacuum with 1,2-dichloroethane to yield 37 mg of 40 in the form of a light yellow solid with a yield of 70%.

IR (CHCl₃) υ(cm⁻¹): 2965 (C—H); 1729 (C═O ester); 1696 (C═O); 1670 (C═N and C═C); 1027 (C—O).

### Compound 41: (20S)-3β-isobutyrylamino-20-dimethylamino-9,19-cyclo-4β,14α-dimethyl-4α-acetylmethyl-5α,9β-pregnan-16α-ol-11-one 41

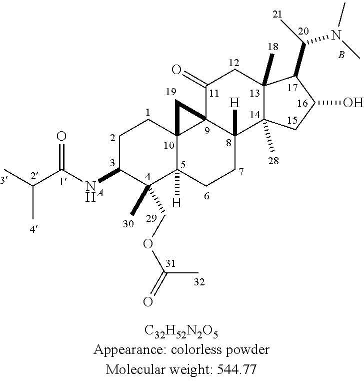

To a solution cooled to 5° C. of 200 mg (0.39 mmol, 1.0 eq) of N-3-isobutyrylcycloxobuxidine-F 2 in 25 ml of glacial acetic acid are added 5 ml (0.31 mmol, 3.0 eq) of 35% hydrochloric acid. After 30 minutes of agitation at room temperature, the solution is evaporated under a vacuum. The residue is alkalinized with 40 ml of a 10% ammonia solution and extracted with 3×40 ml of dichloromethane. The recombined organic phases are dried on anhydrous sodium sulfate and evaporated under a vacuum.

The residue thus obtained is chromatographed on a Merck activity II-III alumina column (eluent: 99.8/0.2 dichloromethane/methanol) to yield 168 mg of 41 in the form of a colorless powder with a yield of 77%.

Elemental analysis: for C₃₂H₅₂N₂O₅

Calculated for C₃₂H₅₂N₂O₅%: C, 70.55; H, 9.62; N, 5.14

Measured %: C, 70.01; H, 9.44; N, 4.83

IR (CHCl₃) υ(cm⁻¹): 3439 (OH, NH); 2966 (CH, CH₂) 1724 (C═O ester); 1667 and 1507 (C═O and CONH); 1095 (C—O).

MS (ESI): m/z: 545.5 ([M+H], 100); 546.5 (10); 624.5 (20).

HRMS (ESI) calculated for C₃₂H₅₂N₂O₅ m/z=545.3954; measured: 545.3607.

¹H NMR (300 MHz, CDCl₃), δppm: 0.67 (3H, s, H₃₀); 0.78 (3H, s, H₁₈); 0.81 (3H, d, J=6.4 Hz, H₂₁); 0.90 (1H, m, H₆β); 0.97 (1H, d, J=3.4 Hz, H₁₉); 1.05 and 1.06 (6H, 2d, J=6.8 Hz, H_{3′} and H_{4′}); 1.16 (3H, s, H₂₈); 1.20 (1H, m, H₁α); 1.25-1.52 (4H, m, H₇α, H₁₅α, H₇β and H₂β); 1.49 (1H, d, J=3.4 Hz, H₁₉α); 1.66 (1H, m, H₂α); 1.89-2.01 (5H, m, H₆α, H₈β, H₁₅β, H₅α and H₁₇α); 2.05 (3H, s, H₃₂); 2.18 (6H, bs, N_{B}—CH₃); 2.22 (1H, dq, J=6.8 Hz, H_{2′}); 2.23 (1H, d, J=17.2 Hz, H₁₂β); 2.31 (1H, m, H₁β); 2.46 (1H, d, J=17.2 Hz, H₁₂β); 2.55 (1H, dq, J=6.4 Hz and 11.8 Hz, H₂₀); 3.65 (1H, d, 11.7 Hz, H₂₉ₐ); 3.71 (1H, d, J=11.7 Hz, H_{29b}); 4.04 (1H, ddd, J=2.6 Hz, 7.2 Hz and 9.2 Hz, H₁₆β); 4.11 (1H, ddd, J=4.1 Hz, 8.1 Hz and 10.6 Hz, H₃α); 5.24 (1H, d, J=10.0 Hz, N_{A}H).

¹³C NMR (75.5 MHz, CDCl₃), δppm: 9.9 (C₂₁); 11.3 (C₃₀); 17.8 (C₁₈); 18.7 (C₆); 19.4 and 19.9 (C_{3′} and C_{4′}); 20.7 (C₂₈); 21.0 (C₃₂); 24.4 (C₇); 27.5 (C₂); 28.1 (C₁); 30.5 (C₁₉); 34.2 (C₉); 35.9 (C_{2′}); 37.3 (C₁₀); 41.6 (C₄); 42.5 (C₈); 42.6 (C₅); 42.7 (C₁₅); 44.3 (C₁₃); 47.0 (C₁₄); 49.5 (C₃); 51.4 (C₁₂); 55.7 (C₁₇); 61.9 (C₂₀); 65.3 (C₂₉); 78.2 (C₁₆); 171.3 (C₃₁); 176.3 (C_{1′}); 211.3 (C₁₁).

### Compound 42: (20S)-16α-acetyl-3β-isobutyrylamino-20-dimethylamino-9,19-cyclo-4β,14α-dimethyl-4α-acetylmethyl-5α,9β-pregnan-11-one 42

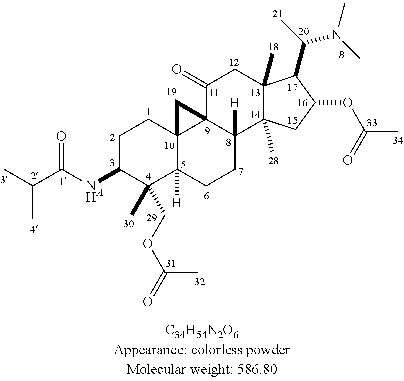

To a solution of 200 mg (0.39 mmol, 1.0 eq) of N-3-isobutyrylcycloxobuxidine-F 2 in 1 ml of pyridine are added 0.11 ml (1.19 mmol, 3.0 eq) of acetic anhydride. After 48 hours of agitation at room temperature, the solution is evaporated under a vacuum in the presence of 1,2-dichloroethane.

The residue thus obtained is chromatographed on a Merck activity II-III alumina column (eluent: 99/1 dichloromethane/methanol) to yield 212 mg of 42 in the form of a colorless powder with a yield of 91%.

IR (CHCl₃) υ(cm⁻¹): 2965 (CH, CH₂); 2359 (C—H); 1729 (C═O ester); 1666 and 1527 (C═O and CONH); 1229 and 1034 (C—O).

MS (ESI): m/z: 587.4 ([M+H], 100); 588.4 (5); 545.4 (20).

HRMS (ESI) calculated for C₃₄H₅₅N₂O₆ m/z=587.4060; measured: 587.4063.

¹H NMR (300 MHz, CDCl₃), δppm: 0.74 (3H, s, H₃₀); 0.82 (3H, d, J=6.4 Hz, H₂₁); 0.85 (3H, s, H₁₈); 0.98 (1H, m, H₆β); 1.05 (1H, d, J=3.8 Hz, H₁₉); 1.13 and 1.14 (6H, 2d, J=6.8 Hz, H_{3′} and H_{4′}); 1.18 (3H, s, H₂₈); 1.26 (1H, m, H₁α); 1.26-1.52 (5H, m, H₆α, H₇α, H₁₅α, H₇β and H₂β); 1.58 (1H, d, J=3.8 Hz, H₁₉α); 1.71-1.78 (2H, m, H₂α, H₈β); 2.00 (3H, s, H₃₄); 2.02-2.07 (2H, m, H₅α and H₁₅β); 2.11 (3H, s, H₃₂); 2.14 (6H, bs, N_{B}—CH₃); 2.23 (1H, dd, J=5.8 Hz and 11.2 Hz, H₁₇α); 2.31 (1H, dq, J=6.8 Hz, H_{2′}); 2.36 (1H, m, H₁β); 2.39 (1H, d, J=17.1 Hz, H₁₂β); 2.47 (1H, dq, J=6.4 Hz and 11.2 Hz, H₂₀); 2.56 (1H, d, J=17.1 Hz, H₁₂α); 3.72 (1H, d, 11.7 Hz, H₂₉ₐ); 3.78 (1H, d, J=11.7 Hz, H_{29b}); 4.18 (1H, ddd, J=4.1 Hz, 10.1 Hz and 12.1 Hz, H₃α); 5.12 (1H, dd, J=5.8 Hz and 7.9 Hz, H₁₆β); 5.18 (1H, d, J=10.4 Hz, N_{A}H).

¹³C NMR (75.5 MHz, CDCl₃), δppm: 9.8 (C₂₁); 11.4 (C₃₀); 17.9 (C₁₈); 18.8 (C₆); 19.5 (C_{3′} and C_{4′}); 20.0 (C₂₈); 21.0 (C₃₂); 21.2 (C₃₄); 24.5 (C₇); 27.5 (C₂); 28.2 (C₁); 30.5 (C₁₉); 33.8 (C₉); 36.0 (C_{2′}); 37.5 (C₁₀); 40.3 (N_{B}—CH₃); 41.5 (C₈); 42.6 (C₅ and C₄); 43.0 (C₁₅); 44.5 (C₁₃); 47.4 (C₁₄); 49.6 (C₃); 51.9 (C₁₂); 55.1 (C₁₇); 59.5 (C₂₀); 65.4 (C₂₉); 78.9 (C₁₆); 170.7 (C₃₃); 171.3 (C₃₁); 176.3 (C_{1′}); 211.0 (C₁₁).

### Compound 43: (20S)-3β-isobutyrylamino-20-methylamino-9,19-cyclo-4β,14α-dimethyl-4α-formyl-16α-pivalyl-5α,9β-pregnan-11-one 43

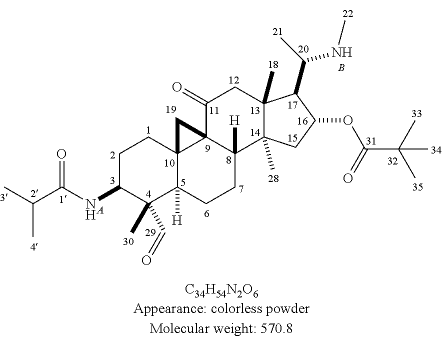

To a solution of 500 mg (0.85 mmol, 1.0 eq) of 16-O-pivalyl-N-3-isobutyrylcycloxobuxidine-F 17 in 32 ml of dichloromethane are added 486 mg (1.11 mmol, 1.3 eq) of the Dess-Martin periodinane under inert atmosphere. The solution clouds and becomes pink. After 16 hours of agitation at room temperature, the mixture is alkalinized with 40 ml of a 10% ammonia solution (pH=10) and extracted with 3×30 ml of dichloromethane. The recombined organic phases are successively dried on anhydrous sodium sulfate and evaporated under a vacuum.

The residue thus obtained is purified by silica gel flash chromatography (eluent: 90/10 dichloromethane/methanol) to yield 130 mg of 43 in the form of a colorless powder with a yield of 22%.

¹H NMR (300 MHz, CDCl₃), δppm:

0.83 (3H, s, H₁₈); 0.86 (3H, s, H₃₀); 1.00 (3H, d, J=6.6 Hz, H₂₁); 1.01 (1H, m, H₆β); 1.01 and 1.02 (6H, 2d, J=6.8 Hz, H_{3′} and H_{4′}); 1.09 (1H, m, H₁₉α); 1.11 (3H, s, H₂₈); 1.13 (9H, s, H₃₃ H₃₄ and H₃₅); 1.22-1.51 (5H, m, H₁α, H₇α, H₁₅α, H₇α and H₂β); 1.53 (1H, d, J=4.1 Hz, H₁₉); 1.71-1.77 (2H, m, H₂α H₆α); 1.96-2.14 (3H, m, H₈β, H₁₅β and H₅α); 2.18 (1H, dq, J=6.8 Hz, H_{2′}); 2.28 (3H, s, N_{B}—CH₃); 2.43 (1H, d, J=17.3 Hz, H₁₂β); 2.34-2.51 (3H, m, H₂₀, H₁₇α and H₁β); 2.53 (1H, d, J=17.3 Hz, H₁₂α); 4.25 (1H, ddd, J=4.2 Hz, 8.7 Hz and 12.2 Hz, H₃α); 5.97 (1H, dd, J=6.0 Hz, 7.5 Hz, H₁₆β); 5.20 (1H, d, J=9.0 Hz, N_{A}H); 9.33 (1H, s, H₂₉).

¹³C NMR (75.5 MHz, CDCl₃), δppm: 7.6 (C₃₀); 18.0 (C₁₈); 18.5 (C₂₁); 19.2 and 19.3 (C_{3′} and C_{4′}); 19.8 (C₂₈); 21.0 (C₆); 24.0 (C₇); 27.0 (C₃₃, C₃₄ and C₃₅); 27.2 (C₂); 27.3 (C₁); 29.4 (C₁₉); 33.2 (N_{B}—CH₃); 33.5 (C₉); 35.6 (C_{2′}); 35.9 (C₁₀); 38.5 (C₃₂); 40.9 (C₅); 41.8 (C₈); 43.6 (C₁₅); 45.4 (C₁₃); 47.0 (C₁₄); 49.5 (C₃); 51.6 (C₁₂); 56.3 (C₁₇); 56.7 (C₄); 56.9 (C₂₀); 78.9 (C₁₆); 176.3 (C₃₁); 178.1 (C_{1′}); 198.9 (C₂₉); 203.3 (C₁₁).

### Compound 44: (20S)-3β-isobutyrylamino-20-dimethylamino-9,19-cyclo-4β,14α-dimethyl-4α-formyl-5α,9β-pregnan-16α-ol-11-one 44

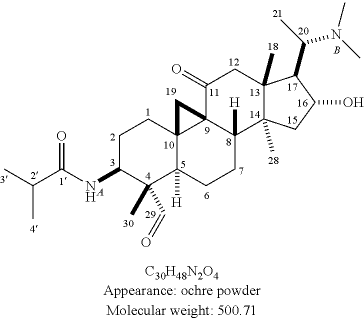

To a solution of 1000 mg (1.84 mmol, 1.0 eq) of aldehyde 14 in 23.0 ml of methanol are added a solution of 1953 mg (18.42 mmol, 10.0 eq) of sodium carbonate and 2321 mg (27.63 mmol, 15.0 eq) of sodium hydrogen carbonate in 25.0 ml of water. A precipitate is formed. After 3 hours of agitation at room temperature, the solution is diluted with 50 ml of water and is extracted with 3×45 ml of dichloromethane. The recombined organic phases are dried on anhydrous sodium sulfate and evaporated under a vacuum.

The residue is triturated in ethyl acetate to yield 567 mg of 44 in the form of an ochre powder with a yield of 61%.

IR (diamond) υ(cm⁻¹): 3284 (OH, NH); 2936 (CH, CH₂); 2791 (CH═O); 1716 (C═O aldehyde); 1661, 1636 and 1549 (C═O and CONH); 1095 (C—O).

MS (ESI): m/z: 501.4 ([M+H], 100); 502.4 (15).

HRMS (ESI) calculated for C₃₀H₄₉N₂O₄ m/z=501.3692; measured: 501.3678.

¹H NMR (300 MHz, CDCl₃), δppm: 0.83 (3H, s, H₁₈); 0.87 (3H, d, J=6.4 Hz, H₂₁); 0.92 (3H, s, H₃₀); 1.06 and 1.07 (6H, 2d, J=7.0 Hz, H_{3′} and H_{4′}); 1.15 (1H, m, H₆β); 1.21 (1H, d, J=4.0 Hz, H₁₉); 1.23 (3H, s, H₂₈); 1.35 (1H, m, H₁α); 1.37-1.53 (3H, m, H₇α, H₁₅α and H₇β); 1.56 (1H, d, J=4.0 Hz, H₁₉α); 1.53-1.58 (3H, m, H₂β); 1.95-2.19 (3H, m, H₆α, H₈β, H₁₅β, H₁₇α and H₅α); 2.24 (6H, bs, N_{B}—CH₃); 2.26 (1H, dq, J=7.0 Hz, H_{2′}); 2.31 (1H, d, J=17.1 Hz, H₁₂β); 2.42 (1H, m, H₁β); 2.52 (1H, d, J=17.3 Hz, H₁₂α); 2.62 (1H, dq, J=6.4 Hz and 10.9 Hz, H₂₀); 4.09 (1H, ddd, J=2.1 Hz, 7.3 Hz and 9.7 Hz, H₁₆β); 4.31 (1H, ddd, J=4.2 Hz, 8.9 Hz and 12.4 Hz, H₃α); 5.29 (1H, d, J=9.1 Hz, NAH); 9.40 (1H, s, H₂₉).

¹³C NMR (75.5 MHz, CDCl₃), δppm: 7.8 (C₃₀); 10.1 (C₂₁); 18.0 (C₁₈); 19.4 and 19.9 (C_{3′} and C_{4′}); 20.8 (C₂₃); 21.2 (C₆); 24.1 (C₇); 27.5 (C₂); 27.6 (C₁); 29.6 (C₁₉); 34.1 (C₉); 35.7 (C_{2′}); 35.9 (C₁₀); 41.5 (C₈); 41.9 (C₅); 42.8 (C₁₅); 44.6 (C₁₃); 47.2 (C₁₄); 49.7 (C₃); 51.5 (C₁₂); 56.0 (C₁₇); 57.0 (C₄); 62.2 (C₂₀); 78.3 (C₁₆); 176.4 (C_{1′}); 203.4 (C₂₉); 210.9 (C₁₁).

### Compound 45: (20S)-20-dimethylamino-3β-(isobutyrylamino)-9,19-cyclo-4α-benzyliminomethyl-4β,14α-dimethyl-5α,9β-pregnan-16α-ol-11-one 45

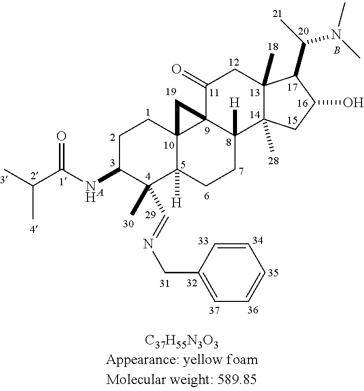

To a solution of 0.89 g (1.78 mmol, 1.0 eq) of aldehyde 44 and 900 mg of anhydrous magnesium sulfate in 10 ml of dichloromethane are added 253 μl (2.31 mmol, 1.3 eq) of benzylamine. After 17 hours of agitation at 55° C., the solution is filtered on celite and evaporated under a vacuum to yield 1.05 g of 45 in the form of a yellow foam with a quantitative yield.

IR (diamond) υ(cm⁻¹): 3386 (OH, NH); 2958 (CH, CH₂); 1649 (C═O, C═N); 1649 and 1529 (CONH); 1451 (C═C Ar); 1227 and 1091 (C—OH).

MS (ESI): m/z: 590.4 ([M+H], 100); 591.4 (10).

HRMS (ESI) calculated for C₃₇H₅₆N₃O₃ m/z=590.4322; measured: 590.4304.

¹H NMR (300 MHz, CDCl₃), δppm:

0.77 (3H, s, H₁₈); 0.81 (3H, d, J=6.6 Hz, H₂₁); 0.93 (1H, m, H₆β); 0.93 and 0.96 (6H, 2d, J=6.8 Hz, H_{3′} and H_{4′}); 0.97 (3H, s, H₃₀); 1.02 (1H, d, J=4.0 Hz, H₁₉α); 1.16 (3H, s, H₂₈); 1.21-1.44 (5H, m, H₁α, H₂β, H₇β, H₇α and H₁₅α); 1.50 (1H, m, H₆α); 1.53 (1H, d, J=4.0 Hz, H₁₉); 1.76 (1H, m, H₂α); 1.82-2.07 (4H, m, H₁₅β, H₁₇α, H₅α and H₈β); 2.10 (1H, dq, J=6.8 Hz, H_{2′}); 2.19 (6H, s, N_{B}—CH₃); 2.25 (1H, d, J=17.3 Hz, H₁₂β); 2.31 (1H, ddd, J=3.3 Hz, 3.6 Hz and 13.4 Hz, H₁β); 2.45 (1H, d, J=17.3 Hz, H₁₂α); 2.55 (1H, dd, J=6.8 Hz and 10.8 Hz, H₂₀); 4.03 (2H, m, H₃α and H₁₆β); 4.45 and 4.49 (2H, 2d system AB, 15.2 Hz, H₃₁); 5.26 (1H, d, J=9.2 Hz, N_{A}H); 7.14-7.27 (5H, m, H_{Ar=33-37}); 7.49 (1H, s, H₂₉).

¹³C NMR (75.5 MHz, CDCl₃), δppm:

10.0 (C₂₁); 10.2 (C₃₀); 17.9 (C₆ and C18); 19.3 and 19.6 (C_{3′} and C_{4′}); 20.7 (C₂₈); 24.2 (C₇); 27.6 (C₂); 27.7 (C₁); 29.8 (C₁₉); 34.3 (C₉); 35.7 (C_{2′}); 36.7 (C₁₀); 41.3 (C₈); 42.7 (C₁₅); 44.5 (C₁₃); 44.8 (C₅); 47.2 (C₁₄); 50.0 (C₄); 51.4 (C₁₂); 52.0 (C₃); 55.9 (C₁₇); 62.1 (C₂₀); 64.7 (C₃₁); 78.2 (C₁₆); 126.7 (C₃₅); 127.7 and 128.3 (C₃₄₋₃₆ and C₃₃₋₃₇); 139.5 (C₃₂); 170.7 (C₂₉); 176.0 (C_{1′}); 210.9 (C₁₁).

### Compound 46: (20S)-20-dimethylamino-3β-(isobutyrylamino)-9,19-cyclo-4α-benzylaminomethyl-4β,14α-dimethyl-5α,9β-pregnan-16α-ol-11-one 46

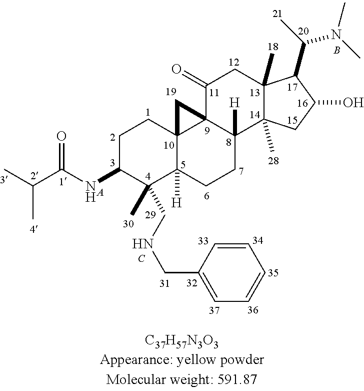

To a suspension of 2.98 g (5.05 mmol, 1.0 eq) of imine 45 in 30 ml of methanol are successively added 381 mg (6.06 mmol, 1.2 eq) of sodium cyanoborohydride and 289 μl (5.05 mmol, 1.05 eq) of glacial acetic acid. After 3 hours of agitation at room temperature, the mixture is alkalinized with 50 ml of a 10% ammonia solution (pH=10) and extracted with 3×50 ml of dichloromethane. The recombined organic phases are successively dried on anhydrous sodium sulfate and evaporated under a vacuum.

The residue is chromatographed on silica gel (eluent: 97.5/0.5/2 dichloromethane/methanol/ammonia) to yield 2.64 g of 46 in the form of a yellow powder with a yield of 88%, in two steps.

Elemental analysis: for C₃₇H₅₇N₃O₃.0.5H₂O

Calculated for C₃₇H₅₇N₃O₃.0.5H₂O %: C, 73.96; H, 9.73; N, 6.99; O, 9.32.

Measured %: C, 74.14; H, 9.63; N, 7.13; O, 9.11.

IR (diamond) υ(cm⁻¹): 3312 (OH, NH); 2933 (CH, CH₂); 1660 (C═O); 1660 and 1534 (CONH); 1452 (C═C Ar); 1226 and 1095 (C—OH).

MS (ESI): m/z: 592.5 ([M+H], 100); 593.5 (10); 296.7 (5).

HRMS (ESI) calculated for C₃₇H₅₈N₃O₃ m/z=592.4478; measured: 592.4451.

¹H NMR (300 MHz, CDCl₃), δppm:

0.51 (3H, s, H₃₀); 0.65 (1H, m, H₆β); 0.76 (3H, s, H₁₈); 0.80 (3H, d, J=6.6 Hz, H₂₁); 1.07 (6H, 2d, J=7.0 Hz, H_{3′} and H_{4′}); 0.92 (1H, d, J=3.4 Hz, H₁₉α); 1.17 (3H, s, H₂₈); 1.22-1.48 (4H, m, H₁α, H₇, H₂β and H₁₅α); 1.45 (1H, d, J=3.4 Hz, H₁₉); 1.60 (1H, m, H₂α); 1.87-2.15 (5H, m, H₁₅β, H₁₇α, H₆α, H₅α and H₈β); 2.02 (1H, d, J=13.0 Hz, H₂₉); 2.19 (6H, s, N_{B}—CH₃); 2.14 (1H, d, J=13.0 Hz, H₂₉); 2.22 (1H, d, J=17.1 Hz, H₁₂β); 2.24 (1H, dq, J=7.0 Hz, H_{2′}); 2.32 (1H, m, H₁β); 2.44 (1H, d, J=17.1 Hz, H₁₂α); 2.55 (1H, dd, J=6.6 Hz and 10.8 Hz, H₂₀); 3.56 and 3.71 (2H, 2d system AB, 13.8 Hz, H₃₁); 4.00 (1H, m, H₃α); 4.03 (1H, m, H₁₆β); 5.10 (1H, d, J=10.0 Hz, NAH); 7.19-7.28 (5H, m, H_{Ar=33-37}).

¹³C NMR (75.5 MHz, CDCl₃), δppm:

9.9 (C₂₁); 13.7 (C₃₀); 17.8 (C₁₈); 18.5 (C₆); 19.5 and 20.2 (C_{3′} and C_{4′}); 21.0 (C₂₈); 24.5 (C₇); 27.6 (C₂); 28.4 (C₁); 31.0 (C₁₉); 34.6 (C₉); 36.0 (C_{2′}); 38.2 (C₁₀); 41.7 (C₈); 42.1 (C₅); 42.9 (C₁₅); 43.2 (C₁₈); 44.5 (C₄); 47.2 (C₁₄); 50.3 (C₃); 51.5 (C₁₂); 52.9 (C₂₉); 54.5 (C₃₁); 55.8 (C₁₇); 62.1 (C₂₀); 78.4 (C₁₆); 126.5 (C₃₅); 128.0 and 128.4 (C₃₄₋₃₆ and C₃₃₋₃₇); 141.2 (C₃₂); 176.6 (C_{1′}); 211.6 (C₁₁).

### Compound 47: (20S)-3β-isobutyrylamino-20-dimethylamino-9,19-cyclo-4α-benzylaminomethyl-4β,14α-dimethyl-16α-pivalyl-5α,9β-pregnan-11-one 47

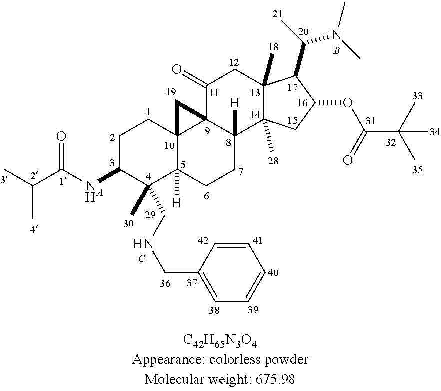

To a solution of 100 mg (0.15 mmol, 1.0 eq) of imine 19 in 0.5 ml of methanol are added 12 mg (0.18 mmol, 1.2 eq) of sodium cyanoborohydride and 9 μl (0.16 mmol, 1.05 eq) of glacial acetic acid. After 1 hour of agitation at room temperature, the mixture is alkalinized with 30 ml of a 10% ammonia solution (pH=10) and extracted with 3×20 ml of dichloromethane. The recombined organic phases are successively dried on anhydrous sodium sulfate and evaporated under a vacuum.

The residue thus obtained is chromatographed on a Merck activity II-III alumina column (eluent: 99.9/0.1 dichloromethane/40% ammonia) to yield 58 mg of 47 in the form of a colorless powder with a yield of 58%.

IR (diamond) υ(cm⁻¹): 2964 (NH); 1720 (C═O ester); 1664 and 1530 (C═O and CONH); 1453 1365 1287 1229 1159 (C═C_{Ar} C—N C—O).

MS (ESI): m/z: 676.5 ([M+H], 100); 586.5 (5); 339.3 (25); 338.8 (100).

HRMS (ESI) calculated for C₄₂H₆₆N₃O₄ m/z=676.5053; measured: 676.5053.

¹H NMR (300 MHz, CDCl₃), δppm: NCH can not be seen: No correlation is visible between this proton and the H₃₆ protons on the COSY spectra.

0.58 (3H, s, H₃₀); 0.82 (3H, s, H₁₈); 0.82 (1H, m, H₆β); 0.84 (3H, d, J=6.4 Hz, H₂₁); 1.14 and 1.20 (6H, 2d, J=6.8 Hz, H_{3′} and H_{4′}); 1.19 (3H, s, H₂₈); 1.21 (9H, s, H₃₃, H₃₄ and H₃₅); 1.13 (1H, m, H₁₉α); 1.24-1.56 (5H, m, H₁α, H₇α, H₁₅α, H₇β and H₂β); 1.55 (1H, d, J=3.8 Hz, H₁₉); 1.72-1.85 (2H, m, H₂α, H₆α); 2.14 (6H, s, N_{B}—CH₃); 2.08 and 2.16 (2H, 2d, J=12.4 Hz, H₂₉); 1.90-2.20 (3H, m, H₈β, H₁₅β and H₅α); 2.37 (1H, m, H_{2′}); 2.20-2.49 (4H, m, H₁₇α, H₂₀, H₁β and H₁₂β); 2.56 (1H, d, J=17.3 Hz, H₁₂α); 3.63 (2H, system AB, H₃₆); 3.97 (1H, ddd, J=4.5 Hz, 11.1 Hz and 12.7 Hz, H₃α); 4.96 (1H, dd, J=5.6 Hz and 7.5 Hz, H₁₆β); 5.08 (1H, d, J=9.8 Hz, N_{A}H); 7.10-7.25 (5H, m, H_{Ar}=38, 39, 40, 41 and 42).

¹³C NMR (75.5 MHz, CDCl₃), δppm:

13.7 (C₃₀); 17.8 (C₁₈); 18.4 (C₆); 19.5 (C₂₁); 19.5 and 19.7 (C_{3′} and C_{4′}); 20.2 (C₂₈); 24.5 (C₇); 27.2 (C₃₃₋₃₅); 27.6 (C₂); 28.4 (C₁); 30.9 (C₁₉); 34.1 (C₉); 36.0 (C_{2′}); 36.8 (C₁₀); 38.6 (C₃₂); 40.6 (N_{B}—CH₃); 41.3 (C₈); 42.1 (C₅); 43.2 (C₁₃); 43.6 (C₁₅); 44.7 (C₁₄); 47.4 (C₄); 50.3 (C₃); 52.0 (C₂₉); 52.9 (C₁₂); 54.6 (C₃₆); 54.9 (C₁₇); 59.5 (C₂₀); 79.2 (C₁₆); 126.6 (C_{Ar}=40); 127.7 and 128.0 (C_{Ar}=38, 39, 40 and 41); 139.5 (C₃₇); 170.7 (C₃₁); 176.0 (C_{1′}); 211.4 (C₁₁).

### Compound 48: (20S)-3β-isobutyrylamino-20-methylamino-9,19-cyclo-4α-benzylaminomethyl-4β,14α-dimethyl-16α-pivalyl-5α,9β-pregnan-11-one 48

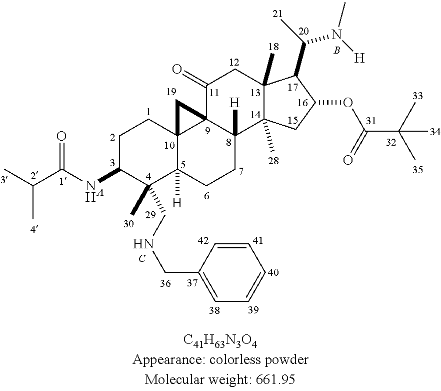

To a solution of 100 mg (0.15 mmol, 1.0 eq) of imine 19 in 0.5 ml of methanol are added 12 mg (0.18 mmol, 1.2 eq) of sodium cyanoborohydride and 9 μl (0.16 mmol, 1.05 eq) of glacial acetic acid. After 1 hour of agitation at room temperature, the mixture is alkalinized with 30 ml of a 10% ammonia solution (pH=10) and extracted with 3×20 ml of dichloromethane. The recombined organic phases are successively dried on anhydrous sodium sulfate and evaporated under a vacuum.

The residue thus obtained is chromatographed on a Merck activity II-III alumina column (eluent: 99/1 dichloromethane/40% ammonia) to yield 35 mg of 48 in the form of a colorless powder with a yield of 35%.

IR (diamond) υ(cm⁻¹): 3316 (NH and NH—CH₃); 1721 (C═O ester); 1662 and 1532 (C═O and CONH); 1453 (C═C Ar); 1159-1029 (C—O).

MS (ESI): m/z: 676.5 ([M+H], 100); 586.5 (5); 339.3 (25); 338.8 (100).

HRMS (ESI) calculated for C₄₂H₆₆N₃O₄ m/z=676.5053; measured: 676.5053.

### Compound 49: (20S)-20-dimethylamino-3β-(isobutyrylamino)-9,19-cyclo-4α-aminomethyl-4β,14α-dimethyl-5α,9β-pregnan-16α-ol-11-one 49

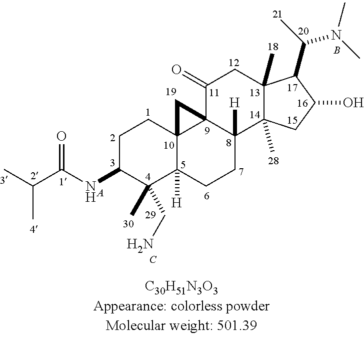

To a suspension of 817 mg (1.38 mmol, 1.0 eq) of N-benzylated amine 46 in 30 ml of anhydrous methanol degassed with argon, are successively added 408 mg (50% by weight with respect to X) of 30% palladium on carbon and 435 mg (6.90 mmol, 5.0 eq) of ammonium formate. After 3 hours of agitation at 40° C., the mixture is filtered on celite and evaporated under a vacuum. The residue obtained is alkalinized with 40 ml of a 10% sodium carbonate solution (pH=8) and extracted with 3×30 ml of dichloromethane. The recombined organic phases are successively dried on anhydrous sodium sulfate and evaporated under a vacuum.

The residue is chromatographed on a silica column (eluent: 96/2/2 dichloromethane/methanol/ammonia) to yield 588 mg of 49 in the form of a colorless powder with a yield of 85%, in two steps.

MP (° C.): 232° C.

IR (diamond) υ(cm⁻¹): 3313 (OH, NH); 2934 (CH, CH₂); 1650 (C═O); 1650 and 1536 (CONH); 1226 and 1094 (C—OH).

MS (ESI): m/z: 502.4 ([M+H], 100); 503.4 (10); 296.7 (15); 251.7 ([M+2H]/2.10).

HRMS (ESI) calculated for C₃₀H₅₂N₃O₃ m/z=502.4009; measured: 502.4008.

¹H NMR (300 MHz, CDCl₃), δppm:

0.56 (3H, s, H₃₀); 0.78 (3H, s, H₁₈); 0.80 (3H, d, J=6.6 Hz, H₂₁); 0.87 (1H, m, H₆β); 0.97 (1H, d, J=3.8 Hz, H₁₉α); 1.08 and 1.10 (6H, 2d, J=6.6 Hz, H_{3′} and H_{4′}); 1.15 (3H, s, H₂₈); 1.21-1.48 (4H, m, H₁α, H₇, H₂β and H₁₅α); 1.49 (1H, d, J=3.8 Hz, H₁₉); 1.62 (1H, m, H₂α); 1.88-2.00 (5H, m, H₁₅β, H₁₇α, H₆α, H₅α and H₈β); 2.18 (6H, s, N_{B}—CH₃); 2.24 (1H, d, J=17.1 Hz, H₁₂β); 2.26 (1H, d, J=13.9 Hz, H₂₉ₐ); 2.28 (1H, dq, J=6.6 Hz, H_{2′}); 2.38 (1H, d, J=13.9 Hz, H_{29b}); 2.39 (1H, m, H₁β); 2.44 (1H, d, J=17.1 Hz, H₁₂α); 2.55 (1H, dd, J=6.6 Hz and 10.9 Hz, H₂₀); 3.99 (1H, m, H₃α); 4.03 (1H, m, H16β); 5.13 (1H, d, J=9.8 Hz, N_{A}H).

¹³C NMR (75.5 MHz, CDCl₃), δppm:

9.9 (C₂₁); 13.7 (C₃₀); 17.8 (C₁₈); 18.5 (C₆); 19.5 and 20.1 (C_{3′} and C_{4′}); 21.0 (C₂₈); 24.6 (C₇); 27.7 (C₂); 28.4 (C₁); 31.2 (C₁₉); 34.4 (C₉); 35.9 (C_{2′}); 38.1 (C₁₀); 41.7 (C₈); 42.0 (C₅); 42.9 (C₁₅); 43.7 (C₁₃); 44.5 (C₄); 46.2 (C₂₉); 47.1 (C₁₄); 49.5 (C₃); 51.5 (C₁₂); 55.8 (C₁₇); 62.0 (C₂₀); 78.3 (C₁₆); 176.8 (C_{1′}); 211.5 (C₁₁).

### Compound 50: (20S)-3β-isobutyrylamino-20-dimethylamino-9,19-cyclo-4α-aminomethyl-4β,14α-dimethyl-16α-pivalyl-5α,9β-pregnan-11-one 50

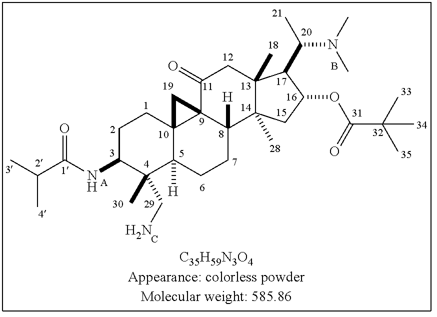

First Method: Benzylamine Route, Deprotection Under Pressure of Hydrogen

To a solution of 82 mg (0.12 mmol, 1.0 eq) of benzylamine 47 in 2 ml of methanol are successively added 40 mg (50% by weight) of palladium hydroxide on carbon. The pH of the solution is adjusted to 3 with glacial acetic acid. After 48 hours of agitation at room temperature under a pressure of 2 bar of hydrogen (H₂), in a Parr apparatus, the mixture is filtered on celite and the solvents evaporated under a vacuum. The residue is alkalinized with 20 ml of a 10% ammonia solution (pH=10) and extracted with 3×20 ml of dichloromethane. The recombined organic phases are successively dried on anhydrous sodium sulfate and evaporated under a vacuum.

The residue thus obtained is chromatographed on a Merck activity II-III alumina column (eluent: 99.9/0.1 dichloromethane/40% ammonia) to yield 23 mg of 50 in the form of a colorless powder with a yield of 32%.

Second Method: Benzylamine Route, Deprotection Under Ammonium Formate

Under argon, to a solution of 227 mg (0.33 mmol, 1.0 eq) of benzylamine 47 in 5 ml of methanol are successively added 113 mg (50% by weight) of 30% palladium on carbon and 109 mg (1.73 mmol, 5.0 eq) of ammonium formate. After 5 hours of agitation at 65° C., the mixture is filtered on celite and the solvents evaporated under a vacuum. The residue is alkalinized with 20 ml of a 10% sodium hydrogen carbonate solution (pH=8) and extracted with 3×20 ml of dichloromethane. The recombined organic phases are successively dried on anhydrous sodium sulfate and evaporated under a vacuum.

The residue thus obtained is chromatographed on a Merck activity II-III alumina column (eluent: 99.9/0.1 dichloromethane/40% ammonia) to yield 24 mg of 50 in the form of a colorless powder with a yield of 12%.

Third Method: Bis(Trimethylsilyl)Methylamine Route

Under argon, to a solution of 65 mg (0.08 mmol, 1.0 eq) of amine 56 in 4 ml of methanol are successively added 371 mg (0.67 mmol, 8.0 eq) of cerium IV ammonium nitrate. The solution is red. After 4 days of agitation at room temperature, the solution becomes colorless and the mixture is alkalinized with 20 ml of a 10% ammonia solution (pH=10) and extracted with 3×25 ml of dichloromethane. The recombined organic phases are successively dried on anhydrous sodium sulfate and evaporated under a vacuum.

The residue thus obtained is chromatographed on a Merck activity II-III alumina column (eluent: 99.7/0.3 dichloromethane/methanol) to yield 37 mg of 50 in the form of a colorless powder with a yield of 55%.

IR (diamond) υ(cm⁻¹): 3320 (NH); 1720 (C═O ester); 1665 and 1530 (C═O and CONH); 1455 (C—H groupes alkyles); 1377 (CH); 1159 (C—O).

MS (ESI): m/z: 586.4 ([M+H], 100); 591.4 (40); 559.4 (40); 394.6 (40).

HRMS (ESI) calculated for C₃₅H₆₀N₃O₄ m/z=586.4584; measured: 586.4579.

¹H NMR (300 MHz, CDCl₃), δppm:

0.66 (3H, s, H30); 0.82 (3H, d, J=6.4 Hz, H21); 0.85 (3H, s, H18); 1.03 (1H, d, J=3.6 Hz, H19a); 1.06 (1H, m, H6b); 1.14 and 1.15 (6H, 2d, J=6.8 Hz, H3′ and H4′); 1.17 (3H, s, H28); 1.19 (9H, s, H33, H34 and H35); 1.24 (1H, m, H1a); 1.35 (1H, bd, J=14.3 Hz, H15a); 1.36 (1H, m, H7a); 1.43 (1H, m, H7b); 1.49 (1H, m, H2b); 1.55 (1H, d, J=3.6 Hz, H19b); 1.68 (1H, m, H2a); 1.77 (1H, m, H6a); 1.98 (1H, m, H15b); 2.02 (1H, m, H5a); 2.05 (1H, m, H8b); 2.13 (6H, s, NB—CH3); 2.24 (1H, d, J=12.3 Hz, H29a); 2.25 (1H, dd, J=5.3 Hz and 11.2 Hz, H17a); 2.28 (1H, d, 12.3 Hz, H29b); 2.38 (1H, d, J=17.0 Hz, H12b); 2.38 (1H, m, H1b); 2.42 (1H, dq, J=6.8 Hz, H2′); 2.44 (1H, dd, J=6.2 Hz and 11.2 Hz, H20); 2.54 (1H, d, J=17.0 Hz, H12a); 3.98 (1H, ddd, J=4.0 Hz, 10.3 Hz and 10.8 Hz, H3a); 5.05 (1H, dd, J=5.3 Hz and 7.8 Hz, H16b); 5.24 (1H, d, J=8.9 Hz, NAH).

¹³C NMR (75.5 MHz, CDCl₃), δppm:

9.9 (C21); 13.9 (C30); 17.8 (C18); 18.6 (C6); 19.5 and 19.8 (C3′ and C4′); 20.2 (C28); 24.7 (C7); 27.2 (C33-35); 27.6 (C2); 28.4 (C1); 31.5 (C19); 34.0 (C9); 36.0 (C2′); 38.0 (C10); 38.6 (C32); 40.6 (NB—CH3); 41.4 (C8); 41.8 (C5); 43.6 (C15); 43.7 (C13); 44.7 (C14); 44.9 (C29); 47.4 (C4); 50.4 (C3); 52.0 (C12); 54.9 (C17); 59.5 (C20); 79.2 (C16); 170.7 (C31); 177.9 (C1′); 211.5 (C11).

### Compound 51: (20S)-20-dimethylamino-9,19-cyclo-4β,14α-dimethyl-(2-isopropyl-5β-methyl-1,4,5,6-tetrahydro-pyrimidin-5-yl)-5α,9β-pregnan-16α-ol-11-one 51

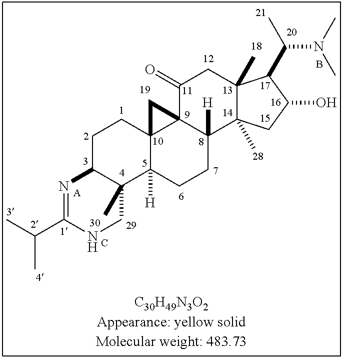

To a solution of 537 mg (1.07 mmol, 1.0 eq) of amine 49 in 50 ml of n-butanol is added 299 μl (2.14 mmol, 2.0 eq) of triethylamine. After 16 hours of agitation at 120° C., the mixture is evaporated under a vacuum.

The residue is chromatographed on a Merck activity II-III alumina column (eluent: 95/5 dichloromethane/methanol) to yield 331 mg of 51 in the form of a yellow solid with a yield of 64%.

MP (° C.): 255° C.

IR (diamond) υ(cm⁻¹): 3375-3143 (OH, NH); 2932 (CH, CH₂); 1662 (C═O); 1629 and 1530 (C—N, C═N); 1039 (C—OH).

MS (ESI): m/z: 484.4 ([M+H], 100); 485.4 (5); 439.3 (5); 242.7 ([M+2H/2], 30), 243.2 (5).

HRMS (ESI) calculated for C₃₀H₅₀N₃O₂ m/z=484.3903; measured: 484.3904.

¹H NMR (500 MHz, CDCl₃), δppm:

0.67 (3H, s, H₃₀); 0.77 (3H, s, H₁₈); 0.80 (3H, d, J=6.3 Hz, H₂₁); 1.00 (1H, m, H₆β); 1.01 (1H, d, J=3.8 Hz, H₁₉α); 1.06 and 1.07 (6H, 2d, J=6.9 Hz, H_{3′} and H_{4′}); 1.15 (3H, s, H₂₈); 1.16-1.37 (3H, m, H₁α and H₇); 1.42 (1H, dd, J=3.2 Hz and 14.2 Hz, H₁₅α); 1.49 (1H, m, H₂β); 1.49 (1H, d, J=3.8 Hz, H₁₉); 1.62 (2H, m, H₆α and H₂α); 1.89-1.99 (3H, m, H₁₅β, H₁₇α and H₅α); 2.03 (1H, m, H₈β); 2.18 (6H, s, N_{B}—CH₃); 2.25 (1H, d, J=17.0 Hz, H₁₂β); 2.30 (1H, dq, J=6.6 Hz, H_{2′}); 2.31 (1H, m, H₁β); 2.42 (1H, d, J=17.0 Hz, H₁₂α); 2.55 (1H, dd, J=6.3 Hz and 10.8 Hz, H₂₀); 2.92 (1H, d, J=13.0 Hz, H₂₉ₐ); 3.10 (1H, d, J=13.0 Hz, H_{29b}); 3.11 (1H, m, H₃α); 4.02 (1H, ddd, J=2.3 Hz, 7.6 Hz and 14.2 Hz, H₁₆β); 4.23 (1H, bs, N_{C}H/N_{A}H exchange).

¹³C NMR (125.8 MHz, CDCl₃), δppm:

9.9 (C₂₁); 11.6 (C₃₀); 17.8 (C₁₈); 18.2 (C₆); 20.5 (C_{3′} and C_{4′}); 20.6 (C₂₈); 24.2 (C₇); 27.4 (C₂ and C₁); 30.3 (C₁₉); 34.3 (C₅); 34.8 (C_{2′}); 34.9 (C₉); 38.0 (C₁₀); 41.0 (C₈); 42.7 (C₁₅); 44.6 (C₁₃); 47.2 (C₁₄); 47.3 (C₄); 51.5 (C₁₂); 54.2 (mass with low C3 integration); 55.9 (C₁₇); 58.3 (mass with low C29 integration); 62.0 (C₂₀); 78.3 (C₁₆); 160.6 (C_{1′}); 210.9 (C₁₁).

### Compound 52: (20S)-20-dimethylamino-9,19-cyclo-4β,14α-dimethyl-16α-pivalyl-(2′-isopropyl-1′/3′,3,29-tetrahydropyrimidine)-5α,9β-pregnan-11-one 52

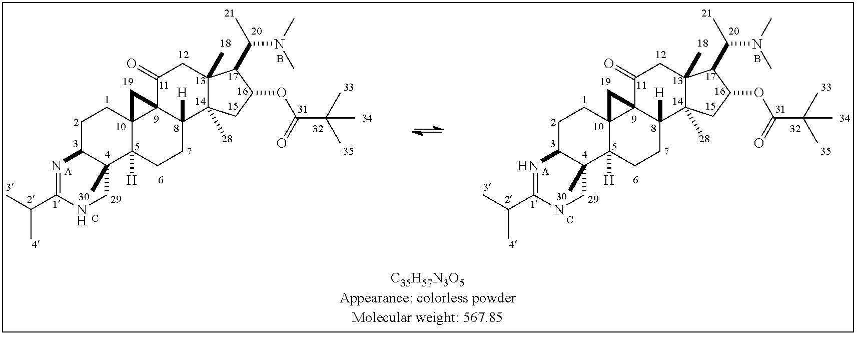

First Method: Ammonium Formate

Under argon, to a solution of 66 mg (0.10 mmol, 1.0 eq) of benzylamine 47 in 2 ml of methanol are successively added 66 mg (100% by weight) of 30% palladium on carbon and 62 mg (1.00 mmol, 10.0 eq) of ammonium formate. After 16 hours of agitation at 70° C., the mixture is filtered on celite and the solvents evaporated under a vacuum. The residue is alkalinized with 20 ml of a 10% ammonia solution (pH=10) and extracted with 3×40 ml of dichloromethane. The recombined organic phases are successively dried on anhydrous sodium sulfate and evaporated under a vacuum.

The residue thus obtained is chromatographed on a Merck activity II-III alumina column (eluent: 93.9/0.1/6 dichloromethane/40% ammonia/MeOH) to yield 18 mg of 52 in the form of a colorless powder with a yield of 33%.

Second Method: in Solution in n-Butanol

To a solution of 50 mg (0.08 mmol, 1.0 eq) of amine 50 in 2 ml of n-butanol is added 24 ml (0.17 mmol, 2.0 eq) of triethylamine. After 16 hours of agitation at 120° C., the mixture is evaporated under a vacuum.

The residue is chromatographed on a Merck activity II-III alumina column (eluent: 95/5 dichloromethane/methanol) to yield 29.5 mg of 52 in the form of a yellow solid with a yield of 61%.

MP (° C.): 146

IR (diamond) υ(cm⁻¹): 2932-2967 (CH); 1716 (C═O ester); 1663 and 1631 (C═O and C═N); 1459 (C—H groupes alkyles); 1366 (CH₃); 1160 (C—O).

MS (ESI): m/z: 568.4 ([M+H], 100); 569.4 (10); 484.4 (5).

HRMS (ESI) calculated for C₃₅H₅₈N₃O₃ m/z=568.4478; measured: 568.4507.

Note: The tetrahydro-pyrimidines are in equilibrium with each of their tautomeric forms. Indeed, the ¹³C NMR study shows that carbons C-3, C-4 and C-29 resonate in the form of masses of very low intensity. However, the chemical shift of these carbons is determined using their correlations with the protons of the methyl at position 30, on the HMBC spectrum.

¹H NMR (300 MHz, CDCl₃), δppm:

0.74 (3H, s, H30); 0.83 (3H, d, J=6.4 Hz, H21); 0.86 (3H, s, H18); 1.05 (1H, m, H6b); 1.13 and 1.14 (6H, 2d, J=7.0 Hz, H3′ and H4′); 1.16 (3H, s, H28); 1.18 (9H, s, H33, H34 and H35); 1.13 (1H, m, H19a); 1.25 (1H, m, H1a); 1.28 (1H, m, H7a); 1.31 (1H, m, H7b); 1.37 (1H, bd, J=14.5 Hz, H15a); 1.45 (1H, m, H2b); 1.50 (1H, m, H2a); 1.65 (1H, d, J=4.0 Hz, H19b); 1.67 (1H, m, H6a); 1.99 (1H, m, H5a); 2.02 (1H, m, H15b); 2.10 (1H, m, H8b); 2.14 (6H, s, NB—CH3); 2.25 (1H, dd, J=5.6 Hz and 11.2 Hz, H17a); 2.37 (1H, dq, J=7.0 Hz, H2′); 2.38 (1H, m, H1b); 2.43 (1H, J=16.9 Hz, H12b); 2.47 (1H, dd, J=6.4 Hz and 11.2 Hz, H20); 2.53 (1H, d, J=16.9 Hz, H12a); 2.97 (1H, d, J=13.0 Hz, H29a); 2.95 (1H, m, H3a); 3.16 (1H, d, J=13.0 Hz, H29b); 5.06 (1H, dd, J=5.6 Hz and 7.7 Hz, H16b).

¹³C NMR (75.5 MHz, CDCl₃), δppm:

9.9 (C21); 11.6 (C30); 17.8 (C18); 18.2 (C6); 19.4 and 20.5 (C3′ and C4′); 20.6 (C28); 24.2 (C7); 27.2 (C33-35); 27.3 (C2); 27.3 (C1); 30.2 (C19); 33.9 (C5); 33.9 (C9); 35.0 (C2′); 38.1 (C10); 38.6 (C32); 40.6 (NB—CH3); 40.7 (C8); 43.4 (C15); 44.8 (C14); 47.3 (C13); 47.4 (C4); 51.9 (C12); 54.2 (C3); 55.0 (C17); 58.3 (C29); 59.5 (C20); 79.1 (C16); 160.5 (C1′); 177.8 (C31); 210.7 (C11).

### Compound 54: (20S)-3β-isobutyrylamino-20-dimethylamino-9,19-cyclo-4β,14α-dimethyl-4α-(bis-(trimethylsilanyl)methylimino)methyl-16α-pivalyl-5α,9β-pregnan-11-one 54

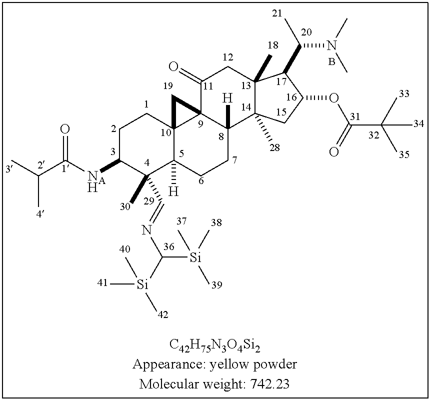

To a solution of 123 mg (0.21 mmol, 1.0 eq) of aldehyde 18 in 0.7 ml of dichloromethane are added 101 mg of an activated 4 Å molecular sieve (83% by weight with respect to the starting product) and 53 mg (0.28 mmol, 1.3 eq) of bis(trimethylsilyl)methylamine. After 24 hours of agitation at 65° C., the solution is filtered on celite and evaporated under a vacuum to yield 151 mg of 54 in the form of a yellow powder with a yield of 97%.

IR (diamond) υ(cm⁻¹): 2959 (CH, CH₂); 1720 (C═O ester); 1668 and 1526 (C═O, C═N and CONH); 1030 (C—O); 836 (Si—C).

MS (ESI): m/z: 742.5 ([M+H], 100); 743.5 (40); 744.5 (5); 371.8 ([M+2H]/2, 80); 372.3 (35); 372.8 (10).

HRMS (ESI) calculated for C₄₂H₇₆N₃O₄Si₂ m/z=742.5374; measured: 742.5353.

¹H NMR (300 MHz, CDCl₃), δppm: 0.00 (18H, s, H₃₇₋₄₂); 0.75 (3H, d, J=6.4 Hz, H₂₁); 0.76 (3H, s, H₁₈); 0.91 (3H, s, H₃₀); 0.92 (1H, m, H₆β); 0.98 (1H, m, H₁₉); 0.99 and 1.02 (6H, 2d, J=7.0 Hz, H_{3′} and H_{4′}); 1.07 (3H, s, H₂₈); 1.11 (9H, s, H₃₃₋₃₅); 1.16-1.40 (5H, m, H₁α, H₇α, H₁₅α, H₇β and H₂β); 1.51 (1H, d, J=3.8 Hz, H₁₉α); 1.59 (1H, s, H₃₆); 1.68 (1H, m, H₂α); 1.86-2.02 (4H, m, H₆α, H₈β, H₁₅β and H₅α); 2.05 (6H, bs, N_{B}—CH₃); 2.08-2.20 (2H, m, H_{2′} and H₁₇α); 2.32 (1H, d, J=17.0 Hz, H₁₂β); 2.33 (1H, m, H₁β); 2.38 (1H, dq, J=6.4 Hz and 11.8 Hz, H₂₀); 2.48 (1H, d, J=17.0 Hz, H₁₂α); 3.89 (1H, ddd, J=3.4 Hz, 9.8 Hz and 9.8 Hz, H₃α); 4.96 (1H, dd, J=5.3 Hz, 7.7 Hz, H₁₆β); 5.29 (1H, d, J=8.3 Hz, N_{A}H); 7.19 (1H, s, H₂₉).

¹³C NMR (75.5 MHz, CDCl₃), δppm: −1.1 and −0.9 (C₃₇₋₄₂); 10.1 (C₂₁); 11.3 (C₃₀); 17.9 (C₁₈); 18.9 and 19.5 (C_{3′} and C_{4′}); 20.2 (C₆); 20.5 (C₂₈); 24.4 (C₇); 27.3 (C₃₃₋₃₅); 27.4 (C₂); 27.8 (C₁); 29.7 (C₁₉); 31.7 (C₃₆); 34.2 (C₉); 35.8 (C_{2′}); 37.7 (C₁₀); 38.7 (C₃₂); 40.8 (N_{B}—CH₃); 41.1 (C₈); 43.5 (C₁₅); 44.9 (C₁₃); 45.5 (C₅); 47.5 (C₁₄); 48.4 (C₄); 52.1 (C₁₂); 53.9 (C₃); 55.1 (C₁₇); 59.7 (C₂₀); 79.3 (C₁₆); 164.7 (C₂₉); 176.3 (C₃₁); 178.0 (C_{1′}); 210.8 (C₁₁).

### Compound 55: (20S)-3β-isobutyrylamino-20-dimethylamino-9,19-cyclo-4β,14α-dimethyl-4α-(bis-(trimethylsilanyl)methylamino)methyl-5α,9β-pregnan-16α-ol-11-one 55

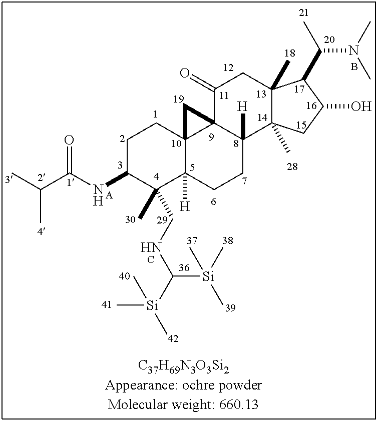

Under argon, to a solution of 500 mg (0.92 mmol, 1.0 eq) of 16-O-acetyl aldehyde 14 in 0.5 ml of dichloromethane are added 415 mg of an activated 4 Å molecular sieve (83% by weight with respect to the starting product) and 210 mg (1.20 mmol, 1.3 eq) of bis(trimethylsilyl)methylamine. After 3 days of agitation at 65° C., the solution is filtered and evaporated under a vacuum. To a solution of the residue obtained in 8 ml of methanol are added 138 mg (2.08 mmol, 2.3 eq) of sodium cyanoborohydride and 150 μl (2.62 mmol, 2.8 eq) of glacial acetic acid. After 16 hours of agitation at room temperature, the mixture is alkalinized with 30 ml of a 10% ammonia solution (pH=10), which saponifies the acetyl group into 16, and extract with 3×40 ml of dichloromethane. The recombined organic phases are successively dried on anhydrous sodium sulfate and evaporated under a vacuum.

The residue is chromatographed on a Merck activity II-III alumina column (eluent: 95/5 dichloromethane/methanol) to yield 137 mg of 55 in the form of an ochre powder with a yield of 25%, in three steps.

IR (diamond) υ(cm⁻¹): 3310 (OH, NH); 2948 (CH, CH₂); 1660 and 1535 (C═O and CONH); 1094 (C—O); 837 (Si—C).

MS (ESI): m/z: 660.5 ([M+H], 40); 661.5 (10); 330.7 ([M+2H]/2, 100); 331.3 (35); 331.8 (5).

HRMS (ESI) calculated for C₃₇H₇₀N₃O₃Si₂ m/z=660.4956; measured: 660.4974.

¹H NMR (500 MHz, CDCl₃), δppm: 0.04 and 0.06 (18H, 2s, H₃₇₋₄₂); 0.59 (3H, s, H₃₀); 0.80 (3H, d, J=6.4 Hz, H₂₁); 0.82 (3H, s, H₁₈); 0.88 (1H, m, H₆β); 1.00 (1H, d, J=3.8 Hz, H₁₉); 1.13 and 1.14 (6H, 2d, J=6.8 Hz, H_{3′} and H_{4′}); 1.21 (3H, s, H₂₈); 1.24 (1H, s, H₃₆); 1.26 (1H, m, H₁α); 1.40-1.53 (4H, m, H₇α, H₁₅α, H₇β and H₂β); 1.54 (1H, d, J=3.8 Hz, H₁₉α); 1.67 (1H, m, H₂α); 1.98-2.06 (3H, m, H₆α, H₁₅β and H₁₇α); 2.05 (1H, d, 12.9 Hz, H₂₉ₐ); 2.13 (6H, bs, N_{B}—CH₃); 2.22-2.34 (3H, m, H₈β, H₅α and H₁β); 2.28 (1H, d, J=17.0 Hz, H₁₂β); 2.31 (1H, dq, J=6.8 Hz and 11.2 Hz, H_{2′}); 2.50 (1H, d, J=17.0 Hz, H₁₂α); 2.64 (1H, m, H₂₀); 2.68 (1H, d, J=12.9 Hz, H_{29b}); 4.00 (1H, ddd, J=4.1 Hz, 10.2 Hz and 11.0 Hz, H₃α); 4.12 (1H, m, H₁₆β); 5.13 (1H, d, J=9.8 Hz, N_{A}H).

¹³C NMR (125.8 MHz, CDCl₃), δppm: −0.2 and 0.7 (C₃₇₋₄₂); 10.2 (C₂₁); 13.8 (C₃₀); 18.0 (C₁₈); 19.0 (C₆); 20.0 (C_{3′} and C_{4′}); 20.8 (C₂₈); 24.1 (C₇); 27.8 (C₁); 28.7 (C₂); 30.3 (C₁₉); 34.9 (C₉); 36.1 (C_{2′}); 38.4 (C₁₀); 41.2 (C₈); 41.7 (C₅); 41.9 (C₃₆); 43.1 (C₄); 44.0 (C₁₅); 44.8 (C₁₃); 47.5 (C₁₄); 50.5 (C₃); 51.7 (C₁₂); 56.1 (C₁₇); 58.5 (C₂₉); 59.7 (C₂₀); 79.1 (C₁₆); 176.3 (C_{1′}); 211.5 (C₁₁).

### Compound 56: (20S)-3β-isobutyrylamino-20-dimethylamino-9,19-cyclo-4β,14α-dimethyl-4α-(bis-(trimethylsilanyl)methylamino)methyl-16α-pivalyl-5α,9β-pregnan-11-one 56

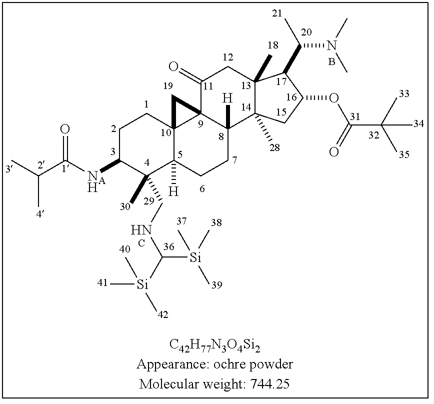

To a solution of 150 mg (0.20 mmol, 1.0 eq) of imine 54 in 0.7 ml of methanol are added 41 mg (0.61 mmol, 3.1 eq) of sodium cyanoborohydride and 35 μl (0.60 mmol, 3.0 eq) of glacial acetic acid. After 44 hours of agitation at room temperature, the mixture is alkalinized with 30 ml of a 10% ammonia solution (pH=10) and extracted with 3×25 ml of dichloromethane. The recombined organic phases are successively dried on anhydrous sodium sulfate and evaporated under a vacuum.

The residue is chromatographed on a Merck activity II-III alumina column (eluent: 99.7/0.3 dichloromethane/methanol) to yield 84 mg of 56 in the form of an ochre powder with a yield of 54%, in two steps.

IR (diamond) υ(cm⁻¹): 3307 (NH), 2959 (CH, CH₂); 1722 (C═O ester); 1658 and 1528 (C═O and CONH); 1095 (C—O); 838 (Si—C).

MS (ESI): m/z: 744.6 ([M+H], 70); 745.6 (30); 746.6 (5); 372.8 ([M+2H]/2, 100); 373.3 (70); 373.8 (15).

HRMS (ESI) calculated for C₄₂H₇₈N₃O₄Si₂ m/z=744.5531; measured: 744.5562.

¹H NMR (300 MHz, CDCl₃), δppm: 0.04 and 0.05 (18H, 2s, H₃₇₋₄₂); 0.60 (3H, s, H₃₀); 0.83 (3H, d, J=6.4 Hz, H₂₁); 0.85 (3H, s, H₁₈); 0.90 (1H, m, HO); 1.02 (1H, d, J=3.8 Hz, H₁₉); 1.13 and 1.14 (6H, 2d, J=6.8 Hz, H_{3′} and H_{4′}); 1.15 (3H, s, H₂₈); 1.19 (9H, s, H₃₃₋₃₅); 1.24 (1H, s, H₃₆); 1.22-1.52 (5H, m, H₁α, H₇α, H₁₅α, H₇β and H₂β); 1.57 (1H, d, J=3.8 Hz, H₁₉α); 1.67 (1H, m, H₂α); 1.98-2.08 (4H, m, H₆α, H₈β, H₁₅β and H₅α); 2.01 (1H, d, 12.8 Hz, H₂₉ₐ); 2.13 (6H, bs, N_{B}—CH₃); 2.22-2.34 (3H, m, H_{2′}, H₁β and H₁₇α); 2.38 (1H, d, J=17.2 Hz, H₁₂β); 2.46 (1H, dq, J=6.4 Hz and 11.0 Hz, H₂₀); 2.55 (1H, d, J=17.2 Hz, H₁₂α); 2.69 (1H, d, J=12.8 Hz, H_{29b}); 4.01 (1H, ddd, J=4.0 Hz, 10.9 Hz and 11.0 Hz, H₃α); 5.05 (1H, dd, J=5.5 Hz, 7.7 Hz, H₁₆β); 5.12 (1H, d, J=10.0 Hz, N_{A}H).

¹³C NMR (75.5 MHz, CDCl₃), δppm: −0.3 and 0.7 (C₃₇₋₄₂); 10.1 (C₂₁); 13.7 (C₃₀); 17.9 (C₁₈); 18.9 (C₆); 19.6 and 20.0 (C_{3′} and C_{4′}); 20.0 (C₂₈); 24.1 (C₇); 27.3 (C₃₃₋₃₅); 27.8 (C₁); 28.7 (C₂); 30.0 (C₁₉); 34.4 (C₉); 36.1 (C_{2′}); 38.3 (C₁₀); 38.7 (C₃₂); 40.8 (C₃₆ and N_{B}—CH₃); 41.6 (C₈); 41.9 (C₅); 43.6 (C₁₅); 44.0 (C₄); 45.0 (C₁₃); 47.6 (C₁₄); 50.5 (C₃); 52.2 (C₁₂); 55.1 (C₁₇); 58.5 (C₂₉); 59.7 (C₂₀); 79.4 (C₁₆); 176.3 (C_{1′}); 178.0 (C₃₁); 211.3 (C₁₁).

### Compound 57: (20S)-3β-isobutyrylamino-20-dimethylamino-9,19-cyclo-4β,14α-dimethyl-4α-(bis-(trimethylsilanyl)methylamino)methyl-16α-(tert-butyl-dimethyl-silanyloxy)-5α,9β-pregnan-11-one 57

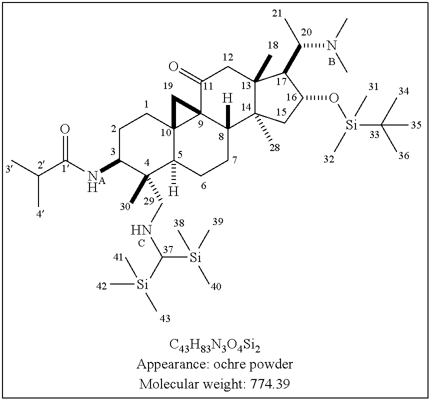

Under argon, to a solution of 93 mg (0.14 mmol, 1.0 eq) of amine 55 in 2.0 ml of tetrahydrofurane are added 276 mg (0.39 mmol, 2.8 eq) of imidazol and 36 mg (0.23 mmol, 1.7 eq) of tert-butyldimethylsilyle chloride. After 16 hours of agitation at room temperature, the mixture is alkalinized with 40 ml of a 10% sodium hydrogen carbonate solution (pH=8) and extracted with 3×40 ml of dichloromethane. The recombined organic phases are successively dried on anhydrous sodium sulfate and evaporated under a vacuum.

The residue is chromatographed on a Merck activity II-III alumina column (eluent: 99.9/0.1 dichloromethane/methanol) to yield 84 mg of 57 in the form of a colorless powder with a yield of 77%.

IR (diamond) υ(cm⁻¹): 3391 and 3112 (NH); 1666, 1653 and 1521 (C═O and CONH); 1093 (C—O); 1028 (Si—O—C); 833 (Si—C, Si—O).

MS (ESI): m/z: 774.6 ([M+H], 10); 387.8 ([M+2H]/2, 100); 388.3 (50); 388.8 (15).

HRMS (ESI) calculated for C₄₃H₈₄N₃O₃Si₃ m/z=774.5821; measured: 774.5862.

¹H NMR (500 MHz, CDCl₃), δppm: 0.01 and 0.02 (6H, 2s, H₃₁₋₃₂); 0.05 and 0.07 (18H, 2s, H₃₈₋₄₃); 0.60 (3H, s, H₃₀); 0.79 (3H, s, H₁₈); 0.80 (3H, d, J=6.3 Hz, H₂₁); 0.88 (9H, s, H₃₄₋₃₆); 0.90 (1H, m, H₆β); 0.98 (1H, d, J=3.8 Hz, H₁₉); 1.15 and 1.16 (6H, 2d, J=6.9 Hz, H_{3′} and H_{4′}); 1.20 (3H, s, H₂₈); 1.25 (1H, s, H₃₇); 1.27 (1H, m, H₁α); 1.39 (1H, m, H₁₅α); 1.43-1.48 (3H, m, H₇α, H₇β and H₂β); 1.56 (1H, d, J=3.8 Hz, H₁₉α); 1.67 (1H, m, H₂α); 1.81 (1H, dd, J=7.6 Hz and 13.6 Hz; H₁₅β); 2.01 (1H, dd, J=9.5 Hz and 8.8 Hz, H₈β); 2.06 (1H, m, H₅α, H₁₇α); 2.08 (1H, d, 12.9 Hz, H₂₉ₐ); 2.13 (6H, bs, N_{B}—CH₃); 2.19-2.22 (2H, m, H₁β and H₆α); 2.31 (1H, dq, J=6.9 Hz, H_{2′}); 2.34 (1H, d, J=17.0 Hz, H₁₂β); 2.41 (1H, dq, J=6.3 Hz and 12.0 Hz, H₂₀); 2.52 (1H, d, J=17.0 Hz, H₁₂α); 2.69 (1H, d, J=12.9 Hz, H_{29b}); 4.01 (1H, ddd, J=4.1 Hz, 10.1 Hz and 12.2 Hz, H₃α); 4.14 (1H, dd, J=4.7 Hz, 7.6 Hz, H₁₆β); 5.10 (1H, d, J=9.8 Hz, N_{A}H).

¹³C NMR (125.7 MHz, CDCl₃), δppm: −0.2 and 0.7 (C₃₇₋₄₂); 10.3 (C₂₁); 13.8 (C₃₀); 17.9 (C₁₈); 19.0 (C₆); 19.6 (C₂₈); 20.0 (C_{3′} and C_{4′}); 24.1 (C₇); 26.1 (C₃₄₋₃₆); 27.8 (C₁); 28.7 (C₂); 29.4 (C₁₉); 34.6 (C₉); 36.1 (C_{2′}); 37.9 (C₁₀); 40.8 (C₄ and N_{B}—CH₃); 41.4 (C₃₇ and C₅); 41.9 (C₈); 44.0 (C₁₃); 45.3 (C₁₄); 46.0 (C₁₅); 50.6 (C₃); 52.5 (C₁₂); 58.5 (C₂₉); 59.5 (C₁₇); 60.2 (C₂₀); 76.9 (C₁₆); 176.3 (C_{1′}); 212.1 (C₁₁).

### Compound 59: (20S)-16α-acetyl-3β-((S)-2-methylbutyrylamino)-20-dimethylamino-9,19-cyclo-4α-formyl-4β,14α-dimethyl-5α,9β-pregnan-11-one 59

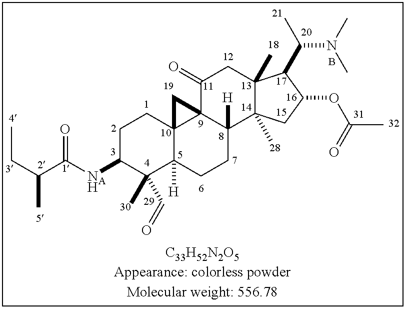

To a solution of 496 mg (0.96 mmol, 1.0 eq) of amide 6e in a mixture of 48 ml of dichloromethane and 14 ml of pyridine are added 136 μl (1.44 mmol, 1.5 eq) of acetic anhydride. After 16 hours of agitation at room temperature, the mixture is co-evaporated with 1,2-dichloroethane. The residue is alkalinized with 40 ml of a 10% ammonia solution (pH=10) and extracted with 3×30 ml of dichloromethane. The recombined organic phases are successively dried on anhydrous sodium sulfate and evaporated under a vacuum.

To a solution of 519 mg (0.93 mmol, 1.0 eq) of the residue previously obtained in 30 ml of dichloromethane are added 511 mg (1.21 mmol, 1.3 eq) of the Dess-Martin periodinane. After 1 hour of agitation at room temperature, the mixture is alkalinized with 40 ml of a 10% ammonia solution (pH=10) and extracted with 3×30 ml of dichloromethane. The recombined organic phases are successively dried on anhydrous sodium sulfate and evaporated under a vacuum.

The residue is chromatographed on a Merck activity II-III alumina column (eluent: 95/5 dichloromethane/methanol) to yield 500 mg of 59 in the form of a colorless powder after trituration in acetone and with a yield of 91%, in two steps.

MP (° C.): 265° C.

IR (diamond) υ(cm⁻¹): 3254 (OH, NH); 2926 (CH, CH₂); 2856 (CH═O); 1718 (C═O ester); 1666 (C═O); 1638 and 1538 (CONH); 1255, 1017 (C—OH).

MS (ESI): m/z: 557.4 ([M+H], 100); 558.4 (15); 515.4 (20).

HRMS (ESI) calculated for C₃₃H₅₃N₂O₅ m/z=557.3954; measured: 557.3948.

¹H NMR (300 MHz, CDCl₃), δppm:

0.81 (3H, d, J=6.4 Hz, H₂₁); 0.83 (3H, s, H₁₈); 0.84 (3H, t, J=7.3 Hz, H_{4′}); 0.92 (3H, s, H₃₀); 1.06 (1H, m, H₆β); 1.06 (3H, d, J=6.8 Hz, H_{5′}); 1.07 (1H, d, J=3.6 Hz, H₁₉α); 1.18 (3H, s, H₂₈); 1.24-1.59 (5H, m, H₁α, H₂β, H₇β, H₇α and H₁₅α); 1.58 (1H, d, J=4.2 Hz, H₁₉) 1.73-1.82 (3H, m, H_{3′} and H₂α); 1.99 (3H, s, H₃₂); 2.04 (1H, m, H₁₅β); 2.13 (6H, s, N_{B}—CH₃); 1.95-2.16 (4H, m, H_{2′}, H₆α, H₅α and H₈β); 2.22 (1H, dd, J=6.2 Hz and 10.0 Hz, H₁₇α); 2.38 (1H, d, J=17.0 Hz, H₁₂β); 2.45 (2H, m, H₂₀ and H₁β); 2.56 (1H, d, J=17.1 Hz, H₁₂α); 4.34 (1H, ddd, J=4.1 Hz, 8.9 Hz and 12.4 Hz, H₃α); 5.11 (1H, dd, J=6.2 Hz and 8.0 Hz, H₁₆β); 5.28 (1H, d, J=9.1 Hz, N_{A}H); 9.41 (1H, s, H₂₉).

¹³C NMR (75.5 MHz, CDCl₃), δppm:

7.8 (C₃₀); 10.0 (C₂₁); 11.8 (C_{4′}); 17.4 (C_{5′}); 18.0 (C₁₈); 19.5 (C₂₈); 21.1 (C₆); 21.2 (C₃₁); 24.2 (C₇); 27.0 (C₂); 27.5 (C_{3′}); 29.6 (C₁); 29.6 (C₁₉); 33.7 (C₉); 36.0 (C₁₀); 40.4 (N_{B}—CH₃); 41.3 (C_{2′}); 41.8 (C₅); 43.0 (C₁₅); 43.4 (C₈); 44.6 (C₁₃); 47.5 (C₁₄); 50.0 (C₃); 51.9 (C₁₂); 55.3 (C₁₇); 56.9 (C₄); 59.6 (C₂₀); 78.9 (C₁₆); 170.7 (C₃₁); 175.9 (C_{1′}); 203.5 (C₂₉); 211.6 (C₁₁).

### Compound 60: (20S)-20-dimethylamino-3β-((S)-2-methylbutyrylamino)-9,19-cyclo-4α-formyl-4β,14α-dimethyl-5α,9β-pregnan-16α-ol-11-one 60

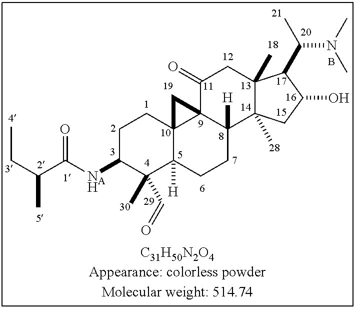

To a solution of 1127 mg (2.02 mmol, 1.0 eq) of amide 59 in 26 ml of methanol, heated beforehand at 40° C. for total dissolution, is added a solution of 2542 mg (30.30 mmol, 15.0 eq) of sodium hydrogen carbonate and 2138 mg (20.20 mmol, 10.0 eq) of sodium carbonate in 28 ml of permuted water. A precipitate is formed. After 6 hours of agitation at 60° C., the mixture is diluted with 50 ml of water and extracted with 3×50 ml of dichloromethane. The recombined organic phases are successively dried on anhydrous sodium sulfate and evaporated under a vacuum.

The residue is chromatographed on a Merck activity II-III alumina column (eluent: 98/2 dichloromethane/methanol) to yield 710 mg of 60 in the form of a colorless powder with a yield of 68%, in two steps.

MP (° C.): 265° C.

Elemental analysis: for C₃₃H₅₂N₂O₅

Calculated for C₃₃H₅₂N₂O₅%: C, 71.19; H, 9.41; N, 5.03; O, 14.37.

Actual %: C H N O

IR (diamond) υ(cm⁻¹): 3287 (OH, NH); 2935 (CH, CH₂); 2709 (CH═O); 1717 (C═O aldehyde); 1659 (C═O); 1640 and 1548 (CONH); 1229 and 1093 (C—OH).

MS (ESI): m/z: 515.4 ([M+H], 100); 516.4 (5).

HRMS (ESI) calculated for C₃₁H₅₁N₂O₄ m/z=515.3849; measured: 515.3831.

¹H NMR (300 MHz, CDCl₃), δppm:

0.74 (3H, s, H₁₈); 0.77 (3H, t, J=7.5 Hz, H_{4′}); 0.81 (3H, d, J=6.4 Hz, H₂₁); 0.87 (3H, s, H₃₀); 0.98 (1H, m, H₆β); 1.00 (3H, d, J=6.8 Hz, H_{5′}); 1.01 (1H, m, H₁₉α); 1.18 (3H, s, H₂₈); 1.24-1.59 (7H, m, H₁α, H₂β, H_{3′}, H₇β, H₇α and H₁₅α); 1.50 (1H, d, J=4.1 Hz, H₁₉); 1.69-1.78 (2H, m, H₈β and H₂α); 1.90 (1H, dd, J=7.4 Hz and 10.8 Hz, H₁₇α); 1.95-2.16 (4H, m, H_{2′}, H₆α, H₅α and H₁₅β); 2.18 (6H, s, N_{B}—CH₃); 2.25 (1H, d, J=17.3 Hz, H₁₂β); 2.36 (1H, ddd, J=3.0 Hz, 4.7 Hz and 12.6 Hz, H₁β); 2.47 (1H, d, J=17.3 Hz, H₁₂α); 2.55 (1H, dd, J=6.8 Hz and 10.8 Hz, H₂₀); 4.03 (1H, ddd, J=2.8 Hz, 7.4 Hz and 10.0 Hz, H₁₆β); 4.28 (1H, ddd, J=4.2 Hz, 8.7 Hz and 12.4 Hz, H₃α); 5.28 (1H, d, J=9.1 Hz, N_{A}H); 9.36 (1H, s, H₂₉).

¹³C NMR (75.5 MHz, CDCl₃), δppm:

7.7 (C₃₀); 10.0 (C₂₁); 11.7 (C_{4′}); 17.3 (C_{5′}); 17.9 (C₁₈); 20.7 (C₂₈); 21.0 (C₆); 24.0 (C₇); 27.3 (C₁); 27.5 (C_{3′} and C₂); 29.6 (C₁₉); 34.0 (C₉); 35.8 (C₁₀); 41.3 (C₅); 41.7 (C₈); 41.8 (N_{B}—CH₃); 42.6 (C₁₅); 43.2 (C_{2′}); 44.4 (C₁₃); 47.1 (C₁₄); 49.8 (C₃); 51.4 (C₁₂); 55.8 (C₁₇); 56.8 (C₄); 62.0 (C₂₀); 78.2 (C₁₆); 175.8 (C_{1′}); 203.4 (C₂₉); 210.9 (C₁₁).

### Compound 61: (20S)-20-dimethylamino-3β-((S)-2-methylbutyrylamino)-9,19-cyclo-4α-benzyliminomethyl-4β,14α-dimethyl-5α,9β-pregnan-16α-ol-11-one 61

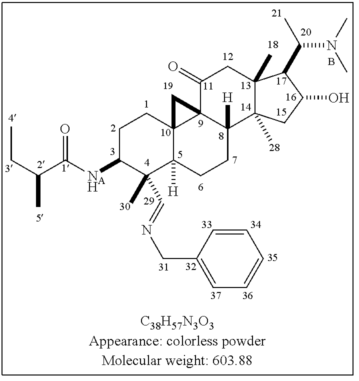

Under argon, to a suspension of 650 mg (1.26 mmol, 1.0 eq) of aldehyde 60 and 540 mg of anhydrous magnesium sulfate in 5 ml of dichloromethane are added 179 μl (1.63 mmol, 1.3 eq) of benzylamine. After 10 hours of agitation at 55° C., the solution is filtered on celite and evaporated under a vacuum to yield 793 mg of 61 in the form of a colorless powder with a quantitative yield.

IR (diamond) υ(cm⁻¹): 3386 (OH, NH); 2958 (CH, CH₂); 1649 (C═O, C═N); 1649 and 1529 (CONH); 1451 (C═C Ar); 1227 and 1091 (C—OH).

MS (ESI): m/z: 604.4 ([M+H], 100); 605.4 (55); 590.4 (30).

HRMS (ESI) calculated for C₃₈H₅₈N₃O₃ m/z=604.4478; measured: 604.4468.

¹H NMR (300 MHz, CDCl₃), δppm:

0.73 (3H, t, J=7.5 Hz, H_{4′}); 0.77 (3H, s, H₁₈); 0.81 (3H, d, J=6.6 Hz, H₂₁); 0.93 (1H, m, H₆β); 0.95 (3H, d, J=6.8 Hz, H_{5′}); 0.98 (3H, s, H₃₀); 1.01 (1H, d, J=4.3 Hz, H₁₉α); 1.16 (3H, s, H₂₈); 1.24-1.59 (7H, m, H₁α, H₂β, H_{3′}, H₇β, H₇α and H₁₅α); 1.52 (1H, d, J=4.3 Hz, H₁₉); 1.75 (1H, m, H₂α); 1.82-2.07 (6H, m, H₁₅β, H₁₇α, H_{2′}, H₆α, H₅α and H₈β); 2.19 (6H, s, N_{B}—CH₃); 2.24 (1H, d, J=17.3 Hz, H₁₂β); 2.31 (1H, ddd, J=3.2 Hz, 3.3 Hz and 13.2 Hz, H₁β); 2.46 (1H, d, J=17.3 Hz, H₁₂α); 2.56 (1H, dd, J=6.6 Hz and 12.0 Hz, H₂₀); 4.03 (2H, m, H₃α and H₁₆β); 4.43 and 4.50 (2H, 2d system AB, 14.1 Hz, H₃₁); 5.25 (1H, d, J=9.2 Hz, N_{A}H); 7.14-7.25 (5H, m, H_{Ar=33-37}); 7.50 (1H, s, H₂₉).

¹³C NMR (75.5 MHz, CDCl₃), δppm: 10.0 (C₃₀); 10.3 (C₂₁); 11.8 (C_{4′}); 17.1 (C_{5′}); 17.9 (C₁₈); 20.6 (C₆); 20.7 (C₂₈); 24.2 (C₇); 27.2 (C_{3′}); 27.5 (C₁); 27.7 (C₂); 29.8 (C₁₉); 34.3 (C₉); 36.7 (C₁₀); 41.3 (C₈); 42.7 (C₁₅); 43.3 (C_{2′}); 44.5 (C₁₃); 44.7 (C₅); 47.1 (C₁₄); 49.9 (C₄); 51.4 (C₁₂); 52.2 (C₃); 55.8 (C₁₇); 62.1 (C₂₀); 64.7 (C₃₁); 78.2 (C₁₆); 126.7 (C₃₅); 127.7 and 128.3 (C₃₄₋₃₆ and C₃₃₋₃₇); 139.5 (C₃₂); 170.9 (C₂₉); 175.6 (C_{1′}); 211.0 (C₁₁).

### Compound 62: (20S)-20-dimethylamino-3β-((S)-2-methylbutyrylamino)-9,19-cyclo-4α-benzylaminomethyl-4β,14α-dimethyl-5α,9β-pregnan-16α-ol-11-one 62

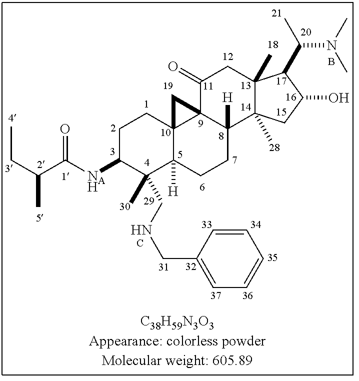

To a suspension of 770 mg (1.26 mmol, 1.0 eq) of imine 61 in 7 ml of methanol are successively added 102 mg (1.54 mmol, 1.2 eq) of sodium cyanoborohydride and 39 μl (1.27 mmol, 1.05 eq) of glacial acetic acid. After 3 hours of agitation at room temperature, the mixture is alkalinized with 50 ml of a 10% ammonia solution (pH=10) and extracted with 3×50 ml of dichloromethane. The recombined organic phases are successively dried on anhydrous sodium sulfate and evaporated under a vacuum.

The residue is chromatographed on a Merck activity II-III alumina column (eluent: 98/1 dichloromethane/methanol) to yield 618 mg of 62 in the form of a colorless powder with a yield of 81%, in two steps.

IR (diamond) υ(cm⁻¹): 3314 (OH, NH); 2932 (CH, CH₂); 1660 (C═O); 1660 and 1529 (CONH); 1452 (C═C Ar); 1226 and 1094 (C—OH).

MS (ESI): m/z: 606.5 ([M+H], 100); 607.5 (10).

HRMS (ESI) calculated for C₃₈H₆₀N₃O₃ m/z=606.4635; measured: 606.4634.

¹H NMR (300 MHz, CDCl₃), δppm:

0.59 (3H, s, H₁₈); 0.83 (3H, s, H₃₀); 0.88 (3H, d, J=6.6 Hz, H₂₁); 0.90 (3H, t, J=7.3 Hz, H_{4′}); 0.95 (1H, m, H₆β); 1.09 (1H, d, J=3.4 Hz, H₁₉α); 1.13 (3H, d, J=6.8 Hz, H_{5′}); 1.25 (3H, s, H₂₈); 1.25-1.50 (5H, m, H₁α, H_{3a′}, H₇, H₂β and H₁₅α); 1.52 (1H, d, J=3.4 Hz, H₁₉); 1.58-1.75 (2H, m, H_{3b′} and H₂α); 1.94-2.19 (6H, m, H₁₅β, H₁₇α, H_{2′}, H₆α, H₅α and H₈β); 2.12 (1H, d, J=12.2 Hz, H₂₉); 2.25 (6H, s, N_{B}—CH₃); 2.27 (1H, d, J=12.2 Hz, H₂₉); 2.29 (1H, d, J=17.1 Hz, H₁₂β); 2.36 (1H, m, H₁β); 2.52 (1H, d, J=17.1 Hz, H₁₂α); 2.62 (1H, dd, J=6.6 Hz and 10.8 Hz, H₂₀); 3.62 and 3.81 (2H, 2d system AB, 13.7 Hz, H₃₁); 4.07 (1H, m, H₃α); 4.11 (1H, m, H₁₆β); 5.19 (1H, d, J=9.8 Hz, N_{A}H); 7.18-7.35 (5H, m, H_{Ar=33-37}).

¹³C NMR (75.5 MHz, CDCl₃), δppm:

9.9 (C₂₁); 12.1 (C_{4′}); 13.7 (C₃₀); 17.7 (C_{5′}); 17.8 (C₁₈); 18.4 (C₆); 21.0 (C₂₈); 24.4 (C₇); 27.5 (C_{3′}); 27.6 (C₁); 28.5 (C₂); 31.0 (C₁₉); 34.6 (C₉); 38.2 (C₁₀); 41.6 (C₈); 42.0 (C₅); 42.9 (C₁₅); 43.1 (C₄); 43.7 (C_{2′}); 44.5 (C₁₃); 47.1 (C₁₄); 50.5 (C₃); 51.5 (C₁₂); 52.9 (C₂₉); 54.5 (C₃₁); 55.8 (C₁₇); 62.0 (C₂₀); 78.4 (C₁₆); 126.5 (C₃₅); 128.0 and 128.4 (C₃₄₋₃₆ and C₃₃₋₃₇); 141.3 (C₃₂); 176.1 (C_{1′}); 211.6 (C₁₁).

### Compound 63: (20S)-20-dimethylamino-3β-((S)-2-methylbutyrylamino)-9,19-cyclo-4α-aminomethyl-4β,14α-dimethyl-5α,9β-pregnan-16α-ol-11-one 63

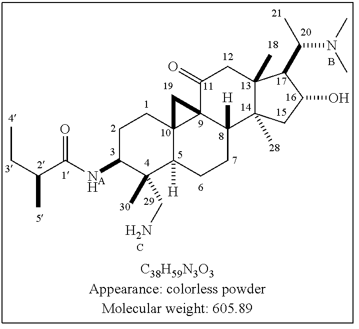

Under argon, to a suspension of 580 mg (0.96 mmol, 1.0 eq) of N-benzylated amine 62 in 20 ml of anhydrous methanol degassed with argon, are successively added 290 mg (50% by weight with respect to 62) of 30% palladium on carbon and 311 mg (4.79 mmol, 5.0 eq) of ammonium formate. After 3 hours of agitation at 40° C., the mixture is filtered on celite and evaporated under a vacuum. The residue obtained is alkalinized with 40 ml of a 10% ammonia solution (pH=10) and extracted with 3×30 ml of dichloromethane. The recombined organic phases are successively dried on anhydrous sodium sulfate and evaporated under a vacuum.

The residue is chromatographed on a silica column (eluent: 98/1.9/0.1 dichloromethane/methanol/ammonia) to yield 414 mg of 63 in the form of a colorless powder with a yield of 84%, in two steps.

IR (diamond) υ(cm⁻¹): 3314 (OH, NH); 2932 (CH, CH₂); 1660 (C═O); 1660 and 1529 (CONH); 1452 (C═C Ar); 1226 and 1094 (C—OH).

MS (ESI): m/z: 606.5 ([M+H], 100); 607.5 (10).

HRMS (ESI) calculated for C₃₈H₆₀N₃O₃ m/z=606.4635; measured: 606.4634.

¹H NMR (300 MHz, CDCl₃), δppm:

0.63 (3H, s, H₃₀); 0.84 (3H, s, H₁₈); 0.87 (3H, d, J=6.6 Hz, H₂₁); 0.92 (3H, t, J=7.4 Hz, H_{4′}); 0.93 (1H, m, H₆β); 1.04 (1H, d, J=3.8 Hz, H₁₉α); 1.13 (3H, d, J=6.8 Hz, H_{5′}); 1.22 (3H, s, H₂₈); 1.26-1.57 (5H, m, H₁α, H_{3a′}, H₇, H₂β and H₁₅α); 1.56 (1H, d, J=3.8 Hz, H₁₉); 1.59-1.74 (2H, m, H_{3b′} and H₂α); 1.95-2.13 (6H, m, H₁₅β, H₁₇α, H_{2′}, H₆α, H₅α and H₈β); 2.24 (6H, s, N_{B}—CH₃); 2.31 (1H, d, J=17.5 Hz, H₁₂β); 2.36 (1H, d, J=13.9 Hz, H₂₉ₐ); 2.41 (1H, m, H₁β); 2.46 (1H, d, J=13.9 Hz, H_{29b}); 2.51 (1H, d, J=17.5 Hz, H₁₂α); 2.61 (1H, dd, J=6.6 Hz and 10.9 Hz, H₂₀); 4.06 (1H, m, H₃α); 4.10 (1H, m, H₁₆β); 5.24 (1H, d, J=9.8 Hz, N_{A}H).

¹³C NMR (75.5 MHz, CDCl₃), δppm:

9.9 (C₂₁); 12.1 (C_{4′}); 13.7 (C₃₀); 17.6 (C_{5′}); 17.8 (C₁₈); 18.5 (C₆); 21.0 (C₂₈); 24.6 (C₇); 27.5 (C_{3′}); 27.7 (C₁); 28.4 (C₂); 31.2 (C₁₉); 34.4 (C₉); 38.1 (C₁₀); 41.7 (C₈); 41.9 (C₅); 42.9 (C₁₅); 43.6 (C_{2′} and C₄); 44.4 (C₁₃); 46.2 (C₂₉); 47.1 (C₁₄); 49.7 (C₃); 51.5 (C₁₂); 55.8 (C₁₇); 62.0 (C₂₀); 78.3 (C₁₆); 176.3 (C_{1′}); 211.5 (C₁₁).

### Compound 64: (20S)-20-dimethylamino-9,19-cyclo-4β,14α-dimethyl-(2-(S)-sec-butyl-5β-methyl-1,4,5,6-tetrahydro-pyrimidin-5-yl)-5α,9β-pregnan-16α-ol-11-one 64

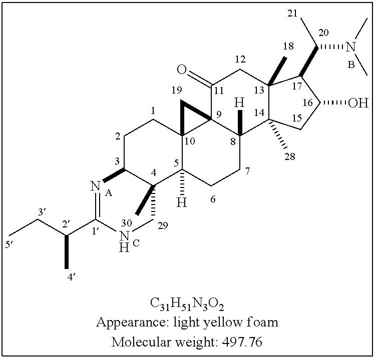

To a solution of 300 mg (0.58 mmol, 1.0 eq) of amine 63 in 30 ml of n-butanol is added 162 μl (1.15 mmol, 2.0 eq) of triethylamine. After 16 hours of agitation at 120° C., the mixture is evaporated under a vacuum. The residue obtained is alkalinized with 30 ml of a 10% ammonia solution (pH=10) and extracted with 3×40 ml of dichloromethane. The recombined organic phases are successively dried on anhydrous sodium sulfate and evaporated under a vacuum.

The residue is chromatographed on a Merck activity II-III alumina column (eluent: 95/5 dichloromethane/methanol) to yield 210 mg of 64 in the form of a light yellow foam with a yield of 73%.

IR (diamond) υ(cm⁻¹): 3312 (OH, NH); 2932 (CH, CH₂); 1665 (C═O); 1625 and 1518 (C—N, C═N); 1039 (C—OH).

MS (ESI): m/z: 498.4 ([M+H], 100); 499.4 (10).

HRMS (ESI) calculated for C₃₁H₅₂N₃O₂ m/z=498.4060; measured: 498.4049.

Note: This is a mixture of two tautomeric forms. Nitrogens N_{A} and N_{C} exchange a proton. In ¹³C NMR, carbons C3 and C29 give masses of low integration.

¹H NMR (500 MHz, CDCl₃), δppm:

0.69 (3H, s, H₃₀); 0.77 (3H, s, H₁₈); 0.80 (3H, d, J=6.6 Hz, H₂₁); 0.85 (3H, t, J=7.6 Hz, H_{4′}); 1.01 (1H, m, H₆β); 1.04 (3H, d, J=6.8 Hz, H_{5′}); 1.07 (1H, d, J=3.8 Hz, H₁₉α); 1.14 (3H, s, H₂₈); 1.16-1.43 (6H, m, H₁α, H_{3a′}, H₇, H₂β and H₁₅α); 1.50 (1H, m, H_{3a′}); 1.55 (1H, d, J=3.8 Hz, H₁₉); 1.61 (1H, m, H₂₀); 1.98-2.02 (2H, m, H₆α and H₈β); 1.90 (1H, dd, J=7.6 Hz and 10.7 Hz, H₁₇α); 1.96 (1H, dd, J=10.4 Hz and 14.0 Hz, H₁₅β); 2.01-2.05 (2H, m, H₅α and H_{2′}); 2.18 (6H, s, N_{B}—CH₃); 2.25 (1H, d, J=17.7 Hz, H₁₂β); 2.40 (1H, m, H₁β); 2.42 (1H, d, J=17.7 Hz, H₁₂α); 2.55 (1H, dd, J=6.6 Hz and 10.7 Hz, H₂₀); 2.92 (1H, d, J=13.9 Hz, H₂₉ₐ); 3.09 (1H, m, H₃α); 3.11 (1H, d, J=13.9 Hz, H_{29b}); 4.02 (1H, m, H16β); 4.23 (1H, bs, N_{AH} or N_{C}H).

¹³C NMR (125.8 MHz, CDCl₃), δppm:

9.9 (C₂₁); 11.8 (C₃₀); 12.2 (C_{4′}); 17.9 (C₁₈); 18.2 (C₆); 18.4 (C_{5′}); 20.6 (C₂₈); 24.2 (C₇); 27.5 (C_{3′}); 28.1 (C₁ and C₂); 30.3 (C₁₉); 34.4 (C₅ and C₉); 38.0 (C₁₀); 41.0 (C₈); 42.4 (C_{2′}); 42.7 (C₁₅); 44.6 (C₁₃); 47.2 (C₄); 47.3 (C₁₄); 51.5 (C₁₂); 55.5 (C₃); 55.9 (C₁₇); 58.1 (C₂₉); 62.1 (C₂₀); 78.3 (C₁₆); 159.8 (C_{1′}); 211.0 (C₁₁).

### Compound 65: (20S)-10(9->1)abeo-16α-acetyl-3β-((S))-2-methylbutyrylamino)-20-dimethylamino-4β,14α-dimethyl-4α-hydroxymethyl-5α,9β-pregn-1(10)-en-11-one 65

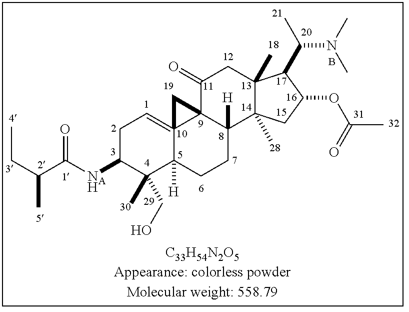

To a solution of 735.5 mg (1.42 mmol, 1.0 eq) of amide 7e in 71 ml of dichloromethane and 21 ml of pyridine are added 202 μl (2.13 mmol, 1.5 eq) of acetic anhydride. The solution is light yellow. After 16 hours of agitation at room temperature, the mixture is co-evaporated in the presence of 1,2-dichloroethane. The residue is alkalinized with 30 ml of a 10% ammonia solution (pH=10) and extracted with 3×20 ml of dichloromethane. The recombined organic phases are successively dried on anhydrous sodium sulfate and evaporated under a vacuum.

The residue thus obtained is crystallized in acetone to yield 169 mg of 65 in the form of colorless crystals. The mother liquors are evaporated under a vacuum and the residue is chromatographed on a Merck activity II-III alumina column (eluent: 9/1 dichloromethane/methanol) to yield 378 mg of 65 in the form of a colorless powder after recrystallization in acetone with a total yield of 90%.

MP (° C.): MISSING

Elemental analysis: for C₃₃H₅₄N₂O₅

Calculated for C₃₃H₅₄N₂O₅%: C, 70.93; H, 9.74; N, 5.01; O, 14.32.

Measured %: C, 70.82; H, 10.05; N, 4.95; O, 4.16.

IR (diamond) υ(cm⁻¹): 3238 (OH, NH); 1731 (C═O ester) and 1698 (C═O); 1632 and 1552 (CONH); 1454 1377 1243 1034 (C—OH).

MS (ESI): m/z: 559.4 ([M+H], 100); 560.4 (15).

HRMS (ESI) calculated for C₃₃H₅₅N₂O₅ m/z=559.4111; measured: 559.4150.

¹H NMR (300 MHz, CDCl₃), δppm:

0.47 (3H, s, H₃₀); 0.70 (3H, s, H₁₈); 0.82 (3H, d, J=6.2 Hz, H₂₁); 0.92 (1H, m, H₆β); 0.95 (3H, t, J=7.5 Hz, H_{4′}); 1.16 (3H, d, J=6.8 Hz, H_{5′}); 1.22 (3H, s, H₂₈); 1.40-1.96 (7H, m, H₂β, H_{2′}, H_{3′}, H₇β, H₇α and H₁₅α); 1.90 (1H, m, H₁₉); 2.01 (6H, s, N_{B}—CH₃); 2.35-2.45 (6H, m, H₂α, H₆α, H₁₇α, H₅α, H₈β, H₉α and H₁₅β); 2.13 (3H, s, H₃₂); 2.41 (1H, d, J=15.6 Hz, H₁₂β); 2.45 (1H, m, H₂₀); 2.61 (1H, d, J=15.6 Hz, H₁₂α); 3.16 (1H, dd, J=4.5 Hz and 12.4 Hz, H₂₉ₐ); 3.36 (1H, d, J=14.3 Hz, H₁₉α); 3.40 (1H, d, J=10.9 Hz and 12.4 Hz, H_{29b}); 4.08 (1H, ddd, J=6.1 Hz, 9.0 Hz and 10.9 Hz, H₃α); 4.66 (1H, dd, J=4.5 Hz and 10.9 Hz, OH); 5.12 (1H, dd, J=6.1 Hz and 8.0 Hz, H₁₆β); 5.39 (1H, d, J=8.9 Hz, N_{A}H); 5.48 (1H, s, H₁).

¹³C NMR (75.5 MHz, CDCl₃), δppm:

9.9 (C₃₀); 10.0 (C₂₁); 12.1 (C_{4′}); 17.6 (C_{5′}); 17.9 (C₁₈); 18.0 (C₂₈); 21.2 (C₃₂); 25.0 (C₆); 27.5 (C_{3′}); 30.1 (C₂); 33.3 (C₇); 37.5 (C₁₉); 40.1 (N_{B}—CH₈); 42.0 (C₄); 42.7 (C₁₅); 43.3 (C_{2′}); 43.5 (C₅); 46.2 (C₁₃); 47.3 (C₁₄); 48.0 (C₃); 49.7 (C₈); 49.9 (C₉); 50.3 (C₁₂); 54.2 (C₁₇); 59.6 (C₂₀); 63.8 (C₂₉); 78.7 (C₁₆); 117.9 (C₁); 139.9 (C₁₀); 170.6 (C₃₁); 178.4 (C_{1′}); 211.6 (C₁₁).

### Compound 66: (20S)-10(9->1)abeo-16α-acetyl-3β-((S)-2-methylbutyrylamino)-20-dimethylamino-4α-formyl-4β,14α-dimethyl-5α,9β-pregn-1(10)-en-11-one 66

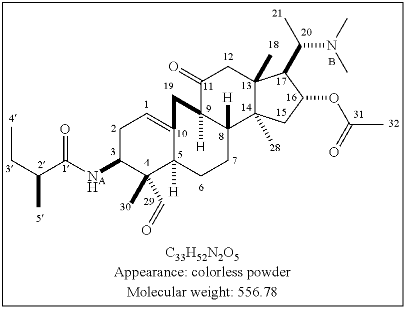

To a solution of 159.5 mg (0.28 mmol, 1.0 eq) of amide 65 in 3 ml of dichloromethane are added 162 mg (0.37 mmol, 1.3 eq) of the Dess-Martin periodinane. After 1 hour of agitation at room temperature, the mixture is alkalinized with 30 ml of a 10% ammonia solution (pH=10) and extracted with 3×25 ml of dichloromethane. The recombined organic phases are successively dried on anhydrous sodium sulfate and evaporated under a vacuum.

The residue is chromatographed on a solid-deposit silica column (eluent: 95/5 dichloromethane/methanol) to yield 122 mg of 66 in the form of a colorless powder after recrystallization in acetone with a yield of 77%.

MP (° C.): 215° C.

Elemental analysis: for C₃₃H₅₂N₂O₅.0.5 H₂O

Calculated for C₃₃H₅₂N₂O₅.0.5 H₂O %: C, 70.05; H, 9.44; N, 4.95.

Actual %: C, 70.06; H, 9.61; N, 4.55.

IR (diamond) υ(cm⁻¹): 3374 (OH, NH); 2965 (CH, CH₂); 2790 (CH═O); 1726 (C═O ester); 1697 (C═O); 1666 and 1529 (C═C and CONH); 1245, 1030 (C—OH).

MS (ESI): m/z: 557.4 ([M+H], 100); 558.4 (15); 515.4 (15); 501.4 (5).

HRMS (ESI) calculated for C₃₃H₅₃N₂O₅ m/z=557.3954; measured: 557.3957.

¹H NMR (300 MHz, CDCl₃), δppm:

0.68 (3H, s, H₁₈); 0.81 (3H, d, J=6.4 Hz, H₂₁); 0.82 (3H, t, J=7.3 Hz, H_{4′}); 0.83 (3H, s, H₃₀); 1.06 (3H, d, J=6.9 Hz, H_{5′}); 1.21 (3H, s, H₂₈); 1.26-1.42 (3H, m, H₆β, H₇α and H₁₅α); 1.51-1.65 (4H, m, H_{3′}, H₈β and H₆α); 1.77-1.90 (3H, m, H₇β, H₅α and H₂β); 1.99 (3H, s, H₃₂); 2.12 (6H, s, N_{B}—CH₈); 1.95-2.16 (3H, m, H₁₉, H₁₅β and H₉); 2.24 (1H, dd, J=5.8 Hz and 10.8 Hz, H₁₇α); 2.41 (1H, d, J=15.1 Hz, H₁₂β); 2.33-2.48 (3H, m, H_{2′}, H₂₀ and H₂α); 2.60 (1H, d, J=15.1 Hz, H₁₂α); 3.40 (1H, d, J=14.3 Hz, H₁₉α); 4.47 (1H, ddd, J=6.4 Hz, 9.3 Hz and 10.0 Hz, H₃α); 5.10 (1H, dd, J=6.2 Hz and 7.9 Hz, H₁₆β); 5.27 (1H, d, J=8.5 Hz, N_{A}H); 5.54 (1H, s, H₁); 9.47 (1H, s, H₂₉).

¹³C NMR (75.5 MHz, CDCl₃), δppm:

6.9 (C₃₀); 10.1 (C₂₁); 11.9 (C_{4′}); 17.4 (C_{5′}); 18.0 (C₁₈ and C₂₈); 21.3 (C₃₁); 27.1 (C₆); 27.4 (C_{3′}); 29.3 (C₂); 33.2 (C₇); 36.9 (C₁₉); 40.4 (N_{B}—CH₃); 42.8 (C₁₅); 43.3 (C_{2′}); 44.7 (C₅); 46.4 (C₁₄); 47.0 (C₉); 47.4 (C₁₄); 49.6 (C₃); 50.0 (C₈); 50.4 (C₁₂); 54.4 (C₁₇); 55.5 (C₄); 59.7 (C₂₀); 78.7 (C₁₆); 120.1 (C₁); 136.9 (C₁₀); 170.6 (C₃₁); 176.2 (C_{1′}); 204.5 (C₂₉); 211.0 (C₁₁).

### Compound 67: (20S)-10(9->1)-abeo-20-dimethylamino-3-((S)-2-methylbutyrylamino)-4α-formyl-4β,14α-dimethyl-5α,9β-pregn-1(10)-en-16α-ol-11-one 67

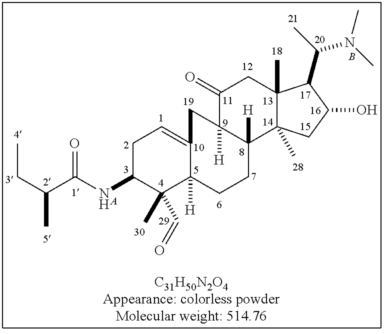

To a solution of 304 mg (0.54 mmol, 1.0 eq) of amide 66 in 7 ml of methanol, heated beforehand at 40° C. for total dissolution, is added a solution of 688 mg (8.18 mmol, 15.0 eq) of sodium hydrogen carbonate and 579 mg (5.46 mmol, 10.0 eq) of sodium carbonate in 11.5 ml of permuted water. A precipitate is formed. After 7 hours of agitation at 55° C., the mixture is diluted with 40 ml of water and extracted with 3×40 ml of dichloromethane. The recombined organic phases are successively dried on anhydrous sodium sulfate and evaporated under a vacuum.

The residue is chromatographed on a Merck activity II-III alumina column (eluent: 98/2 dichloromethane/methanol) to yield 237 mg of 67 in the form of a colorless powder with a yield of 84%, in two steps.

MP (° C.): 265° C.

Elemental analysis: for C₃₃H₅₂N₂O₅

Calculated for C₃₃H₅₂N₂O₅%: C, 71.19; H, 9.41; N, 5.03; O, 14.37.

Actual %: C H N O

IR (diamond) υ(cm⁻¹): 3319 (OH, NH); 2931 (CH, CH₂); 2787 (CH═O); 1722 (C═O aldehyde); 1695 (C═O); 1649 and 1528 (CONH); 1214 and 1016 (C—OH).

MS (ESI): m/z: 515.3 ([M+H], 100); 516.4 (45); 501.4 (5).

HRMS (ESI) calculated for C₃₁H₅₁N₂O₄ m/z=515.3849; measured: 515.3844.

¹H NMR (300 MHz, CDCl₃), δppm:

0.62 (3H, s, H₁₈); 0.76 (3H, s, H₃₀); 0.78 (3H, t, J=7.4 Hz, H_{4′}); 0.81 (3H, d, J=6.4 Hz, H₂₁); 1.00 (3H, d, J=6.8 Hz, H_{5′}); 1.20 (3H, s, H₂₈); 1.22-1.60 (6H, m, H_{3′}, H₆α, H₆β, H₇β, and H₁₅α); 1.69-1.83 (4H, m, H₂β, H7β, H₈β, and H₅α); 1.84-2.01 (5H, m, H_{2′}, H₉α, H₁₉, H₁₅β and H₇α); 2.17 (6H, s, N_{B}—CH₃); 2.29 (1H, d, J=15.8 Hz, H₁₂β); 2.36 (1H, m, H₂₀); 2.52 (1H, d, J=15.8 Hz, H₁₂α); 2.55 (1H, m, H₂₀); 3.34 (1H, d, J=14.3 Hz, H₁₉α); 4.01 (1H, ddd, J=2.5 Hz, 8.4 Hz and 8.5 Hz, H₁₆β); 4.40 (1H, ddd, J=6.4 Hz, 7.1 Hz and 12.1 Hz, H₃α); 5.22 (1H, d, J=10.0 Hz, NAH); 5.48 (1H, s, H₁); 9.42 (1H, s, H₂₉).

¹³C NMR (75.5 MHz, CDCl₃), δppm:

6.8 (C₃₀); 10.0 (C₂₁); 11.8 (C_{4′}); 17.3 (C_{5′}); 17.8 (C₁₈); 18.8 (C₂₈); 27.1 (C₆); 27.3 (C_{3′}); 29.2 (C₂); 33.2 (C₇); 37.0 (C₁₉); 42.4 (C₁₅); 43.2 (C_{2′}); 44.6 (C₈); 46.2 (C₁₃); 46.8 (C₅); 47.0 (C₁₄); 49.6 (C₃); 50.0 (C₁₂); 50.3 (C₉); 55.4 (C₁₇ and C₄); 62.0 (C₂₀); 78.3 (C₁₆); 119.9 (C₁); 136.8 (C₁₀); 176.1 (C_{1′}); 204.5 (C₂₉); 211.4 (C₁₁).

### Compound 68: (20S)-10(9->1)-abeo-20-dimethylamino-3-((S)-2-methylbutyrylamino)-4α-benzyliminomethyl-4β,14α-dimethyl-5α,9β-pregn-1(10)-en-16α-ol-11-one 68

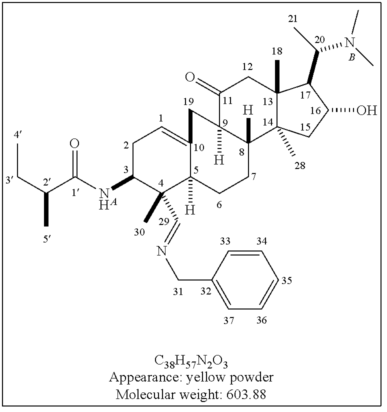

Under argon, to a suspension of 200 mg (0.39 mmol, 1.0 eq) of aldehyde 67 and 160 mg of anhydrous magnesium sulfate in 2 ml of dichloromethane are added 55 μl (0.50 mmol, 1.3 eq) of benzylamine. After 7 hours of agitation at 55° C., the solution is filtered on celite and evaporated under a vacuum to yield 272 mg of 68 in the form of a yellow powder with a quantitative yield.

IR (diamond) υ(cm⁻¹): 3309 (OH, NH); 2929 (CH, CH₂); 1728 (C═O); 1695 (C═N); 1649 and 1532 (C═C, CONH); 1451 (C═C Ar); 1227 and 1091 (C—OH).

MS (ESI): m/z: 604.4 ([M+H], 100); 605.4 (10); 646.5 (5).

HRMS (ESI) calculated for C₃₈H₅₈N₃O₃ m/z=604.4478; measured: 604.4460.

¹H NMR (300 MHz, CDCl₃), δppm:

0.61 (3H, s, H₁₈); 0.75 (3H, t, J=7.4 Hz, H_{4′}); 0.80 (3H, d, J=6.4 Hz, H₂₁); 0.86 (3H, s, H₃₀); 0.96 (3H, d, J=7.0 Hz, H_{5′}); 1.19 (3H, s, H₂₈); 1.21-1.62 (6H, m, H_{3′}, H₆α, H₆β, H₇β, and H₁₅α); 1.67-1.83 (4H, m, H₂β, H₇β, H₈β and H₅α); 1.86-1.97 (4H, m, H_{2′}, H₉α, H₁₅β and H₇α); 2.01 (1H, m, H₁₉); 2.06 (6H, s, N_{B}—CH₃); 2.28 (1H, d, J=16.0 Hz, H₁₂β); 2.33 (1H, m, H₂α); 2.51 (1H, d, J=16.0 Hz, H₁₂α); 2.54 (1H, m, H₂₀); 3.30 (1H, d, J=14.5 Hz, H₁₉α); 4.02 (1H, ddd, J=2.0 Hz, 7.5 Hz and 7.9 Hz, H₁₆β); 4.19 (1H, ddd, J=6.2 Hz, 7.0 Hz and 13.0 Hz, H₃α); 4.44 and 4.56 (2H, 2d system AB, J=13.4 Hz, H₃₁); 5.22 (1H, d, J=9.0 Hz, N_{A}H); 5.47 (1H, s, H₁); 7.15-7.28 (5H, m, H_{Ar=33-37}); 7.56 (1H, s, H₂₉).

¹³C NMR (75.5 MHz, CDCl₃), δppm: [data missing]

### Compound 69: (20S)-10(9->1)abeo-20-dimethylamino-3β-((S)-2-methylbutyrylamino)-4α-benzylaminomethyl-4β,14α-dimethyl-5α,9β-pregn-1(10)-en-16α-ol-11-one 69

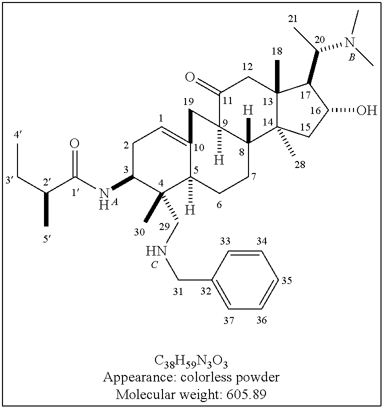

To a suspension of 272 mg (0.39 mmol, 1.0 eq) of imine 68 in 2 ml of methanol are successively added 36 mg (0.54 mmol, 1.4 eq) of sodium cyanoborohydride and 26 μl (0.45 mmol, 1.15 eq) of glacial acetic acid. After 3 hours of agitation at room temperature, the mixture is alkalinized with 40 ml of a 10% ammonia solution (pH=10) and extracted with 3×40 ml of dichloromethane. The recombined organic phases are successively dried on anhydrous sodium sulfate and evaporated under a vacuum.

The residue is chromatographed on a Merck activity II-III alumina column (eluent: 98/1 dichloromethane/methanol) to yield 173 mg of 69 in the form of a colorless powder with a yield of 73%, in two steps.

IR (diamond) υ(cm⁻¹): 3313 (OH, NH); 2961 (CH, CH₂); 1695 (C═O); 1644 and 1530 (CONH); 1452 (C═C Ar); 1237 and 1095 (C—OH).

MS (ESI): m/z: 606.4 ([M+H], 100); 607.5 (20).

HRMS (ESI) calculated for C₃₈H₆₀N₃O₃ m/z=606.4635; measured: 606.4636.

¹H NMR (300 MHz, CDCl₃), δppm:

0.45 (3H, s, H₃₀); 0.61 (3H, s, H₁₈); 0.80 (3H, d, J=6.4 Hz, H₂₁); 0.86 (3H, t, J=7.4 Hz, H_{4′}); 1.06 (3H, d, J=6.8 Hz, H_{5′}); 1.24 (3H, s, H₂₈); 1.28-1.62 (6H, m, H_{3′}, H₆α, H₆β, H₇β, and H15α); 1.67-1.76 (4H, m, H₂β, H₇β, H₈β and H₅α); 1.82 (1H, m, H₁₉); 1.88-2.08 (4H, m, H_{2′}, H₉α, H₁₅β and H₇α); 2.17 (6H, s, N_{B}—CH₃); 2.20 (1H, d, J=12.5 Hz, H₂₉); 2.27 (1H, d, J=15.6 Hz, H₁₂β); 2.32 (1H, d, J=12.5 Hz, H₂₉); 2.33 (1H, m, H₂α); 2.52 (1H, d, J=15.6 Hz, H₁₂α); 2.62 (1H, m, H₂₀); 3.23 (1H, d, J=14.1 Hz, H₁₉α); 3.48 and 3.78 (2H, 2d system AB, J=13.6 Hz, H₃₁); 4.02 (2H, m, H₁₆β and H₃α); 5.23 (1H, d, J=9.6 Hz, N_{A}H); 5.37 (1H, s, H₁); 7.12-7.27 (5H, m, H_{Ar=33-37}).

¹³C NMR (75.5 MHz, CDCl₃), δppm:

10.0 (C₂₁); 12.0 (C_{4′}); 12.4 (C₃₀); 17.6 (C₁₈); 17.7 (C_{5′}); 19.0 (C₂₈); 25.2 (C₆); 27.4 (C_{3′}); 30.7 (C₂); 33.3 (C₇); 37.5 (C₁₉); 40.5 (C₄); 42.5 (C₁₅); 43.7 (C_{2′}); 44.7 (C₅); 46.3 (C₁₃); 47.1 (C₁₄); 48.0 (C₃); 49.7 (C₈); 50.1 (C₁₂); 50.3 (C₉); 52.6 (C₂₉); 54.3 (C₃₁); 55.4 (C₁₇); 62.1 (C₂₀); 78.4 (C₁₆); 118.3 (C₁); 126.6 (C₃₅); 128.0 and 128.4 (C₃₄₋₃₆ and C₃₃₋₃₇); 139.8 (C₁₀); 141.2 (C₃₂); 176.4 (C_{1′}); 212.0 (C₁₁).

### Compound 70: (20S)-10(9->1)-abeo-20-dimethylamino-3-((S)-2-methylbutyrylamino)-4α-aminomethyl-4β,14α-dimethyl-5α,9β-pregn-1(10)-en-16α-ol-11-one 70

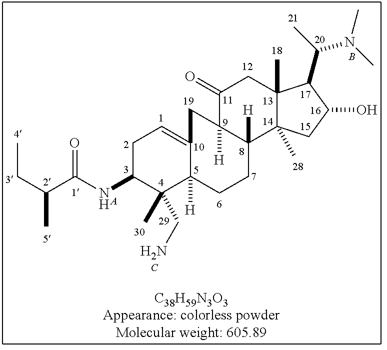

Under argon, to a suspension of 296 mg (0.49 mmol, 1.0 eq) of N-benzylated amine 69 in 10 ml of anhydrous methanol degassed with argon, are successively added 148 mg (50% by weight with respect to 69) of 30% palladium on carbon and 159 mg (2.43 mmol, 5.0 eq) of ammonium formate. After 3 hours of agitation at 40° C., the mixture is filtered on celite and evaporated under a vacuum. The residue obtained is alkalinized with 40 ml of a 10% ammonia solution (pH=10) and extracted with 3×30 ml of dichloromethane. The recombined organic phases are successively dried on anhydrous sodium sulfate and evaporated under a vacuum.

The residue is chromatographed on a silica column (eluent: 90/9.9/0.1 dichloromethane/methanol/ammonia) to yield 90 mg of 70 in the form of a colorless powder with a yield of 36%, in two steps.

IR (diamond) υ(cm⁻¹): 3314 (OH, NH); 2932 (CH, CH₂); 1660 (C═O); 1660 and 1529 (CONH); 1452 (C═C Ar); 1226 and 1094 (C—OH).

MS (ESI): m/z: 606.5 ([M+H], 100); 607.5 (10).

HRMS (ESI) calculated for C₃₈H₆₀N₃O₃ m/z=606.4635; measured: 606.4634.

¹H NMR (300 MHz, CDCl₃), δppm:

0.50 (3H, s, H₃₀); 0.62 (3H, s, H₁₈); 0.81 (3H, d, J=6.6 Hz, H₂₁); 0.86 (3H, t, J=7.4 Hz, H_{4′}); 1.05 (3H, d, J=6.8 Hz, H_{5′}); 1.21 (3H, s, H₂₈); 1.17-1.25 (2H, m, H₆); 1.26-1.64 (4H, m, H_{3′}, H₇β); 1.45 (1H, dd, J=1.7 Hz and 13.8 Hz, H₁₅α); 1.65-1.81 (3H, m, H₂β, H₈β and H₅α); 1.82 (1H, m, H₁₉); 1.82-2.11 (3H, m, H₉α, H₁₇α, H₁₅β and H₇α); 2.04 (1H, m, H_{2′}); 2.19 (6H, s, N_{B}—CH₃); 2.28 (1H, d, J=15.3 Hz, H₁₂β); 2.27 (1H, m, H₂α); 2.46 (1H, d, J=12.5 Hz, H₂₉ₐ); 2.50 (1H, d, J=15.3 Hz, H₁₂α); 2.56 (1H, d, J=13.0 Hz, H_{29b}); 2.57 (1H, m, H₂₀); 3.27 (1H, d, J=13.8 Hz, H₁₉α); 4.04 (1H, m, H₁₆β); 4.06 (1H, m, H₃α); 5.30 (1H, d, J=9.6 Hz, N_{A}H); 5.41 (1H, s, H₁).

¹³C NMR (75.5 MHz, CDCl₃), δppm:

10.0 (C₂₁); 12.0 (C_{4′}); 12.4 (C₃₀); 17.6 (C_{5′}); 17.8 (C₁₈); 19.0 (C₂₈); 25.0 (C₆); 27.4 (C_{3′}); 30.7 (C₂); 33.4 (C₇); 37.5 (C₁₉); 41.1 (C₄); 42.5 (C₁₅); 43.6 (C_{2′}); 43.9 (C₅); 45.6 (C₂₉); 46.3 (C₁₃); 47.1 (C₁₄); 47.2 (C₃); 49.8 (C₁₂); 50.0 (C₈); 50.2 (C₉); 55.3 (C₁₇); 62.1 (C₂₀); 78.2 (C₁₆); 118.8 (C₁); 139.2 (C₁₀); 176.8 (C_{1′}); 211.8 (C₁₁).

### Compound 71: (20S)-10(9->1)-abeo-20-dimethylamino-4β,14α-dimethyl-(2-(S)-sec-butyl-5β-methyl-1,4,5,6-tetrahydro-pyrimidin-5-yl)-5α,9β-pregnan-1(10)-en-16α-ol-11-one 71

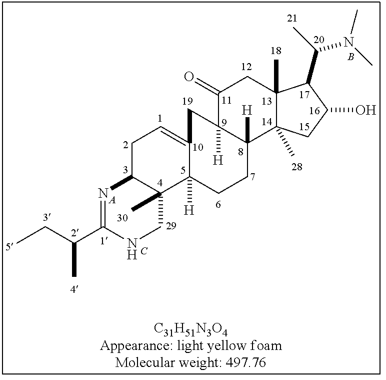

To a solution of 61 mg (0.12 mmol, 1.0 eq) of amine 70 in 30 ml of n-butanol is added 37 μl (0.26 mmol, 2.0 eq) of triethylamine. After 16 hours of agitation at 120° C., the mixture is evaporated under a vacuum. The residue obtained is alkalinized with 30 ml of a 10% ammonia solution (pH=10) and extracted with 3×40 ml of dichloromethane. The recombined organic phases are successively dried on anhydrous sodium sulfate and evaporated under a vacuum.

The residue is chromatographed on a Merck activity II-III alumina column (eluent: 95/5 dichloromethane/methanol) to yield 29 mg of 71 in the form of a light yellow foam with a yield of 49%.

MP (° C.): 225° C.

IR (diamond) υ(cm⁻¹): 3315 (OH, NH); 2930 (CH, CH₂); 1694 (C═O); 1627 and 1518 (C═C, C—N); 1039 (C—OH).

MS (ESI): m/z: 498.4 ([M+H], 100); 499.4 (10).

HRMS (ESI) calculated for C₃₁H₅₂N₃O₄ m/z=498.4060; measured: 498.4043.

Note: This is a mixture of two tautomeric forms. Nitrogens N_{A} and N_{C} exchange a proton. In ¹³C NMR, carbons C3 and C29 give masses of low integration.

¹H NMR (500 MHz, CDCl₃), δppm:

0.63 (3H, s, H₁₈); 0.67 (3H, s, H₃₀); 0.80 (3H, d, J=6.3 Hz, H₂₁); 0.85 (3H, t, J=7.5 Hz, H_{4′}); 1.04 (3H, d, J=6.9 Hz, H_{5′}); 1.17 (3H, s, H₂₈); 1.19-1.36 (3H, m, H_{3a′} and H₆); 1.48-1.62 (3H, m, H_{3b′}, H₇); 1.44 (1H, dd, J=1.9 Hz and 13.9 Hz, H₁₅α); 1.67-1.75 (2H, m, H₂); 1.83-1.93 (3H, m, H₉α, H₁₇α and H₁₅β); 1.97-2.04 (4H, m, H_{2′}, H₅α, H₈β and H₁₉); 2.19 (7H, s, N_{B}—CH₃ and H₂α); 2.28 (1H, d, J=15.8 Hz, H₁₂β); 2.48 (1H, d, J=15.8 Hz, H₁₂α); 2.57 (1H, dd, J=6.3 Hz and 10.7 Hz, H₂₀); 2.95 (1H, d, J=12.9 Hz, H₂₉ₐ); 3.24 (1H, d, J=15.8 Hz, H₁₉α); 3.25 (1H, d, J=12.9 Hz, H_{29b}); 4.04 (1H, dd, J=6.9 Hz and 7.3 Hz, H₁₆β); 4.17 (1H, bs, N_{A}H or N_{C}H); 5.47 (1H, s, H₁).

¹³C NMR (128.5 MHz, CDCl₃), δppm:

10.0 (C₂₁); 10.9 (C₃₀); 12.2 (C_{4′}); 17.8 (C₁₈); 18.6 (C_{5′}); 18.8 (C₂₈); 25.0 (C₆); 28.2 (C_{3′}); 29.7 (C₂); 33.1 (C₄); 33.8 (C₇); 37.5 (C₁₉); 42.4 (C_{2′}); 42.5 (C₁₅); 46.2 (C₁₃); 47.1 (C₁₄); 49.8 (C₅); 50.1 (C₁₂); 50.4 (C₉); 50.8 (C₃); 51.0 (C₈); 54.2 (C₂₉); 55.5 (C₁₇); 62.0 (C₂₀); 78.4 (C₁₆); 120.1 (C₁); 138.5 (C₁₀); 159.9 (C_{1′}); 211.9 (C₁₁).

### Compound 75: (20S)-20-dimethylamino-16α-acetyl-3β-isobutyrylamino-9,19-cyclo-4α-methylethanethioate-4β,14α-dimethyl-5α,9β-pregnan-11-one 75

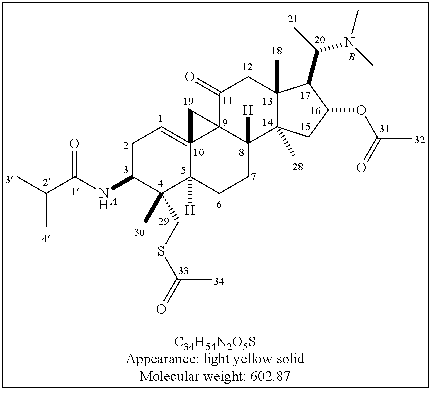

To a solution of 100 mg (1.78 mmol, 1.0 eq) of potassium hydroxide in 1.5 ml of ethanol are added 126.8 μl (1.78 mmol, 1.0 eq) of thiolacetic acid. After minutes of agitation at room temperature, the reaction mixture is evaporated and the yellow powder obtained is washed with tetrahydrofuran to yield 203 mg of potassium thioacetate in the form of a light yellow powder.

166.8 mg (1.46 mmol, 7.0 eq) of potassium thioacetate thus prepared are added to 145.8 mg (0.21 mmol, 1.0 eq) of (20S)-16α-acetyl-3β-isobutyrylamino-9,19-cyclo-4α-hydroxytosylmethyl-4β,14α-dimethyl-20-dimethylamino-5α,9β-pregnan-11-one dissolved in 5 ml of anhydrous 1,3-dimethyl-3,4,5,6-tetrahydro-2(1H)-pyrimidinone (DMPU). After 5 hours of agitation at 100° C., the reaction mixture is alkalinized with 20 ml of a 10% sodium hydrogen carbonate solution (pH=8) and extracted with 3×20 ml of ether. The recombined organic phases are successively dried on anhydrous sodium sulfate and evaporated under a vacuum.

The residue obtained is purified by silica gel flash chromatography (eluent: 9.8/0.2 dichloromethane/methanol) to yield 70.1 mg of 75 in the form of a light yellow powder with a yield of 56%.

IR (diamond) υ(cm⁻¹): 2930 (NH); 1730 (C═O); 1666 and 1529 (C═O ester, CONH and COS).

MS (ESI): m/z: 603.4 ([M+H].

### Compound 76: (20S)-20-dimethylamino-16α-acetyl-3β-isobutyrylamino-9,19-cyclo-4α-mercaptomethyl-4β,14α-dimethyl-5α,9β-pregnan-11-one 76

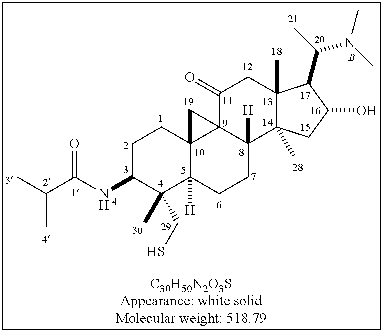

To a solution of 318.2 mg (0.53 mmol, 1.0 eq) of (209)-20-dimethylamino-16α-acetyl-3b-isobutyrylamino-9,19-cyclo-4α-methylethanethioate-4b,14α-dimethyl-5α,9b-pregnan-11-one in 6 ml of methanol is added a solution of 559.4 mg (5.27 mmol, 10.0 eq) of sodium carbonate and 665.1 mg (7.92 mmol, 15.0 eq) of sodium hydrogen carbonate in 6 ml of permuted water, heated beforehand at 40° C. for total dissolution. A precipitate is formed. After 6 hours of agitation at 65° C., the mixture is diluted with 40 ml of water and extracted with 3×40 ml of dichloromethane. The recombined organic phases are successively dried on anhydrous sodium sulfate and evaporated under a vacuum.

The residue obtained is purified by silica gel flash chromatography (eluent: 9/1 dichloromethane/methanol) to yield 229.9 mg of (20S)-20-dimethylamino-16α-acetyl-3b-isobutyrylamino-9,19-cyclo-4α-mercaptomethyl-4-b,14α-dimethyl-5α,9b-pregnan-11-one in the form of a white powder with a yield of 84%.

IR (diamond) υ(cm⁻¹): 3317 (OH); 2932 (NH); 2359 (SH); 1659 (C═O); 1659 and 1531 (CONH); 1039 (C—OH).

MS (ESI): m/z: 519.4 ([M+H].

### Example 9

### Sulfurated Molecule

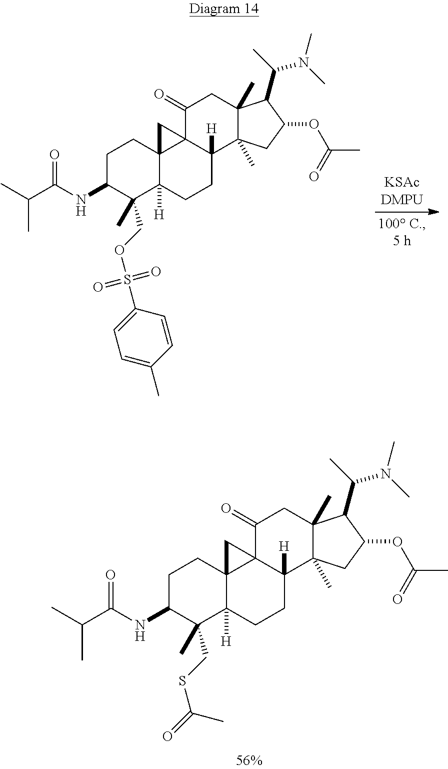

### (20S)-20-dimethylamino-16α-acetyl-3β-acetylamino-9,19-cyclo-4α-thioacetylmethyl-4β,14α-dimethyl-5α,9β-pregnan-11-one 77

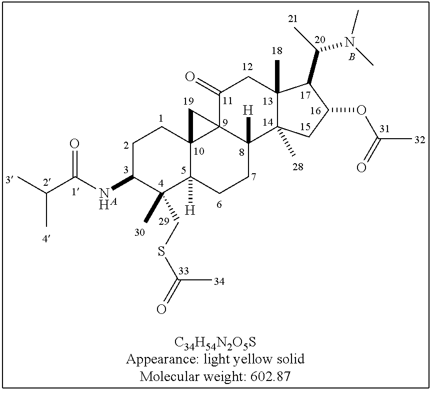

To a solution of 100 mg (1.78 mmol, 1.0 eq) of potassium hydroxide in 1.5 ml of ethanol are added 126.8 μl (1.78 mmol, 1.0 eq) of thiolacetic acid. After minutes of agitation at room temperature, the reaction mixture is evaporated and the yellow powder obtained is washed with tetrahydrofuran to yield 203 mg of potassium thioacetate in the form of a light yellow powder.

166.8 mg (1.46 mmol, 7.0 eq) of potassium thioacetate thus prepared are added to 145.8 mg (0.21 mmol, 1.0 eq) of ?? dissolved in 5 ml of anhydrous 1,3-dimethyl-3,4,5,6-tetrahydro-2(1H)-pyrimidinone (DMPU). After 5 hours of agitation at 100° C., the reaction mixture is alkalinized with 20 ml of a 10% sodium hydrogen carbonate solution (pH=8) and extracted with 3×20 ml of ether. The recombined organic phases are successively dried on anhydrous sodium sulfate and evaporated under a vacuum.

The residue obtained is purified by silica gel flash chromatography (eluent: 9.8/0.2 dichloromethane/methanol) to yield 70.1 mg of product in the form of a light yellow powder with a yield of 56%.

IR (diamond) υ(cm⁻¹): 2930 (NH); 1730 (C═O); 1666 and 1529 (C═O ester, CONH and COS).

MS (ESI): m/z: 603.4 ([M+H].

### Example 10

### Additional Biological Results (See Example 6 for Details)

TABLE 2

| | AChE^{a} | | AChE |
|---|---|---|---|
| Compound | IC₅₀ (nM) | Compound | IC₅₀ (nM) |
| 33 | 106 | 54 | >100,000 |
| 34 | >100,000 | 55 | >100,000 |
| 35 | >100,000 | 56 | >100,000 |
| 36 | >100,000 | 57 | >100,000 |
| 37 | 100,000 | 59 | >100,000 |
| 38 | 181 | 60 | >100,000 |
| 39 | 198 | 61 | >100,000 |
| 40 | 112 | 62 | 100,000 |
| 41 | >100,000 | 63 | 637 |
| 42 | >100,000 | 64 | 14 |
| 43 | >100,000 | 65 | 7,937 |
| 44 | >100,000 | 66 | >100,000 |
| 45 | >100,000 | 67 | >100,000 |
| 46 | 33,873 | 68 | 780 |
| 47 | 20,057 | 69 | >100,000 |
| 48 | 17,522 | 70 | 368 |
| 49 | 1,342 | 71 | 28 |
| 50 | 1,330 | 1 | 32 |
| 51 | 18 | tacrine | 74 |
| 52 | 29 | | |
| 76 | 43,000 | | |

1.-32. (canceled)

33. A triterpenic alkaloid of following general formula I: 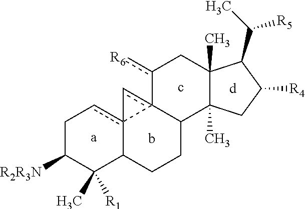
wherein
one and only one of the three dotted lines present in rings a and b represents a bond;
R₁ represents —CH₂X₁R₇, —CH═NR₈ or —CH═O wherein
X₁ represents O, NH or S;
R₇ represents a hydrogen atom, a C₁-C₁₀ alkyl, an alkyl (C₁-C₁₀) phenyl, an alkyl (C₁-C₁₀) carbonyl, an alkyl (C₁-C₁₀)-bis[trialkyl (C₁-C₁₀) silyl] or SO₂R₉, R₉ representing a C₁-C₁₀ alkyl or an alkyl (C₁-C₁₀) phenyl, provided that R₇ does not represent SO₂R₉ when X₁ represents S;
R₈ represents a hydrogen atom, a C₁-C₁₀) alkyl, an alkyl (C₁-C₁₀) phenyl, an alkyl (C₁-C₁₀) carbonyl, an alkyl (C₁-C₁₀)-bis[trialkyl (C₁-C₁₀) silyl] or SO₂R₉, R₉ representing a C₁-C₁₀ alkyl or an alkyl (C₁-C₁₀) phenyl;
R₂ and R₃ represent independently of each other a hydrogen atom or 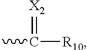
wherein X₂ represents O, NH or S and R₁₀ represents a hydrogen atom, a C₁-C₁₀ alkyl, a phenyl, a C₃-C₁₀ cycloalkyl, or an alkyl (C₁-C₁₀) phenyl, provided that at least one and only one of R₂ or R₃ represents a hydrogen atom,
or R₂ is absent, R₁ represents —CH₂X₁R₇, and —NR₃ and —X₁R₇ taken together represent 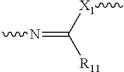
wherein R₁₁ represents a hydrogen atom, a C₁-C₁₀ alkyl, a phenyl, a C₃-C₁₀ cycloalkyl, an alkyl (C₁-C₁₀) phenyl,
or R₂ represents a hydrogen atom, R₁ represents —CH₂X₁R₇, and —NHR₃ and —X₁R₇ taken together represent 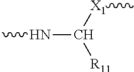
wherein R₁₁ represents a hydrogen atom, a C₁-C₁₀ alkyl, a phenyl, a C₃-C₁₀ cycloalkyl or an alkyl (C₁-C₁₀) phenyl,
or R₂ represents a hydrogen atom, R₁ represents —CH₂═NR₈, and —NHR₃ and ═NR₈ taken together represents 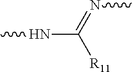
wherein R₁₁ represents a hydrogen atom, a C₁-C₁₀ alkyl, a phenyl, a C₃-C₁₀ cycloalkyl or an alkyl (C₁-C₁₀) phenyl;
R₄ represents a hydrogen atom, a C₁-C₁₀ alkyl or a —X₃R₁₂ radical wherein X₃ represents O, NH or S, and R₁₂ represents a hydrogen atom, an alkyl (C₁-C₁₀) carbonyl, an alkyl (C₁-C₁₀) phenyl, an alkyl (C₁-C₁₀) sulphonyl, an alkyl (C₁-C₁₀) phenyl sulphonyl or an alkyl (C₁-C₁₀)-dialkyl (C₁-C₁₀) silyl;
R₅ represents the N-oxide group N⁺OCH₃R₁₃ or the group NCH₃R₁₃ wherein
R₁₃ represents a hydrogen atom, a C₃-C₁₀ cycloalkyl, a C₁-C₁₀ alkyl, a phenyl or a phthalimide, and [symbol]
represents ═O or —OR₁₄ wherein R₁₄ represents a hydrogen atom, a C₁-C₁₀ alkyl, an alkyl (C₁-C₁₀) carbonyl, an alkyl (C₁-C₁₀) phenyl or an alkyl (C₁-C₁₀) sulphonyl;
or pharmaceutically acceptable addition salts, isomers, enantiomers or diastereoisomers thereof, as well as mixtures thereof,
except for compounds of the following formulas:

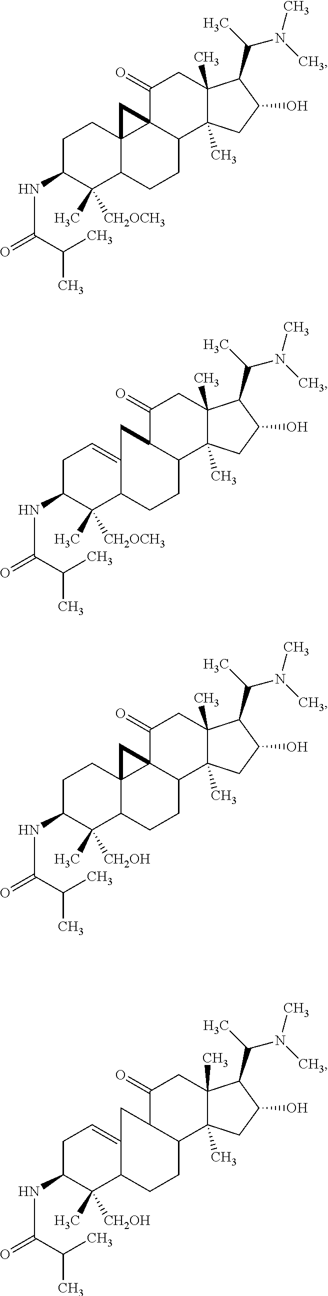

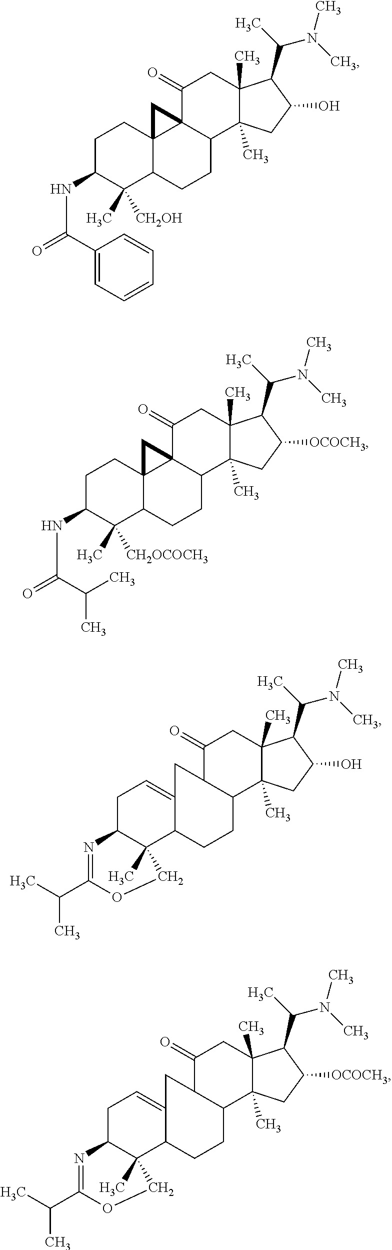

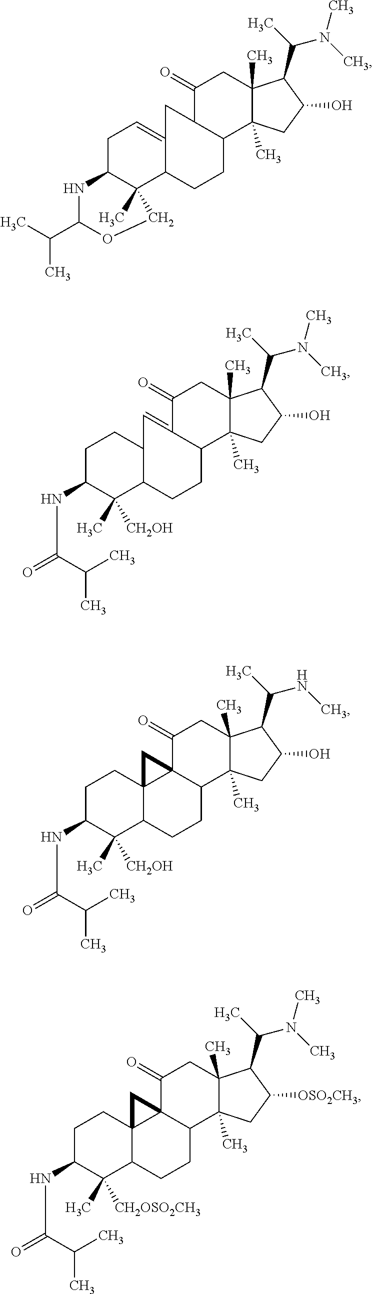

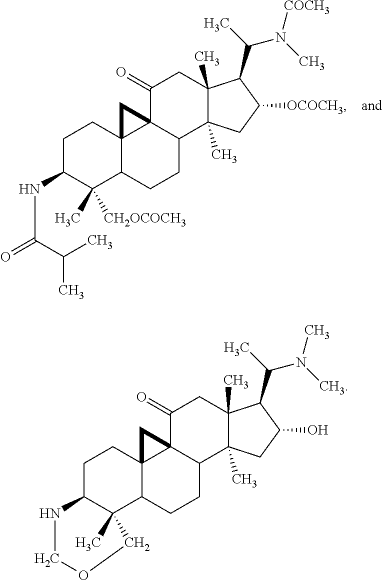

34. A triterpenic alkaloid according to claim 33, wherein R₁ represents —CH₂X₁R₇.

35. A triterpenic alkaloid according to claim 33, wherein X₁ represents O.

36. A triterpenic alkaloid according to claim 33, wherein said triterpenic alkaloid is represented by following general formula II: 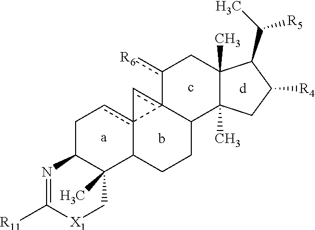
wherein one and only one of the three dotted lines present in rings a and b represents a bond;
R₄, R₅, R₁₁, [symbol] and X₁ are as defined in claim 33, or the pharmaceutically acceptable addition salts, isomers, enantiomers and diastereoisomers thereof, as well as mixtures thereof.

37. A triterpenic alkaloid according to claim 33, wherein [symbol] represents ═O.

38. A triterpenic alkaloid according to claim 33, wherein said triterpenic alkaloid is represented by following general formula III: 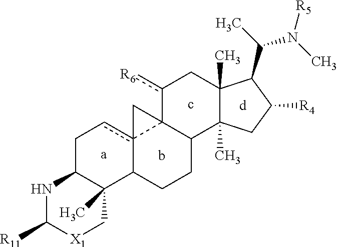
wherein one and only one of the two dotted lines present in rings a and b represents a bond; R₄, R₅, R₁₁, X₁ and [symbol] are as defined in claim 33,
or the pharmaceutically acceptable addition salts, isomers, enantiomers and diastereoisomers thereof, as well as mixtures thereof.

39. A triterpenic alkaloid according to claim 33, wherein said triterpenic alkaloid is represented by following general formula VIII: 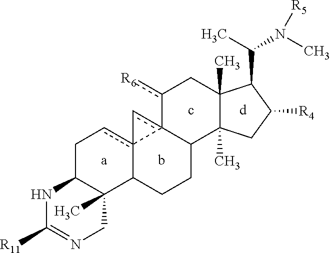
wherein one and only one of the three dotted lines present in rings a and b represents a bond; R₄, R₅ and R₁₁ and [symbol] are as defined in claim 33;
or the pharmaceutically acceptable addition salts, isomers, enantiomers and diastereoisomers thereof, as well as mixtures thereof.

40. A triterpenic alkaloid according to claim 33, wherein said triterpenic alkaloid is represented by following general formula IV: 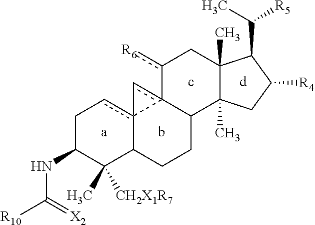
wherein one and only one of the two dotted lines present in rings a and b represents a bond, R₄, R₅, R₇, X₂, R₁₀, X₁ and [symbol] are as defined in claim 33, or the pharmaceutically acceptable addition salts, isomers, enantiomers and diastereoisomers thereof, as well as mixtures thereof.

41. A triterpenic alkaloid according to claim 33, wherein X₂ represents O.

42. A triterpenic alkaloid according to claim 33, wherein R₁₀ or R₁₁ represents independently a C₁-C₁₀ alkyl, straight or branched.

43. A triterpenic alkaloid according to claim 33, wherein said triterpenic alkaloid is selected among the compounds of the following formulas:

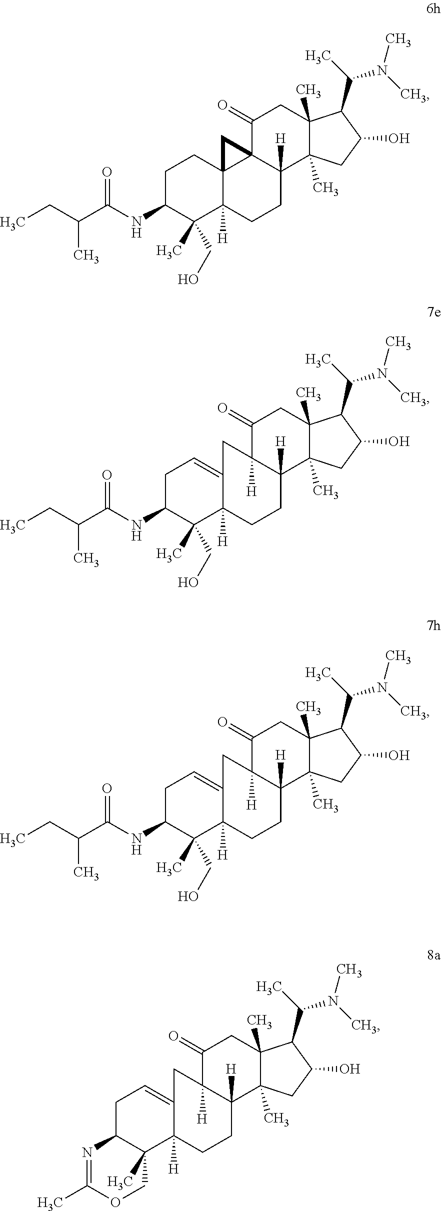

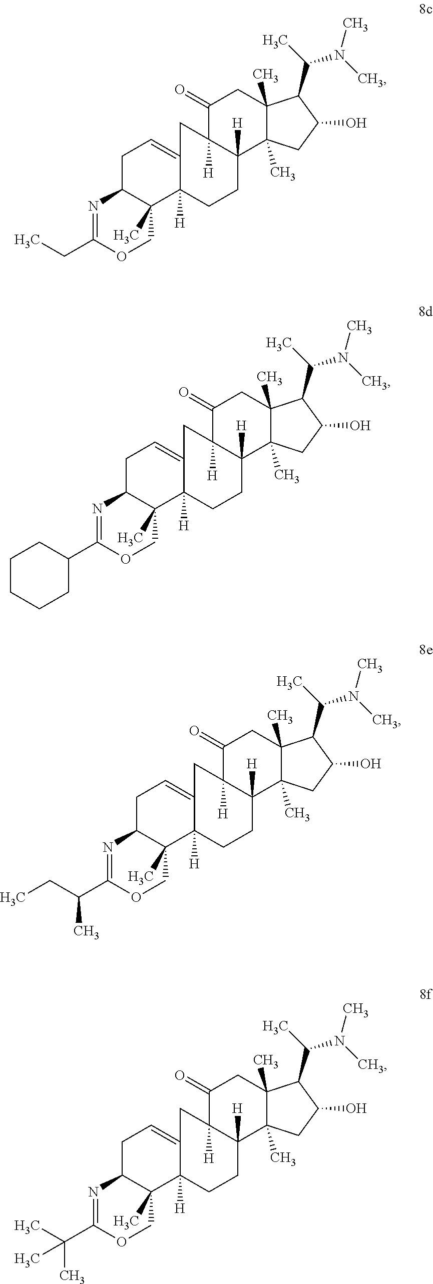

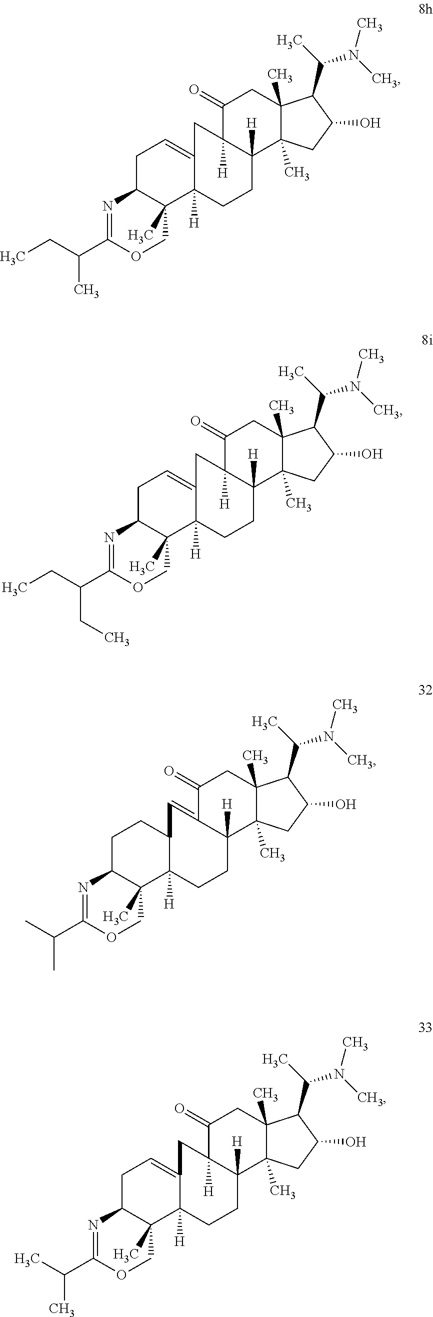

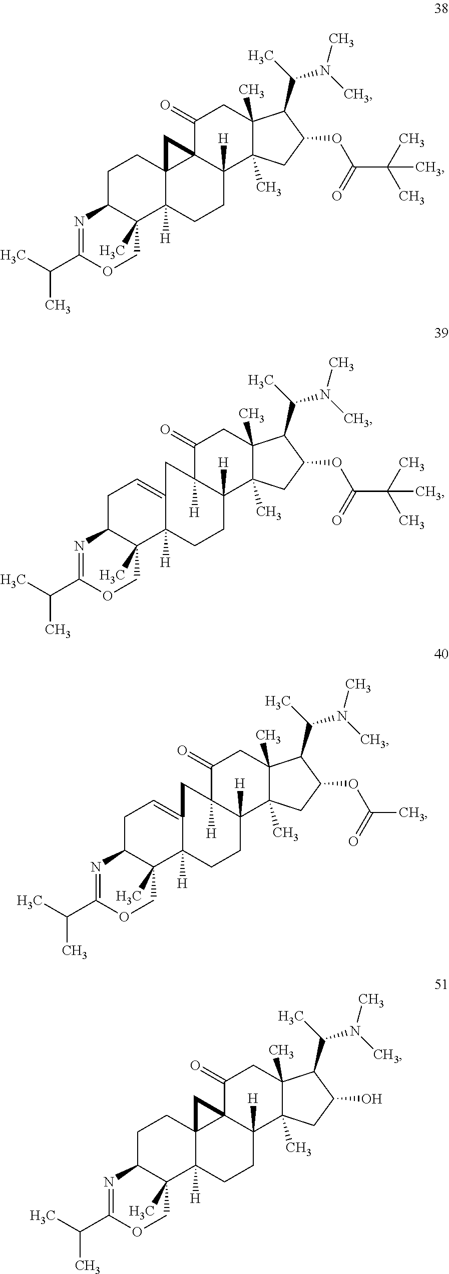

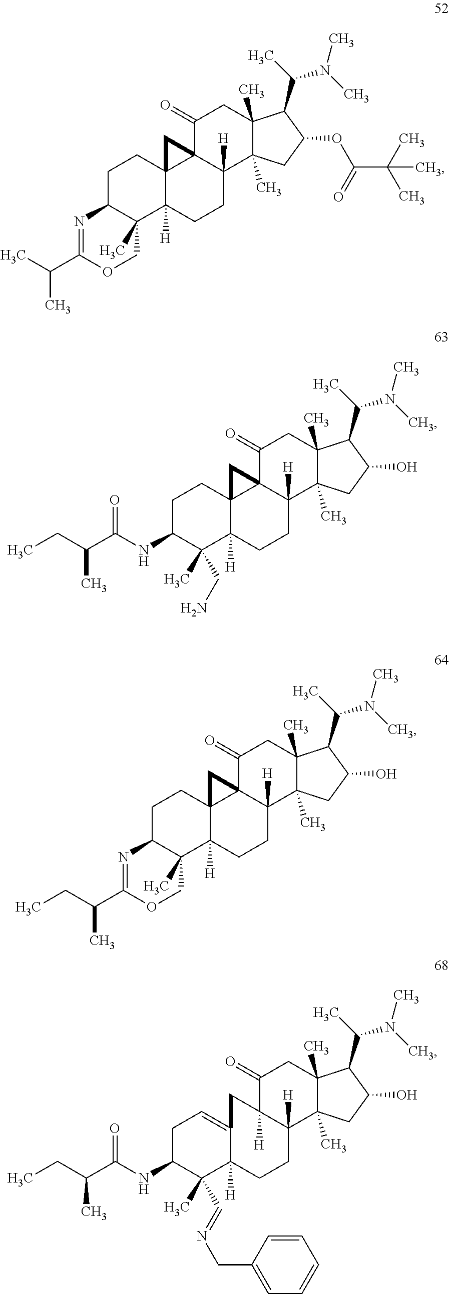

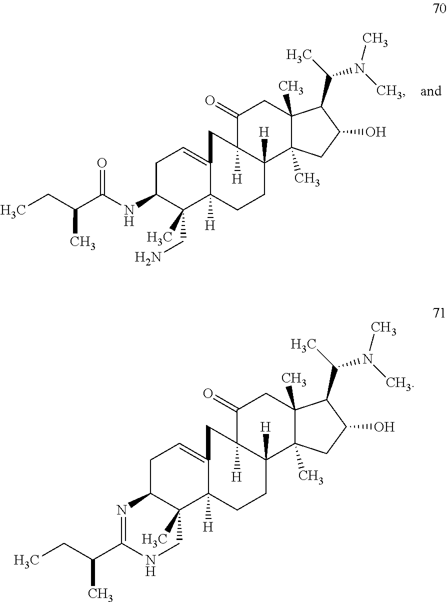

44. A method for manufacturing a compound of the following formula (2), 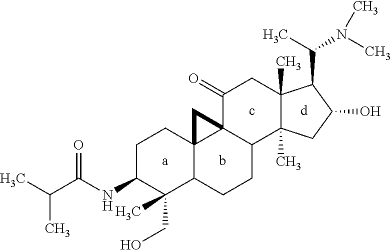
comprising the following successive steps:
a) alkalization of the ground, dried leaves of Buxus balearica Wild,
b) extraction and maceration in a CH₂Cl₂/EtOH mixture to obtain a solid extract,
c) extraction in dichloromethane with a pH between 5.8 and 6 to obtain a crude extract of the compound of formula (2),
d) purification of the compound of formula (2).

45. A method for manufacturing the compound of following general formula IIa: 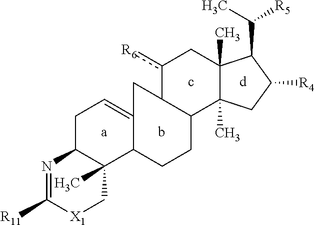
wherein R₄, R₅, R₁₁, X₁ and [symbol] are as defined in claim 33, comprising the following successive steps:
a) pyrolysis of the compound of following general formula IVb, 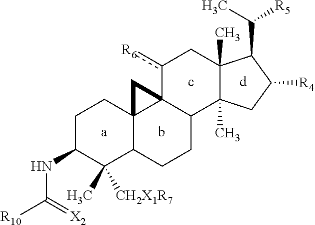
wherein R₅, R₁₀ and [symbol] are as defined in claim 33, R₄ represents —OR₁₂, wherein R₁₂ is as defined in claim 33, X₁ represents NH, R₇ represents H and X₂ represents O, at a pressure of 0.03 mmHg, at a temperature between 235° C. and 270° C. for 3 hours.
b) thermolysis of the mixture obtained in step a) at a temperature between 235° C. and 270° C., and in the presence of tetraalkylammonium hydroxide.

46. A method according to claim 45, wherein the compound of formula IVb is obtained by a method comprising the following successive steps:
reaction of the compound of following general formula IX: 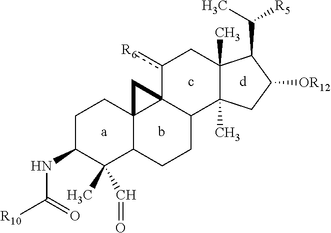
wherein R₅, R₁₀, R₁₂ and [symbol] are as defined in general formula IVb in claim 45, with benzylamine in the presence of anhydrous magnesium sulfate in dichloromethane to obtain the compound of following formula X: 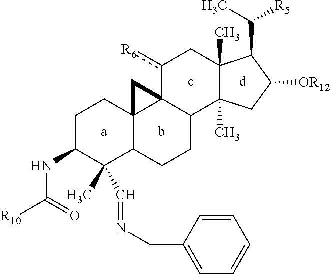
wherein R₅, R₁₀, R₁₂ and [symbol] are as defined in general formula IX above;
reaction of the compound of formula X with sodium cyanoborohydride and glacial acetic acid in methanol to obtain a compound of following formula XI: 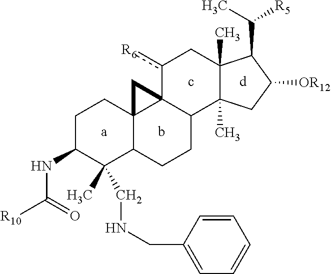
wherein R₅, R₁₀, R₁₂ and [symbol] are as defined in general formula X above;
reaction of the compound of formula XI, in the presence of 30% Pd/C catalyst in methanol, with ammonium formate or under hydrogen pressure with glacial acetic acid at pH 3 followed by alkalization with an ammonia solution to obtain the compound of formula IVb.

47. A method according to claim 46, wherein the compound of formula IX, wherein R₁₂ does not represent a hydrogen atom, is obtained by the method comprising the following successive steps:
protection of the hydroxyl group of the compound of following general formula IVb: 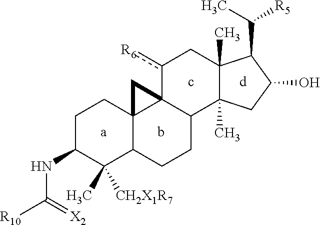
wherein R₅, R₁₀, and [symbol] are as defined in claim 46, R₇ represents H, and X₁ and X₂ represent O, by reaction with the compound of formula (R₁₂)₂O, wherein R₁₂ is as defined in claim 46, but does not represent a hydrogen atom, in a pyridine/dichloromethane mixture to obtain the compound of following general formula IVb: 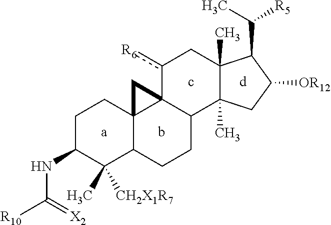
wherein R₅, R₁₀, and [symbol] are as defined in claim 46, R₇ represents H, R₁₂ is as defined in claim 46, but does not represent a hydrogen atom, and X₁ and X₂ represent O;
reaction of the compound of formula IVb as defined above with the Dess-Martin periodinane in dichloromethane to obtain the compound of formula IX, wherein R₁₂ does not represent a hydrogen.

48. A method according to claim 47, wherein the compound of formula IX, wherein R₁₂ represents a hydrogen atom, is obtained by the deprotection reaction of the compound of formula IX, wherein R₁₂ does not represent a hydrogen atom.

49. A pharmaceutical composition comprising a triterpenic alkaloid of following general formula I: 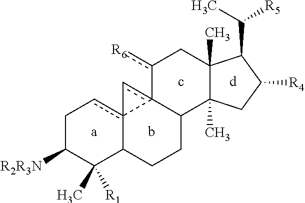
wherein
one and only one of the three dotted lines present in rings a and b represents a bond;
R₁ represents —CH₂X₁R₇, —CH═NR₈ or —CH═O wherein
X₁ represents O, NH or S;
R₇ represents a hydrogen atom, a C₁-C₁₀ alkyl, an alkyl (C₁-C₁₀) phenyl, an alkyl (C₁-C₁₀) carbonyl, an alkyl (C₁-C₁₀)-bis[trialkyl (C₁-C₁₀) silyl] or SO₂R₉, R₉ representing a C₁-C₁₀ alkyl or an alkyl (C₁-C₁₀) phenyl, provided that R₇ does not represent SO₂R₉ when X₁ represents S;
R₈ represents a hydrogen atom, a C₁-C₁₀ alkyl, an alkyl (C₁-C₁₀) phenyl, an alkyl (C₁-C₁₀) carbonyl, an alkyl (C₁-C₁₀)-bis[trialkyl (C₁-C₁₀) silyl] or SO₂R₉, R₉ representing a C₁-C₁₀ alkyl or an alkyl (C₁-C₁₀) phenyl;
R₂ and R₃ represent independently of each other a hydrogen atom or 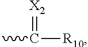
wherein X₂ represents O, NH or S and R₁₀ represents a hydrogen atom, a C₁-C₁₀ alkyl, a phenyl, a C₃-C₁₀ cycloalkyl, or an alkyl (C₁-C₁₀) phenyl, provided that at least one and only one of R₂ or R₃ represents a hydrogen atom,
or R₂ is absent, R₁ represents —CH₂X₁R₇, and —NR₃ and —X₁R₇ taken together represent 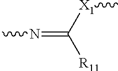
wherein R₁₁ represents a hydrogen atom, a C₁-C₁₀ alkyl, a phenyl, a C₃-C₁₀ cycloalkyl, an alkyl (C₁-C₁₀) phenyl,
or R₂ represents a hydrogen atom, R₁ represents —CH₂X₁R₇, and —NHR₃ and —X₁R₇ taken together represent 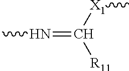
wherein R₁₁ represents a hydrogen atom, a C₁-C₁₀ alkyl, a phenyl, a C₃-C₁₀ cycloalkyl or an alkyl (C₁-C₁₀) phenyl,
or R₂ represents a hydrogen atom, R₁ represents —CH₂═NR₈, and —NHR₃ and ═NR₈ taken together represents 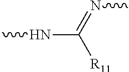
wherein R₁₁ represents a hydrogen atom, a C₁-C₁₀ alkyl, a phenyl, a C₃-C₁₀ cycloalkyl or an alkyl (C₁-C₁₀) phenyl;
R₄ represents a hydrogen atom, a C₁-C₁₀ alkyl or a —X₃R₁₂ radical wherein X₃ represents O, NH or S, and R₁₂ represents a hydrogen atom, an alkyl (C₁-C₁₀) carbonyl, an alkyl (C₁-C₁₀) phenyl, an alkyl (C₁-C₁₀) sulphonyl, an alkyl (C₁-C₁₀) phenyl sulphonyl or an alkyl (C₁-C₁₀)-dialkyl (C₁-C₁₀) silyl;
R₅ represents the group N⁺OCH₃R₁₃ or the group NCH₃R₁₃ wherein
R₁₃ represents a hydrogen atom, a C₃-C₁₀ cycloalkyl, a C₁-C₁₀ alkyl, a phenyl or a phthalimide, and [symbol]
represents ═O or —OR₁₄ wherein R₁₄ represents a hydrogen atom, a C₁-C₁₀ alkyl, an alkyl (C₁-C₁₀) carbonyl, an alkyl (C₁-C₁₀) phenyl or an alkyl (C₁-C₁₀) sulphonyl;
or pharmaceutically acceptable addition salts, isomers, enantiomers or diastereoisomers thereof, as well as mixtures thereof, for use as a medicament.

50. A pharmaceutical composition according to claim 49, wherein said triterpenic alkaloid is selected among the compounds of the following formulas:

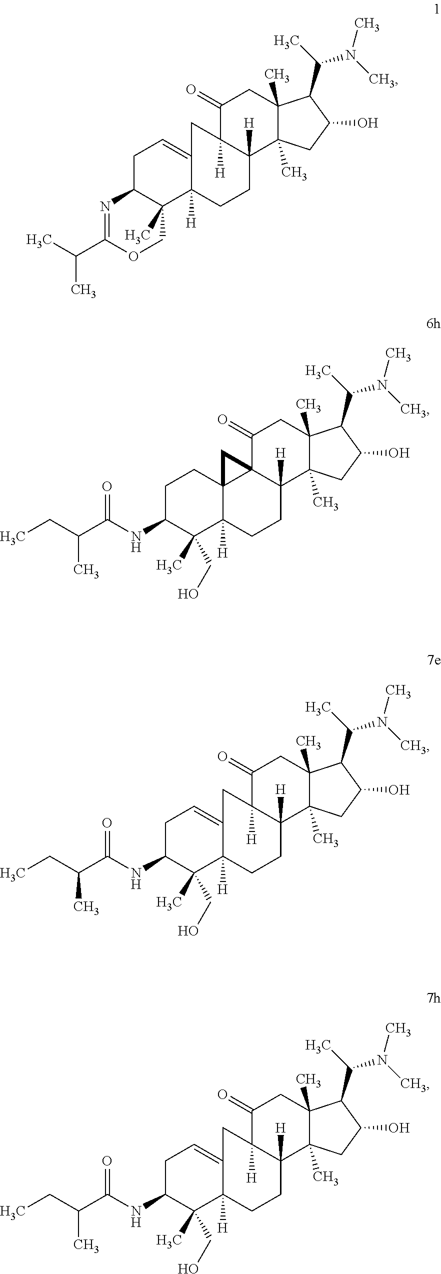

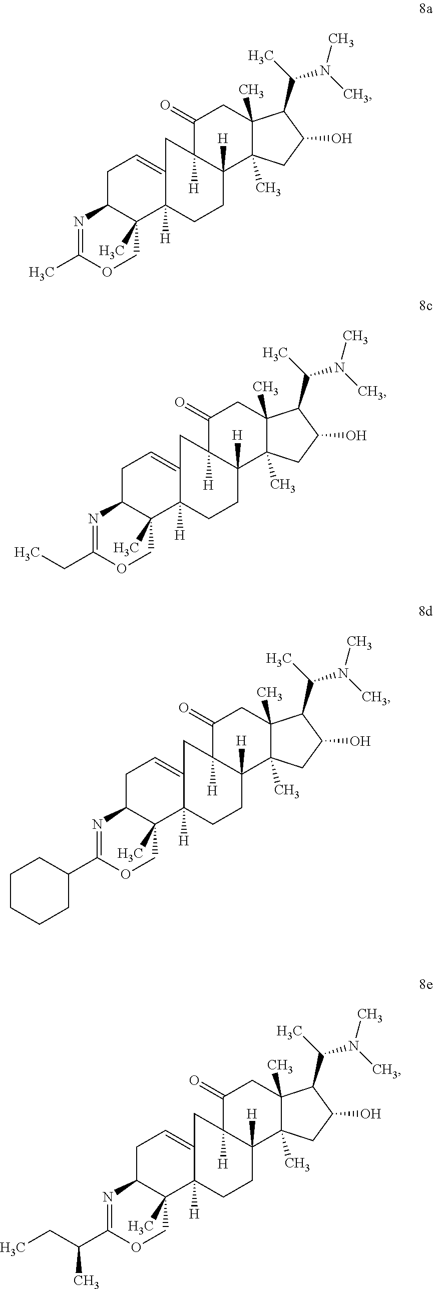

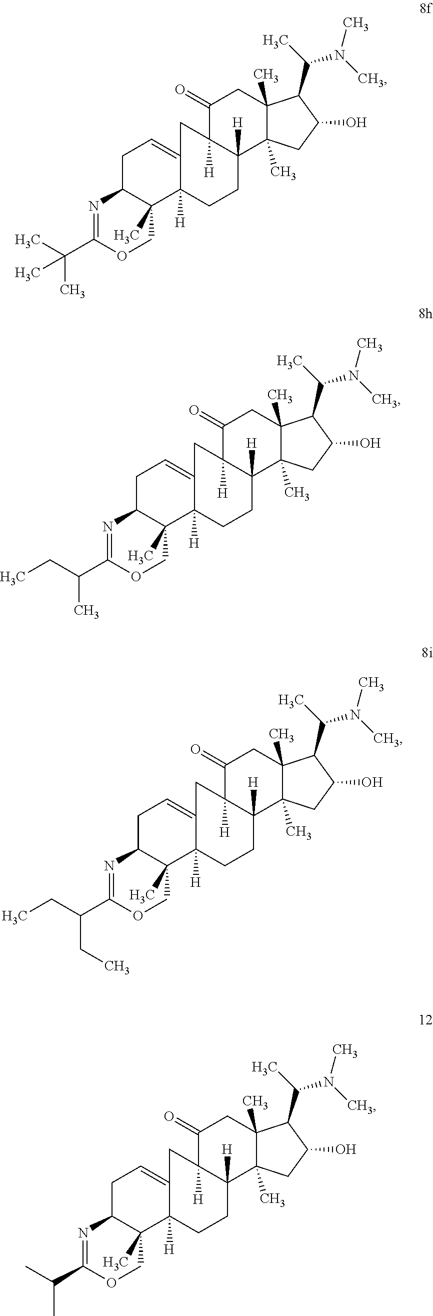

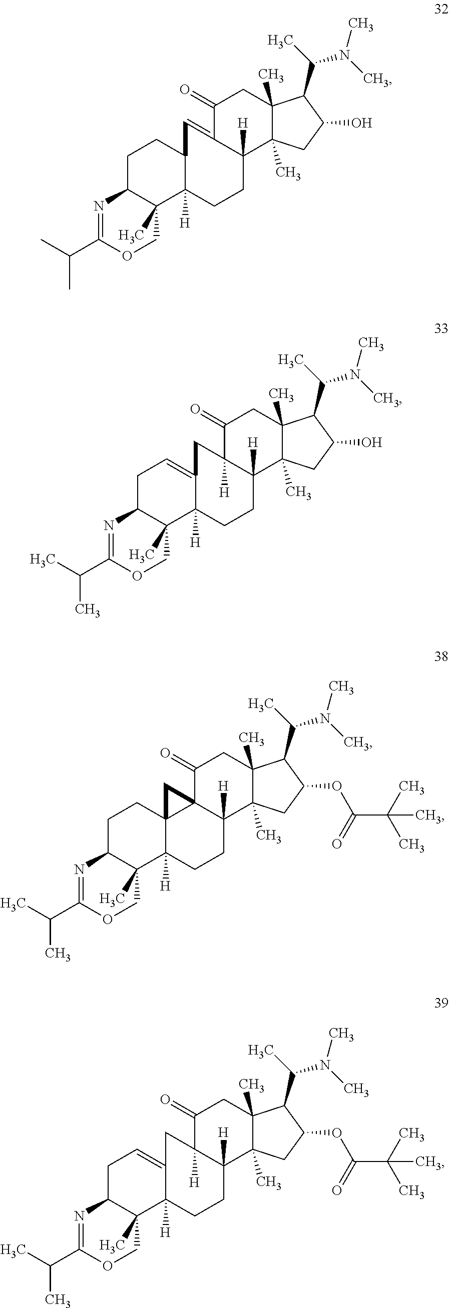

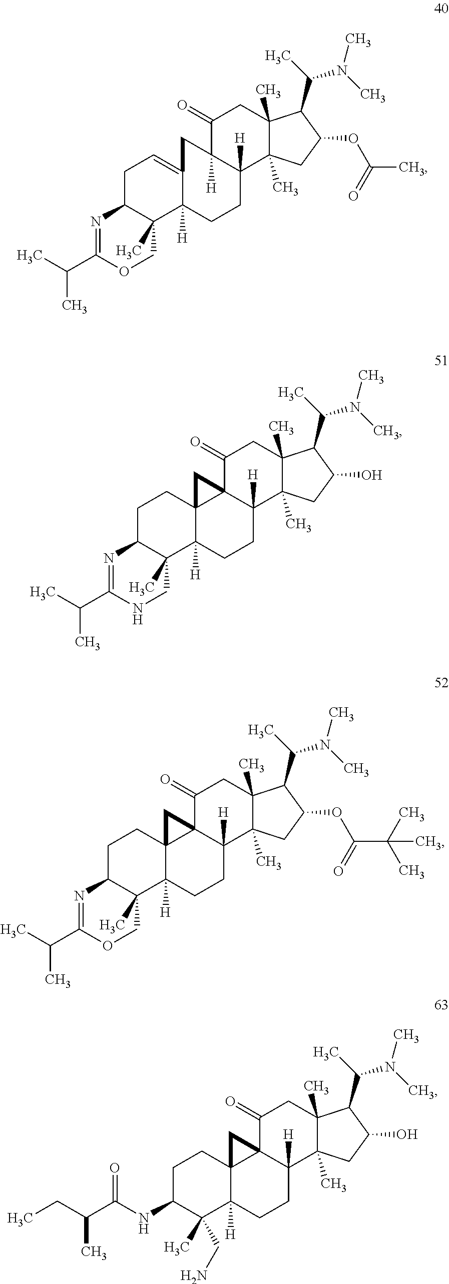

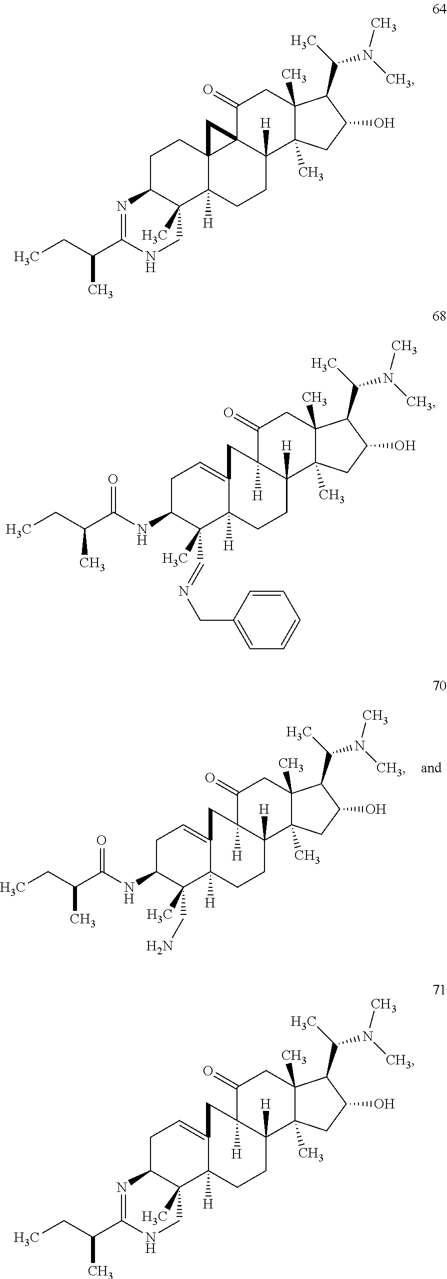

51. A method for treating a subject suffering from a disease of the central or peripheral nervous system which includes administration of a pharmaceutical composition according to claim 49 to the subject in need thereof.

52. A method for treating a subject suffering from a disease of the central or peripheral nervous system which includes administration of a pharmaceutical composition according to claim 50 to the subject in need thereof.

53. A method for treating according to claim 51, wherein said disease of the central or peripheral nervous system is selected from the group comprising Alzheimer's disease, memory disorders associated with aging or with Alzheimer's disease, disorders associated with trisomy 21, Lewy body dementia, Parkinson's dementia, vascular dementia, delirium, traumatic brain injuries, myasthenia of congenital or autoimmune origin to release neuromuscular blocking agents acting on muscle post-synaptic receptors and any cholinergic syndrome wherein there is a reduction in the quantity of acetylcholinesterase released, epilepsy, brain tumor, neurodegenerative disease, Lesch-Nyhan syndrome, Huntington's disease, amyotrophic lateral sclerosis (Lou Gehrig's disease), Down syndrome, or peripheral neuropathies.

54. A method for treating according to claim 52, wherein said disease of the central or peripheral nervous system is selected from the group comprising Alzheimer's disease, memory disorders associated with aging or with Alzheimer's disease, disorders associated with trisomy 21, Lewy body dementia, Parkinson's dementia, vascular dementia, delirium, traumatic brain injuries, myasthenia of congenital or autoimmune origin to release neuromuscular blocking agents acting on muscle post-synaptic receptors and any cholinergic syndrome wherein there is a reduction in the quantity of acetylcholinesterase released, epilepsy, brain tumor, neurodegenerative disease, Lesch-Nyhan syndrome, Huntington's disease, amyotrophic lateral sclerosis (Lou Gehrig's disease), Down syndrome, or peripheral neuropathies.
